SEQUENCE LISTING (396 sequences)

SEQ ID NO: 1 - protein, 447 aa, synthetic construct
  source 1..447: note = Exemplary Heavy chain of Anti-CD37 antibody T26B373
QVQLQESGPGLVKPSETLSLTCTVSGGSISSGVYYWAWIRQPPGKGLELIGTFYYSGSTYYDSSLRSRVTISVDTSKNQFSLKLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 447 aa, synthetic construct
  source 1..447: note = Exemplary Heavy chain of Anti-CD37 antibody T26B374
QLQLQESGPGLVKPSETLSLTCTVSGGSISSSPYYWGWIRQPPGKGLEWIGSFYYGGSNYYNPSLKSRVTISADTSKNQFSLKLSSVTAADTAVYYCARQAGDWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 447 aa, synthetic construct
  source 1..447: note = Exemplary Heavy chain of Anti-CD37 antibody T26B375
QLQLQESGPGLVKPSETLSLTCTVSGGSISSGIYNWGWIRQPPGKGLEWIGNFQYSGITYYNPSLKSRVTISVDTSKNQFSLQLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 447 aa, synthetic construct
  source 1..447: note = Exemplary Heavy chain of Anti-CD37 antibody T26B379
QVQLQESGPGLVKPSETLSLTCTVSGGSISSSIYYWAWIRQPPGKGLEWIGTIYYGGSPYYSPSLKSRVTISIDTSKSQFSLRLTSVTVADTAVYYCARRAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 451 aa, synthetic construct
  source 1..451: note = Exemplary Heavy chain of Anti-CD37 antibody T26B382
QVQLVESGGGLVKPGGSLRLSCAASGFTFSDYYMTWIRQAPGKGLEWVSYISSSGITKYYADFVKGRFTISRDNAKNSLYLQMNSLRAEDTAVYYCARDRDRQWLLEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 447 aa, synthetic construct
  source 1..447: note = Exemplary Heavy chain of Anti-CD37 antibody T26B385
QVQLQESGPGLVKPSETLSLTCIVSGGSVSSRNYYWGWIRQPPGKGLEWIGRIYYSGNTNYNPSLKSRVTISVDTSKNQFSLKLSSVTAADTAVYYCARWAGEIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 452 aa, synthetic construct
  source 1..452: note = Exemplary Heavy chain of Anti-CD37 antibody T26B386
QVQLQESGPGLVKPSETLSLSCTVSGGSLNSYNYYWGWVRQPPGKGLEWIGTIYSSGSAYYNPSLKSRFTISVATSKNQFSLRLSSVTAADTAVYYCARGYRNSWYALFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 454 aa, synthetic construct
  source 1..454: note = Exemplary Heavy chain of Anti-CD37 antibody T26B388
QVQLQQWGAGLLKPSETLSLTCAVYGGSFSDYYWSWIRQPPGKGLEWIGEIDHSGSTDYNPSLKSRVTISVDTSKNQFSLKLSSVTAADTAVYYCARSMYYDIWTGYHGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 212 aa, synthetic construct
  source 1..212: note = Exemplary Light chain of Anti-CD37 antibody T26B373
DIQMTQSPSTLSASVGDRVTITCRASQSTSSWLAWYQQKPGKAPKLLIYKTSSLESGVPSRFSGSGSGTEFTLTISSLQPDDFATYYCQQYNSYSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 212 aa, synthetic construct
  source 1..212: note = Exemplary Light chain of Anti-CD37 antibody T26B374
DIQMTQSPSTLSASVGDRVTITCRASQSISSWLAWYQQKPGKAPKLLIYKASSLESGVPSRFSGSRSGTEFTLTISSLQPDDFATYYCQQYNSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 212 aa, synthetic construct
  source 1..212: note = Exemplary Light chain of Anti-CD37 antibody T26B375
DIQMTQSPSTLSASVGDRVTITCRASQSISSWLAWYQQKPGKAPKLLIYKASSLESGVPSRFSGSGSGTEFTLTISSLQPDDFATYYCQQYNSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 212 aa, synthetic construct
  source 1..212: note = Exemplary Light chain of Anti-CD37 antibody T26B379
DIQMTQSPSTLSASVGDRVTITCRASQSISSWLAWYQQKPGKAPKLLIFKTSSLESGVPSRFSGSGSGTEFTLTISSLQPDDFATYYCQQYNSYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 217 aa, synthetic construct
  source 1..217: note = Exemplary Light chain of Anti-CD37 antibody T26B382
QSALTQPASVSGSPGQSITISCTGTSSDVGGYNYVSWYQQHPSKAPKLMIYDVSNRPSGVSNRFSGSKSGNTASLTISGLQAEDEADYYCSSYTSSSTLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - protein, 212 aa, synthetic construct
  source 1..212: note = Exemplary Light chain of Anti-CD37 antibody T26B385
DIQMTQSPSTLSASVGDRVTITCRASQSLSSWLAWYQQKPGKAPKLLIFKTSSLESGVPSRFSGSGSGTEFTLTISSLQPDDFAIYYCQQYNSYIFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 214 aa, synthetic construct
  source 1..214: note = Exemplary Light chain of Anti-CD37 antibody T26B386
SSELTQDPAVSVALGQTVRITCQGDSLRNYYASWYQQKPGQAPVLVFYGKDNRPSGIPDRFSGSTSGNTASLTITGAQAQDEADYYCNSRDSSGDHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 214 aa, synthetic construct
  source 1..214: note = Exemplary Light chain of Anti-CD37 antibody T26B388
DIQMTQSPSSLSASVGDRVTITCRASQGIRNDLGWYQQKPGKAPKRLIYAASSLQGGVPSRFSGSGSGTEFTLTISSLQPEDFATYYCLQHYTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 12 aa, synthetic construct
  source 1..12: note = Exemplary anti-CD37 antibody T26B373 HCDR1 (AbM)
GGSISSGVYYWA

SEQ ID NO: 18 - protein, 9 aa, synthetic construct
  source 1..9: note = Exemplary anti-CD37 antibody T26B373 HCDR2 (AbM)
TFYYSGSTY

SEQ ID NO: 19 - protein, 7 aa, synthetic construct
  source 1..7: note = Exemplary anti-CD37 antibody T26B373 HCDR3 (AbM)
QAGDFDY

SEQ ID NO: 20 - protein, 12 aa, synthetic construct
  source 1..12: note = Exemplary anti-CD37 antibody T26B374 HCDR1 (AbM)
GGSISSSPYYWG

SEQ ID NO: 21 - protein, 9 aa, synthetic construct
  source 1..9: note = Exemplary anti-CD37 antibody T26B374 HCDR2 (AbM)
SFYYGGSNY

SEQ ID NO: 22 - protein, 7 aa, synthetic construct
  source 1..7: note = Exemplary anti-CD37 antibody T26B374 HCDR3 (AbM)
QAGDWDY

SEQ ID NO: 23 - protein, 12 aa, synthetic construct
  source 1..12: note = Exemplary anti-CD37 antibody T26B375 HCDR1 (AbM)
GGSISSGIYNWG

SEQ ID NO: 24 - protein, 9 aa, synthetic construct
  source 1..9: note = Exemplary anti-CD37 antibody T26B375 HCDR2 (AbM)
NFQYSGITY

SEQ ID NO: 25 - protein, 7 aa, synthetic construct
  source 1..7: note = Exemplary anti-CD37 antibody T26B375 HCDR3 (AbM)
QAGDFDY

SEQ ID NO: 26 - protein, 12 aa, synthetic construct
  source 1..12: note = Exemplary anti-CD37 antibody T26B379 HCDR1 (AbM)
GGSISSSIYYWA

SEQ ID NO: 27 - protein, 9 aa, synthetic construct
  source 1..9: note = Exemplary anti-CD37 antibody T26B379 HCDR2 (AbM)
TIYYGGSPY

SEQ ID NO: 28 - protein, 7 aa, synthetic construct
  source 1..7: note = Exemplary anti-CD37 antibody T26B379 HCDR3 (AbM)
RAGDFDY

SEQ ID NO: 29 - protein, 10 aa, synthetic construct
  source 1..10: note = Exemplary anti-CD37 antibody T26B382 HCDR1 (AbM)
GFTFSDYYMT

SEQ ID NO: 30 - protein, 10 aa, synthetic construct
  source 1..10: note = Exemplary anti-CD37 antibody T26B382 HCDR2 (AbM)
YISSSGITKY

SEQ ID NO: 31 - protein, 12 aa, synthetic construct
  source 1..12: note = Exemplary anti-CD37 antibody T26B382 HCDR3 (AbM)
DRDRQWLLEFDY

SEQ ID NO: 32 - protein, 12 aa, synthetic construct
  source 1..12: note = Exemplary anti-CD37 antibody T26B385 HCDR1 (AbM)
GGSVSSRNYYWG

SEQ ID NO: 33 - protein, 9 aa, synthetic construct
  source 1..9: note = Exemplary anti-CD37 antibody T26B385 HCDR2 (AbM)
RIYYSGNTN

SEQ ID NO: 34 - protein, 7 aa, synthetic construct
  source 1..7: note = Exemplary anti-CD37 antibody T26B385 HCDR3 (AbM)
WAGEIDY

SEQ ID NO: 35 - protein, 12 aa, synthetic construct
  source 1..12: note = Exemplary anti-CD37 antibody T26B386 HCDR1 (AbM)
GGSLNSYNYYWG

SEQ ID NO: 36 - protein, 9 aa, synthetic construct
  source 1..9: note = Exemplary anti-CD37 antibody T26B386 HCDR2 (AbM)
TIYSSGSAY

SEQ ID NO: 37 - protein, 12 aa, synthetic construct
  source 1..12: note = Exemplary anti-CD37 antibody T26B386 HCDR3 (AbM)
GYRNSWYALFEY

SEQ ID NO: 38 - protein, 10 aa, synthetic construct
  source 1..10: note = Exemplary anti-CD37 antibody T26B388 HCDR1 (AbM)
GGSFSDYYWS

SEQ ID NO: 39 - protein, 9 aa, synthetic construct
  source 1..9: note = Exemplary anti-CD37 antibody T26B388 HCDR2 (AbM)
EIDHSGSTD

SEQ ID NO: 40 - protein, 16 aa, synthetic construct
  source 1..16: note = Exemplary anti-CD37 antibody T26B388 HCDR3 (AbM)
SMYYDIWTGYHGAFDI

SEQ ID NO: 41 - protein, 7 aa, synthetic construct
  source 1..7: note = Exemplary anti-CD37 antibody T26B373 HCDR1 (Kabat)
SGVYYWA

SEQ ID NO: 42 - protein, 16 aa, synthetic construct
  source 1..16: note = Exemplary anti-CD37 antibody T26B373 HCDR2 (Kabat)
TFYYSGSTYYDSSLRS

SEQ ID NO: 43 - protein, 7 aa, synthetic construct
  source 1..7: note = Exemplary anti-CD37 antibody T26B373 HCDR3 (Kabat)
QAGDFDY

SEQ ID NO: 44 - protein, 7 aa, synthetic construct
  source 1..7: note = Exemplary anti-CD37 antibody T26B374 HCDR1 (Kabat)
SSPYYWG

SEQ ID NO: 45 - protein, 16 aa, synthetic construct
  source 1..16: note = Exemplary anti-CD37 antibody T26B374 HCDR2 (Kabat)
SFYYGGSNYYNPSLKS

SEQ ID NO: 46 - protein, 7 aa, synthetic construct
  source 1..7: note = Exemplary anti-CD37 antibody T26B374 HCDR3 (Kabat)
QAGDWDY

SEQ ID NO: 47 - protein, 7 aa, synthetic construct
  source 1..7: note = Exemplary anti-CD37 antibody T26B375 HCDR1 (Kabat)
SGIYNWG

SEQ ID NO: 48 - protein, 16 aa, synthetic construct
  source 1..16: note = Exemplary anti-CD37 antibody T26B375 HCDR2 (Kabat)
NFQYSGITYYNPSLKS

SEQ ID NO: 49 - protein, 7 aa, synthetic construct
  source 1..7: note = Exemplary anti-CD37 antibody T26B375 HCDR3 (Kabat)
QAGDFDY

SEQ ID NO: 50 - protein, 7 aa, synthetic construct
  source 1..7: note = Exemplary anti-CD37 antibody T26B379 HCDR1 (Kabat)
SSIYYWA

SEQ ID NO: 51 - protein, 16 aa, synthetic construct
  source 1..16: note = Exemplary anti-CD37 antibody T26B379 HCDR2 (Kabat)
TIYYGGSPYYSPSLKS

SEQ ID NO: 52 - protein, 7 aa, synthetic construct
  source 1..7: note = Exemplary anti-CD37 antibody T26B379 HCDR3 (Kabat)
RAGDFDY

SEQ ID NO: 53 - protein, 5 aa, synthetic construct
  source 1..5: note = Exemplary anti-CD37 antibody T26B382 HCDR1 (Kabat)
DYYMT

SEQ ID NO: 54 - protein, 17 aa, synthetic construct
  source 1..17: note = Exemplary anti-CD37 antibody T26B382 HCDR2 (Kabat)
YISSSGITKYYADFVKG

SEQ ID NO: 55 - protein, 12 aa, synthetic construct
  source 1..12: note = Exemplary anti-CD37 antibody T26B382 HCDR3 (Kabat)
DRDRQWLLEFDY

SEQ ID NO: 56 - protein, 7 aa, synthetic construct
  source 1..7: note = Exemplary anti-CD37 antibody T26B385 HCDR1 (Kabat)
SRNYYWG

SEQ ID NO: 57 - protein, 16 aa, synthetic construct
  source 1..16: note = Exemplary anti-CD37 antibody T26B385 HCDR2 (Kabat)
RIYYSGNTNYNPSLKS

SEQ ID NO: 58 - protein, 7 aa, synthetic construct
  source 1..7: note = Exemplary anti-CD37 antibody T26B385 HCDR3 (Kabat)
WAGEIDY

SEQ ID NO: 59 - protein, 7 aa, synthetic construct
  source 1..7: note = Exemplary anti-CD37 antibody T26B386 HCDR1 (Kabat)
SYNYYWG

SEQ ID NO: 60 - protein, 16 aa, synthetic construct
  source 1..16: note = Exemplary anti-CD37 antibody T26B386 HCDR2 (Kabat)
TIYSSGSAYYNPSLKS

SEQ ID NO: 61 - protein, 12 aa, synthetic construct
  source 1..12: note = Exemplary anti-CD37 antibody T26B386 HCDR3 (Kabat)
GYRNSWYALFEY

SEQ ID NO: 62 - protein, 5 aa, synthetic construct
  source 1..5: note = Exemplary anti-CD37 antibody T26B388 HCDR1 (Kabat)
DYYWS

SEQ ID NO: 63 - protein, 16 aa, synthetic construct
  source 1..16: note = Exemplary anti-CD37 antibody T26B388 HCDR2 (Kabat)
EIDHSGSTDYNPSLKS

SEQ ID NO: 64 - protein, 16 aa, synthetic construct
  source 1..16: note = Exemplary anti-CD37 antibody T26B388 HCDR3 (Kabat)
SMYYDIWTGYHGAFDI

SEQ ID NO: 65 - protein, 9 aa, synthetic construct
  source 1..9: note = Exemplary anti-CD37 antibody T26B373 HCDR1 (Chothia)
GGSISSGVY

SEQ ID NO: 66 - protein, 5 aa, synthetic construct
  source 1..5: note = Exemplary anti-CD37 antibody T26B373 HCDR2 (Chothia)
YYSGS

SEQ ID NO: 67 - protein, 7 aa, synthetic construct
  source 1..7: note = Exemplary anti-CD37 antibody T26B373 HCDR3 (Chothia)
QAGDFDY

SEQ ID NO: 68 - protein, 9 aa, synthetic construct
  source 1..9: note = Exemplary anti-CD37 antibody T26B374 HCDR1 (Chothia)
GGSISSSPY

SEQ ID NO: 69 - protein, 5 aa, synthetic construct
  source 1..5: note = Exemplary anti-CD37 antibody T26B374 HCDR2 (Chothia)
YYGGS

SEQ ID NO: 70 - protein, 7 aa, synthetic construct
  source 1..7: note = Exemplary anti-CD37 antibody T26B374 HCDR3 (Chothia)
QAGDWDY

SEQ ID NO: 71 - protein, 9 aa, synthetic construct
  source 1..9: note = Exemplary anti-CD37 antibody T26B375 HCDR1 (Chothia)
GGSISSGIY

SEQ ID NO: 72 - protein, 5 aa, synthetic construct
  source 1..5: note = Exemplary anti-CD37 antibody T26B375 HCDR2 (Chothia)
QYSGI

SEQ ID NO: 73 - protein, 7 aa, synthetic construct
  source 1..7: note = Exemplary anti-CD37 antibody T26B375 HCDR3 (Chothia)
QAGDFDY

SEQ ID NO: 74 - protein, 9 aa, synthetic construct
  source 1..9: note = Exemplary anti-CD37 antibody T26B379 HCDR1 (Chothia)
GGSISSSIY

SEQ ID NO: 75 - protein, 5 aa, synthetic construct
  source 1..5: note = Exemplary anti-CD37 antibody T26B379 HCDR2 (Chothia)
YYGGS

SEQ ID NO: 76 - protein, 7 aa, synthetic construct
  source 1..7: note = Exemplary anti-CD37 antibody T26B379 HCDR3 (Chothia)
RAGDFDY

SEQ ID NO: 77 - protein, 7 aa, synthetic construct
  source 1..7: note = Exemplary anti-CD37 antibody T26B382 HCDR1 (Chothia)
GFTFSDY

SEQ ID NO: 78 - protein, 6 aa, synthetic construct
  source 1..6: note = Exemplary anti-CD37 antibody T26B382 HCDR2 (Chothia)
SSSGIT

SEQ ID NO: 79 - protein, 12 aa, synthetic construct
  source 1..12: note = Exemplary anti-CD37 antibody T26B382 HCDR3 (Chothia)
DRDRQWLLEFDY

SEQ ID NO: 80 - protein, 9 aa, synthetic construct
  source 1..9: note = Exemplary anti-CD37 antibody T26B385 HCDR1 (Chothia)
GGSVSSRNY

SEQ ID NO: 81 - protein, 5 aa, synthetic construct
  source 1..5: note = Exemplary anti-CD37 antibody T26B385 HCDR2 (Chothia)
YYSGN

SEQ ID NO: 82 - protein, 7 aa, synthetic construct
  source 1..7: note = Exemplary anti-CD37 antibody T26B385 HCDR3 (Chothia)
WAGEIDY

SEQ ID NO: 83 - protein, 9 aa, synthetic construct
  source 1..9: note = Exemplary anti-CD37 antibody T26B386 HCDR1 (Chothia)
GGSLNSYNY

SEQ ID NO: 84 - protein, 5 aa, synthetic construct
  source 1..5: note = Exemplary anti-CD37 antibody T26B386 HCDR2 (Chothia)
YSSGS

SEQ ID NO: 85 - protein, 12 aa, synthetic construct
  source 1..12: note = Exemplary anti-CD37 antibody T26B386 HCDR3 (Chothia)
GYRNSWYALFEY

SEQ ID NO: 86 - protein, 7 aa, synthetic construct
  source 1..7: note = Exemplary anti-CD37 antibody T26B388 HCDR1 (Chothia)
GGSFSDY

SEQ ID NO: 87 - protein, 5 aa, synthetic construct
  source 1..5: note = Exemplary anti-CD37 antibody T26B388 HCDR2 (Chothia)
DHSGS

SEQ ID NO: 88 - protein, 16 aa, synthetic construct
  source 1..16: note = Exemplary anti-CD37 antibody T26B388 HCDR3 (Chothia)
SMYYDIWTGYHGAFDI

SEQ ID NO: 89 - protein, 10 aa, synthetic construct
  source 1..10: note = Exemplary anti-CD37 antibody T26B373 HCDR1 (IMGT)
GGSISSGVYY

SEQ ID NO: 90 - protein, 7 aa, synthetic construct
  source 1..7: note = Exemplary anti-CD37 antibody T26B373 HCDR2 (IMGT)
FYYSGST

SEQ ID NO: 91 - protein, 9 aa, synthetic construct
  source 1..9: note = Exemplary anti-CD37 antibody T26B373 HCDR3 (IMGT)
ARQAGDFDY

SEQ ID NO: 92 - protein, 10 aa, synthetic construct
  source 1..10: note = Exemplary anti-CD37 antibody T26B374 HCDR1 (IMGT)
GGSISSSPYY

SEQ ID NO: 93 - protein, 7 aa, synthetic construct
  source 1..7: note = Exemplary anti-CD37 antibody T26B374 HCDR2 (IMGT)
FYYGGSN

SEQ ID NO: 94 - protein, 9 aa, synthetic construct
  source 1..9: note = Exemplary anti-CD37 antibody T26B374 HCDR3 (IMGT)
ARQAGDWDY

SEQ ID NO: 95 - protein, 10 aa, synthetic construct
  source 1..10: note = Exemplary anti-CD37 antibody T26B375 HCDR1 (IMGT)
GGSISSGIYN

SEQ ID NO: 96 - protein, 7 aa, synthetic construct
  source 1..7: note = Exemplary anti-CD37 antibody T26B375 HCDR2 (IMGT)
FQYSGIT

SEQ ID NO: 97 - protein, 9 aa, synthetic construct
  source 1..9: note = Exemplary anti-CD37 antibody T26B375 HCDR3 (IMGT)
ARQAGDFDY

SEQ ID NO: 98 - protein, 10 aa, synthetic construct
  source 1..10: note = Exemplary anti-CD37 antibody T26B379 HCDR1 (IMGT)
GGSISSSIYY

SEQ ID NO: 99 - protein, 7 aa, synthetic construct
  source 1..7: note = Exemplary anti-CD37 antibody T26B379 HCDR2 (IMGT)
IYYGGSP

SEQ ID NO: 100 - protein, 9 aa, synthetic construct
  source 1..9: note = Exemplary anti-CD37 antibody T26B379 HCDR3 (IMGT)
ARRAGDFDY

SEQ ID NO: 101 - protein, 8 aa, synthetic construct
  source 1..8: note = Exemplary anti-CD37 antibody T26B382 HCDR1 (IMGT)
GFTFSDYY

SEQ ID NO: 102 - protein, 8 aa, synthetic construct
  source 1..8: note = Exemplary anti-CD37 antibody T26B382 HCDR2 (IMGT)
ISSSGITK

SEQ ID NO: 103 - protein, 14 aa, synthetic construct
  source 1..14: note = Exemplary anti-CD37 antibody T26B382 HCDR3 (IMGT)
ARDRDRQWLLEFDY

SEQ ID NO: 104 - protein, 10 aa, synthetic construct
  source 1..10: note = Exemplary anti-CD37 antibody T26B385 HCDR1 (IMGT)
GGSVSSRNYY

SEQ ID NO: 105 - protein, 7 aa, synthetic construct
  source 1..7: note = Exemplary anti-CD37 antibody T26B385 HCDR2 (IMGT)
IYYSGNT

SEQ ID NO: 106 - protein, 9 aa, synthetic construct
  source 1..9: note = Exemplary anti-CD37 antibody T26B385 HCDR3 (IMGT)
ARWAGEIDY

SEQ ID NO: 107 - protein, 10 aa, synthetic construct
  source 1..10: note = Exemplary anti-CD37 antibody T26B386 HCDR1 (IMGT)
GGSLNSYNYY

SEQ ID NO: 108 - protein, 7 aa, synthetic construct
  source 1..7: note = Exemplary anti-CD37 antibody T26B386 HCDR2 (IMGT)
IYSSGSA

SEQ ID NO: 109 - protein, 14 aa, synthetic construct
  source 1..14: note = Exemplary anti-CD37 antibody T26B386 HCDR3 (IMGT)
ARGYRNSWYALFEY

SEQ ID NO: 110 - protein, 8 aa, synthetic construct
  source 1..8: note = Exemplary anti-CD37 antibody T26B388 HCDR1 (IMGT)
GGSFSDYY

SEQ ID NO: 111 - protein, 7 aa, synthetic construct
  source 1..7: note = Exemplary anti-CD37 antibody T26B388 HCDR2 (IMGT)
IDHSGST

SEQ ID NO: 112 - protein, 18 aa, synthetic construct
  source 1..18: note = Exemplary anti-CD37 antibody T26B388 HCDR3 (IMGT)
ARSMYYDIWTGYHGAFDI

SEQ ID NO: 113 - protein, 8 aa, synthetic construct
  source 1..8: note = Exemplary anti-CD37 antibody T26B373 HCDR1 (Contact)
SSGVYYWA

SEQ ID NO: 114 - protein, 12 aa, synthetic construct
  source 1..12: note = Exemplary anti-CD37 antibody T26B373 HCDR2 (Contact)
LIGTFYYSGSTY

SEQ ID NO: 115 - protein, 8 aa, synthetic construct
  source 1..8: note = Exemplary anti-CD37 antibody T26B373 HCDR3 (Contact)
ARQAGDFD

SEQ ID NO: 116 - protein, 8 aa, synthetic construct
  source 1..8: note = Exemplary anti-CD37 antibody T26B374 HCDR1 (Contact)
SSSPYYWG

SEQ ID NO: 117 - protein, 12 aa, synthetic construct
  source 1..12: note = Exemplary anti-CD37 antibody T26B374 HCDR2 (Contact)
WIGSFYYGGSNY

SEQ ID NO: 118 - protein, 8 aa, synthetic construct
  source 1..8: note = Exemplary anti-CD37 antibody T26B374 HCDR3 (Contact)
ARQAGDWD

SEQ ID NO: 119 - protein, 8 aa, synthetic construct
  source 1..8: note = Exemplary anti-CD37 antibody T26B375 HCDR1 (Contact)
SSGIYNWG

SEQ ID NO: 120 - protein, 12 aa, synthetic construct
  source 1..12: note = Exemplary anti-CD37 antibody T26B375 HCDR2 (Contact)
WIGNFQYSGITY

SEQ ID NO: 121 - protein, 8 aa, synthetic construct
  source 1..8: note = Exemplary anti-CD37 antibody T26B375 HCDR3 (Contact)
ARQAGDFD

SEQ ID NO: 122 - protein, 8 aa, synthetic construct
  source 1..8: note = Exemplary anti-CD37 antibody T26B379 HCDR1 (Contact)
SSSIYYWA

SEQ ID NO: 123 - protein, 12 aa, synthetic construct
  source 1..12: note = Exemplary anti-CD37 antibody T26B379 HCDR2 (Contact)
WIGTIYYGGSPY

SEQ ID NO: 124 - protein, 8 aa, synthetic construct
  source 1..8: note = Exemplary anti-CD37 antibody T26B379 HCDR3 (Contact)
ARRAGDFD

SEQ ID NO: 125 - protein, 6 aa, synthetic construct
  source 1..6: note = Exemplary anti-CD37 antibody T26B382 HCDR1 (Contact)
SDYYMT

SEQ ID NO: 126 - protein, 13 aa, synthetic construct
  source 1..13: note = Exemplary anti-CD37 antibody T26B382 HCDR2 (Contact)
WVSYISSSGITKY

SEQ ID NO: 127 - protein, 13 aa, synthetic construct
  source 1..13: note = Exemplary anti-CD37 antibody T26B382 HCDR3 (Contact)
ARDRDRQWLLEFD

SEQ ID NO: 128 - protein, 8 aa, synthetic construct
  source 1..8: note = Exemplary anti-CD37 antibody T26B385 HCDR1 (Contact)
SSRNYYWG

SEQ ID NO: 129 - protein, 12 aa, synthetic construct
  source 1..12: note = Exemplary anti-CD37 antibody T26B385 HCDR2 (Contact)
WIGRIYYSGNTN

SEQ ID NO: 130 - protein, 8 aa, synthetic construct
  source 1..8: note = Exemplary anti-CD37 antibody T26B385 HCDR3 (Contact)
ARWAGEID

SEQ ID NO: 131 - protein, 8 aa, synthetic construct
  source 1..8: note = Exemplary anti-CD37 antibody T26B386 HCDR1 (Contact)
NSYNYYWG

SEQ ID NO: 132 - protein, 12 aa, synthetic construct
  source 1..12: note = Exemplary anti-CD37 antibody T26B386 HCDR2 (Contact)
WIGTIYSSGSAY

SEQ ID NO: 133 - protein, 13 aa, synthetic construct
  source 1..13: note = Exemplary anti-CD37 antibody T26B386 HCDR3 (Contact)
ARGYRNSWYALFE

SEQ ID NO: 134 - protein, 6 aa, synthetic construct
  source 1..6: note = Exemplary anti-CD37 antibody T26B388 HCDR1 (Contact)
SDYYWS

SEQ ID NO: 135 - protein, 12 aa, synthetic construct
  source 1..12: note = Exemplary anti-CD37 antibody T26B388 HCDR2 (Contact)
WIGEIDHSGSTD

SEQ ID NO: 136 - protein, 17 aa, synthetic construct
  source 1..17: note = Exemplary anti-CD37 antibody T26B388 HCDR3 (Contact)
ARSMYYDIWTGYHGAFD

SEQ ID NO: 137 - protein, 11 aa, synthetic construct
  source 1..11: note = Exemplary anti-CD37 antibody T26B373 LCDR1 (AbM)
RASQSTSSWLA

SEQ ID NO: 138 - protein, 7 aa, synthetic construct
  source 1..7: note = Exemplary anti-CD37 antibody T26B373 LCDR2 (AbM)
KTSSLES

SEQ ID NO: 139 - protein, 7 aa, synthetic construct
  source 1..7: note = Exemplary anti-CD37 antibody T26B373 LCDR3 (AbM)
QQYNSYS

SEQ ID NO: 140 - protein, 11 aa, synthetic construct
  source 1..11: note = Exemplary anti-CD37 antibody T26B374 LCDR1 (AbM)
RASQSISSWLA

SEQ ID NO: 141 - protein, 7 aa, synthetic construct
  source 1..7: note = Exemplary anti-CD37 antibody T26B374 LCDR2 (AbM)
KASSLES

SEQ ID NO: 142 - protein, 7 aa, synthetic construct
  source 1..7: note = Exemplary anti-CD37 antibody T26B374 LCDR3 (AbM)
QQYNSWT

SEQ ID NO: 143 - protein, 11 aa, synthetic construct
  source 1..11: note = Exemplary anti-CD37 antibody T26B375 LCDR1 (AbM)
RASQSISSWLA

SEQ ID NO: 144 - protein, 7 aa, synthetic construct
  source 1..7: note = Exemplary anti-CD37 antibody T26B375 LCDR2 (AbM)
KASSLES

SEQ ID NO: 145 - protein, 7 aa, synthetic construct
  source 1..7: note = Exemplary anti-CD37 antibody T26B375 LCDR3 (AbM)
QQYNSYT

SEQ ID NO: 146 - protein, 11 aa, synthetic construct
  source 1..11: note = Exemplary anti-CD37 antibody T26B379 LCDR1 (AbM)
RASQSISSWLA

SEQ ID NO: 147 - protein, 7 aa, synthetic construct
  source 1..7: note = Exemplary anti-CD37 antibody T26B379 LCDR2 (AbM)
KTSSLES

SEQ ID NO: 148 - protein, 7 aa, synthetic construct
  source 1..7: note = Exemplary anti-CD37 antibody T26B379 LCDR3 (AbM)
QQYNSYS

SEQ ID NO: 149 - protein, 14 aa, synthetic construct
  source 1..14: note = Exemplary anti-CD37 antibody T26B382 LCDR1 (AbM)
TGTSSDVGGYNYVS

SEQ ID NO: 150 - protein, 7 aa, synthetic construct
  source 1..7: note = Exemplary anti-CD37 antibody T26B382 LCDR2 (AbM)
DVSNRPS

SEQ ID NO: 151 - protein, 11 aa, synthetic construct
  source 1..11: note = Exemplary anti-CD37 antibody T26B382 LCDR3 (AbM)
SSYTSSSTLVV

SEQ ID NO: 152 - protein, 11 aa, synthetic construct
  source 1..11: note = Exemplary anti-CD37 antibody T26B385 LCDR1 (AbM)
RASQSLSSWLA

SEQ ID NO: 153 - protein, 7 aa, synthetic construct
  source 1..7: note = Exemplary anti-CD37 antibody T26B385 LCDR2 (AbM)
KTSSLES

SEQ ID NO: 154 - protein, 7 aa, synthetic construct
  source 1..7: note = Exemplary anti-CD37 antibody T26B385 LCDR3 (AbM)
QQYNSYI

SEQ ID NO: 155 - protein, 11 aa, synthetic construct
  source 1..11: note = Exemplary anti-CD37 antibody T26B386 LCDR1 (AbM)
QGDSLRNYYAS

SEQ ID NO: 156 - protein, 7 aa, synthetic construct
  source 1..7: note = Exemplary anti-CD37 antibody T26B386 LCDR2 (AbM)
GKDNRPS

SEQ ID NO: 157 - protein, 11 aa, synthetic construct
  source 1..11: note = Exemplary anti-CD37 antibody T26B386 LCDR3 (AbM)
NSRDSSGDHLV

SEQ ID NO: 158 - protein, 11 aa, synthetic construct
  source 1..11: note = Exemplary anti-CD37 antibody T26B388 LCDR1 (AbM)
RASQGIRNDLG

SEQ ID NO: 159 - protein, 7 aa, synthetic construct
  source 1..7: note = Exemplary anti-CD37 antibody T26B388 LCDR2 (AbM)
AASSLQG

SEQ ID NO: 160 - protein, 9 aa, synthetic construct
  source 1..9: note = Exemplary anti-CD37 antibody T26B388 LCDR3 (AbM)
LQHYTYPLT

SEQ ID NO: 161 - protein, 11 aa, synthetic construct
  source 1..11: note = Exemplary anti-CD37 antibody T26B373 LCDR1 (Kabat)
RASQSTSSWLA

SEQ ID NO: 162 - protein, 7 aa, synthetic construct
  source 1..7: note = Exemplary anti-CD37 antibody T26B373 LCDR2 (Kabat)
KTSSLES

SEQ ID NO: 163 - protein, 7 aa, synthetic construct
  source 1..7: note = Exemplary anti-CD37 antibody T26B373 LCDR3 (Kabat)
QQYNSYS

SEQ ID NO: 164 - protein, 11 aa, synthetic construct
  source 1..11: note = Exemplary anti-CD37 antibody T26B374 LCDR1 (Kabat)
RASQSISSWLA

SEQ ID NO: 165 - protein, 7 aa, synthetic construct
  source 1..7: note = Exemplary anti-CD37 antibody T26B374 LCDR2 (Kabat)
KASSLES

SEQ ID NO: 166 - protein, 7 aa, synthetic construct
  source 1..7: note = Exemplary anti-CD37 antibody T26B374 LCDR3 (Kabat)
QQYNSWT

SEQ ID NO: 167 - protein, 11 aa, synthetic construct
  source 1..11: note = Exemplary anti-CD37 antibody T26B375 LCDR1 (Kabat)
RASQSISSWLA

SEQ ID NO: 168 - protein, 7 aa, synthetic construct
  source 1..7: note = Exemplary anti-CD37 antibody T26B375 LCDR2 (Kabat)
KASSLES

SEQ ID NO: 169 - protein, 7 aa, synthetic construct
  source 1..7: note = Exemplary anti-CD37 antibody T26B375 LCDR3 (Kabat)
QQYNSYT

SEQ ID NO: 170 - protein, 11 aa, synthetic construct
  source 1..11: note = Exemplary anti-CD37 antibody T26B379 LCDR1 (Kabat)
RASQSISSWLA

SEQ ID NO: 171 - protein, 7 aa, synthetic construct
  source 1..7: note = Exemplary anti-CD37 antibody T26B379 LCDR2 (Kabat)
KTSSLES

SEQ ID NO: 172 - protein, 7 aa, synthetic construct
  source 1..7: note = Exemplary anti-CD37 antibody T26B379 LCDR3 (Kabat)
QQYNSYS

SEQ ID NO: 173 - protein, 14 aa, synthetic construct
  source 1..14: note = Exemplary anti-CD37 antibody T26B382 LCDR1 (Kabat)
TGTSSDVGGYNYVS

SEQ ID NO: 174 - protein, 7 aa, synthetic construct
  source 1..7: note = Exemplary anti-CD37 antibody T26B382 LCDR2 (Kabat)
DVSNRPS

SEQ ID NO: 175 - protein, 11 aa, synthetic construct
  source 1..11: note = Exemplary anti-CD37 antibody T26B382 LCDR3 (Kabat)
SSYTSSSTLVV

SEQ ID NO: 176 - protein, 11 aa, synthetic construct
  source 1..11: note = Exemplary anti-CD37 antibody T26B385 LCDR1 (Kabat)
RASQSLSSWLA

SEQ ID NO: 177 - protein, 7 aa, synthetic construct
  source 1..7: note = Exemplary anti-CD37 antibody T26B385 LCDR2 (Kabat)
KTSSLES

SEQ ID NO: 178 - protein, 7 aa, synthetic construct
  source 1..7: note = Exemplary anti-CD37 antibody T26B385 LCDR3 (Kabat)
QQYNSYI

SEQ ID NO: 179 - protein, 11 aa, synthetic construct
  source 1..11: note = Exemplary anti-CD37 antibody T26B386 LCDR1 (Kabat)
QGDSLRNYYAS

SEQ ID NO: 180 - protein, 7 aa, synthetic construct
  source 1..7: note = Exemplary anti-CD37 antibody T26B386 LCDR2 (Kabat)
GKDNRPS

SEQ ID NO: 181 - protein, 11 aa, synthetic construct
  source 1..11: note = Exemplary anti-CD37 antibody T26B386 LCDR3 (Kabat)
NSRDSSGDHLV

SEQ ID NO: 182 - protein, 11 aa, synthetic construct
  source 1..11: note = Exemplary anti-CD37 antibody T26B388 LCDR1 (Kabat)
RASQGIRNDLG

SEQ ID NO: 183 - protein, 7 aa, synthetic construct
  source 1..7: note = Exemplary anti-CD37 antibody T26B388 LCDR2 (Kabat)
AASSLQG

SEQ ID NO: 184 - protein, 9 aa, synthetic construct
  source 1..9: note = Exemplary anti-CD37 antibody T26B388 LCDR3 (Kabat)
LQHYTYPLT

SEQ ID NO: 185 - protein, 11 aa, synthetic construct
  source 1..11: note = Exemplary anti-CD37 antibody T26B373 LCDR1 (Chothia)
RASQSTSSWLA

SEQ ID NO: 186 - protein, 7 aa, synthetic construct
  source 1..7: note = Exemplary anti-CD37 antibody T26B373 LCDR2 (Chothia)
KTSSLES

SEQ ID NO: 187 - protein, 7 aa, synthetic construct
  source 1..7: note = Exemplary anti-CD37 antibody T26B373 LCDR3 (Chothia)
QQYNSYS

SEQ ID NO: 188 - protein, 11 aa, synthetic construct
  source 1..11: note = Exemplary anti-CD37 antibody T26B374 LCDR1 (Chothia)
RASQSISSWLA

SEQ ID NO: 189 - protein, 7 aa, synthetic construct
  source 1..7: note = Exemplary anti-CD37 antibody T26B374 LCDR2 (Chothia)
KASSLES

SEQ ID NO: 190 - protein, 7 aa, synthetic construct
  source 1..7: note = Exemplary anti-CD37 antibody T26B374 LCDR3 (Chothia)
QQYNSWT

SEQ ID NO: 191 - protein, 11 aa, synthetic construct
  source 1..11: note = Exemplary anti-CD37 antibody T26B375 LCDR1 (Chothia)
RASQSISSWLA

SEQ ID NO: 192 - protein, 7 aa, synthetic construct
  source 1..7: note = Exemplary anti-CD37 antibody T26B375 LCDR2 (Chothia)
KASSLES

SEQ ID NO: 193 - protein, 7 aa, synthetic construct
  source 1..7: note = Exemplary anti-CD37 antibody T26B375 LCDR3 (Chothia)
QQYNSYT

SEQ ID NO: 194 - protein, 11 aa, synthetic construct
  source 1..11: note = Exemplary anti-CD37 antibody T26B379 LCDR1 (Chothia)
RASQSISSWLA

SEQ ID NO: 195 - protein, 7 aa, synthetic construct
  source 1..7: note = Exemplary anti-CD37 antibody T26B379 LCDR2 (Chothia)
KTSSLES

SEQ ID NO: 196 - protein, 7 aa, synthetic construct
  source 1..7: note = Exemplary anti-CD37 antibody T26B379 LCDR3 (Chothia)
QQYNSYS

SEQ ID NO: 197 - protein, 14 aa, synthetic construct
  source 1..14: note = Exemplary anti-CD37 antibody T26B382 LCDR1 (Chothia)
TGTSSDVGGYNYVS

SEQ ID NO: 198 - protein, 7 aa, synthetic construct
  source 1..7: note = Exemplary anti-CD37 antibody T26B382 LCDR2 (Chothia)
DVSNRPS

SEQ ID NO: 199 - protein, 11 aa, synthetic construct
  source 1..11: note = Exemplary anti-CD37 antibody T26B382 LCDR3 (Chothia)
SSYTSSSTLVV

SEQ ID NO: 200 - protein, 11 aa, synthetic construct
  source 1..11: note = Exemplary anti-CD37 antibody T26B385 LCDR1 (Chothia)
RASQSLSSWLA

SEQ ID NO: 201 - protein, 7 aa, synthetic construct
  source 1..7: note = Exemplary anti-CD37 antibody T26B385 LCDR2 (Chothia)
KTSSLES

SEQ ID NO: 202 - protein, 7 aa, synthetic construct
  source 1..7: note = Exemplary anti-CD37 antibody T26B385 LCDR3 (Chothia)
QQYNSYI

SEQ ID NO: 203 - protein, 11 aa, synthetic construct
  source 1..11: note = Exemplary anti-CD37 antibody T26B386 LCDR1 (Chothia)
QGDSLRNYYAS

SEQ ID NO: 204 - protein, 7 aa, synthetic construct
  source 1..7: note = Exemplary anti-CD37 antibody T26B386 LCDR2 (Chothia)
GKDNRPS

SEQ ID NO: 205 - protein, 11 aa, synthetic construct
  source 1..11: note = Exemplary anti-CD37 antibody T26B386 LCDR3 (Chothia)
NSRDSSGDHLV

SEQ ID NO: 206 - protein, 11 aa, synthetic construct
  source 1..11: note = Exemplary anti-CD37 antibody T26B388 LCDR1 (Chothia)
RASQGIRNDLG

SEQ ID NO: 207 - protein, 7 aa, synthetic construct
  source 1..7: note = Exemplary anti-CD37 antibody T26B388 LCDR2 (Chothia)
AASSLQG

SEQ ID NO: 208 - protein, 9 aa, synthetic construct
  source 1..9: note = Exemplary anti-CD37 antibody T26B388 LCDR3 (Chothia)
LQHYTYPLT

SEQ ID NO: 209 - protein, 6 aa, synthetic construct
  source 1..6: note = Exemplary anti-CD37 antibody T26B373 LCDR1 (IMGT)
QSTSSW

SEQ ID NO: 210 - skipped

SEQ ID NO: 211 - protein, 7 aa, synthetic construct
  source 1..7: note = Exemplary anti-CD37 antibody T26B373 LCDR3 (IMGT)
QQYNSYS

SEQ ID NO: 212 - protein, 6 aa, synthetic construct
  source 1..6: note = Exemplary anti-CD37 antibody T26B374 LCDR1 (IMGT)
QSISSW

SEQ ID NO: 213 - skipped

SEQ ID NO: 214 - protein, 7 aa, synthetic construct
  source 1..7: note = Exemplary anti-CD37 antibody T26B374 LCDR3 (IMGT)
QQYNSWT

SEQ ID NO: 215 - protein, 6 aa, synthetic construct
  source 1..6: note = Exemplary anti-CD37 antibody T26B375 LCDR1 (IMGT)
QSISSW

SEQ ID NO: 216 - skipped

SEQ ID NO: 217 - protein, 7 aa, synthetic construct
  source 1..7: note = Exemplary anti-CD37 antibody T26B375 LCDR3 (IMGT)
QQYNSYT

SEQ ID NO: 218 - protein, 6 aa, synthetic construct
  source 1..6: note = Exemplary anti-CD37 antibody T26B379 LCDR1 (IMGT)
QSISSW

SEQ ID NO: 219 - skipped

SEQ ID NO: 220 - protein, 7 aa, synthetic construct
  source 1..7: note = Exemplary anti-CD37 antibody T26B379 LCDR3 (IMGT)
QQYNSYS

SEQ ID NO: 221 - protein, 9 aa, synthetic construct
  source 1..9: note = Exemplary anti-CD37 antibody T26B382 LCDR1 (IMGT)
SSDVGGYNY

SEQ ID NO: 222 - skipped

SEQ ID NO: 223 - protein, 11 aa, synthetic construct
  source 1..11: note = Exemplary anti-CD37 antibody T26B382 LCDR3 (IMGT)
SSYTSSSTLVV

SEQ ID NO: 224 - protein, 6 aa, synthetic construct
  source 1..6: note = Exemplary anti-CD37 antibody T26B385 LCDR1 (IMGT)
QSLSSW

SEQ ID NO: 225 - skipped

SEQ ID NO: 226 - protein, 7 aa, synthetic construct
  source 1..7: note = Exemplary anti-CD37 antibody T26B385 LCDR3 (IMGT)
QQYNSYI

SEQ ID NO: 227 - protein, 6 aa, synthetic construct
  source 1..6: note = Exemplary anti-CD37 antibody T26B386 LCDR1 (IMGT)
SLRNYY

SEQ ID NO: 228 - skipped

SEQ ID NO: 229 - protein, 11 aa, synthetic construct
  source 1..11: note = Exemplary anti-CD37 antibody T26B386 LCDR3 (IMGT)
NSRDSSGDHLV

SEQ ID NO: 230 - protein, 6 aa, synthetic construct
  source 1..6: note = Exemplary anti-CD37 antibody T26B388 LCDR1 (IMGT)
QGIRND

SEQ ID NO: 231 - skipped

SEQ ID NO: 232 - protein, 9 aa, synthetic construct
  source 1..9: note = Exemplary anti-CD37 antibody T26B388 LCDR3 (IMGT)
LQHYTYPLT

SEQ ID NO: 233 - protein, 7 aa, synthetic construct
  source 1..7: note = Exemplary anti-CD37 antibody T26B373 LCDR1 (Contact)
SSWLAWY

SEQ ID NO: 234 - protein, 10 aa, synthetic construct
  source 1..10: note = Exemplary anti-CD37 antibody T26B373 LCDR2 (Contact)
LLIYKTSSLE

SEQ ID NO: 235 - protein, 6 aa, synthetic construct
  source 1..6: note = Exemplary anti-CD37 antibody T26B373 LCDR3 (Contact)
QQYNSY

SEQ ID NO: 236 - protein, 7 aa, synthetic construct
  source 1..7: note = Exemplary anti-CD37 antibody T26B374 LCDR1 (Contact)
SSWLAWY

SEQ ID NO: 237 - protein, 10 aa, synthetic construct
  source 1..10: note = Exemplary anti-CD37 antibody T26B374 LCDR2 (Contact)
LLIYKASSLE

SEQ ID NO: 238 - protein, 6 aa, synthetic construct
  source 1..6: note = Exemplary anti-CD37 antibody T26B374 LCDR3 (Contact)
QQYNSW

SEQ ID NO: 239 - protein, 7 aa, synthetic construct
  source 1..7: note = Exemplary anti-CD37 antibody T26B375 LCDR1 (Contact)
SSWLAWY

SEQ ID NO: 240 - protein, 10 aa, synthetic construct
  source 1..10: note = Exemplary anti-CD37 antibody T26B375 LCDR2 (Contact)
LLIYKASSLE

SEQ ID NO: 241 - protein, 6 aa, synthetic construct
  source 1..6: note = Exemplary anti-CD37 antibody T26B375 LCDR3 (Contact)
QQYNSY

SEQ ID NO: 242 - protein, 7 aa, synthetic construct
  source 1..7: note = Exemplary anti-CD37 antibody T26B379 LCDR1 (Contact)
SSWLAWY

SEQ ID NO: 243 - protein, 10 aa, synthetic construct
  source 1..10: note = Exemplary anti-CD37 antibody T26B379 LCDR2 (Contact)
LLIFKTSSLE

SEQ ID NO: 244 - protein, 6 aa, synthetic construct
  source 1..6: note = Exemplary anti-CD37 antibody T26B379 LCDR3 (Contact)
QQYNSY

SEQ ID NO: 245 - protein, 10 aa, synthetic construct
  source 1..10: note = Exemplary anti-CD37 antibody T26B382 LCDR1 (Contact)
VGGYNYVSWY

SEQ ID NO: 246 - protein, 10 aa, synthetic construct
  source 1..10: note = Exemplary anti-CD37 antibody T26B382 LCDR2 (Contact)
LMIYDVSNRP

SEQ ID NO: 247 - protein, 10 aa, synthetic construct
  source 1..10: note = Exemplary anti-CD37 antibody T26B382 LCDR3 (Contact)
SSYTSSSTLV

SEQ ID NO: 248 - protein, 7 aa, synthetic construct
  source 1..7: note = Exemplary anti-CD37 antibody T26B385 LCDR1 (Contact)
SSWLAWY

SEQ ID NO: 249 - protein, 10 aa, synthetic construct
  source 1..10: note = Exemplary anti-CD37 antibody T26B385 LCDR2 (Contact)
LLIFKTSSLE

SEQ ID NO: 250 - protein, 6 aa, synthetic construct
  source 1..6: note = Exemplary anti-CD37 antibody T26B385 LCDR3 (Contact)
QQYNSY

SEQ ID NO: 251 - protein, 7 aa, synthetic construct
  source 1..7: note = Exemplary anti-CD37 antibody T26B386 LCDR1 (Contact)
NYYASWY

SEQ ID NO: 252 - protein, 10 aa, synthetic construct
  source 1..10: note = Exemplary anti-CD37 antibody T26B386 LCDR2 (Contact)
LVFYGKDNRP

SEQ ID NO: 253 - protein, 10 aa, synthetic construct
  source 1..10: note = Exemplary anti-CD37 antibody T26B386 LCDR3 (Contact)
NSRDSSGDHL

SEQ ID NO: 254 - protein, 7 aa, synthetic construct
  source 1..7: note = Exemplary anti-CD37 antibody T26B388 LCDR1 (Contact)
RNDLGWY

SEQ ID NO: 255 - protein, 10 aa, synthetic construct
  source 1..10: note = Exemplary anti-CD37 antibody T26B388 LCDR2 (Contact)
RLIYAASSLQ

SEQ ID NO: 256 - protein, 8 aa, synthetic construct
  source 1..8: note = Exemplary anti-CD37 antibody T26B388 LCDR3 (Contact)
LQHYTYPL

SEQ ID NO: 257 - protein, 454 aa, synthetic construct
  source 1..454: note = Exemplary Heavy chain of Anti-GPRC5D antibody GC5B747
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 258 - protein, 454 aa, synthetic construct
  source 1..454: note = Exemplary Heavy chain of Anti-GPRC5D antibody GC5B752
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPG

SEQ ID NO: 259 - protein, 214 aa, synthetic construct
  source 1..214: note = Exemplary Light chain of Anti-GPRC5D antibody GC5B747
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 214 aa, synthetic construct; identical to SEQ ID NO: 259
  source 1..214: note = Exemplary Light chain of Anti-GPRC5D antibody GC5B752

SEQ ID NO: 261 - protein, 12 aa, synthetic construct
  source 1..12: note = Exemplary anti-GPRC5D antibodies GC5B747 and GC5B752 HCDR1 (AbM)
GGSLSSSSYWWG

SEQ ID NO: 262 - protein, 9 aa, synthetic construct
  source 1..9: note = Exemplary anti-GPRC5D antibodies GC5B747 and GC5B752 HCDR2 (AbM)
TMYYSGNIY

SEQ ID NO: 263 - protein, 15 aa, synthetic construct
  source 1..15: note = Exemplary anti-GPRC5D antibodies GC5B747 and GC5B752 HCDR3 (AbM)
HVGYSYGRRFWYFDL

SEQ ID NO: 264 - protein, 7 aa, synthetic construct
  source 1..7: note = Exemplary anti-GPRC5D antibodies GC5B747 and GC5B752 HCDR1 (Kabat)
SSSYWWG

SEQ ID NO: 265 - protein, 16 aa, synthetic construct
  source 1..16: note = Exemplary anti-GPRC5D antibodies GC5B747 and GC5B752 HCDR2 (Kabat)
TMYYSGNIYYNPSLQS

SEQ ID NO: 266 - protein, 15 aa, synthetic construct
  source 1..15: note = Exemplary anti-GPRC5D antibodies GC5B747 and GC5B752 HCDR3 (Kabat)
HVGYSYGRRFWYFDL

SEQ ID NO: 267 - protein, 9 aa, synthetic construct
  source 1..9: note = Exemplary anti-GPRC5D antibodies GC5B747 and GC5B752 HCDR1 (Chothia)
GGSLSSSSY

SEQ ID NO: 268 - protein, 5 aa, synthetic construct
  source 1..5: note = Exemplary anti-GPRC5D antibodies GC5B747 and GC5B752 HCDR2 (Chothia)
YYSGN

SEQ ID NO: 269 - protein, 15 aa, synthetic construct
  source 1..15: note = Exemplary anti-GPRC5D antibodies GC5B747 and GC5B752 HCDR3 (Chothia)
HVGYSYGRRFWYFDL

SEQ ID NO: 270 - protein, 10 aa, synthetic construct
  source 1..10: note = Exemplary anti-GPRC5D antibodies GC5B747 and GC5B752 HCDR1 (IMGT)
GGSLSSSSYW

SEQ ID NO: 271 - protein, 7 aa, synthetic construct
  source 1..7: note = Exemplary anti-GPRC5D antibodies GC5B747 and GC5B752 HCDR2 (IMGT)
MYYSGNI

SEQ ID NO: 272 - protein, 17 aa, synthetic construct
  source 1..17: note = Exemplary anti-GPRC5D antibodies GC5B747 and GC5B752 HCDR3 (IMGT)
ARHVGYSYGRRFWYFDL

SEQ ID NO: 273 - protein, 8 aa, synthetic construct
  source 1..8: note = Exemplary anti-GPRC5D antibodies GC5B747 and GC5B752 HCDR1 (Contact)
SSSSYWWG

SEQ ID NO: 274 - protein, 12 aa, synthetic construct
  source 1..12: note = Exemplary anti-GPRC5D antibodies GC5B747 and GC5B752 HCDR2 (Contact)
WIGTMYYSGNIY

SEQ ID NO: 275 - protein, 16 aa, synthetic construct
  source 1..16: note = Exemplary anti-GPRC5D antibodies GC5B747 and GC5B752 HCDR3 (Contact)
ARHVGYSYGRRFWYFD

SEQ ID NO: 276 - protein, 11 aa, synthetic construct
  source 1..11: note = Exemplary anti-GPRC5D antibodies GC5B747 and GC5B752 LCDR1 (AbM)
RASQSVSSYLA

SEQ ID NO: 277 - protein, 7 aa, synthetic construct
  source 1..7: note = Exemplary anti-GPRC5D antibodies GC5B747 and GC5B752 LCDR2 (AbM)
DASNRAT

SEQ ID NO: 278 - protein, 9 aa, synthetic construct
  source 1..9: note = Exemplary anti-GPRC5D antibodies GC5B747 and GC5B752 LCDR3 (AbM)
QQRSNWPPT

SEQ ID NO: 279 - protein, 11 aa, synthetic construct
  source 1..11: note = Exemplary anti-GPRC5D antibodies GC5B747 and GC5B752 LCDR1 (Kabat)
RASQSVSSYLA

SEQ ID NO: 280 - protein, 7 aa, synthetic construct
  source 1..7: note = Exemplary anti-GPRC5D antibodies GC5B747 and GC5B752 LCDR2 (Kabat)
DASNRAT

SEQ ID NO: 281 - protein, 9 aa, synthetic construct
  source 1..9: note = Exemplary anti-GPRC5D antibodies GC5B747 and GC5B752 LCDR3 (Kabat)
QQRSNWPPT

SEQ ID NO: 282 - protein, 11 aa, synthetic construct
  source 1..11: note = Exemplary anti-GPRC5D antibodies GC5B747 and GC5B752 LCDR1 (Chothia)
RASQSVSSYLA

SEQ ID NO: 283 - protein, 7 aa, synthetic construct
  source 1..7: note = Exemplary anti-GPRC5D antibodies GC5B747 and GC5B752 LCDR2 (Chothia)
DASNRAT

SEQ ID NO: 284 - protein, 9 aa, synthetic construct
  source 1..9: note = Exemplary anti-GPRC5D antibodies GC5B747 and GC5B752 LCDR3 (Chothia)
QQRSNWPPT

SEQ ID NO: 285 - protein, 6 aa, synthetic construct
  source 1..6: note = Exemplary anti-GPRC5D antibodies GC5B747 and GC5B752 LCDR1 (IMGT)
QSVSSY

SEQ ID NO: 286 - skipped

SEQ ID NO: 287 - protein, 9 aa, synthetic construct
  source 1..9: note = Exemplary anti-GPRC5D antibodies GC5B747 and GC5B752 LCDR3 (IMGT)
QQRSNWPPT

SEQ ID NO: 288 - protein, 7 aa, synthetic construct
  source 1..7: note = Exemplary anti-GPRC5D antibodies GC5B747 and GC5B752 LCDR1 (Contact)
SSYLAWY

SEQ ID NO: 289 - protein, 10 aa, synthetic construct
  source 1..10: note = Exemplary anti-GPRC5D antibodies GC5B747 and GC5B752 LCDR2 (Contact)
LLIYDASNRA

SEQ ID NO: 290 - protein, 8 aa, synthetic construct
  source 1..8: note = Exemplary anti-GPRC5D antibodies GC5B747 and GC5B752 LCDR3 (Contact)
QQRSNWPP

SEQ ID NO: 291 - protein, 454 aa, synthetic construct
  source 1..454: note = Exemplary Heavy chain of Anti-KLK2 antibody KL2B870
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPG

SEQ ID NO: 292 - protein, 454 aa, synthetic construct
  source 1..454: note = Exemplary Heavy chain of Anti-KLK2 antibody KL2B871
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 293 - protein, 454 aa, synthetic construct
  source 1..454: note = Exemplary Heavy chain of Anti-KLK2 antibody KL2B872
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 294 - protein, 214 aa, synthetic construct
  source 1..214: note = Exemplary Light chain of Anti-KLK2 antibody KL2B870
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 214 aa, synthetic construct; identical to SEQ ID NO: 294
  source 1..214: note = Exemplary Light chain of Anti-KLK2 antibody KL2B871

SEQ ID NO: 296 - protein, 214 aa, synthetic construct; identical to SEQ ID NO: 294
  source 1..214: note = Exemplary Light chain of Anti-KLK2 antibody KL2B872

SEQ ID NO: 297 - protein, 10 aa, synthetic construct
  source 1..10: note = Exemplary anti-KLK2 antibodies KL2B870, KL2B871, and KL2B872 HCDR1 (AbM)
GGSISSYYWS

SEQ ID NO: 298 - protein, 9 aa, synthetic construct
  source 1..9: note = Exemplary anti-KLK2 antibodies KL2B870, KL2B871, and KL2B872 HCDR2 (AbM)
YIYYSGSTN

SEQ ID NO: 299 - protein, 17 aa, synthetic construct
  source 1..17: note = Exemplary anti-KLK2 antibodies KL2B870, KL2B871, and KL2B872 HCDR3 (AbM)
TTIFGVVTPNFYYGMDV

SEQ ID NO: 300 - protein, 5 aa, synthetic construct
  source 1..5: note = Exemplary anti-KLK2 antibodies KL2B870, KL2B871, and KL2B872 HCDR1 (Kabat)
SYYWS

SEQ ID NO: 301 - protein, 16 aa, synthetic construct
  source 1..16: note = Exemplary anti-KLK2 antibodies KL2B870, KL2B871, and KL2B872 HCDR2 (Kabat)
YIYYSGSTNYNPSLKS

SEQ ID NO: 302 - protein, 17 aa, synthetic construct
  source 1..17: note = Exemplary anti-KLK2 antibodies KL2B870, KL2B871, and KL2B872 HCDR3 (Kabat)
TTIFGVVTPNFYYGMDV

SEQ ID NO: 303 - protein, 7 aa, synthetic construct
  source 1..7: note = Exemplary anti-KLK2 antibodies KL2B870, KL2B871, and KL2B872 HCDR1 (Chothia)
GGSISSY

SEQ ID NO: 304 - protein, 5 aa, synthetic construct
  source 1..5: note = Exemplary anti-KLK2 antibodies KL2B870, KL2B871, and KL2B872 HCDR2 (Chothia)
YYSGS

SEQ ID NO: 305 - protein, 17 aa, synthetic construct
  source 1..17: note = Exemplary anti-KLK2 antibodies KL2B870, KL2B871, and KL2B872 HCDR3 (Chothia)
TTIFGVVTPNFYYGMDV

SEQ ID NO: 306 - protein, 8 aa, synthetic construct
  source 1..8: note = Exemplary anti-KLK2 antibodies KL2B870, KL2B871, and KL2B872 HCDR1 (IMGT)
GGSISSYY

SEQ ID NO: 307 - protein, 7 aa, synthetic construct
  source 1..7: note = Exemplary anti-KLK2 antibodies KL2B870, KL2B871, and KL2B872 HCDR2 (IMGT)
IYYSGST

SEQ ID NO: 308 - protein, 19 aa, synthetic construct
  source 1..19: note = Exemplary anti-KLK2 antibodies KL2B870, KL2B871, and KL2B872 HCDR3 (IMGT)
AGTTIFGVVTPNFYYGMDV

SEQ ID NO: 309 - protein, 6 aa, synthetic construct
  source 1..6: note = Exemplary anti-KLK2 antibodies KL2B870, KL2B871, and KL2B872 HCDR1 (Contact)
SSYYWS

SEQ ID NO: 310 - protein, 12 aa, synthetic construct
  source 1..12: note = Exemplary anti-KLK2 antibodies KL2B870, KL2B871, and KL2B872 HCDR2 (Contact)
WIGYIYYSGSTN

SEQ ID NO: 311 - protein, 18 aa, synthetic construct
  source 1..18: note = Exemplary anti-KLK2 antibodies KL2B870, KL2B871, and KL2B872 HCDR3 (Contact)
AGTTIFGVVTPNFYYGMD

SEQ ID NO: 312 - protein, 11 aa, synthetic construct
  source 1..11: note = Exemplary anti-KLK2 antibodies KL2B870, KL2B871, and KL2B872 LCDR1 (AbM)
RASQGISSYLA

SEQ ID NO: 313 - protein, 7 aa, synthetic construct
  source 1..7: note = Exemplary anti-KLK2 antibodies KL2B870, KL2B871, and KL2B872 LCDR2 (AbM)
AASTLQS

SEQ ID NO: 314 - protein, 9 aa, synthetic construct
  source 1..9: note = Exemplary anti-KLK2 antibodies KL2B870, KL2B871, and KL2B872 LCDR3 (AbM)
QQLNSYPLT

SEQ ID NO: 315 - protein, 11 aa, synthetic construct
  source 1..11: note = Exemplary anti-KLK2 antibodies KL2B870, KL2B871, and KL2B872 LCDR1 (Kabat)
RASQGISSYLA

SEQ ID NO: 316 - protein, 7 aa, synthetic construct
  source 1..7: note = Exemplary anti-KLK2 antibodies KL2B870, KL2B871, and KL2B872 LCDR2 (Kabat)
AASTLQS

SEQ ID NO: 317 - protein, 9 aa, synthetic construct
  source 1..9: note = Exemplary anti-KLK2 antibodies KL2B870, KL2B871, and KL2B872 LCDR3 (Kabat)
QQLNSYPLT

SEQ ID NO: 318 - protein, 11 aa, synthetic construct
  source 1..11: note = Exemplary anti-KLK2 antibodies KL2B870, KL2B871, and KL2B872 LCDR1 (Chothia)
RASQGISSYLA

SEQ ID NO: 319 - protein, 7 aa, synthetic construct
  source 1..7: note = Exemplary anti-KLK2 antibodies KL2B870, KL2B871, and KL2B872 LCDR2 (Chothia)
AASTLQS

SEQ ID NO: 320 - protein, 9 aa, synthetic construct
  source 1..9: note = Exemplary anti-KLK2 antibodies KL2B870, KL2B871, and KL2B872 LCDR3 (Chothia)
QQLNSYPLT

SEQ ID NO: 321 - protein, 6 aa, synthetic construct
  source 1..6: note = Exemplary anti-KLK2 antibodies KL2B870, KL2B871, and KL2B872 LCDR1 (IMGT)
QGISSY

SEQ ID NO: 322 - skipped

SEQ ID NO: 323 - protein, 9 aa, synthetic construct
  source 1..9: note = Exemplary anti-KLK2 antibodies KL2B870, KL2B871, and KL2B872 LCDR3 (IMGT)
QQLNSYPLT

SEQ ID NO: 324 - protein, 7 aa, synthetic construct
  source 1..7: note = Exemplary anti-KLK2 antibodies KL2B870, KL2B871, and KL2B872 LCDR1 (Contact)
SSYLAWY

SEQ ID NO: 325 - protein, 10 aa, synthetic construct
  source 1..10: note = Exemplary anti-KLK2 antibodies KL2B870, KL2B871, and KL2B872 LCDR2 (Contact)
FLIYAASTLQ

SEQ ID NO: 326 - protein, 8 aa, synthetic construct
  source 1..8: note = Exemplary anti-KLK2 antibodies KL2B870, KL2B871, and KL2B872 LCDR3 (Contact)
QQLNSYPL

SEQ ID NO: 327 - protein, 454 aa, synthetic construct
  source 1..454: note = Exemplary Heavy chain of Anti-HLA-G antibody MHGB732
QVQLQQSGPGLVKPSQTLSLTCAISGDSVSSNSAAWNWIRQSPSRGLEWLGRTYYRSKWYNDYAVSVKSRITINPDTSKNQISLQLNSVTPEDTAVYYCAGDRRYGIVGLPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 328 - protein, 450 aa, synthetic construct
  source 1..450: note = Exemplary Heavy chain of Anti-HLA-G antibody MHGB738
QVQLQQSGPGLVKPSQTLSLTCAISGDSVSSNRAAWNWIRQSPSRGLEWLGRTYYRSKWYNDYAVSVKSRITINPDTSKNQISLQLNSVTPEDTAVYYCARVRPGIPFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 329 - protein, 390 aa, synthetic construct
  source 1..390: note = Exemplary Heavy chain of Anti-HLA-G antibody MHGB745
NDYAVSVKSRITINPDTSKNQISLQLNSVTPEDTAVYYCARVRPGIPFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 330 - protein, 454 aa, synthetic construct
  source 1..454: note = Exemplary Heavy chain of Anti-HLA-G antibody MHGB752
QVQLQQSGPGLVKPSQTLSLTCAISGDSVSSNSAAWNWIRQSPSRGLEWLGRTYYRSKWYNDYAVSVKSRITINPDTSKNQISLQLNSVTPEDTAVYYCAGDRRYGIVGLPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPG

SEQ ID NO: 331 - protein, 450 aa, synthetic construct
  source 1..450: note = Exemplary Heavy chain of Anti-HLA-G antibody MHGB758
QVQLQQSGPGLVKPSQTLSLTCAISGDSVSSNRAAWNWIRQSPSRGLEWLGRTYYRSKWYNDYAVSVKSRITINPDTSKNQISLQLNSVTPEDTAVYYCARVRPGIPFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPG

SEQ ID NO: 332 - protein, 220 aa, synthetic construct
  source 1..220: note = Exemplary Light chain of Anti-HLA-G antibody MHGB732
DIVMTQSPDSLAVSLGERATINCKSSQSVLHSSNNKNYLTWFQQKPGQPPKLLIYWASTRESGVPDRFSGSGSGTDFTLTISSLQAEDVAVYYCHQYYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 220 aa, synthetic construct
  source 1..220: note = Exemplary Light chain of Anti-HLA-G antibody MHGB738
DIVMTQSPDSLAVSLGERATINCKSSQSVLFSSNNKNYLAWYQQKPGQPPKLLIYWASTRESGVPDRFSGSVSGTDFTLTISSLQAEDVAVYYCQQYHSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 220 aa, synthetic construct; identical to SEQ ID NO: 333
  source 1..220: note = Exemplary Light chain of Anti-HLA-G antibody MHGB745

SEQ ID NO: 335 - protein, 220 aa, synthetic construct; identical to SEQ ID NO: 332
  source 1..220: note = Exemplary Light chain of Anti-HLA-G antibody MHGB752

SEQ ID NO: 336 - protein, 220 aa, synthetic construct; identical to SEQ ID NO: 333
  source 1..220: note = Exemplary Light chain of Anti-HLA-G antibody MHGB758

SEQ ID NO: 337 - protein, 12 aa, synthetic construct
  source 1..12: note = Exemplary anti-HLA-G antibodies MHGB732 and MHGB752 HCDR1 (AbM)
GDSVSSNSAAWN

SEQ ID NO: 338 - protein, 11 aa, synthetic construct
  source 1..11: note = Exemplary anti-HLA-G antibodies MHGB732 and MHGB752 HCDR2 (AbM)
RTYYRSKWYND

SEQ ID NO: 339 - protein, 13 aa, synthetic construct
  source 1..13: note = Exemplary anti-HLA-G antibodies MHGB732 and MHGB752 HCDR3 (AbM)
DRRYGIVGLPFAY

SEQ ID NO: 340 - protein, 12 aa, synthetic construct
  source 1..12: note = Exemplary anti-HLA-G antibodies MHGB738, MHGB745, and MHGB758 HCDR1 (AbM)
GDSVSSNRAAWN

SEQ ID NO: 341 - protein, 11 aa, synthetic construct
  source 1..11: note = Exemplary anti-HLA-G antibodies MHGB738, MHGB745, and MHGB758 HCDR2 (AbM)
RTYYRSKWYND

SEQ ID NO: 342 - protein, 9 aa, synthetic construct
  source 1..9: note = Exemplary anti-HLA-G antibodies MHGB738, MHGB745, and MHGB758 HCDR3 (AbM)
VRPGIPFDY

SEQ ID NO: 343 - protein, 7 aa, synthetic construct
  source 1..7: note = Exemplary anti-HLA-G antibodies MHGB732 and MHGB752 HCDR1 (Kabat)
SNSAAWN

SEQ ID NO: 344 - protein, 18 aa, synthetic construct
  source 1..18: note = Exemplary anti-HLA-G antibodies MHGB732 and MHGB752 HCDR2 (Kabat)
RTYYRSKWYNDYAVSVKS

SEQ ID NO: 345 - protein, 13 aa, synthetic construct
  source 1..13: note = Exemplary anti-HLA-G antibodies MHGB732 and MHGB752 HCDR3 (Kabat)
DRRYGIVGLPFAY

SEQ ID NO: 346 - protein, 7 aa, synthetic construct
  source 1..7: note = Exemplary anti-HLA-G antibodies MHGB738, MHGB745, and MHGB758 HCDR1 (Kabat)
SNRAAWN

SEQ ID NO: 347 - protein, 18 aa, synthetic construct
  source 1..18: note = Exemplary anti-HLA-G antibodies MHGB738, MHGB745, and MHGB758 HCDR2 (Kabat)
RTYYRSKWYNDYAVSVKS

SEQ ID NO: 348 - protein, 9 aa, synthetic construct
  source 1..9: note = Exemplary anti-HLA-G antibodies MHGB738, MHGB745, and MHGB758 HCDR3 (Kabat)
VRPGIPFDY

SEQ ID NO: 349 - protein, 9 aa, synthetic construct
  source 1..9: note = Exemplary anti-HLA-G antibodies MHGB732 and MHGB752 HCDR1 (Chothia)
GDSVSSNSA

SEQ ID NO: 350 - protein, 7 aa, synthetic construct
  source 1..7: note = Exemplary anti-HLA-G antibodies MHGB732 and MHGB752 HCDR2 (Chothia)
YYRSKWY

SEQ ID NO: 351 - protein, 13 aa, synthetic construct
  source 1..13: note = Exemplary anti-HLA-G antibodies MHGB732 and MHGB752 HCDR3 (Chothia)
DRRYGIVGLPFAY

SEQ ID NO: 352 - protein, 9 aa, synthetic construct
  source 1..9: note = Exemplary anti-HLA-G antibodies MHGB738, MHGB745, and MHGB758 HCDR1 (Chothia)
GDSVSSNRA

SEQ ID NO: 353 - protein, 7 aa, synthetic construct
  source 1..7: note = Exemplary anti-HLA-G antibodies MHGB738, MHGB745, and MHGB758 HCDR2 (Chothia)
YYRSKWY

SEQ ID NO: 354 - protein, 9 aa, synthetic construct
  source 1..9: note = Exemplary anti-HLA-G antibodies MHGB738, MHGB745, and MHGB758 HCDR3 (Chothia)
VRPGIPFDY

SEQ ID NO: 355 - protein, 10 aa, synthetic construct
  source 1..10: note = Exemplary anti-HLA-G antibodies MHGB732 and MHGB752 HCDR1 (IMGT)
GDSVSSNSAA

SEQ ID NO: 356 - protein, 9 aa, synthetic construct
  source 1..9: note = Exemplary anti-HLA-G antibodies MHGB732 and MHGB752 HCDR2 (IMGT)
TYYRSKWYN

SEQ ID NO: 357 - protein, 15 aa, synthetic construct
  source 1..15: note = Exemplary anti-HLA-G antibodies MHGB732 and MHGB752 HCDR3 (IMGT)
AGDRRYGIVGLPFAY

SEQ ID NO: 358 - protein, 10 aa, synthetic construct
  source 1..10: note = Exemplary anti-HLA-G antibodies MHGB738, MHGB745, and MHGB758 HCDR1 (IMGT)
GDSVSSNRAA

SEQ ID NO: 359 - protein, 9 aa, synthetic construct
  source 1..9: note = Exemplary anti-HLA-G antibodies MHGB738, MHGB745, and MHGB758 HCDR2 (IMGT)
TYYRSKWYN

SEQ ID NO: 360 - protein, 11 aa, synthetic construct
  source 1..11: note = Exemplary anti-HLA-G antibodies MHGB738, MHGB745, and MHGB758 HCDR3 (IMGT)
ARVRPGIPFDY

SEQ ID NO: 361 - protein, 8 aa, synthetic construct
  source 1..8: note = Exemplary anti-HLA-G antibodies MHGB732 and MHGB752 HCDR1 (Contact)
SSNSAAWN

SEQ ID NO: 362 - protein, 14 aa, synthetic construct
  source 1..14: note = Exemplary anti-HLA-G antibodies MHGB732 and MHGB752 HCDR2 (Contact)
WLGRTYYRSKWYND

SEQ ID NO: 363 - protein, 14 aa, synthetic construct
  source 1..14: note = Exemplary anti-HLA-G antibodies MHGB732 and MHGB752 HCDR3 (Contact)
AGDRRYGIVGLPFA

SEQ ID NO: 364 - protein, 8 aa, synthetic construct
  source 1..8: note = Exemplary anti-HLA-G antibodies MHGB738, MHGB745, and MHGB758 HCDR1 (Contact)
SSNRAAWN

SEQ ID NO: 365 - protein, 14 aa, synthetic construct
WLGRTYYRSKWYND

SEQ ID NO: 366 - protein, 10 aa, synthetic construct
  source 1..10: note = Exemplary anti-HLA-G antibodies MHGB738, MHGB745, and MHGB758 HCDR2 (Contact); note = Exemplary anti-HLA-G antibodies MHGB738, MHGB745, and MHGB758 HCDR3 (Contact)
ARVRPGIPFD

SEQ ID NO: 367 - protein, 17 aa, synthetic construct
  source 1..17: note = Exemplary anti-HLA-G antibodies MHGB732 and MHGB752 LCDR1 (AbM)
KSSQSVLHSSNNKNYLT

SEQ ID NO: 368 - protein, 7 aa, synthetic construct
  source 1..7: note = Exemplary anti-HLA-G antibodies MHGB732 and MHGB752 LCDR2 (AbM)
WASTRES

SEQ ID NO: 369 - protein, 9 aa, synthetic construct
  source 1..9: note = Exemplary anti-HLA-G antibodies MHGB732 and MHGB752 LCDR3 (AbM)
HQYYSTPPT

SEQ ID NO: 370 - protein, 17 aa, synthetic construct
  source 1..17: note = Exemplary anti-HLA-G antibodies MHGB738, MHGB745, and MHGB758 LCDR1 (AbM)
KSSQSVLFSSNNKNYLA

SEQ ID NO: 371 - protein, 7 aa, synthetic construct
  source 1..7: note = Exemplary anti-HLA-G antibodies MHGB738, MHGB745, and MHGB758 LCDR2 (AbM)
WASTRES

SEQ ID NO: 372 - protein, 9 aa, synthetic construct
  source 1..9: note = Exemplary anti-HLA-G antibodies MHGB738, MHGB745, and MHGB758 LCDR3 (AbM)
QQYHSTPWT

SEQ ID NO: 373 - protein, 17 aa, synthetic construct
  source 1..17: note = Exemplary anti-HLA-G antibodies MHGB732 and MHGB752 LCDR1 (Kabat)
KSSQSVLHSSNNKNYLT

SEQ ID NO: 374 - protein, 7 aa, synthetic construct
  source 1..7: note = Exemplary anti-HLA-G antibodies MHGB732 and MHGB752 LCDR2 (Kabat)
WASTRES

SEQ ID NO: 375 - protein, 9 aa, synthetic construct
  source 1..9: note = Exemplary anti-HLA-G antibodies MHGB732 and MHGB752 LCDR3 (Kabat)
HQYYSTPPT

SEQ ID NO: 376 - protein, 17 aa, synthetic construct
  source 1..17: note = Exemplary anti-HLA-G antibodies MHGB738, MHGB745, and MHGB758 LCDR1 (Kabat)
KSSQSVLFSSNNKNYLA

SEQ ID NO: 377 - protein, 7 aa, synthetic construct
  source 1..7: note = Exemplary anti-HLA-G antibodies MHGB738, MHGB745, and MHGB758 LCDR2 (Kabat)
WASTRES

SEQ ID NO: 378 - protein, 9 aa, synthetic construct
  source 1..9: note = Exemplary anti-HLA-G antibodies MHGB738, MHGB745, and MHGB758 LCDR3 (Kabat)
QQYHSTPWT

SEQ ID NO: 379 - protein, 17 aa, synthetic construct
  source 1..17: note = Exemplary anti-HLA-G antibodies MHGB732 and MHGB752 LCDR1 (Chothia)
KSSQSVLHSSNNKNYLT

SEQ ID NO: 380 - protein, 7 aa, synthetic construct
  source 1..7: note = Exemplary anti-HLA-G antibodies MHGB732 and MHGB752 LCDR2 (Chothia)
WASTRES

SEQ ID NO: 381 - protein, 9 aa, synthetic construct
  source 1..9: note = Exemplary anti-HLA-G antibodies MHGB732 and MHGB752 LCDR3 (Chothia)
HQYYSTPPT

SEQ ID NO: 382 - protein, 17 aa, synthetic construct
  source 1..17: note = Exemplary anti-HLA-G antibodies MHGB738, MHGB745, and MHGB758 LCDR1 (Chothia)
KSSQSVLFSSNNKNYLA

SEQ ID NO: 383 - protein, 7 aa, synthetic construct
  source 1..7: note = Exemplary anti-HLA-G antibodies MHGB738, MHGB745, and MHGB758 LCDR2 (Chothia)
WASTRES

SEQ ID NO: 384 - protein, 9 aa, synthetic construct
  source 1..9: note = Exemplary anti-HLA-G antibodies MHGB738, MHGB745, and MHGB758 LCDR3 (Chothia)
QQYHSTPWT

SEQ ID NO: 385 - protein, 12 aa, synthetic construct
  source 1..12: note = Exemplary anti-HLA-G antibodies MHGB732 and MHGB752 LCDR1 (IMGT)
QSVLHSSNNKNY

SEQ ID NO: 386 - skipped

SEQ ID NO: 387 - protein, 9 aa, synthetic construct
  source 1..9: note = Exemplary anti-HLA-G antibodies MHGB732 and MHGB752 LCDR3 (IMGT)
HQYYSTPPT

SEQ ID NO: 388 - protein, 12 aa, synthetic construct
  source 1..12: note = Exemplary anti-HLA-G antibodies MHGB738, MHGB745, and MHGB758 LCDR1 (IMGT)
QSVLFSSNNKNY

SEQ ID NO: 389 - skipped

SEQ ID NO: 390 - protein, 9 aa, synthetic construct
  source 1..9: note = Exemplary anti-HLA-G antibodies MHGB738, MHGB745, and MHGB758 LCDR3 (IMGT)
QQYHSTPWT

SEQ ID NO: 391 - protein, 13 aa, synthetic construct
  source 1..13: note = Exemplary anti-HLA-G antibodies MHGB732 and MHGB752 LCDR1 (Contact)
LHSSNNKNYLTWF

SEQ ID NO: 392 - protein, 10 aa, synthetic construct
  source 1..10: note = Exemplary anti-HLA-G antibodies MHGB732 and MHGB752 LCDR2 (Contact)
LLIYWASTRE

SEQ ID NO: 393 - protein, 8 aa, synthetic construct
  source 1..8: note = Exemplary anti-HLA-G antibodies MHGB732 and MHGB752 LCDR3 (Contact)
HQYYSTPP

SEQ ID NO: 394 - protein, 13 aa, synthetic construct
  source 1..13: note = Exemplary anti-HLA-G antibodies MHGB738, MHGB745, and MHGB758 LCDR1 (Contact)
LFSSNNKNYLAWY

SEQ ID NO: 395 - protein, 10 aa, synthetic construct
  source 1..10: note = Exemplary anti-HLA-G antibodies MHGB738, MHGB745, and MHGB758 LCDR2 (Contact)
LLIYWASTRE

SEQ ID NO: 396 - protein, 8 aa, synthetic construct
  source 1..8: note = Exemplary anti-HLA-G antibodies MHGB738, MHGB745, and MHGB758 LCDR3 (Contact)
QQYHSTPW

(19) United States
(12) Patent Application Publication
(10) Pub. No.: US 2025/0230254 A1
(43) Pub. Date: Jul. 17, 2025

(54) BIOSYNTHETIC BIOPARATOPIC OR BISPECIFIC BINDING MOLECULES WITH ENHANCED EFFECTOR FUNCTIONS

(71) Applicant: Janssen Biotech, Inc., Horsham, PA (US)

(72) Inventors: Adam ZWOLAK, Bala Cynwyd, PA (US); Jason HO, Collegeville, PA (US); James TESTA, Jr., Sellersville, PA (US); Michael Riis HANSEN, Broomall, PA (US); Xiefan LIN-SCHMIDT, Ambler, PA (US); Ian WHITE, Conshohocken, PA (US); Sanjaya SINGH, Blue Bell, PA (US)

(21) Appl. No.: 18/703,274

(22) PCT Filed: Oct. 19, 2022
(86) PCT No.: PCT/US2022/078355

§ 371 Date: Apr. 19, 2024

Related U.S. Application Data
(60) Provisional application No. 63/270,031, filed on Oct. 20, 2021.

(51) Int. Cl. C07K 16/28; C07K 16/30

(52) U.S. Cl. CPC C07K 16/2896; C07K 16/28; C07K 16/2803; C07K 16/2833; C07K 16/2878; C07K 16/3069; C07K 2317/31; C07K 2317/41; C07K 2317/52; C07K 2317/732; C07K 2317/734; C07K 2317/92; C07K 2317/94

(57) ABSTRACT

Provided herein, in certain aspects, is a binding molecule comprising an antigen binding domain and an Fc region; wherein the antigen binding domain is biparatopic or bispecific and the Fc region comprises K248E and T437R mutations (RE mutations), wherein amino acid residue numbering is according to the EU numbering system; wherein the binding molecule has increased capability of hexamerization on a cell surface, and/or increased capability of engaging C1q.

### CROSS-REFERENCE TO RELATED APPLICATIONS

This application claims the benefit of priority to U.S. Ser. No. 63/270,031 filed Oct. 20, 2021, the content of which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “14620-722-228_SEQ_LISTING.xml”, was created on Oct. 3, 2022 and is 431,738 bytes in size.

### 1. FIELD

Provided herein are biparatopic or bispecific binding molecules with enhanced antibody-dependent cellular cytotoxicity (ADCC), enhanced complement-dependent cytotoxicity (CDC), and/or enhanced antibody-dependent cellular phagocytosis (ADCP).

### 2. BACKGROUND

Therapeutic antibodies can bind Fc receptors expressed on immune effector cells, such as natural killer (NK) cells and macrophages, resulting in anti-tumor activity via ADCC and ADCP. Therapeutic antibodies can also activate CDC and achieve anti-tumor efficacy.

### 3. SUMMARY

In one aspect, provided herein is a binding molecule comprising an antigen binding domain and an Fc region, wherein (i) the antigen binding domain is biparatopic or bi-specific; and (ii) the Fc region comprises K248E and T437R mutations (RE mutations), wherein amino acid residue numbering is according to the EU numbering system; wherein the binding molecule has increased capability of hexamerization on a cell surface, and/or increased capability of engaging C1q.

In some embodiments, the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s). In some embodiments, wherein: (i) each of the first domain and the second domain is a Fab fragment, a scFv, a single domain antigen binding fragment, or an engineered binding fragment; (ii) the first domain is a Fab fragment and the second domain is a scFv; (iii) the first domain is a Fab fragment and the second domain is a single domain antigen binding fragment; (iv) the first domain is a Fab fragment and the second domain is an engineered binding fragment; (v) the first domain is a scFv and the second domain is a single domain antigen binding fragment; (vi) the first domain is a scFv and the second domain is an engineered binding fragment; or (vii) the first domain is a single domain antigen binding fragment and the second domain is an engineered binding fragment.

In some embodiments, the oligosaccharide covalently attached to the Fc region via the N297 residue thereof does not comprise a core fucose residue.

In some embodiments, the binding molecule is a biparatopic or bispecific antibody.

In some embodiments, the binding molecule has enhanced ADCC, enhanced CDC, and/or enhanced ADCP.

In another aspect, provided herein is a population of binding molecules comprising the binding molecule disclosed herein. In some embodiments, each binding molecule comprising an antigen binding domain and an Fc region; wherein: (i) the antigen binding domain is biparatopic or bispecific; (ii) the Fc region comprises K248E and T437R mutations (RE mutations), wherein amino acid residue numbering is according to the EU numbering system; and (iii) less than 80% of the oligosaccharides covalently attached to the population of the binding molecules via the N297 residue of the Fc region comprise a core fucose residue.

In another aspect, provided herein is a population of binding molecules, wherein each binding molecule comprises an antigen binding domain and an Fc region; wherein the antigen binding domain of at least 10% of the binding molecules in the population is biparatopic or bispecific; wherein the Fc region of each binding molecule comprises K248E and T437R mutations (RE mutations), wherein amino acid residue numbering is according to the EU numbering system; and wherein less than 80% of the oligosaccharides covalently attached to the population of the binding molecules via the N297 residue of the Fc region comprise a core fucose residue.

In some embodiments, the antigen binding domain of at least 20% of the binding molecules in the population is biparatopic or bispecific. In some embodiments, the antigen binding domain of at least 30% of the binding molecules in the population is biparatopic or bispecific. In some embodiments, the antigen binding domain of at least 40% of the binding molecules in the population is biparatopic or bispecific. In some embodiments, wherein the antigen binding domain of at least 50% of the binding molecules in the population is biparatopic or bispecific. In some embodiments, the antigen binding domain of at least 60% of the binding molecules in the population is biparatopic or bispecific. In some embodiments, wherein the antigen binding domain of at least 70% of the binding molecules in the population is biparatopic or bispecific. In some embodiments, wherein the antigen binding domain of at least 80% of the binding molecules in the population is biparatopic or bispecific. In some embodiments, the antigen binding domain of at least 90% of the binding molecules in the population is biparatopic or bispecific.

In some embodiments, less than 70% of the oligosaccharides covalently attached to the population of the binding molecules via the N297 residue of the Fc region comprise a core fucose residue. In some embodiments, less than 60% of the oligosaccharides covalently attached to the population of the binding molecules via the N297 residue of the Fc region comprise a core fucose residue. In some embodiments, less than 50% of the oligosaccharides covalently attached to the population of the binding molecules via the N297 residue of the Fc region comprise a core fucose residue. In some embodiments, less than 40% of the oligosaccharides covalently attached to the population of the binding molecules via the N297 residue of the Fc region comprise a core fucose residue. In some embodiments, less than 30% of the oligosaccharides covalently attached to the population of the binding molecules via the N297 residue of the Fc region comprise a core fucose residue. In some embodiments, less than 20% of the oligosaccharides covalently attached to the population of the binding molecules via the N297 residue of the Fc region comprise a core fucose residue. In some embodiments, less than 10% of the oligosaccharides covalently attached to the population of the binding molecules via the N297 residue of the Fc region comprise a core fucose residue.

In some embodiments, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s). In some embodiments, wherein: (i) each of the first domain and the second domain is a Fab fragment, a scFv, a single domain antigen binding fragment, or an engineered binding fragment; the first domain is a Fab fragment and the second domain is a scFv; the first domain is a Fab fragment and the second domain is a single domain antigen binding fragment; the first domain is a Fab fragment and the second domain is an engineered binding fragment; the first domain is a scFv and the second domain is a single domain antigen binding fragment; the first domain is a scFv and the second domain is an engineered binding fragment; or the first domain is a single domain antigen binding fragment and the second domain is an engineered binding fragment.

In some embodiments, the binding molecules are biparatopic or bispecific antibodies.

In some embodiments, the binding molecules are produced by expressing a polynucleotide encoding the binding molecules or a fragment thereof in a host cell that is deficient in adding a fucose to the oligosaccharide attached to the Fc region of an antibody. In some embodiments, the host cell has reduced GDP-mannose 4,6-dehydratase (GMD) activity or reduced α-1,6 fucosyltransferase activity. In some embodiments, wherein the gene encoding α-1,6 fucosyltransferase is mutated, expressed at a lower than normal level, or knocked out in the host cell. In some embodiments, the gene encoding GDP-mannose 4,6-dehydratase is mutated, expressed at a lower than normal level, or knocked out in the host cell.

In some embodiments, the population of the binding molecules has enhanced ADCC, enhanced CDC, and/or enhanced ADCP.

In another aspect, provided herein is a nucleic acid encoding the binding molecule disclosed herein.

In another aspect, provided herein is a vector comprising the nucleic acid.

In yet one aspect, provided herein is a pharmaceutical composition comprising a binding molecule and a pharmaceutically acceptable excipient, wherein the binding molecule comprises an antigen binding domain and an Fc region; wherein: (i) the antigen binding domain is biparatopic or bispecific; and (ii) the Fc region comprises K248E and T437R mutations (RE mutations), wherein amino acid residue numbering is according to the EU numbering system; wherein the binding molecule has increased capability of hexamerization on a cell surface, and/or increased capability of engaging C1q.

In another aspect, provided herein is a pharmaceutical composition comprising a population of binding molecules and a pharmaceutically acceptable excipient, wherein each binding molecule comprises an antigen binding domain and an Fc region; wherein: (i) the antigen binding domain is biparatopic or bispecific; (ii) the Fc region comprises K248E and T437R mutations (RE mutations), wherein amino acid residue numbering is according to the EU numbering system; and (iii) less than 80% of the oligosaccharides covalently attached to the population of the binding molecules via the N297 residue of the Fc region comprise a core fucose residue.

In another aspect, provided herein is a pharmaceutical composition comprising a population of binding molecules and a pharmaceutically acceptable excipient, wherein each binding molecule comprises an antigen binding domain and an Fc region, wherein the antigen binding domain of at least 10% of the binding molecules in the population is biparatopic or bispecific; wherein the Fc region of each binding molecule comprises K248E and T437R mutations (RE mutations), wherein amino acid residue numbering is according to the EU numbering system; and wherein less than 80% of the oligosaccharides covalently attached to the population of the binding molecules via the N297 residue of the Fc region comprise a core fucose residue.

In yet one aspect, provided herein is a method of making a population of binding molecules, comprising expressing a polynucleotide encoding the binding molecules or a fragment thereof in a host cell that is deficient in adding a fucose residue to an oligosaccharide attached to an antibody via the N297 residue, wherein the population of the binding molecules comprises a binding molecule comprising an antigen binding domain and an Fc region, wherein (i) the antigen binding domain is biparatopic or bispecific; and (ii) the Fc region comprises K248E and T437R mutations (RE mutations), wherein amino acid residue numbering is according to the EU numbering system; wherein the binding molecule has increased capability of hexamerization on a cell surface, and/or increased capability of engaging C1q.

In another aspect, provided herein is a method of making a population of binding molecules, comprising expressing a polynucleotide encoding the binding molecules or a fragment thereof in a host cell that is deficient in adding a fucose residue to an oligosaccharide attached to an antibody via N297 residue, wherein each binding molecule comprises an antigen binding domain and an Fc region; wherein: (i) the antigen binding domain is biparatopic or bispecific; (ii) the Fc region comprises K248E and T437R mutations (RE mutations), wherein amino acid residue numbering is according to the EU numbering system; and (iii) less than 80% of the oligosaccharides covalently attached to the population of the binding molecules via the N297 residue of the Fc region comprise a core fucose residue.

In another aspect, provided herein is a method of making a population of binding molecules, comprising expressing a polynucleotide encoding the binding molecules or a fragment thereof in a host cell that is deficient in adding a fucose residue to an oligosaccharide attached to an antibody via N297 residue, wherein each binding molecule comprises an antigen binding domain and an Fc region; wherein the antigen binding domain of at least 10% of the binding molecules in the population is biparatopic or bispecific; wherein the Fc region of each binding molecule comprises K248E and T437R mutations (RE mutations), wherein amino acid residue numbering is according to the EU numbering system; and wherein less than 80% of the oligosaccharides covalently attached to the population of the binding molecules via the N297 residue of the Fc region comprise a core fucose residue.

In yet one aspect, provided herein is a method for enhancing CDC, ADCP, and/or ADCC in a system, or a method of modulating an immunity in a host, or a method of treating a disease or disorder in a subject, comprising contacting the system with a binding molecule comprising an antigen binding domain and an Fc region, wherein (i) the antigen binding domain is biparatopic or bispecific; and (ii) the Fc region comprises K248E and T437R mutations (RE mutations), wherein amino acid residue numbering is according to the EU numbering system, wherein the binding molecule has increased capability of hexamerization on a cell surface, and/or increased capability of engaging C1q.

In another aspect, provided herein is a method for enhancing CDC, ADCP, and/or ADCC in a system, or a method of modulating an immunity in a host, or a method of treating a disease or disorder in a subject, comprising contacting the system with a population of binding molecules, wherein each binding molecule comprising an antigen binding domain and an Fc region; wherein: (i) the antigen binding domain is biparatopic or bispecific; (ii) the Fc region comprises K248E and T437R mutations (RE mutations), wherein amino acid residue numbering is according to the EU numbering system; and (iii) less than 80% of the oligosaccharides covalently attached to the population of the binding molecules via the N297 residue of the Fc region comprise a core fucose residue.

In another aspect, provided herein is a method for enhancing CDC, ADCP, and/or ADCC in a system, or a method of modulating an immunity in a host, or a method of treating a disease or disorder in a subject, comprising contacting the system with a population of binding molecules, wherein each binding molecule comprises an antigen binding domain and an Fc region; wherein the antigen binding domain of at least 10% of the binding molecules in the population is biparatopic or bispecific; wherein the Fc region of each binding molecule comprises K248E and T437R mutations (RE mutations), wherein amino acid residue numbering is according to the EU numbering system; and wherein less than 80% of the oligosaccharides covalently attached to the population of the binding molecules via the N297 residue of the Fc region comprise a core fucose residue.

In yet one aspect, provided herein is a binding molecule comprising a first means for enhancing CDC and a second means for enhancing CDC. In some embodiments, the binding molecule further comprising a third means for enhancing ADCC. In some embodiments, the binding molecule further comprising a fourth means for enhancing ADCP. In some embodiments, the first means increases hexamerization of the binding molecule on a cell surface, and/or increases C1q engagement by the binding molecule. In some embodiments, the first means enhances CDC, ADCP, and/or ADCC.

In another aspect, provided herein is a method of making a population of binding molecules, comprising (i) a step for introducing K248E and T437R mutations (RE mutations) in the Fc region of the binding molecules; and (ii) a step for producing the population of binding molecules with reduced amount of core fucoses in the oligosaccharides attached to the binding molecules via the N297 residue, wherein the binding molecules comprise a biparatopic or bispecific antigen binding domain.

### 4. BRIEF DESCRIPTION OF THE FIGURES

FIGS. 1A, 1B, 1C, 1D, 1E, 1F, 1G and 1H depict CDC activity against target cells expressing high levels of CD37 (CARNAVAL, FIGS. 1A, 1C, 1E and 1G) and low levels of CD37 (JEKO-1, FIGS. 1B, 1D, 1F and 1H) of different types of anti-CD37 antibodies with low fucosylation (FIGS. 1A and 1B), with Xencor mutations (FIGS. 1C and 1D), with RE mutations (FIGS. 1E and 1F), and with low fucosylation and RE mutations (FIGS. 1G and 1H). Target cells were incubated with titrating concentrations of antibodies as indicated for 30 minutes. Baby rabbit serum was then added to the mixture to a final concentration of 10% to provide a source of complement components. After 4 hours of incubation, cell viability was measured by addition of Cell Titer-Glo reagent (Promega) and measurement of the resulting luminescence and reported in Relative Luminescence Units (RLU).

FIG. 2 depicts CDC activity against H929 target cells of anti-GPRC5D antibodies with low fucosylation in wildtype and with RE mutations. Target cells were incubated with titrating concentrations of antibodies as indicated for 30 minutes. Baby rabbit serum was then added to the mixture to a final concentration of 10% to provide a source of complement components. After 4 hours of incubation, cell viability was measured by addition of Cell Titer-Glo reagent (Promega) and measurement of the resulting luminescence and reported in Relative Luminescence Units (RLU).

FIGS. 3A, 3B, 3C, 3D and 3E show the in vitro ADCC kinetic killing mediated by KLK2 antibodies with PBMC on VCaP cells. Briefly, VCaP cells stably transfected with Nuclight Red (Incucyte®, Essen Bioscience) were plated at 10,000 cells per well in a 384-well plate (Perkin Elmer ViewPlate) in clear media (RPMI 1641+10% FBS, Thermo Fisher Scientific) to allow for cell adherence overnight. ADCC assay was performed with freshly thawed PBMC (Hemcare, PB009C-3). The ratio of effector to target cell per well was 34:1 for PBMCs as effector cells. KLK2 antibodies were tested with final concentrations ranging from 100 nM to 0.01 nM. After effector cells and antibodies were added to target cells, real time imaging was performed under Incucyte© S3 instrument (Essen BioScience). Total red intergraded signal per well was quantified with Incucyte© software. Data analysis were performed by Incucyte© software and Prism (GraphPad Software) based on values of quadruplicates. The percentage of cell killing was calculated as: (1−KLK2 mAb/no mAb control)×100%.

FIG. 4 shows the in vitro ADCC dose-response killing by PBMC on VCaP at 48 hour. The dose-response curve was generated 48 hours after effector cells and antibodies were added to target cells.

FIGS. 5A and 5B depict the ADCC activity against JEG-3 and RERF-LC-Ad-1 cells of anti-HLA-G antibodies MHGB732 and MHGB738, and their respective counterparts with low fucosylation, with RE mutations, and with low fucosylation and RE mutations. The percentage of lysis was compared to the maximum lysis of JEG-3 or RERF-LC-Ad-1 cells by Triton-X 100 detergent and calculated as (sample value−target alone value)/(maximum value−target alone value)×100%.

FIGS. 5C and 5D depict the CDC activity of anti-HLA-G antibodies NMGB732 and MHGB738, and their respective counterparts with low fucosylation, with RE mutations, and with low fucosylation and RE mutations. Target cells were incubated with antibodies as indicated for 30 minutes at 37° C. 15-20% (stock concentration) of rabbit complement and heat inactivated complement was added to the wells respectively to a volume of 25 μl/well. The mixture was incubated for 4-12 hours at 37° C. Target cell lysis was measured by addition of Cell Titer-Glo reagent (Promega) and measurement of the resulting luminescence and reported in Relative Luminescence Units (RLU).

FIGS. 6A, 6B, 6C, and 6D show the in vitro ADCC dose-response killing mediated by anti-PSMA antibodies PSMB896 and PSMB898 with low fucosylation and RE mutations on C42B and LNCaP cells. Specifically, FIG. 6A shows the in vitro ADCC dose-response killing mediated by the antibodies with PBMC on C42B cells at 6 hour; FIG. 6B shows the in vitro ADCC dose-response killing mediated by the antibodies with PBMC on LNCaP cells at 6 hour;

FIG. 6C shows the in vitro ADCC dose-response killing mediated by the antibodies with NK cells on C42B cells at 24 hour; FIG. 6D shows the in vitro ADCC dose-response killing mediated by the antibodies with NK cells on LNCaP cells at 24 hour. The dose-response curves were generated 6 or 24 hours after effector cells and antibodies were added to target cells.

FIG. 7 depicts CDC activities against LNCaP target cells of biparatopic anti-PSMA antibodies using IgG1 constant domains with or without the RE mutations in the presence of 40% normal human serum. The CDC activity against LNCaP target cells of an isotype control antibody was also tested.

FIG. 8 depicts ADCC activities against BATDA-labeled LNCaP target cells of the same antibodies as those tested in FIG. 7 in the presence of purified NK cells from a normal donor.

### 5. DETAILED DESCRIPTION

Antibodies represent an important and growing class of therapeutic biologics for various diseases or disorders including oncology indications. The mechanisms of their action include blocking signals that inhibit anti-tumor immune responses, agonism of signals that augment anti-tumor responses, targeted delivery of anti-tumor drugs by conjugated antibodies, and target-specific depletion of cells expressing tumor-associated antigens through ADCC, ADCP, and CDC. Among these mechanisms of action, ADCC and ADCP are mediated through interactions of the fragment crystallizable region (Fc) of the antibodies with Fc7 receptors on effector cells (natural killer cells as in the case of ADCC or macrophages as in the case of ADCP). CDC is mediated via interaction between the Fc and complement component C1q. These mechanisms of target cell killing are collectively referred to as antibody effector functions. Numerous oncological therapeutics exploit these antibody effector functions to specifically deplete or suppress growth of tumor cells. Strategies that can increase Fc-mediated activation of these pathways can therefore lead to more potent cancer therapeutics. Strategies that can augment ADCC or ADCP effector functions have been described. However, they are often incompatible with the other functions, such as CDC activity. Here, we describe the combined application of several strategies that can enable the simultaneous augmentation of multiple antibody effector functions such as CDC, ADCC and/or ADCP. The combined improvement in effector functions could potentially lead to better therapeutics that can overcome dysfunction in one of these compartments in cancer patients.

CDC activity requires the activation of the complement cascade, which in turn requires activation of complement component C1q by a hexamer of Fc regions co-planar to the surface of the target cell. This in turn requires concurrent binding of five or six antibody molecules in close proximity on the cell surface. Due to the requirement for Fc-mediated antibody oligomerization CDC activity requires relatively high target receptor densities. With natural IgG antibodies, the stoichiometry of antibody-to-receptor binding can vary from 1:2 to 1:1 depending on antibody concentration, with the former highly favored due to avidity. To maximize the number of antibodies that can bind a given number of cell surface receptors, the present disclosure incorporates engineered biparatopic or bispecific antibody design to force 1:1 antibody-to-receptor binding stoichiometry. This allows more Fc regions to be brought to the surface of the cell with a given number of receptors compared to the natural bivalent IgG. Thus, engineered biparatopic or bispecific antibodies may have more potent CDC activity.

Another way to increase CDC activity is to enhance the ability of IgG to hexamerize through mutagenesis. One strategy, referred to as RE mutations, involves the incorporation of two point mutations (K248E and T437R) in the Fc regions. As demonstrated, enhancement of CDC activity through RE mutations is compatible with enhancement of ADCC and ADCP activity mediated by antibodies produced in a low fucose host. The biparatopic or bispecific antibodies featuring the RE mutations provided herein are compatible with enhancement of ADCC/ADCP via low fucose production, among other advanced properties, as shown in Section 7 below.

The present disclosure is based in part on the surprising finding that a population of antibodies possess both enhanced ADCC and enhanced CDC activities when less than 80% of the oligosaccharides covalently attached to the population of the antibodies comprise a fucose residue and the Fc region of the antibodies comprises K248E and T437R mutations (RE mutations). In addition, the low fucosylation does not interfere with the effect of the RE mutations on enhancing CDC, and RE mutations do not interfere with the enhanced ADCC activity conferred by low fucosylation, as demonstrated in Section 7 below. In one aspect, provided herein is an antibody that comprises an RE mutation in the Fc region and does not comprise a core fucose residue in the oligosaccharides covalently attached to the Fc region of the antibody. In another aspect, provided herein is a population of antibodies that comprise an antibody that comprises an RE mutation in the Fc region and does not comprise a core fucose residue in the oligosaccharides covalently attached to the Fc region of the antibody. In some embodiments, provided herein is a population of antibodies comprising K248E and T437R mutations (RE mutations), wherein less than 80% of the oligosaccharides covalently attached to the population of the antibodies via N297 residues thereof comprise a core fucose residue. In some embodiments, less than 70% of the oligosaccharides covalently attached to the population of the antibodies via N297 residues thereof comprise a core fucose residue. In some embodiments, less than 60% of the oligosaccharides covalently attached to the population of the antibodies via N297 residues thereof comprise a core fucose residue. In some embodiments, less than 50% of the oligosaccharides covalently attached to the population of the antibodies via N297 residues thereof comprise a core fucose residue. In some embodiments, less than 40% of the oligosaccharides covalently attached to the population of the antibodies via N297 residues thereof comprise a core fucose residue. In some embodiments, less than 30% of the oligosaccharides covalently attached to the population of the antibodies via N297 residues thereof comprise a core fucose residue. In some embodiments, less than 20% of the oligosaccharides covalently attached to the population of the antibodies via N297 residues thereof comprise a core fucose residue. In some embodiments, less than 10% of the oligosaccharides covalently attached to the population of the antibodies via N297 residues thereof comprise a core fucose residue. In some embodiments, less than 5% of the oligosaccharides covalently attached to the population of the antibodies via N297 residues thereof comprise a core fucose residue.

The present disclosure is also based in part on the surprising finding that the combination of biparatopic or bispecific configuration with the RE mutations provides further enhanced CDC activity via increased hexamerization of such a binding molecule and thus increased capability of engaging C1q on a cell surface, among other advanced properties as demonstrated in Section 7 below. Thus, in another aspect, the present disclosure provides a biparatopic or bispecific binding molecule that carries RE mutations in the Fc region. In some embodiments, such binding molecule does not comprise a core fucose residue in the oligosaccharides covalently attached to the Fc region of the binding molecule. In some embodiments, provided herein is a population of biparatopic or bispecific antibodies each carrying RE mutations, wherein less than 80% of the oligosaccharides covalently attached to the population of the biparatopic or bispecific antibodies via N297 residues thereof comprise a core fucose residue. In some embodiments, less than 70% of the oligosaccharides covalently attached to the population of the biparatopic or bispecific antibodies via N297 residues thereof comprise a core fucose residue. In some embodiments, less than 60% of the oligosaccharides covalently attached to the population of the biparatopic or bispecific antibodies via N297 residues thereof comprise a core fucose residue. In some embodiments, less than 50% of the oligosaccharides covalently attached to the population of the biparatopic or bispecific antibodies via N297 residues thereof comprise a core fucose residue. In some embodiments, less than 40% of the oligosaccharides covalently attached to the population of the biparatopic or bispecific antibodies via N297 residues thereof comprise a core fucose residue. In some embodiments, less than 30% of the oligosaccharides covalently attached to the population of the biparatopic or bispecific antibodies via N297 residues thereof comprise a core fucose residue. In some embodiments, less than 20% of the oligosaccharides covalently attached to the population of the biparatopic or bispecific antibodies via N297 residues thereof comprise a core fucose residue. In some embodiments, less than 10% of the oligosaccharides covalently attached to the population of the biparatopic or bispecific antibodies via N297 residues thereof comprise a core fucose residue. In some embodiments, less than 5% of the oligosaccharides covalently attached to the population of the biparatopic or bispecific antibodies via N297 residues thereof comprise a core fucose residue.

In certain embodiments, any antibodies with any antigen binding structure or targeting any antigen are included in the present disclosure, as long as the antibodies have a Fc region. Pharmaceutical compositions comprising the present antibodies, method of making, and uses thereof are also included in the present disclosure.

### 5.1 Definitions

Techniques and procedures described or referenced herein include those that are generally well understood and/or commonly employed using conventional methodology by those skilled in the art, such as, for example, the widely utilized methodologies described in Molecular Cloning: A Laboratory Manual (Sambrook, et al., 3d ed. 2001); Current Protocols in Molecular Biology (Ausubel, et al. eds., 2003); Therapeutic Monoclonal Antibodies: From Bench to Clinic (An, ed. 2009); Monoclonal Antibodies: Methods and Protocols (Albitar, ed. 2010); and Antibody Engineering Vols 1 and 2 (Kontermann and Dubel, eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multispecific antibodies (e.g., bispecific antibodies so long as they exhibit the desired biological activity), formed from at least two intact antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies or their humanized variants, intrabodies, and anti-idiotypic (anti-Id) antibodies. The term “antibody” as used herein also comprises any binding molecule having a Fc region and a functional fragment (e.g., an antigen-binding fragment) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers, ed., 1995); Huston, et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “biparatopic antibodies” refers a subset of bispecific antibodies where each of the two antigen binding domains recognize unique, non-overlapping epitopes on the same target of interest.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen binding site as well as a constant domain (CL) and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both koₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody described herein, the terms such as “bind to,” “that specifically bind to,” and analogous terms are also used interchangeably herein and refer to antibodies of antigen binding domains that specifically bind to an antigen, such as a polypeptide. An antibody or antigen binding domain that binds to or specifically binds to an antigen may be cross-reactive with related antigens. In certain embodiments, an antibody or antigen binding domain that binds to or specifically binds to an antigen does not cross-react with other antigens. An antibody or antigen binding domain that binds to or specifically binds to an antigen can be identified, for example, by immunoassays, Octet®, Biacore®, or other techniques known to those of skill in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul, ed., 2d ed. 1989) for a discussion regarding binding specificity. In certain embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. With regard to terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding domain that binds to an antigen includes one that is capable of binding the antigen with sufficient affinity such that the antibody is useful, for example, as a diagnostic or therapeutic agent in targeting the antigen. In certain embodiments, an antibody or antigen binding domain that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding domain binds to an epitope of an antigen that is conserved among the antigen from different species (e.g., between human and cynomolgus macaque species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen, et al., J. Mol Biol, 1999, 293:865-81). The K_{D} or K_{D} value may also be measured by using biolayer interferometry (BLI) or surface plasmon resonance (SPR) assays by Octet®, using, for example, an Octet® Red96 system, or by Biacore®, using, for example, a Biacore® 2000 or a Biacore® 3000. An “on-rate” or “rate of association” or “association rate” or “kₒₙ” may also be determined with the same biolayer interferometry (BLI) or surface plasmon resonance (SPR) techniques described above using, for example, the Octet® Red96, the Biacore® 2000, or the Biacore® 3000 system.

In certain embodiments, the antibodies can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (see U.S. Pat. No. 4,816,567; and Morrison, et al., Proc. Natl. Acad. Sci. USA, 1984, 81:6851-55).

In certain embodiments, the antibodies can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones, et al., Nature, 1986, 321:522-25; Riechmann, et al., Nature, 1988, 332:323-29; Presta, Curr. Op. Struct. Biol., 1992, 2:593-96; Carter, et al., Proc. Natl. Acad. Sci. USA, 1992, 89:4285-89; U.S. Pat. Nos. 6,800,738; 6,719,971; 6,639,055; 6,407,213; and 6,054,297.

In certain embodiments, the antibodies can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat, et al. (see Kabat, et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 1991, 227:381; Marks, et al., 1991, J. Mol. Biol., 1991, 222:581) and yeast display libraries (Chao, et al., Nature Protocols, 2006, 1: 755-68). Also available for the preparation of human monoclonal antibodies are methods described in Cole, et al., Monoclonal Antibodies and Cancer Therapy 77 (1985); Boerner, et al., J. Immunol., 1991, 147(1):86-95; and van Dijk and van de Winkel, Curr. Opin. Pharmacol., 2001, 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol., 1995, 6(5):561-66; Bruggemann and Taussing, Curr. Opin. Biotechnol., 1997, 8(4):455-58; and U.S. Pat. Nos. 6,075,181 and 6,150,584 regarding XENOMOUSE™ technology). See also, for example, Li, et al., Proc. Natl. Acad. Sci. USA, 2006, 103:3557-62, regarding human antibodies generated via a human B-cell hybridoma technology.

In certain embodiments, the antibodies can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D., et al., Nucl. Acids Res., 1992 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson, et al., Nature, 1991, 352:624-28 and Marks, et al., J. Mol. Biol., 1991, 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for and F isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites, et al. eds., 8th ed. 1994); and Immunobiology (Janeway, et al. eds., 5^{th} ed. 2001).

The term “core fucose,” “core fucose residue,” “fucose,” or “fucose residue” as used herein refers to a fucose residue in an α1,6-linkage to the first GlcNAc of the Asn-297-linked N-oligosaccharide. Ferrara et al., Proc Natl Acad Sci USA, 2011, 108:12669-74. “Core fucose,” “core fucose residue,” “fucose,” and “fucose residue” are used interchangeably in the present disclosure.

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a R sheet configuration, connected by three hypervariable regions, which form loops connecting, and in some cases form part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in ADCC and CDC. The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat, et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat, et al., supra). The “EU numbering system” or “EU index” is generally used when referring to a residue in an immunoglobulin heavy chain constant region (e.g., the EU index reported in Kabat, et al., supra). The “EU index as in Kabat” refers to the residue numbering of the human IgG1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL j-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat, et al., supra). Chothia refers instead to the location of the structural loops (see, e.g., Chothia and Lesk, J. Mol. Biol., 1987, 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel, eds., 2d ed. 2010)). The “contact” hypervariable regions are based on an analysis of the available complex crystal structures. Another universal numbering system that has been developed and widely adopted is ImMunoGeneTics (IMGT) Information System© (Lafranc, et al., Dev. Comp. Immunol., 2003, 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol., 2001, 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc, et al., supra). The residues from each of these hypervariable regions or CDRs are noted below.

TABLE 1

| Loop | Kabat | AbM | Chothia | Contact | IMGT |
|---|---|---|---|---|---|
| CDR L1 | L24--L34 | L24-- | L24-- | L30-- | L27-- |
| | | L34 | L34 | L36 | L38 |
| CDR L2 | L50--L56 | L50-- | L50-- | L46-- | L56-- |
| | | L56 | L56 | L55 | L65 |
| CDR L3 | L89--L97 | L89-- | L89-- | L89-- | L105- |
| | | L97 | L97 | L96 | L117 |
| CDR | H31--H35B | H26-- | H26-- | H30-- | H27-- |
| H1 | (Kabat | H35B | H32 . . . 34 | H35B | H38 |
| | Numbering) | | | | |
| CDR | H31--H35 | H26-- | H26-- | H30-- | |
| H1 | (Chothia | H35 | H32 | H35 | |
| | Numbering) | | | | |
| CDR | H50--H65 | H50-- | H52-- | H47-- | H56-- |
| H2 | | H58 | H56 | H58 | H65 |
| CDR | H95--H102 | H95-- | H95-- | H93-- | H105- |
| H3 | | H102 | H102 | H101 | H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues. There are typically four FR regions in each of VH and VL regions. The FR regions in VH are VH FR1, VH FR2, VH FR3, and VH FR4 (or FR H1, FR H2, FR H3 and FR H4). The FR regions in VL are VL FR1, VL FR2, VL FR3 and VL FR4 (or FR L1, FR L2, FR L3 and FR L4).

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a CD37 variant may result from one or more (such as, for example, about 1 to about 25, about 1 to about 20, about 1 to about 15, about 1 to about 10, or about 1 to about 5) changes to an amino acid sequence of a native CD37. Also by way of example, a variant of an anti-CD37 antibody may result from one or more (such as, for example, about 1 to about 25, about 1 to about 20, about 1 to about 15, about 1 to about 10, or about 1 to about 5) changes to an amino acid sequence of a native or previously unmodified anti-CD37 antibody. Variants may be naturally occurring, such as allelic or splice variants, or may be artificially constructed. Polypeptide variants may be prepared from the corresponding nucleic acid molecules encoding the variants. In specific embodiments, the CD37 variant or anti-CD37 antibody variant at least retains CD37 or anti-CD37 antibody functional activity, respectively. In specific embodiments, an anti-CD37 antibody variant binds CD37 and/or is antagonistic to CD37 activity. In certain embodiments, the variant is encoded by a single nucleotide polymorphism (SNP) variant of a nucleic acid molecule that encodes CD37 or anti-CD37 antibody VH or VL regions or subregions, such as one or more CDRs.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

As used herein, an “epitope” is a term in the art and refers to a localized region of an antigen to which an antibody can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding an antibody as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide,” “nucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete)), or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain an antibody, for example, in isolated or purified form, together with a suitable amount of excipients.

The term “effective amount” or “therapeutically effective amount” as used herein refers to the amount of an antibody or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular, subcutaneous delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 Fc Mutations with Enhanced Effector Functions

The antibodies provided herein have mutations at the lysine at position 248 (K248) (EU numbering) and the threonine at position 437 (T437) (EU numbering) in the Fc region.

Lysine at position 248 (K248) (EU numbering) and threonine at position 437 (T437) (EU numbering) are both conserved residues in the Fc regions among different IgG subtypes (Zhang, D., et al., supra). Fc mutations, T437R and K248E (EU numbering), were shown to facilitate oligomerization of antibodies upon binding antigens at the cell surface, and possess enhanced effector functions (Zhang, D., et al., supra). T437R and K248E double mutations (“RE mutations”) were shown to confer CDC activity on wildtype IgG1 antibodies that did not possess CDC activity in a dose-dependent manner (Zhang, D., et al., supra).

The “EU numbering” or “EU index” is generally used when referring to a residue in an immunoglobulin heavy chain constant region. It refers to the residue numbering of the human IgG1 EU antibody. It is computed by alignment of an antibody sequence with the Eu antibody sequence (Edelman, G. M., et al., Proc Natl Acad Sci USA, 1969, 63(1):78-85; Kabat, et al., supra), so that each residue that is homologous to a residue in the Eu antibody will have the same residue number as that Eu residue.

Antibodies comprising other Fc region mutations, e.g., S239D/I332E and S239D/I332E/A330L (EU numbering) have also been shown to exhibit significantly enhanced ADCC via enhanced binding to FcγRs (Lazar, G. A. et al., Proc Natl Acad Sci USA, 2006, 103(11):4005-10).

### 5.3 Biparatopic or Bispecific Antibodies

In one aspect, provided herein are biparatopic or bispecific binding molecules (such as biparatopic or bispecific antibodies) that carry RE mutations in the Fc region of the biparatopic or bispecific binding molecules. As demonstrated below, the combination of the biparatopic or bispecific format with the RE mutations further enhances the CDC activity as compared with the RE mutations alone, among other advantages.

CDC activity requires the activation of the complement cascade, which in turn requires activation of complement component C1q by a hexamer of Fc regions co-planar to the surface of the target cell. This in turn requires concurrent binding of five or six antibody molecules in close proximity on the cell surface. Due to the requirement for Fc-mediated antibody oligomerization, CDC activity requires relatively high target receptor densities. With natural IgG antibodies, the stoichiometry of antibody-to-receptor binding can vary from 1:2 to 1:1 depending on antibody concentration, with the former highly favored due to avidity.

To maximize the number of antibodies that can bind a given number of cell surface receptors, the antibodies provided herein are formatted as biparatopic or bispecific antibodies to force 1:1 antibody-to-receptor binding stoichiometry. This allows more Fc regions to be brought to the surface of the cell with a given number of receptors compared to the natural bivalent IgG. The biparatopic or bispecific antibodies provided herein have more potent CDC activity than their natural bivalent counterparts.

Standard techniques known to those of skill in the art can be used to format an antibody as biparatopic or bispecific antibody, including, for example, in vitro expression of recombinant proteins.

Any biparatopic or bispecific antibody format may be applied in the present constructs as long as the format confers further enhanced CDC activity via increased oligomerization (e.g., hexamerization) of the binding molecules at a cell surface and/or increased C1q engagement.

In some embodiment, the biparatopic or bispecific antibody comprises a first antigen binding domain comprising a first VH region and a first VL region, a first single domain antigen binding fragment, and/or a first engineered binding fragment; and a second antigen binding domain comprising a second VH region and a second VL region, a second single domain antigen binding fragment, and/or a second engineered binding fragment; and wherein the first antigen binding domain and the second antigen binding domain bind to two different epitopes of the same antigen or different antigen(s).

In some embodiment, each of the first antigen binding domain and the second antigen binding domain is a Fab fragment, a scFv, a single domain antigen binding fragment, or an engineered binding fragment. In some embodiment, the first antigen binding domain is a Fab fragment and the second antigen binding domain is a scFv. In some embodiment, the first antigen binding domain is a Fab fragment and the second antigen binding domain is a single domain antigen binding fragment. In some embodiment, the first antigen binding domain is a Fab fragment and the second antigen binding domain is an engineered binding fragment. In some embodiment, the first antigen binding domain is a scFv and the second antigen binding domain is a single domain antigen binding fragment. In some embodiment, the first antigen binding domain is a scFv and the second antigen binding domain is an engineered binding fragment. In some embodiment, the first antigen binding domain is a single domain antigen binding fragment and the second antigen binding domain is an engineered binding fragment.

In some embodiment, the biparatopic or bispecific antibody comprises two Fabs and a Fc domain; wherein the first Fab binds to a first epitope of an antigen and the second Fab binds to a second epitope of the antigen, wherein the biparatopic or bispecific antibody comprises two heavy chains and two light chains; wherein the first heavy chain and the first light chain form the first Fab; wherein the second heavy chain and the second light chain form the second Fab; wherein the C-terminus of the two heavy chains form the Fc domain.

In some embodiment, the biparatopic or bispecific antibody comprises two single-chain variable fragments (scFv) and a Fc domain; wherein the first scFv binds to a first epitope of an antigen and the second scFv binds to a second epitope of the antigen, wherein the biparatopic or bispecific antibody comprises two polypeptides; wherein the first polypeptide comprises the first scFv at its N-terminus; wherein the second polypeptide comprises the second scFv at its N-terminus; wherein the C-terminus of the two polypeptides form the Fc domain.

In some embodiment, the biparatopic or bispecific antibody comprises two single domain antigen binding fragments and a Fc domain; wherein the first single domain antigen binding fragment binds to a first epitope of an antigen and the second single domain antigen binding fragment binds to a second epitope of the antigen, wherein the biparatopic or bispecific antibody comprises two polypeptides; wherein the first polypeptide comprises the first single domain antigen binding fragment at its N-terminus; wherein the second polypeptide comprises the second single domain antigen binding fragment at its N-terminus; wherein the C-terminus of the two polypeptides form the Fc domain.

In some embodiment, the biparatopic or bispecific antibody comprises two engineered binding fragments and a Fc domain; wherein the first engineered binding fragment binds to a first epitope of an antigen and the second engineered binding fragment binds to a second epitope of the antigen, wherein the biparatopic or bispecific antibody comprises two polypeptides; wherein the first polypeptide comprises the first engineered binding fragment at its N-terminus; wherein the second polypeptide comprises the second engineered binding fragment at its N-terminus; wherein the C-terminus of the two polypeptides form the Fc domain.

In some embodiment, the biparatopic or bispecific antibody comprises a Fab, a single-chain variable fragment (scFv), and a Fc domain; wherein the Fab binds to a first epitope of an antigen and the scFv binds to a second epitope of the antigen, wherein the biparatopic or bispecific antibody comprises a heavy chain, a light chain, and a polypeptide; wherein the heavy chain and the light chain form the Fab; wherein the polypeptide comprises the scFv at the N-terminus and a domain at the C-terminus that forms the Fc domain with the heavy chain.

In some embodiment, the biparatopic or bispecific antibody comprises a Fab, a single domain antigen binding fragment, and a Fc domain; wherein the Fab binds to a first epitope of an antigen and the single domain antigen binding fragment binds to a second epitope of the antigen, wherein the biparatopic or bispecific antibody comprises a heavy chain, a light chain, and a polypeptide; wherein the heavy chain and the light chain form the Fab; wherein the polypeptide comprises the single domain antigen binding fragment at the N-terminus and a domain at the C-terminus that forms the Fc domain with the heavy chain.

In some embodiment, the biparatopic or bispecific antibody comprises a Fab, an engineered binding fragment, and a Fc domain; wherein the Fab binds to a first epitope of an antigen and the engineered binding fragment binds to a second epitope of the antigen, wherein the biparatopic or bispecific antibody comprises a heavy chain, a light chain, and a polypeptide; wherein the heavy chain and the light chain form the Fab; wherein the polypeptide comprises the engineered binding fragment at the N-terminus and a domain at the C-terminus that forms the Fc domain with the heavy chain.

In some embodiment, the biparatopic or bispecific antibody comprises a single-chain variable fragment (scFv), a single domain antigen binding fragment and a Fc domain; wherein the scFv binds to a first epitope of an antigen and the single domain antigen binding fragment binds to a second epitope of the antigen, wherein the biparatopic or bispecific antibody comprises two polypeptides; wherein the first polypeptide comprises the scFv at its N-terminus; wherein the second polypeptide comprises the single domain antigen binding fragment at its N-terminus; wherein the C-terminus of the two polypeptides form the Fc domain.

In some embodiment, the biparatopic or bispecific antibody comprises a single-chain variable fragment (scFv), an engineered binding fragment and a Fc domain; wherein the scFv binds to a first epitope of an antigen and the engineered binding fragment binds to a second epitope of the antigen, wherein the biparatopic or bispecific antibody comprises two polypeptides; wherein the first polypeptide comprises the scFv at its N-terminus; wherein the second polypeptide comprises the engineered binding fragment at its N-terminus; wherein the C-terminus of the two polypeptides form the Fc domain.

In some embodiment, the biparatopic or bispecific antibody comprises a single domain antigen binding fragment, an engineered binding fragment and a Fc domain; wherein the single domain antigen binding fragment binds to a first epitope of an antigen and the engineered binding fragment binds to a second epitope of the antigen, wherein the biparatopic or bispecific antibody comprises two polypeptides; wherein the first polypeptide comprises the single domain antigen binding fragment at its N-terminus; wherein the second polypeptide comprises the engineered binding fragment at its N-terminus; wherein the C-terminus of the two polypeptides form the Fc domain.

### 5.4 Low Fucosylation

In another aspect, the biparatopic or bispecific binding molecule provided herein does not comprise a fucose in the oligosaccharides covalently attached to the Fc region of the binding molecule. In another aspect, provided herein is a population of biparatopic or bispecific binding molecules wherein less than 80% of the oligosaccharides covalently attached to the Fc region of the binding molecules comprise a fucose.

Antibody glycosylation is a type of posttranslational modification that may occur via the addition of oligosaccharides to antibodies through two types of covalent linkages: linkages on asparagine residues (N-oligosaccharides)s or on serine/threonine residues (O-oligosaccharides) (Alter, G., et al., Semin Immunol., 2018, 39:102-10), and profoundly affect therapeutic functions of antibodies (Walsh, G. and Jefferis, R., Nat. Biotechnol., 2006, 24:1241-52; Jefferis, R., Nat. Rev. Drug Discov., 2009, 8(3):226-34; Dalziel, M., et al., Science, 2014, 343(6166):1235681). Notably, all IgG antibodies are glycosylated in the Fc region thereof on a conserved Asn-297 residue (Alter G., et al., supra).

An Asn-297-linked N-oligosaccharide is comprised of a conserved biantennary core structure (Liu, L., J Pharm Sci., 2015, 104(6):1866-84) consisting of two covalently-linked N-acetylglucosamine (GlcNAc) residues, further linked to a mannose, which links in a 1,3- and 1,6-branching manner to two other mannose residues (Alter, G., et al., supra). Additional monosaccharides, including two galactoses, a fucose, a bisecting GlcNAc, and two sialic acids (Alter, G., et al., supra), may extend the core structure, giving rise to considerable structural and functional heretogeneity (Jefferis, R., Biochem J., 1990, 268(3):529-37; Rudd, P. M., Science, 2001, 291(5512):2370-6; Liu, L., supra). At least 30 structures (glycoforms) for IgG Asn-297-linked N-oligosaccharides have been reported (Alter, G., et al., supra).

Antibodies expressed in mammalian cells are usually more than 80% fucosylated (Kamoda, S., et al., J Chromatogr A., 2004, 1050(2):211-6; Shinkawa, T., et al., J Biol Chem., 2003, 278(5):3466-73). For example, normal Chinese Hamster Ovary (CHO) cells and HEK293 cells add fucose to 80-98% of Asn-297-linked N-oligosaccharides to IgG antibodies (Shields, R. L. et al., J Biol Chem., 2002, 277(30):26733-40).

In one aspect, provided herein is an antibody having no fucose in the oligossacharide attached to its Fc region and having RE mutations in the Fc region. In another aspect, provided herein is a population of antibodies comprising an antibody having no fucose in the oligossacharide attached to its Fc region and having RE mutations in the Fc region. In yet another aspect, provided herein is a population of antibodies wherein less than 80% of the oligosaccharides covalently attached to the population of the antibodies comprise a fucose residue and the Fc region of these antibodies comprises K248E and T437R mutations (RE mutations).

In some embodiments, less than 70% of the oligosaccharides covalently attached to the population of the antibodies comprise a fucose residue. In some embodiment, less than 60% of the oligosaccharides covalently attached to the population of the antibodies comprise a fucose residue. In some embodiments, less than 50% of the oligosaccharides covalently attached to the population of the antibodies comprise a fucose residue. In other embodiments, less than 40% of the oligosaccharides covalently attached to the population of the antibodies comprise a fucose residue. In yet other embodiments, less than 30% of the oligosaccharides covalently attached to the population of the antibodies comprise a fucose residue. In yet other embodiments, less than 20% of the oligosaccharides covalently attached to the population of the antibodies comprise a fucose residue. In yet other embodiments, less than 10% of the oligosaccharides covalently attached to the population of the antibodies comprise a fucose residue.

Standard techniques known to those of skill in the art, e.g., mass spectrometry, can be used to characterize the Asn297-linked N-oligosaccharides on the antibodies (Pereira, N. A., et al., supra; Shields, R. L. et al., supra). For example, in a matrix-assisted laser desorption/ionization time-of-flight mass spectral (MALDI-TOF-MS) analysis, 50 mg of IgG antibodies were immobilized in MultiScreen 96-well IP plates (Millipore) to polyvinylidene difluoride membranes. Proteins were then reduced using 50 mL of a 0.1 M solution of DTT in RCM buffer (pH 8.6, 3.2 mM EDTA, 360 mM Tris, and 8 M urea). Next, they were incubated in the dark for 30 minutes at 25° C. in RCM buffer containing 0.1 M iodoacetic acid, in order to carboxymethylate the free sulfhydryl groups resulting from the reduction step. Membrane-bound proteins were then incubated for 1 hour at 25° C. in a 1% solution of polyvinylpyrrolidone 360 (Sigma) in water, and their oligosaccharides were cleaved from the proteins by a three-step process: incubation for 3 hours at 37° C. in pH 8.4 Tris acetate buffer (25 mL) containing 32 units of peptide:N-glycosidase F (New England Biolabs, Beverly, MA), addition of 1.5 M acetic acid (2.5 mL) to lower the pH, and incubation for 3 hours at 25° C. (Shields, R. L. et al., J Biol Chem., 2001, 276(9):6591-604).

In some embodiments, the antibodies provided herein are produced by expressing a polynucleotide encoding the antibodies or a fragment thereof in a host cell that is deficient in adding a fucose to an oligosaccharide attached to an antibody.

In mammalian cells, FUT8 encodes the only enzyme, α-1,6 fucosyltransferase, that catalyzes core fucosylation, the transfer of a GDP-fucose residue to the innermost GlcNAc via α-1,6-linkage (Imai-Nishiya, H., et al., BMC Biotechnol., 2007, 7:84). Oligosaccharide fucosylation requires intracellular GDP-fucose as substrate, which is synthesized via the de novo pathway or the salvage pathway in the cytoplasm. In the de novo pathway, GDP-mannose 4,6-dehydratase (GMD) mediates the synthesis of GDP-4-keto-6-deoxy-mannose (GKDM) from GDP-mannose, followed by the synthesis of GDP-fucose mediated by GDP-keto-6-deoxymannose 3,5-epimerase, 4-reductase (FX) (Imai-Nishiya, H., et al., supra). As such, cell lines with deficient GMD enzymes, e.g., CHO Lec13 cells, or reduced α-1,6 fucosyltransferase activity resulting from mutated FUT8 genes, have been shown to generate afucosylated antibodies (Pereira, N. A., et al., Mabs, 2018, 10(5):693-711). For example, antibodies with approximately 10% fucosylation (Shields, R. L. et al., supra) or less can be consistently produced in Lec13 cells (Shields, R. L. et al., supra; Kanda Y., Biotechnol Bioeng., 2006, 94(4):680-8), while increased fucosylation may occur when cells are cultured in a static flask to confluence (Pereira, N. A., et al., supra).

The addition of a bisecting GlcNAc to the oligosaccharide core structure creates steric hindrance for fucosylation (Alter, G., et al., supra). As such, overexpression of β-1,4-mannosyl-glycoprotein 4-β-N-acetylglucosaminyltransferase (GnT-III), which catalyzes the addition of a bisecting GlcNAc to the innermost mannose, was shown to dramatically reduce Fc fucosylation (Pereira, N. A., et al., supra).

Moreover, inactivated Golgi GDP-fucose transporter (GFT) gene (Slc35c1) has been shown to produce afucosylated antibodies, e.g., in CHO-gmt3 cells (Pereira, N. A., et al., supra). Use of biochemical inhibitors of fucosylation, e.g., fucose analogs such as 2-fluorofucose and 5-alkynylfucose, can also generate afucosylated antibodies (Pereira, N. A., et al., supra). The intermediate GKDM in the de novo fucose synthesis pathway in mammalian cells can be reduced by bacteria GDP-4-keto-6-deoxy mannose reductase (RMD) to GDP-rhamnose, thus bypassing the fucose biosynthesis pathway. Afucosylated antibodies can also be generated in cells in which bacterial RMD is heterologously expressed in the cytosol (Pereira, N. A., et al., supra).

In some embodiments, the antibodies provided herein are produced by expressing the antibodies in a host cell having a deficiency in any of the above mentioned enzymes. In some embodiments, the host cell has reduced GDP-mannose 4,6-dehydratase (GMD) activity. In some embodiments, the host cell has reduced α-1,6 fucosyltransferase activity.

### 5.5 ADCC, CDC, and ADCP Effector Functions

The antibody or population of antibodies provided herein has enhanced ADCC activity, enhanced CDC activity, and/or enhanced ADCP activity.

Therapeutic antibodies bind Fc receptors on the cell surface of effector cells, such as NK cells, macrophages, mononuclear phagocytes, neutrophils and eosinophils (Saunder, K. O., Front Immunol., 2019, 10:1296), giving rise to important antibody-dependent effector functions, such as ADCC and ADCP. A family of receptors for IgG Fc regions was referred to as the Fc7 receptors (FcγRs) (Cohen-Solal, J. F., Immunol Lett., 2004, 92(3):199-205), and is comprised of FcγRI; FcγRII, including isoforms FcγRIIa, FcγRIIb, and FcγRIIc; and FcγRIII, including isoforms FcγRIIIa and FcγRIIIb (Jefferis, R. and Lund, J., Immunol Lett., 2002, 82(1-2):57-65).

Among various effector functions, ADCC and ADCP have been shown to possess clinically significant anti-tumor efficacy. For example, ADCC was shown to be an important mechanism for the anti-tumor efficacy of trastuzumab in vitro, as evidenced by NK cells' capability to kill trastuzumab-coated tumor cells via a FcγRIII receptor (CD16)-mediated ADCC mechanism (Cooley, S., et al., Exp Hematol., 1999; 27(10):1533-41; Carson, W. E., et al., Eur J Immunol., 2001, 31(10):3016-3025; Kubo, M., et al., Anticancer Res., 2003, 23(6a):4443-9) and in vivo, as evidenced by increased numbers of NK cells in tumor infiltrates after trastuzumab treatment (Clynes, R. A., et al., Nat Med., 2000, 6(4):443-6; Arnould, L., et al., Br J Cancer, 2006, 94(2):259-67). Additionally, macrophage-mediated ADCP has been shown to be important in the anti-tumor efficacy of trastuzumab (Shi, Y., et al., J Immunol., 2015, 194(9):4379-86).

Antibodies with no or low fucosylation have shown dramatically enhanced ADCC activity due to the enhancement in their binding capacity to FcγRIIIa binding without any detectable change in CDC or antigen binding capability (Okazaki, A., et al., J Mol Biol., 2004, 336(5):1239-49; Kanda, Y., et al., Glycobiology, 2007, 17(1):104-18). N-oligosaccharides of antibody Fc regions are essential for binding to FcγR, which engages antibody effector functions (Yamane-Ohnuki, N. and Satoh, M., Mabs, 2009, 1(3):230-6).

The absence of fucose on N-oligosaccharides of antibody Fc regions have been shown to dramatically enhance antibodies' binding capacity to FcγRIIIa receptors present on immune effector cells such as NK cells and macrophages, giving rise to anti-tumor therapeutic effect (Pereira, N. A., et al., supra). The FcγRIIIa receptors bind Fc regions via interactions with the hinge region and the CH2 domain of the Fc (Radaev, S., et al., J Biol Chem., 2001, 276:16469-77; Sondermann, P., et al., Nature, 2000, 406:267-73). The absence of fucose thus eliminates the steric hindrance and enhances the Fc-FcγRIIIa interaction, leading to enhanced effector functions (Pereira, N. A., et al., supra).

CDC is another important antibody effector function. In the antibody-dependent classical complement activation pathway, binding between the complement C1q heterohexameric headpiece and an oligomeric antibody complex initiates the proteolytic complement cascade (Wang, G. et al., Mol Cell, 2016, 63:135-45; Diebolder, C. A. et al., Science, 2014, 343:1260-3), which leads to the opsonization of target cells by C3-derived opsonins (e.g., C3b) and generation of potent inflammation mediators (C3a and C5a), ultimately resulting in the formation of membrane attack complex (MAC), C5b-C9, on the target cell membrane (Reis, E. S., et al., Nat Rev Immunol., 2018, 18:5-18). CDC has also been shown to possess clinically significant anti-tumor efficacy, e.g., in the anti-CD20 mAb rituximab and anti-CD38 mAb daratumumab (de Weers, M., et al., J Immunol., 2011, 186:1840-8; Lokhorst, H. M., et al., N Engl J Med., 2015, 373:1207-19; Taylor, R. P. and Lindorfer, M. A., Semin Immunol., 2016, 28:309-16).

Mutations in the Fc region that facilitate antibody oligomerization, such as the RE mutations and E345R (EU numbering), have been demonstrated to significantly enhance antibody CDC activity (Diebolder, C. A. et al., supra; Zhang, D., et al., supra).

Biparatopic or bispecific antibodies disclosed herein allows more Fc regions to be brought to the surface of the cell with a given number of receptors compared to the natural bivalent IgG. Thus, engineered biparatopic or bispecific antibodies have more potent CDC activity.

In some embodiments, the ADCC activity of the present antibodies is 10% higher than antibodies with normal fucosylation. In some embodiments, the ADCC activity of the present antibodies is 20% higher than antibodies with normal fucosylation. In some embodiments, the ADCC activity of the present antibodies is 30% higher than antibodies with normal fucosylation. In some embodiments, the ADCC activity of the present antibodies is 40% higher than antibodies with normal fucosylation. In some embodiments, the ADCC activity of the present antibodies is 50% higher than antibodies with normal fucosylation. In some embodiments, the ADCC activity of the present antibodies is 60% higher than antibodies with normal fucosylation. In some embodiments, the ADCC activity of the present antibodies is 70% higher than antibodies with normal fucosylation. In some embodiments, the ADCC activity of the present antibodies is 80% higher than antibodies with normal fucosylation. In some embodiments, the ADCC activity of the present antibodies is 90% higher than antibodies with normal fucosylation. In some embodiments, the ADCC activity of the present antibodies is more than 2 fold of that of antibodies with normal fucosylation. In some embodiments, the ADCC activity of the present antibodies is more than 3 fold of that of antibodies with normal fucosylation. In some embodiments, the ADCC activity of the present antibodies is more than 4 fold of that of antibodies with normal fucosylation. In some embodiments, the ADCC activity of the present antibodies is more than 5 fold of that of antibodies with normal fucosylation. In some embodiments, the ADCC activity of the present antibodies is more than 6 fold of that of antibodies with normal fucosylation. In some embodiments, the ADCC activity of the present antibodies is more than 7 fold of that of antibodies with normal fucosylation. In some embodiments, the ADCC activity of the present antibodies is more than 8 fold of that of antibodies with normal fucosylation. In some embodiments, the ADCC activity of the present antibodies is more than 9 fold of that of antibodies with normal fucosylation. In some embodiments, the ADCC activity of the present antibodies is more than 10 fold of that of antibodies with normal fucosylation.

In some embodiments, the antibodies described above also have higher CDC activities. In some embodiments, the CDC activity of the present antibodies is 10% higher than antibodies without RE mutations. In some embodiments, the CDC activity of the present antibodies is 20% higher than antibodies without RE mutations. In some embodiments, the CDC activity of the present antibodies is 30% higher than antibodies without RE mutations. In some embodiments, the CDC activity of the present antibodies is 40% higher than antibodies without RE mutations. In some embodiments, the CDC activity of the present antibodies is 50% higher than antibodies without RE mutations. In some embodiments, the CDC activity of the present antibodies is 60% higher than antibodies without RE mutations. In some embodiments, the CDC activity of the present antibodies is 70% higher than antibodies without RE mutations. In some embodiments, the CDC activity of the present antibodies is 80% higher than antibodies without RE mutations. In some embodiments, the CDC activity of the present antibodies is 90% higher than antibodies without RE mutations. In some embodiments, the CDC activity of the present antibodies is more than 2 fold of that of antibodies without RE mutations. In some embodiments, the CDC activity of the present antibodies is more than 3 fold of that of antibodies without RE mutations. In some embodiments, the CDC activity of the present antibodies is more than 4 fold of that of antibodies without RE mutations. In some embodiments, the CDC activity of the present antibodies is more than 5 fold of that of antibodies without RE mutations. In some embodiments, the CDC activity of the present antibodies is more than 6 fold of that of antibodies without RE mutations. In some embodiments, the CDC activity of the present antibodies is more than 7 fold of that of antibodies without RE mutations. In some embodiments, the CDC activity of the present antibodies is more than 8 fold of that of antibodies without RE mutations. In some embodiments, the CDC activity of the present antibodies is more than 9 fold of that of antibodies without RE mutations. In some embodiments, the CDC activity of the present antibodies is more than 10 fold of that of antibodies without RE mutations.

In some embodiments, the antibodies described above also have higher CDC activities. In some embodiments, the CDC activity of the present antibodies is 10% higher than antibodies without biparatopic or bispecific formatting. In some embodiments, the CDC activity of the present antibodies is 20% higher than antibodies without biparatopic or bispecific formatting. In some embodiments, the CDC activity of the present antibodies is 30% higher than antibodies without biparatopic or bispecific formatting. In some embodiments, the CDC activity of the present antibodies is 40% higher than antibodies without biparatopic or bispecific formatting. In some embodiments, the CDC activity of the present antibodies is 50% higher than antibodies without biparatopic or bispecific formatting. In some embodiments, the CDC activity of the present antibodies is 60% higher than antibodies without biparatopic or bispecific formatting. In some embodiments, the CDC activity of the present antibodies is 70% higher than antibodies without biparatopic or bispecific formatting. In some embodiments, the CDC activity of the present antibodies is 80% higher than antibodies without biparatopic or bispecific formatting. In some embodiments, the CDC activity of the present antibodies is 90% higher than antibodies without biparatopic or bispecific formatting. In some embodiments, the CDC activity of the present antibodies is more than 2 fold of that of antibodies without biparatopic or bispecific formatting. In some embodiments, the CDC activity of the present antibodies is more than 3 fold of that of antibodies without biparatopic or bispecific formatting. In some embodiments, the CDC activity of the present antibodies is more than 4 fold of that of antibodies without biparatopic or bispecific formatting. In some embodiments, the CDC activity of the present antibodies is more than 5 fold of that of antibodies without biparatopic or bispecific formatting. In some embodiments, the CDC activity of the present antibodies is more than 6 fold of that of antibodies without biparatopic or bispecific formatting. In some embodiments, the CDC activity of the present antibodies is more than 7 fold of that of antibodies without biparatopic or bispecific formatting. In some embodiments, the CDC activity of the present antibodies is more than 8 fold of that of antibodies without biparatopic or bispecific formatting. In some embodiments, the CDC activity of the present antibodies is more than 9 fold of that of antibodies without biparatopic or bispecific formatting. In some embodiments, the CDC activity of the present antibodies is more than 10 fold of that of antibodies without biparatopic or bispecific formatting.

### 5.6 Exemplary Biparatopic or Bispecific Antibodies

In one aspect, provided herein are biparatopic or bispecific antibodies derived from one or more conventional bivalent antibodies.

In yet another aspect, provided herein are biparatopic or bispecific antibodies that compete with one of the known biparatopic or bispecific antibodies. Such biparatopic or bispecific antibodies may bind to the same epitope as one of the known biparatopic or bispecific antibodies, or an overlapping epitope. Antibodies that compete with or bind to the same epitope are expected to show similar functional properties.

In certain embodiments, a biparatopic or bispecific antibody described herein comprises amino acid sequences with certain percent identity relative to one of the known biparatopic or bispecific antibodies or the exemplary biparatopic or bispecific antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A. 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, provided herein is a biparatopic or bispecific antibody comprising an Fc region that has RE mutations but no fucose residue.

Standard techniques known to those of skill in the art can be used to introduce mutations in the nucleotide sequence encoding a biparatopic or bispecific provided herein, including, for example, site-directed mutagenesis and PCR-mediated mutagenesis which results in amino acid mutations.

In some embodiments, glutamic acid is substituted for lysine at position 248 (K248E) (EU numbering) of the Fc region of the biparatopic or bispecific antibody provided herein. In some embodiments, glutamic acid is substituted for alanine at position 338 (K338A) (EU numbering) of the Fc region of the biparatopic or bispecific antibody provided herein. In some embodiments, arginine is substituted for threonine at position 437 (T437R) (EU numbering) of the Fc region of the biparatopic or bispecific antibody provided herein. In some embodiments, glutamic acid is substituted for lysine at position 248, and arginine is substituted for threonine at position 437 (K248E/T437R) (EU numbering), of the Fc region of the biparatopic or bispecific antibody provided herein. In some embodiments, glutamic acid is substituted for alanine at position 338, and arginine is substituted for threonine at position 437 (K248E/T437R) (EU numbering), of the Fc region of the biparatopic or bispecific antibody provided herein.

In some embodiments, the biparatopic or bispecific antibody provided herein comprises a heavy chain comprising the T437R mutation. In some embodiments, the biparatopic or bispecific antibody provided herein comprises a heavy chain comprising the K248E mutation. In some embodiments, the biparatopic or bispecific antibody provided herein comprises a heavy chain comprising the K338A mutation. In some embodiments, the biparatopic or bispecific antibody provided herein comprises a heavy chain comprising the K248E/T437R mutation. In some embodiments, the biparatopic or bispecific antibody provided herein comprises a heavy chain comprising the K338A/T437R mutation.

Standard techniques known to those of skill in the art can be used to generate a biparatopic or bispecific antibody provided herein with a heavy chain comprising no fucose residue on the Asn-297-linked N-oligosaccharide thereof. For example, mammalian cell lines with deficient GMD enzymes (e.g., CHO Lec13 cells), with reduced α-1,6 fucosyltransferase activity resulting from mutated FUT8 genes, with overexpression of β-1,4-mannosyl-glycoprotein 4-β-N-acetylglucosaminyltransferase (GnT-III), with inactivated Golgi GDP-fucose transporter (GFT) gene Slc35c1 (e.g., CHO-gmt3 cells), with heterologous expression of bacterial RMD, or with use of biochemical inhibitors of fucosylation (e.g., fucose analogs such as 2-fluorofucose and 5-alkynylfucose) have been shown to produce antibodies with no fucose residue on the Asn-297-linked N-oligosaccharide on the heavy chain (Pereira, N. A., et al., supra; Shields, R. L. et al., supra; Kanda Y., supra).

In some embodiments, the biparatopic or bispecific antibody comprises a heavy chain comprising no fucose residue on the Asn-297-linked N-oligosaccharide thereof.

Standard techniques known to those of skill in the art can be used to format an antibody as biparatopic or bispecific antibody, including, for example, in vitro expression of recombinant proteins.

In some embodiment, the biparatopic or bispecific antibody comprises a first antigen binding domain comprising a first VH region and a first VL region, a first single domain antigen binding fragment, and/or a first engineered binding fragment; and a second antigen binding domain comprising a second VH region and a second VL region, a second single domain antigen binding fragment, and/or a second engineered binding fragment; and wherein the first antigen binding domain and the second antigen binding domain bind to two different epitopes of the same antigen or different antigen(s).

In some embodiment, each of the first antigen binding domain and the second antigen binding domain is a Fab fragment, a scFv, a single domain antigen binding fragment, or an engineered binding fragment. In some embodiment, the first antigen binding domain is a Fab fragment and the second antigen binding domain is a scFv. In some embodiment, the first antigen binding domain is a Fab fragment and the second antigen binding domain is a single domain antigen binding fragment. In some embodiment, the first antigen binding domain is a Fab fragment and the second antigen binding domain is an engineered binding fragment. In some embodiment, the first antigen binding domain is a scFv and the second antigen binding domain is a single domain antigen binding fragment. In some embodiment, the first antigen binding domain is a scFv and the second antigen binding domain is an engineered binding fragment. In some embodiment, the first antigen binding domain is a single domain antigen binding fragment and the second antigen binding domain is an engineered binding fragment.

In some embodiment, the biparatopic or bispecific antibody comprises two Fabs and a Fc domain; wherein the first Fab binds to a first epitope of an antigen and the second Fab binds to a second epitope of the antigen, wherein the biparatopic or bispecific antibody comprises two heavy chains and two light chains; wherein the first heavy chain and the first light chain form the first Fab; wherein the second heavy chain and the second light chain form the second Fab; wherein the C-terminus of the two heavy chains form the Fc domain.

In some embodiment, the biparatopic or bispecific antibody comprises two single-chain variable fragments (scFv) and a Fc domain; wherein the first scFv binds to a first epitope of an antigen and the second scFv binds to a second epitope of the antigen, wherein the biparatopic or bispecific antibody comprises two polypeptides; wherein the first polypeptide comprises the first scFv at its N-terminus; wherein the second polypeptide comprises the second scFv at its N-terminus; wherein the C-terminus of the two polypeptides form the Fc domain.

In some embodiment, the biparatopic or bispecific antibody comprises two single domain antigen binding fragments and a Fc domain; wherein the first single domain antigen binding fragment binds to a first epitope of an antigen and the second single domain antigen binding fragment binds to a second epitope of the antigen, wherein the biparatopic or bispecific antibody comprises two polypeptides; wherein the first polypeptide comprises the first single domain antigen binding fragment at its N-terminus; wherein the second polypeptide comprises the second single domain antigen binding fragment at its N-terminus; wherein the C-terminus of the two polypeptides form the Fc domain.

In some embodiment, the biparatopic or bispecific antibody comprises two engineered binding fragments and a Fc domain; wherein the first engineered binding fragment binds to a first epitope of an antigen and the second engineered binding fragment binds to a second epitope of the antigen, wherein the biparatopic or bispecific antibody comprises two polypeptides; wherein the first polypeptide comprises the first engineered binding fragment at its N-terminus; wherein the second polypeptide comprises the second engineered binding fragment at its N-terminus; wherein the C-terminus of the two polypeptides form the Fc domain.

In some embodiment, the biparatopic or bispecific antibody comprises a Fab, a single-chain variable fragment (scFv), and a Fc domain; wherein the Fab binds to a first epitope of an antigen and the scFv binds to a second epitope of the antigen, wherein the biparatopic or bispecific antibody comprises a heavy chain, a light chain, and a polypeptide; wherein the heavy chain and the light chain form the Fab; wherein the polypeptide comprises the scFv at the N-terminus and a domain at the C-terminus that forms the Fc domain with the heavy chain.

In some embodiment, the biparatopic or bispecific antibody comprises a Fab, a single domain antigen binding fragment, and a Fc domain; wherein the Fab binds to a first epitope of an antigen and the single domain antigen binding fragment binds to a second epitope of the antigen, wherein the biparatopic or bispecific antibody comprises a heavy chain, a light chain, and a polypeptide; wherein the heavy chain and the light chain form the Fab; wherein the polypeptide comprises the single domain antigen binding fragment at the N-terminus and a domain at the C-terminus that forms the Fc domain with the heavy chain.

In some embodiment, the biparatopic or bispecific antibody comprises a Fab, an engineered binding fragment, and a Fc domain; wherein the Fab binds to a first epitope of an antigen and the engineered binding fragment binds to a second epitope of the antigen, wherein the biparatopic or bispecific antibody comprises a heavy chain, a light chain, and a polypeptide; wherein the heavy chain and the light chain form the Fab; wherein the polypeptide comprises the engineered binding fragment at the N-terminus and a domain at the C-terminus that forms the Fc domain with the heavy chain.

In some embodiment, the biparatopic or bispecific antibody comprises a single-chain variable fragment (scFv), a single domain antigen binding fragment and a Fc domain; wherein the scFv binds to a first epitope of an antigen and the single domain antigen binding fragment binds to a second epitope of the antigen, wherein the biparatopic or bispecific antibody comprises two polypeptides; wherein the first polypeptide comprises the scFv at its N-terminus; wherein the second polypeptide comprises the single domain antigen binding fragment at its N-terminus; wherein the C-terminus of the two polypeptides form the Fc domain.

In some embodiment, the biparatopic or bispecific antibody comprises a single-chain variable fragment (scFv), an engineered binding fragment and a Fc domain; wherein the scFv binds to a first epitope of an antigen and the engineered binding fragment binds to a second epitope of the antigen, wherein the biparatopic or bispecific antibody comprises two polypeptides; wherein the first polypeptide comprises the scFv at its N-terminus; wherein the second polypeptide comprises the engineered binding fragment at its N-terminus; wherein the C-terminus of the two polypeptides form the Fc domain.

In some embodiment, the biparatopic or bispecific antibody comprises a single domain antigen binding fragment, an engineered binding fragment and a Fc domain; wherein the single domain antigen binding fragment binds to a first epitope of an antigen and the engineered binding fragment binds to a second epitope of the antigen, wherein the biparatopic or bispecific antibody comprises two polypeptides; wherein the first polypeptide comprises the single domain antigen binding fragment at its N-terminus; wherein the second polypeptide comprises the engineered binding fragment at its N-terminus; wherein the C-terminus of the two polypeptides form the Fc domain.

In some embodiments, the target cell is a cancer cell, e.g., a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer. In some embodiments, the cancer is an anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma. In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor, is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

In some embodiments, one antigen bound by the biparatopic or bispecific antibody is a cancer antigen. In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R. In some embodiments, the cancer antigen is angiopoietin. In some embodiments, the cancer antigen is BCMA. In some embodiments, the cancer antigen is CD19. In some embodiments, the cancer antigen is CD20. In some embodiments, the cancer antigen is CD22. In some embodiments, the cancer antigen is CD25 (IL2-R). In some embodiments, the cancer antigen is CD30. In some embodiments, the cancer antigen is CD33. In some embodiments, the cancer antigen is CD37. In some embodiments, the cancer antigen is CD38. In some embodiments, the cancer antigen is CD52. In some embodiments, the cancer antigen is CD56. In some embodiments, the cancer antigen is CD123 (IL-3R). In some embodiments, the cancer antigen is cMET. In some embodiments, the cancer antigen is DLL/Notch. In some embodiments, the cancer antigen is EGFR. In some embodiments, the cancer antigen is EpCAM. In some embodiments, the cancer antigen is FGF. In some embodiments, the cancer antigen is FGF-R. In some embodiments, the cancer antigen is GD2. In some embodiments, the cancer antigen is HER2. In some embodiments, the cancer antigen is Mesothelin. In some embodiments, the cancer antigen is Nectin-4. In some embodiments, the cancer antigen is PDGFRα. In some embodiments, the cancer antigen is RANKL. In some embodiments, the cancer antigen is SLAMF7. In some embodiments, the cancer antigen is TROP2. In some embodiments, the cancer antigen is VEGF. In some embodiments, the cancer antigen is VEGF-R.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1. In some embodiments, the cancer antigen is CEA. In some embodiments, the cancer antigen is immature laminin receptor. In some embodiments, the cancer antigen is TAG-72. In some embodiments, the cancer antigen is HPV E6. In some embodiments, the cancer antigen is HPV E7. In some embodiments, the cancer antigen is BING-4. In some embodiments, the cancer antigen is calcium-activated chloride channel 2. In some embodiments, the cancer antigen is cyclin-B1. In some embodiments, the cancer antigen is 9D7. In some embodiments, the cancer antigen is EpCAM. In some embodiments, the cancer antigen is EphA3. In some embodiments, the cancer antigen is Her2/neu. In some embodiments, the cancer antigen is telomerase. In some embodiments, the cancer antigen is mesothelin. In some embodiments, the cancer antigen is SAP-1. In some embodiments, the cancer antigen is surviving. In some embodiments, the cancer antigen is a BAGE family antigen. In some embodiments, the cancer antigen is CAGE family antigen. In some embodiments, the cancer antigen is GAGE family antigen. In some embodiments, the cancer antigen is MAGE family antigen. In some embodiments, the cancer antigen is SAGE family antigen. In some embodiments, the cancer antigen is XAGE family antigen. In some embodiments, the cancer antigen is NY-ESO-1/LAGE-1. In some embodiments, the cancer antigen is PRAME. In some embodiments, the cancer antigen is SSX-2. In some embodiments, the cancer antigen is Melan-A. In some embodiments, the cancer antigen is MART-1. In some embodiments, the cancer antigen is Gp100. In some embodiments, the cancer antigen is pmel17. In some embodiments, the cancer antigen is tyrosinase. In some embodiments, the cancer antigen is TRP-1. In some embodiments, the cancer antigen is TRP-2. In some embodiments, the cancer antigen is P. polypeptide. In some embodiments, the cancer antigen is MC1R. In some embodiments, the cancer antigen is prostate-specific antigen. In some embodiments, the cancer antigen is β-catenin. In some embodiments, the cancer antigen is BRCA1. In some embodiments, the cancer antigen is BRCA2. In some embodiments, the cancer antigen is CDK4. In some embodiments, the cancer antigen is CML66. In some embodiments, the cancer antigen is fibronectin. In some embodiments, the cancer antigen is MART-2. In some embodiments, the cancer antigen is p53. In some embodiments, the cancer antigen is Ras. In some embodiments, the cancer antigen is TGF-βRII. In some embodiments, the cancer antigen is MUC1.

In some embodiments, the biparatopic or bispecific antibody binds to a B cell antigen. In some embodiments, the B cell antigen is a CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267, CD268, CD269, CD270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362, or CD363 antigen. In some embodiments, the B cell antigen is a CD1a antigen. In some embodiments, the B cell antigen is a CD1b antigen. In some embodiments, the B cell antigen is a CD1c antigen. In some embodiments, the B cell antigen is a CD1d antigen. In some embodiments, the B cell antigen is a CD2 antigen. In some embodiments, the B cell antigen is a CD5 antigen. In some embodiments, the B cell antigen is a CD6 antigen. In some embodiments, the B cell antigen is a CD9 antigen. In some embodiments, the B cell antigen is a CD11a antigen. In some embodiments, the B cell antigen is a CD11b antigen. In some embodiments, the B cell antigen is a CD11c antigen. In some embodiments, the B cell antigen is a CD17 antigen. In some embodiments, the B cell antigen is a CD18 antigen. In some embodiments, the B cell antigen is a CD19 antigen. In some embodiments, the B cell antigen is a CD20 antigen. In some embodiments, the B cell antigen is a CD21 antigen. In some embodiments, the B cell antigen is a CD22 antigen. In some embodiments, the B cell antigen is a CD23 antigen. In some embodiments, the B cell antigen is a CD24 antigen. In some embodiments, the B cell antigen is a CD25 antigen. In some embodiments, the B cell antigen is a CD26 antigen. In some embodiments, the B cell antigen is a CD27 antigen. In some embodiments, the B cell antigen is a CD29 antigen. In some embodiments, the B cell antigen is a CD30 antigen. In some embodiments, the B cell antigen is a CD31 antigen. In some embodiments, the B cell antigen is a CD32a antigen. In some embodiments, the B cell antigen is a CD32b antigen. In some embodiments, the B cell antigen is a CD35 antigen. In some embodiments, the B cell antigen is a CD37 antigen. In some embodiments, the B cell antigen is a CD38 antigen. In some embodiments, the B cell antigen is a CD39 antigen. In some embodiments, the B cell antigen is a CD40 antigen. In some embodiments, the B cell antigen is a CD45 antigen. In some embodiments, the B cell antigen is a CD45RA antigen. In some embodiments, the B cell antigen is a CD45RB antigen. In some embodiments, the B cell antigen is a CD45RC antigen. In some embodiments, the B cell antigen is a CD45RO antigen. In some embodiments, the B cell antigen is a CD46 antigen. In some embodiments, the B cell antigen is a CD47 antigen. In some embodiments, the B cell antigen is a CD48 antigen. In some embodiments, the B cell antigen is a CD49b antigen. In some embodiments, the B cell antigen is a CD49c antigen. In some embodiments, the B cell antigen is a CD49d antigen. In some embodiments, the B cell antigen is a CD50 antigen. In some embodiments, the B cell antigen is a CD52 antigen. In some embodiments, the B cell antigen is a CD53 antigen. In some embodiments, the B cell antigen is a CD54 antigen. In some embodiments, the B cell antigen is a CD55 antigen. In some embodiments, the B cell antigen is a CD58 antigen. In some embodiments, the B cell antigen is a CD60a antigen. In some embodiments, the B cell antigen is a CD62L antigen. In some embodiments, the B cell antigen is a CD63 antigen. In some embodiments, the B cell antigen is a CD68 antigen. In some embodiments, the B cell antigen is a CD69 antigen. In some embodiments, the B cell antigen is a CD70 antigen. In some embodiments, the B cell antigen is a CD72 antigen. In some embodiments, the B cell antigen is a CD73 antigen. In some embodiments, the B cell antigen is a CD74 antigen. In some embodiments, the B cell antigen is a CD75 antigen. In some embodiments, the B cell antigen is a CD75S antigen. In some embodiments, the B cell antigen is a CD77 antigen. In some embodiments, the B cell antigen is a CD79a antigen. In some embodiments, the B cell antigen is a CD79b antigen. In some embodiments, the B cell antigen is a CD80 antigen. In some embodiments, the B cell antigen is a CD81 antigen. In some embodiments, the B cell antigen is a CD82 antigen. In some embodiments, the B cell antigen is a CD83 antigen. In some embodiments, the B cell antigen is a CD84 antigen. In some embodiments, the B cell antigen is a CD85E antigen. In some embodiments, the B cell antigen is a CD85I antigen. In some embodiments, the B cell antigen is a CD85J antigen. In some embodiments, the B cell antigen is a CD86 antigen. In some embodiments, the B cell antigen is a CD92 antigen. In some embodiments, the B cell antigen is a CD95 antigen. In some embodiments, the B cell antigen is a CD97 antigen. In some embodiments, the B cell antigen is a CD98 antigen. In some embodiments, the B cell antigen is a CD99 antigen. In some embodiments, the B cell antigen is a CD100 antigen. In some embodiments, the B cell antigen is a CD102 antigen. In some embodiments, the B cell antigen is a CD108 antigen. In some embodiments, the B cell antigen is a CD119 antigen. In some embodiments, the B cell antigen is a CD120a antigen. In some embodiments, the B cell antigen is a CD120b antigen. In some embodiments, the B cell antigen is a CD121b antigen. In some embodiments, the B cell antigen is a CD122 antigen. In some embodiments, the B cell antigen is a CD124 antigen. In some embodiments, the B cell antigen is a CD125 antigen. In some embodiments, the B cell antigen is a CD126 antigen. In some embodiments, the B cell antigen is a CD130 antigen. In some embodiments, the B cell antigen is a CD132 antigen. In some embodiments, the B cell antigen is a CD137 antigen. In some embodiments, the B cell antigen is a CD138 antigen. In some embodiments, the B cell antigen is a CD139 antigen. In some embodiments, the B cell antigen is a CD147 antigen. In some embodiments, the B cell antigen is a CD148 antigen. In some embodiments, the B cell antigen is a CD150 antigen. In some embodiments, the B cell antigen is a CD152 antigen. In some embodiments, the B cell antigen is a CD162 antigen. In some embodiments, the B cell antigen is a CD164 antigen. In some embodiments, the B cell antigen is a CD166 antigen. In some embodiments, the B cell antigen is a CD167a antigen. In some embodiments, the B cell antigen is a CD170 antigen. In some embodiments, the B cell antigen is a CD171 antigen. In some embodiments, the B cell antigen is a CD175 antigen. In some embodiments, the B cell antigen is a CD175s antigen. In some embodiments, the B cell antigen is a CD180 antigen. In some embodiments, the B cell antigen is a CD184 antigen. In some embodiments, the B cell antigen is a CD185 antigen. In some embodiments, the B cell antigen is a CD192 antigen. In some embodiments, the B cell antigen is a CD196 antigen. In some embodiments, the B cell antigen is a CD197 antigen. In some embodiments, the B cell antigen is a CD200 antigen. In some embodiments, the B cell antigen is a CD205 antigen. In some embodiments, the B cell antigen is a CD201a antigen. In some embodiments, the B cell antigen is a CDw210b antigen. In some embodiments, the B cell antigen is a CD212 antigen. In some embodiments, the B cell antigen is a CD213a1 antigen. In some embodiments, the B cell antigen is a CD213a2 antigen. In some embodiments, the B cell antigen is a CD 215 antigen. In some embodiments, the B cell antigen is a CD217 antigen. In some embodiments, the B cell antigen is a CD218a antigen. In some embodiments, the B cell antigen is a CD218b antigen. In some embodiments, the B cell antigen is a CD220 antigen. In some embodiments, the B cell antigen is a CD221 antigen. In some embodiments, the B cell antigen is a CD222 antigen. In some embodiments, the B cell antigen is a CD224 antigen. In some embodiments, the B cell antigen is a CD225 antigen. In some embodiments, the B cell antigen is a CD226 antigen. In some embodiments, the B cell antigen is a CD227 antigen. In some embodiments, the B cell antigen is a CD229 antigen. In some embodiments, the B cell antigen is a CD230 antigen. In some embodiments, the B cell antigen is a CD232 antigen. In some embodiments, the B cell antigen is a CD252 antigen. In some embodiments, the B cell antigen is a CD252 antigen. In some embodiments, the B cell antigen is a CD254 antigen. In some embodiments, the B cell antigen is a CD255 antigen. In some embodiments, the B cell antigen is a CD256 antigen. In some embodiments, the B cell antigen is a CD257 CD258 antigen. In some embodiments, the B cell antigen is a CD259 antigen. In some embodiments, the B cell antigen is a CD260 antigen. In some embodiments, the B cell antigen is a CD261 antigen. In some embodiments, the B cell antigen is a CD262 antigen. In some embodiments, the B cell antigen is a CD263 antigen. In some embodiments, the B cell antigen is a CD264 antigen. In some embodiments, the B cell antigen is a CD267 antigen. In some embodiments, the B cell antigen is a CD268 antigen. In some embodiments, the B cell antigen is a CD269 antigen. In some embodiments, the B cell antigen is a CD270 antigen. In some embodiments, the B cell antigen is a CD272 antigen. In some embodiments, the B cell antigen is a CD274 antigen. In some embodiments, the B cell antigen is a CD275 antigen. In some embodiments, the B cell antigen is a CD277 antigen. In some embodiments, the B cell antigen is a CD279 antigen. In some embodiments, the B cell antigen is a CD283 antigen. In some embodiments, the B cell antigen is a CD289 antigen. In some embodiments, the B cell antigen is a CD290 antigen. In some embodiments, the B cell antigen is a CD295 antigen. In some embodiments, the B cell antigen is a CD298 antigen. In some embodiments, the B cell antigen is a CD300 antigen. In some embodiments, the B cell antigen is a CD300c antigen. In some embodiments, the B cell antigen is a CD305 antigen. In some embodiments, the B cell antigen is a CD306 antigen. In some embodiments, the B cell antigen is a CD307a antigen. In some embodiments, the B cell antigen is a CD307b antigen. In some embodiments, the B cell antigen is a CD307c antigen. In some embodiments, the B cell antigen is a CD307d antigen. In some embodiments, the B cell antigen is a CD307e antigen. In some embodiments, the B cell antigen is a CD314 antigen. In some embodiments, the B cell antigen is a CD215 antigen. In some embodiments, the B cell antigen is a CD316 antigen. In some embodiments, the B cell antigen is a CD317 antigen. In some embodiments, the B cell antigen is a CD319 antigen. In some embodiments, the B cell antigen is a CD321 antigen. In some embodiments, the B cell antigen is a CD327 antigen. In some embodiments, the B cell antigen is a CD328 antigen. In some embodiments, the B cell antigen is a CD329 antigen. In some embodiments, the B cell antigen is a CD338 antigen. In some embodiments, the B cell antigen is a CD351 antigen. In some embodiments, the B cell antigen is a CD352 antigen. In some embodiments, the B cell antigen is a CD353 antigen. In some embodiments, the B cell antigen is a CD354 antigen. In some embodiments, the B cell antigen is a CD355 antigen. In some embodiments, the B cell antigen is a CD356 antigen. In some embodiments, the B cell antigen is a CD357 antigen. In some embodiments, the B cell antigen is a CD358 antigen. In some embodiments, the B cell antigen is a CD360 antigen. In some embodiments, the B cell antigen is a CD361 antigen. In some embodiments, the B cell antigen is a CD362 antigen. In some embodiments, the B cell antigen is a CD363 antigen.

In one embodiment, the biparatopic or bispecific antibody binds a pathogen. In some embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments, the infectious disease is Acute Flaccid Myelitis (AFM). In some embodiments, the infectious disease is Anaplasmosis. In some embodiments, the infectious disease is Anthrax. In some embodiments, the infectious disease is Babesiosis. In some embodiments, the infectious disease is Botulism. In some embodiments, the infectious disease is Brucellosis. In some embodiments, the infectious disease is Campylobacteriosis. In some embodiments, the infectious disease is Carbapenem-resistant Infection. In some embodiments, the infectious disease is Chancroid. In some embodiments, the infectious disease is Chikungunya Virus Infection. In some embodiments, the infectious disease is Chlamydia. In some embodiments, the infectious disease is Ciguatera. In some embodiments, the infectious disease is Difficile Infection. In some embodiments, the infectious disease is Perfringens. In some embodiments, the infectious disease is Coccidioidomycosis fungal infection. In some embodiments, the infectious disease is coronavirus. In some embodiments, the infectious disease is Covid-19 (SARS-CoV-2). In some embodiments, the infectious disease is Creutzfeldt-Jacob Disease/transmissible spongiform encephalopathy. In some embodiments, the infectious disease is Cryptosporidiosis (Crypto). In some embodiments, the infectious disease is Cyclosporiasis. In some embodiments, the infectious disease is Dengue 1, 2, 3 or 4. In some embodiments, the infectious disease is Diphtheria. In some embodiments, the infectious disease is E. coli infection/Shiga toxin-producing (STEC). In some embodiments, the infectious disease is Eastern Equine Encephalitis. In some embodiments, the infectious disease is Hemorrhagic Fever (Ebola). In some embodiments, the infectious disease is Ehrlichiosis. In some embodiments, the infectious disease is Encephalitis. In some embodiments, the infectious disease is Arboviral or parainfectious. In some embodiments, the infectious disease is Non-Polio Enterovirus. In some embodiments, the infectious disease is D68 Enteroviru (EV-D68). In some embodiments, the infectious disease is Giardiasis. In some embodiments, the infectious disease is Glanders. In some embodiments, the infectious disease is Gonococcal Infection. In some embodiments, the infectious disease is Granuloma inguinale. In some embodiments, the infectious disease is Haemophilus Influenza disease Type B (Hib or H-flu). In some embodiments, the infectious disease is Hantavirus Pulmonary Syndrome (HPS). In some embodiments, the infectious disease is Hemolytic Uremic Syndrome (HUS). In some embodiments, the infectious disease is Hepatitis A (Hep A). In some embodiments, the infectious disease is Hepatitis B (Hep B). In some embodiments, the infectious disease is Hepatitis C (Hep C). In some embodiments, the infectious disease is Hepatitis D (Hep D). In some embodiments, the infectious disease is Hepatitis E (Hep E). In some embodiments, the infectious disease is Herpes. In some embodiments, the infectious disease is Herpes Zoster (Shingles). In some embodiments, the infectious disease is Histoplasmosis infection. In some embodiments, the infectious disease is Human Immunodeficiency Virus/AIDS (HIV/AIDS). In some embodiments, the infectious disease is Human Papillomavirus (HPV). In some embodiments, the infectious disease is Influenza (Flu). In some embodiments, the infectious disease is Legionellosis (Legionnaires Disease). In some embodiments, the infectious disease is Leprosy (Hansens Disease). In some embodiments, the infectious disease is Leptospirosis. In some embodiments, the infectious disease is Listeriosis (Listeria). In some embodiments, the infectious disease is Lyme Disease. In some embodiments, the infectious disease is Lymphogranuloma venereum infection (LGV). In some embodiments, the infectious disease is Malaria. In some embodiments, the infectious disease is Measles. In some embodiments, the infectious disease is Melioidosis. In some embodiments, the infectious disease is Meningitis (Viral). In some embodiments, the infectious disease is Meningococcal Disease (Meningitis (Bacterial)). In some embodiments, the infectious disease is Middle East Respiratory Syndrome Coronavirus (MERS-CoV). In some embodiments, the infectious disease is Mumps. In some embodiments, the infectious disease is Norovirus. In some embodiments, the infectious disease is Pediculosis. In some embodiments, the infectious disease is Pelvic Inflammatory Disease (PID). In some embodiments, the infectious disease is Pertussis (Whooping Cough). In some embodiments, the infectious disease is Plague (Bubonic. In some embodiments, the infectious disease is Septicemic. In some embodiments, the infectious disease is Pneumonic). In some embodiments, the infectious disease is Pneumococcal Disease (Pneumonia). In some embodiments, the infectious disease is Poliomyelitis (Polio). In some embodiments, the infectious disease is Powassan. In some embodiments, the infectious disease is Psittacosis. In some embodiments, the infectious disease is Pthiriasis. In some embodiments, the infectious disease is Pustular Rash diseases (Small pox. In some embodiments, the infectious disease is monkeypox. In some embodiments, the infectious disease is cowpox). In some embodiments, the infectious disease is Q-Fever. In some embodiments, the infectious disease is Rabies. In some embodiments, the infectious disease is Rickettsiosis (Rocky Mountain Spotted Fever). In some embodiments, the infectious disease is Rubella (German Measles). In some embodiments, the infectious disease is Salmonellosis gastroenteritis (Salmonella). In some embodiments, the infectious disease is Scabies. In some embodiments, the infectious disease is Scombroid. In some embodiments, the infectious disease is Sepsis. In some embodiments, the infectious disease is Severe Acute Respiratory Syndrome (SARS). In some embodiments, the infectious disease is Shigellosis gastroenteritis (Shigella). In some embodiments, the infectious disease is Smallpox. In some embodiments, the infectious disease is Staphyloccal Infection Methicillin-resistant (MRSA). In some embodiments, the infectious disease is Staphylococcal Food Poisoning Enterotoxin B Poisoning (Staph Food Poisoning). In some embodiments, the infectious disease is Saphylococcal Infection Vancomycin Intermediate (VISA). In some embodiments, the infectious disease is Staphylococcal Infection Vancomycin Resistant (VRSA). In some embodiments, the infectious disease is Streptococcal Disease Group A (invasive) (Strep A (invasive). In some embodiments, the infectious disease is Streptococcal Disease. In some embodiments, the infectious disease is Group B (Strep-B). In some embodiments, the infectious disease is Streptococcal Toxic-Shock Syndrome STSS Toxic Shock. In some embodiments, the infectious disease is Syphilis (primary. In some embodiments, the infectious disease is secondary. In some embodiments, the infectious disease is early latent. In some embodiments, the infectious disease is late latent. In some embodiments, the infectious disease is congenital). In some embodiments, the infectious disease is Tetanus Infection. In some embodiments, the infectious disease is Trichomoniasis. In some embodiments, the infectious disease is Trichonosis Infection. In some embodiments, the infectious disease is Tuberculosis (TB). In some embodiments, the infectious disease is Tuberculosis Latent (LTBI). In some embodiments, the infectious disease is Tularemia. In some embodiments, the infectious disease is Typhoid Fever Group D. In some embodiments, the infectious disease is Vaginosis. In some embodiments, the infectious disease is Varicella (Chickenpox), Vibrio cholerae (Cholera). In some embodiments, the infectious disease is Vibriosis (Vibrio). In some embodiments, the infectious disease is Ebola Virus Hemorrhagic Fever. In some embodiments, the infectious disease is Lasa Virus Hemorrhagic Fever. In some embodiments, the infectious disease is Marburg Virus Hemorrhagic Fever. In some embodiments, the infectious disease is West Nile Virus. In some embodiments, the infectious disease is Yellow Fever. In some embodiments, the infectious disease is Yersenia. In some embodiments, the infectious disease is and Zika Virus Infection.

In some embodiments, the pathogen is a virus. In some embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picornaviridae, polyomaviridae, poxviridae, reoviridae, retroviridae, rhabdoviridae, or togaviridae family. In some embodiments, the virus is a virus of the adenoviridae family. In some embodiments, the virus is a virus of the arenaviridae family. In some embodiments, the virus is a virus of the astroviridae family. In some embodiments, the virus is a virus of the bunyaviridae family. In some embodiments, the virus is a virus of the caliciviridae family. In some embodiments, the virus is a virus of the coronaviridae family. In some embodiments, the virus is a virus of the filoviridae family. In some embodiments, the virus is a virus of the flaviviridae family. In some embodiments, the virus is a virus of the hepadnaviridae family. In some embodiments, the virus is a virus of the hepeviridae family. In some embodiments, the virus is a virus of the orthomyxoviridae family. In some embodiments, the virus is a virus of the papillomaviridae family. In some embodiments, the virus is a virus of the paramyxoviridae family. In some embodiments, the virus is a virus of the parvoviridae family. In some embodiments, the virus is a virus of the picornaviridae family. In some embodiments, the virus is a virus of the polyomaviridae family. In some embodiments, the virus is a virus of the poxviridae family. In some embodiments, the virus is a virus of the reoviridae family. In some embodiments, the virus is a virus of the retroviridae family. In some embodiments, the virus is a virus of the rhabdoviridae family. In some embodiments, the virus is a virus of the togaviridae family.

In some embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments, the virus is an adenovirus. In some embodiments, the virus is a coronavirus. In some embodiments, the coronavirus virus is Covid-19 (SARS-CoV-2). In some embodiments, the virus is a coxsackievirus. In some embodiments, the virus is a Epstein-Barr virus. In some embodiments, the virus is a hepatitis A virus. In some embodiments, the virus is a hepatitis B virus. In some embodiments, the virus is a hepatitis C virus. In some embodiments, the virus is a herpes simplex virus type 2. In some embodiments, the virus is a cytomegalovirus. In some embodiments, the virus is a human herpes virus type 8. In some embodiments, the virus is a human immunodeficiency virus. In some embodiments, the virus is an influenza virus. In some embodiments, the virus is a measles virus. In some embodiments, the virus is a mumps virus. In some embodiments, the virus is a human papillomavirus. In some embodiments, the virus is a parainfluenza virus. In some embodiments, the virus is a poliovirus. In some embodiments, the virus is a rabies virus. In some embodiments, the virus is a respiratory syncytial virus. In some embodiments, the virus is a rubella virus. In some embodiments, the virus is a varicella-zoster virus.

In some embodiments, the pathogen is a bacteria. In some embodiments, the bacteria is a bacteria of a Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio or Yersinia genus. In some embodiments, the bacteria is a bacteria of the Bacillus genus. In some embodiments, the bacteria is a bacteria of the Bartonella genus. In some embodiments, the bacteria is a bacteria of the Bordetella genus. In some embodiments, the bacteria is a bacteria of the Borrelia genus. In some embodiments, the bacteria is a bacteria of the Brucella genus. In some embodiments, the bacteria is a bacteria of the Campylobacter genus. In some embodiments, the bacteria is a bacteria of the Chlamydia genus. In some embodiments, the bacteria is a bacteria of the Chlamydophila genus. In some embodiments, the bacteria is a bacteria of the Clostridium genus. In some embodiments, the bacteria is a bacteria of the Corynebacterium genus. In some embodiments, the bacteria is a bacteria of the Enterococcus genus. In some embodiments, the bacteria is a bacteria of the Escherichia genus. In some embodiments, the bacteria is a bacteria of the Francisella genus. In some embodiments, the bacteria is a bacteria of the Haemophilus genus. In some embodiments, the bacteria is a bacteria of the Helicobacter genus. In some embodiments, the bacteria is a bacteria of the legionella genus. In some embodiments, the bacteria is a bacteria of the Leptospira genus. In some embodiments, the bacteria is a bacteria of the Listeria genus. In some embodiments, the bacteria is a bacteria of the Mycobacterium genus. In some embodiments, the bacteria is a bacteria of the Mycoplasma genus. In some embodiments, the bacteria is a bacteria of the Neisseria genus. In some embodiments, the bacteria is a bacteria of the Pseudomonas genus. In some embodiments, the bacteria is a bacteria of the Rickettsia genus. In some embodiments, the bacteria is a bacteria of the Salmonella genus. In some embodiments, the bacteria is a bacteria of the shigella genus. In some embodiments, the bacteria is a bacteria of the Staphylococcus genus. In some embodiments, the bacteria is a bacteria of the Streptococcus genus. In some embodiments, the bacteria is a bacteria of the Treponema genus. In some embodiments, the bacteria is a bacteria of the Ureaplasma genus. In some embodiments, the bacteria is a bacteria of the vibrio genus. In some embodiments, the bacteria is a bacteria of the Yersinia genus.

In some embodiments, the pathogen is a parasite. In some embodiments, the parasite is a protozoa, helminth, or ectoparasite. In some embodiments, the protozoa is an entamoeba, giardia, leishmania, balantidium, plasmodium, or cryptosporidium. In some embodiments, the helminth is a trematode, cestode, acanthocephalan, or round worm. In some embodiments, the ectoparasite is a arthropod.

In some specific embodiments, the biparatopic or bispecific antibody provided herein binds CD22. In some specific embodiments, the biparatopic or bispecific antibody provided herein binds CD37. In some specific embodiments, the biparatopic or bispecific antibody provided herein binds GPRC5D. In some specific embodiments, the biparatopic or bispecific antibody provided herein binds KLK2. In some specific embodiments, the biparatopic or bispecific antibody provided herein binds HLA-G. In some specific embodiments, the biparatopic or bispecific antibody provided herein binds PSMA. In some specific embodiments, the biparatopic or bispecific antibody provided herein binds BCMA.

### 5.7 Other Exemplary Binding Molecules

In some embodiments, the biparatopic or bispecific antibodies provided herein are derived from certain known antibodies, wherein the antibody comprises an Fc region with RE mutations and optionally without fucosylation. Such exemplary known antibodies include but not limited to ReoPro (abciximab), Humira (adalimumab), Hyrimoz (adalimumab-adaz), Cyltezo (adalimumab-adbm), Abrilada (adalimumab-afzb), Amjevita (adalimumab-atto), Hadlima (adalimumab-bwwd), Campath, Lemtrada (alemtuzumab), Praluent (alirocumab), Tecentriq (atezolizumab), Bavencio (avelumab), Simulect (basiliximab), Benlysta (belimumab), Benlysta (belimumab), Fasenra (benralizumab), Avastin (bevacizumab), Mvasi (bevacizumab-awwb), Zirabev (bevacizumab-bvzr), Zinplava (bezlotoxumab), Blincyto (blinatumomab), Siliq (brodalumab), Beovu (brolucizumab-dbll), Crysvita (burosumab-twza), Ilaris (canakinumab), Cablivi (caplacizumab-yhdp), Libtayo (cemiplimab-rwlc), Erbitux (cetuximab), Adakveo (crizanlizumab-tmca), Zenapax (daclizumab), Zinbryta (daclizumab), Darzalex (daratumumab), Prolia, Xgeva (denosumab), Unituxin (dinutuximab), Dupixent (dupilumab), Imfinzi (durvalumab), Soliris (eculizumab), Empliciti (elotuzumab), Gamifant (emapalumab-lzsg), Hemlibra (emicizumab-kxwh), Vyepti (eptinezumab-jjmr), Aimovig (erenumab-aooe), Repatha (evolocumab), Ajovy (fremanezumab-vfrm), Emgality (galcanezumab-gnlm), Simponi (golimumab), Simponi Aria (golimumab), Tremfya (guselkumab), Trogarzo (ibalizumab-uiyk), Praxbind (idarucizumab), Remicade (infliximab), Renflexis (infliximab-abda), Avsola (infliximab-axxq), Inflectra (infliximab-dyyb), Ixifi (infliximab-qbtx), Yervoy (ipilimumab), Sarclisa (isatuximab-irfc), Taltz (ixekizumab), Takhzyro (lanadelumab-flyo), Nucala (mepolizumab), Nucala (mepolizumab), Poteligeo (mogamulizumab-kpkc), Tysabri (natalizumab), Portrazza (necitumumab), Opdivo (nivolumab), Anthim (obiltoxaximab), Gazyva (obinutuzumab), Ocrevus (ocrelizumab), Arzerra (ofatumumab), Lartruvo (olaratumab), Xolair (omalizumab), Synagis (palivizumab), Vectibix (panitumumab), Keytruda (pembrolizumab), Perjeta (pertuzumab), Cyramza (ramucirumab), Lucentis (ranibizumab), Ultomiris (ravulizumab-cwvz), raxibacumab (raxibacumab), Cinqair (reslizumab), Skyrizi (risankizumab-rzaa), Rituxan (rituximab), Truxima (rituximab-abbs), Ruxience (rituximab-pvvr), Evenity (romosozumab-aqqg), Kevzara (sarilumab), Cosentyx (secukinumab), Sylvant (siltuximab), Tepezza (teprotumumab-trbw), Ilumya (tildrakizumab-asmn), Actemra (tocilizumab), Actemra (tocilizumab), Herceptin (trastuzumab), Kanjinti (trastuzumab-anns), Ogivri (trastuzumab-dkst), Ontruzant (trastuzumab-dttb), Herzuma (trastuzumab-pkrb), Trazimera (trastuzumab-qyyp), Stelara (ustekinumab), Stelara (ustekinumab) and Entyvio (vedolizumab). The methods for introducing RE mutations and reducing fucosylation are described in more details in other sections provided herein and are well known in the art.

In some embodiments, each of the antigen binding domain of the biparatopic or bispecific binding molecule is a single domain antigen binding fragment. In some embodiments, each of the antigen binding domain of the biparatopic or bispecific binding molecule is an engineered binding fragment.

### 5.8 Polynucleotide

In certain embodiments, the disclosure encompasses polynucleotides that encode the antibodies described herein. The term “polynucleotides that encode a polypeptide” encompasses a polynucleotide that includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In certain embodiments, a polynucleotide comprises the coding sequence for a polypeptide fused in the same reading frame to a marker or tag sequence. For example, in some embodiments, a marker sequence is a hexa-histidine tag supplied by a vector that allows efficient purification of the polypeptide fused to the marker in the case of a bacterial host. In some embodiments, a marker is used in conjunction with other affinity tags.

The present disclosure further relates to variants of the polynucleotides described herein, wherein the variant encodes, for example, fragments, analogs, and/or derivatives of a polypeptide. In certain embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising an antibody described herein.

As used herein, the phrase “a polynucleotide having a nucleotide sequence at least, for example, 95% ‘identical’ to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, a polynucleotide is isolated. In certain embodiments, a polynucleotide is substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises one or more expression vectors comprising polynucleotide molecules. In some embodiments, a host cell comprises a polynucleotide molecule. In some embodiments, a host cell comprises one or more polynucleotide molecules. Construction of the vectors provided herein is exemplified in Section 7 below.

### 5.9 Polyclonal and Monoclonal Antibodies

In some embodiments, a population of the antibodies provided herein comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a polypeptide (such HLA-G, CD37, GPRCSD, KLK2, PSMA, or BCMA or a fragment thereof) or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly (I:C), Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for, e.g., anti-HLA-G, anti-CD37, anti-GPRCSD, anti-KLK2 antibody, anti-PSMA, or anti-BCMA titer. If desired, the mammal can be boosted until the antibody titer increases or plateaus. Additionally or alternatively, lymphocytes may be obtained from the immunized animal for fusion and preparation of monoclonal antibodies from hybridoma as described below.

In some embodiments, the antibodies provided herein comprise monoclonal antibodies. Monoclonal antibodies may be made using the hybridoma method first described by Kohler, et al., Nature, 1975, 256:495-7, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium, which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, Immunol., 1984, 133:3001-05; and Brodeur, et al., Monoclonal Antibody Production Techniques and Applications, 1987, 51-63).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., Anal. Biochem., 1980, 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra, et al., Curr. Opinion in Immunol., 1993, 5:256-62 and Pluckthun, Immunol. Revs., 1992, 130:151-88.

In some embodiments, an antibody that binds an epitope (e.g., on HLA-G, CD37, GPRC5D, KLK2, PSMA, or BCMA) comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, e.g., Current Protocols in Molecular Biology Vol. I, 6.3.1-6.3.6 and 2.10.3 (Ausubel et al. eds., 1989)).

In a further embodiment, monoclonal antibodies can be isolated from antibody phage libraries generated using the techniques described in, for example, Antibody Phage Display: Methods and Protocols (O'Brien and Aitken, eds., 2002). In phage display methods, functional antibody domains are displayed on the surface of phage particles which carry the polynucleotide sequences encoding them. Examples of phage display methods that can be used to make the antibodies described herein include those disclosed in Brinkman, et al., J. Immunol. Methods, 1995, 182:41-50; Ames, et al., J. Immunol. Methods, 1995, 184:177-86; Kettleborough, et al., Eur. J. Immunol., 1994, 24:952-8; Persic, et al., Gene, 1997, 187:9-18; Burton et al., Advances in Immunology, 1994, 57:191-280; PCT Application No. PCT/GB91/01 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 1993, 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 1992, 227:381-88.

Screening of the libraries can be accomplished by various techniques known in the art. For example, HLA-G, CD37, GPRC5D, KLK2, PSMA, or BCMA (e.g., an HLA-G, CD37, GPRC5D, KLK2, PSMA, or BCMA polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass, et al., Proteins, 1990, 8:309-14 and WO 92/09690, and by use of a low coating density of antigen as described in Marks et al., Biotechnol., 1992, 10:779-83.

Antibodies can be obtained by designing a suitable antigen screening procedure to select for the phage clone of interest followed by construction of a full length antibody clone using VH and/or VL sequences (e.g., the Fv sequences), or various CDR sequences from VH and VL sequences, from the phage clone of interest and suitable constant region (e.g., Fc) sequences described in Kabat, et al., supra, including sequences comprising the K248E, T437R, or K248E/T437R mutation.

Antibodies described herein can also, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, Science, 1985, 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies produced using techniques such as those described herein can be isolated using standard, well known techniques. For example, antibodies can be suitably separated from, e.g., culture medium, ascites fluid, serum, cell lysate, synthesis reaction material or the like by conventional immunoglobulin purification procedures such as, for example, protein A-Sepharose, hydroxylapatite chromatography, gel electrophoresis, dialysis, or affinity chromatography. As used herein, an “isolated” or “purified” antibody is substantially free of cellular material or other proteins from the cell or tissue source from which the antibody is derived, or substantially free of chemical precursors or other chemicals when chemically synthesized.

In some specific embodiments, monoclonal antibodies are generated using the methods exemplified in Section 7 below.

### 5.10 Humanized Antibodies

In some embodiments, the antibodies provided herein can be humanized antibodies. A humanized antibody can comprise human framework region and human constant region sequences. For example, a humanized antibody can comprise human constant region sequences. In certain embodiments, a humanized antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). In certain embodiments, a humanized antibody can comprise kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology, 1991, 28(4/5):489-498; Studnicka, et al., Protein Engineering, 1994, 7(6):805-814; and Roguska, et al., Proc. Natl. Acad. Sci. USA, 1994, 91:969-73), chain shuffling (U.S. Pat. No. 5,565,332), and techniques disclosed in, e.g., U.S. Pat. Nos. 6,407,213, 5,766,886, WO 93/17105; Tan, et al., J. Immunol., 2002, 169:1119-25; Caldas, et al., Protein Eng., 2000, 13(5):353-60; Morea et al., Methods, 2000, 20(3):267-79, Baca, et al., J. Biol. Chem., 1997, 272(16):10678-84; Roguska, et al., Protein Eng., 1996, 9(10):895 904; Couto, et al., Cancer Res., 1995, 55 (23 Supp):5973s-5977s; Couto, et al., Cancer Res., 1995, 55(8):1717-22; Sandhu, J. S., Gene, 1994, 150(2):409-10 and Pedersen, et al., J. Mol. Biol., 1994, 235(3):959-73. See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones, et al., 1986, Nature 321:522-5; Riechmann, et al., Nature, 1988, 332:323-7; and Verhoeyen, et al., Science, 1988, 239:1534-6, by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan, et al. determined that only about one third of the residues in the CDRs actually contact the antigen, and termed these the “specificity determining residues,” or SDRs (Padlan, et al., FASEB J., 1995, 9:133-9). In the technique of SDR grafting, only the SDR residues are grafted onto the human antibody framework (see, e.g., Kashmiri, et al., Methods, 2005, 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., J. Immunol., 1993, 151:2296-308; and Chothia et al., J. Mol. Biol., 1987, 196:901-17). Another method uses a particular framework derived from the consensus sequence of all human antibodies of a particular subgroup of light or heavy chains. The same framework may be used for several different humanized antibodies (Carter et al., Proc. Natl. Acad. Sci. USA, 1992, 89:4285-89; and Presta et al., J. Immunol., 1993, 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol., 2002, 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng., 2000, 13:819-24), Modeller (Sali and Blundell, J. Mol. Biol., 1993, 234:779-815), and Swiss PDB Viewer (Guex and Peitsch, Electrophoresis, 1997, 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol., 2007, 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol., 2005, 23:1105-16; Dufner, et al., Trends Biotechnol., 2006, 24:523-9; Feldhaus, et al., Nat. Biotechnol., 2003, 21:163-70; and Schlapschy et al., Protein Eng. Des. Sel., 2004, 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol., 1992, 224:487-99), or from the more limited set of target residues identified by Baca, et al., J. Biol. Chem., 1997, 272:10678-84.

In FR shuffling, whole FRs are combined with the non-human CDRs instead of creating combinatorial libraries of selected residue variants (see, e.g., Dall'Acqua et al., Methods, 2005, 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder, et al., Mol. Immunol., 2007, 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 1994, 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described (see, e.g., Jones, et al., Methods Mol Biol., 2009, 525:405-23; and De Groot, et al., Cell. Immunol., 2006, 244:148-153). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

### 5.11 Antibody Variants

In some embodiments, amino acid sequence modification(s) of the antibodies provided herein are contemplated. For example, it may be desirable to improve the binding affinity and/or other biological properties of the antibody, including but not limited to specificity, thermostability, expression level, effector functions, glycosylation (e.g., fucosylation), reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites.

Accordingly, in one embodiment, an antibody that binds to an CD22, GPRC5D, PSMA, or BCMA epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein, for examples, the antibodies described in Section 7 below.

In one embodiment, an antibody that binds to an CD22, GPRCSD, PSMA, or BCMA epitope comprises an amino acid sequence that is at least 35% identical to the amino acid sequence of an antibody described herein. In one embodiment, an antibody that binds to an CD22, GPRCSD, PSMA, or BCMA epitope comprises an amino acid sequence that is at least 40% identical to the amino acid sequence of an antibody described herein. In one embodiment, an antibody that binds to an CD22, GPRCSD, PSMA, or BCMA epitope comprises an amino acid sequence that is at least 45% identical to the amino acid sequence of an antibody described herein. In one embodiment, an antibody that binds to an CD22, GPRCSD, PSMA, or BCMA epitope comprises an amino acid sequence that is at least 50% identical to the amino acid sequence of an antibody described herein. In one embodiment, an antibody that binds to an CD22, GPRCSD, PSMA, or BCMA epitope comprises an amino acid sequence that is at least 55% identical to the amino acid sequence of an antibody described herein. In one embodiment, an antibody that binds to an CD22, GPRCSD, PSMA, or BCMA epitope comprises an amino acid sequence that is at least 60% identical to the amino acid sequence of an antibody described herein. In one embodiment, an antibody that binds to an CD22, GPRCSD, PSMA, or BCMA epitope comprises an amino acid sequence that is at least 65% identical to the amino acid sequence of an antibody described herein. In one embodiment, an antibody that binds to an CD22, GPRC5D, PSMA, or BCMA epitope comprises an amino acid sequence that is at least 70% identical to the amino acid sequence of an antibody described herein. In one embodiment, an antibody that binds to an CD22, GPRC5D, PSMA, or BCMA epitope comprises an amino acid sequence that is at least 75% identical to the amino acid sequence of an antibody described herein. In one embodiment, an antibody that binds to an CD22, GPRC5D, PSMA, or BCMA epitope comprises an amino acid sequence that is at least 80% identical to the amino acid sequence of an antibody described herein. In one embodiment, an antibody that binds to an CD22, GPRC5D, PSMA, or BCMA epitope comprises an amino acid sequence that is at least 90% identical to the amino acid sequence of an antibody described herein. In one embodiment, an antibody that binds to an CD22, GPRC5D, PSMA, or BCMA epitope comprises an amino acid sequence that is at least 95% identical to the amino acid sequence of an antibody described herein. In one embodiment, an antibody that binds to an CD22, GPRCSD, PSMA, or BCMA comprises an amino acid sequence that is at least 99% identical to the amino acid sequence of an antibody described herein.

### 5.12 Multivalent Antibodies

In yet another aspect, provided herein are methods for making the various antibodies provided herein.

A multivalent antibody may be internalized (and/or catabolized) faster than a bivalent antibody by a cell expressing an antigen to which the antibodies bind. The antibodies of the present disclosure can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### 5.13 Multispecific Antibodies

Multispecific antibodies such as bispecific antibodies are monoclonal antibodies that have binding specificities for at least two different antigens. In certain embodiments, the multispecific antibodies can be constructed based on the sequences of the antibodies provided herein, e.g., the CDR sequences contained in the VL and VH sequences provided herein. In certain embodiments, the multispecific antibodies provided herein are bispecific antibodies. In certain embodiments, bispecific antibodies are mouse, chimeric, human or humanized antibodies. Methods for making multispecific antibodies are known in the art, such as, by co-expression of two immunoglobulin heavy chain-light chain pairs, where the two heavy chains have different specificities (see, e.g., Milstein and Cuello, Nature, 1983, 305:537-40). For further details of generating multispecific antibodies (e.g., bispecific antibodies), see, for example, Bispecific Antibodies (Kontermann, ed., 2011).

### 5.14 Methods of Making the Antibodies

In yet another aspect, provided herein are methods for making the various antibodies provided herein.

In some embodiments, recombinant expression of an antibody provided herein (e.g., a full-length antibody, the heavy and/or light chain of an antibody provided herein, or a mutant thereof) that binds to an antigen (e.g., CD22, GPRC5D, PSMA, or BCMA) requires construction of an expression vector containing a polynucleotide that encodes the antibody. Once a polynucleotide encoding an antibody molecule or the heavy or light chain of an antibody provided herein has been obtained, variants of such polynucleotide that comprise the K248E, T437R or K248E/T437R mutation in the full-length antibody or the heavy chain thereof can be generated by gene synthesis (see, e.g., Zhang, D., et al., supra), and/or site-directed mutagenesis (Clynes, R. A., et al., Nature Medicine, 2000, 6(4):443-6).

The vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. In some embodiments, these methods are, for example, in vitro recombinant DNA techniques, synthetic techniques, and in vivo genetic recombination. In some embodiments, these expression vectors are replicable vectors comprising a nucleotide sequence encoding an antibody molecule provided herein, or a heavy or light chain of an antibody, operably linked to a promoter. In some embodiments, these vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy chain, the entire light chain, or both the entire heavy and light chains.

In some embodiments, the expression vector is transferred to a host cell by conventional techniques and the transfected cells are then cultured by conventional techniques to produce an antibody provided herein. In some embodiments, host cells contain a polynucleotide encoding an antibody provided herein, or a heavy or light chain thereof, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule provided herein in situ. In some embodiments, these host-expression systems are microorganisms such as bacteria (e.g., E. coli and B. subtilis) transformed with recombinant bacteriophage DNA, plasmid DNA or cosmid DNA expression vectors containing antibody coding sequences. In some embodiments, these host-expression systems are yeast (e.g., Saccharomyces pichia) transformed with recombinant yeast expression vectors containing antibody coding sequences. In some embodiments, these host-expression systems are insect cell systems infected with recombinant virus expression vectors (e.g., baculovirus) containing antibody coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing antibody coding sequences. In some embodiments, these host-expression systems are mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, these host-expression systems are bacterial cells such as Escherichia coli, or eukaryotic cells, especially for the expression of whole recombinant antibody molecule, which can be used for the expression of a recombinant antibody molecule. In some embodiments, mammalian cells such as Chinese hamster ovary cells (CHO), in conjunction with a vector such as the major intermediate early gene promoter element from human cytomegalovirus, is an effective expression system for antibodies (Foecking, et al., Gene, 1986, 45:101; and Cockett, et al., Bio/Technology, 1990, 8:2). In some embodiments, antibodies provided herein are produced in CHO cells. In some embodiments, the expression of nucleotide sequences encoding antibodies provided herein which immunospecifically bind to an CD22, GPRCSD, PSMA, or BCMA antigen is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther, et al., EMBO, 1983, 12:1791), in which the antibody coding sequence may be ligated individually into the vector in frame with the lac Z coding region so that a fusion protein is produced; pIN vectors (Inouye & Inouye, Nucleic Acids Res., 1985, 13:3101-3109; Van Heeke & Schuster, J. Biol. Chem., 1989, 24:5503-9); and the like. pGEX vectors may also be used to express foreign polypeptides as fusion proteins with glutathione 5-transferase (GST). In some embodiments, such fusion proteins are soluble and can easily be purified from lysed cells by adsorption and binding to matrix glutathione agarose beads followed by elution in the presence of free glutathione. In some embodiments, the pGEX vectors are designed to include thrombin or factor Xa protease cleavage sites so that the cloned target gene product can be released from the GST moiety.

In some embodiments, Autographa californica nuclear polyhedrosis virus (AcNPV) is used as a vector to express foreign genes in an insect system. In some embodiments, the virus grows in Spodoptera frugiperda cells. In some embodiments, the antibody coding sequence may be cloned individually into non-essential regions (for example the polyhedrin gene) of the virus and placed under control of an AcNPV promoter (for example the polyhedrin promoter).

In some embodiments, viral vectors, e.g., tobacco mosaic virus (TMV)-based viral vectors, are used in plant cells and are systematically delivered to multiple parts of a plant body by Agrobacterium for coexpression of multiple antibodies of interest (Giritch, A., et al., Proc. Natl. Acad. Sci. USA, 2006, 103(40):14701-6). In some embodiments, the use of plant cells as expression systems results in significant increases in production speed and yield of antibodies, and the capability to synthesize mammalian-type complex N-oligosaccharides (Loos, A. and Steinkellner, H., Arch Biochem Biophys., 2012, 526-172(2):167-73).

In mammalian host cells, a number of viral-based expression systems may be utilized. In some embodiments, an adenovirus is used as an expression vector, and the antibody coding sequence of interest may be ligated to an adenovirus transcription/translation control complex, e.g., the late promoter and tripartite leader sequence. In some embodiments, this chimeric gene may then be inserted in the adenovirus genome by in vitro or in vivo recombination. In some embodiments, insertion in a non-essential region of the viral genome (e.g., region E1 or E3) will result in a recombinant virus that is viable and capable of expressing the antibody molecule in infected hosts (see, e.g., Logan & Shenk, Proc. Natl. Acad. Sci. USA, 1984, 8(1):355-9). In some embodiments, specific initiation signals may also be required for efficient translation of inserted antibody coding sequences. In some embodiments, these signals include the ATG initiation codon and adjacent sequences. In some embodiments, the initiation codon must be in phase with the reading frame of the desired coding sequence to ensure translation of the entire insert. In some embodiments, these exogenous translational control signals and initiation codons can be of a variety of origins, both natural and synthetic. In some embodiments, the efficiency of expression may be enhanced by the inclusion of appropriate transcription enhancer elements, transcription terminators, etc. (see, e.g., Bittner, et al., Methods in Enzymol., 1987, 153:51-544).

In some embodiments, a host cell strain may be chosen which modulates the expression of the inserted sequences, or modifies and processes the gene product in the specific fashion desired. Such modifications (e.g., glycosylation, including fucosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells. In some embodiments, fully human monoclonal antibodies provided herein are produced in mammalian cells, such as CHO cells.

Standard techniques known to those of skill in the art can be used to generate an antibody provided herein with a heavy chain comprising no fucose residue on the Asn-297-linked N-oligosaccharide thereof. In some embodiments, cells with deficient GMD enzymes (e.g., CHO Lec13 cells) are used to generate an antibody with no fucose residue on the Asn-297-linked N-oligosaccharide on the heavy chain. In some embodiments, cells with reduced α-1,6 fucosyltransferase activity resulting from mutated or inactivated FUT8 genes (e.g., rat hybridoma YB2/0 cell lines) are used to generate an antibody with no fucose residue on the Asn-297-linked N-oligosaccharide on the heavy chain. In some embodiments, cells with overexpression of 0-1,4-mannosyl-glycoprotein 4-β-N-acetylglucosaminyltransferase (GnT-III) are used to generate an antibody with no fucose residue on the Asn-297-linked N-oligosaccharide on the heavy chain. In some embodiments, cells with inactivated Golgi GDP-fucose transporter (GFT) gene Slc35c1 (e.g., CHO-gmt3 cells) are used to generate an antibody with no fucose residue on the Asn-297-linked N-oligosaccharide on the heavy chain. In some embodiments, cells with heterologous expression of bacterial RMD are used to generate an antibody with no fucose residue on the Asn-297-linked N-oligosaccharide on the heavy chain. In some embodiments, biochemical inhibitors of fucosylation (e.g., fucose analogs such as 2-fluorofucose and 5-alkynylfucose) are used in expression systems to produce antibodies with no fucose residue on the Asn-297-linked N-oligosaccharide on the heavy chain (Pereira, N. A., et al., supra; Shields, R. L. et al., supra; Kanda Y., supra). In some embodiments, plant cells with disrupted α1,3-fucosyltransferase (FucT) and β1,2-xylosyltransferase (XylT) are used to generate an antibody with no fucose residue on the Asn-297-linked N-oligosaccharide on the heavy chain (Pereira, N. A., et al., supra). In some embodiments, chemoenzymic remodeling is used to generate an antibody with no fucose residue on the Asn-297-linked N-oligosaccharide on the heavy chain (Pereira, N. A., et al., supra). For example, an exoglycosidase such as fucosidase may be used to remove the fucose on the Asn-297-linked N-oligosaccharide on the heavy chain of an antibody (Pereira, N. A., et al., supra).

For long-term, high-yield production of recombinant proteins, stable expression can be utilized. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. In some embodiments, the selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the antibody molecule. Such engineered cell lines may be particularly useful in screening and evaluation of compositions that interact directly or indirectly with the antibody molecule.

A number of selection systems may be used, including, but not limited to, the herpes simplex virus thymidine kinase (Wigler, et al., Cell, 1977, 11:223), hypoxanthineguanine phosphoribosyltransferase (Szybalska & Szybalski, Proc. Natl. Acad. Sci. USA, 1992, 48:202), and adenine phosphoribosyltransferase (Lowy, et al., Cell, 1980, 22:8-17) genes can be employed in tk-, hgprt- or aprt-cells, respectively. Also, antimetabolite resistance may be used as the basis of selection for the following genes: dhfr, which confers resistance to methotrexate (Wigler, et al., Natl. Acad. Sci. USA, 1980, 77:357; O'Hare, et al., Proc. Natl. Acad. Sci. USA, 1981, 78:1527); gpt, which confers resistance to mycophenolic acid (Mulligan & Berg, Proc. Natl. Acad. Sci. USA, 1981, 78:2072); neo, which confers resistance to the aminoglycoside G-418 (Wu and Wu, Biotherapy, 1991, 3:87-95; Tolstoshev, Ann. Rev. Pharmacol. Toxicol., 1993, 32:573-96; Mulligan, Science, 1993, 260:926-32; and Morgan and Anderson, Ann. Rev. Biochem., 1993, 62:191-217); and hygro, which confers resistance to hygromycin (Santerre, et al., Gene, 1984, 30:147). Methods commonly known in the art of recombinant DNA technology may be routinely applied to select the desired recombinant clone, and such methods are described, for example, in Ausubel, et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli, et al. (eds.), Current Protocols in Human Genetics, John Wiley & Sons, NY (1994); Colberre-Garapin, et al., J. Mol. Biol., 1981, 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). In some embodiments, a marker in the vector system expressing antibody is amplifiable, and the increase in the level of inhibitor present in culture of the host cell will increase the number of copies of the marker gene and the production of the antibody since the amplified region is associated with the antibody gene (Crouse et al., 1983, Mol. Cell. Biol. 3:257).

The host cell may be co-transfected with two expression vectors provided herein, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 1986, 322:52; and Kohler, Proc. Natl. Acad. Sci. USA, 1980, 77:2197-9). The coding sequences for the heavy and light chains may comprise cDNA or genomic DNA.

Once an antibody molecule provided herein has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies provided herein can be fused to heterologous polypeptide sequences described herein or otherwise known in the art to facilitate purification. Certain methods of making the antibodies provided herein are described in Section 7 below.

### 5.15 Pharmaceutical Compositions

In one aspect, the present disclosure further provides pharmaceutical compositions comprising at least one antibody of the present disclosure. In some embodiments, a pharmaceutical composition comprises therapeutically effective amount of an antibody or antigen binding fragment thereof provided herein and a pharmaceutically acceptable excipient.

In some embodiments, pharmaceutical compositions comprising an antibody are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody of the present disclosure may be formulated in any suitable form for delivery to a target cell/tissue, e.g., as microcapsules or macroemulsions (Remington, supra; Park, et al., Molecules, 2005, 10:146-61; Malik, et al., Curr. Drug. Deliv., 2007, 4:141-51), as sustained release formulations (Putney and Burke, Nature Biotechnol., 1998, 16:153-57), or in liposomes (Maclean, et al., Int. J. Oncol., 1997, 11:325-32; Kontermann, Curr. Opin. Mol. Ther., 2006, 8:39-45).

An antibody provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody as described herein, including, but not limited to, encapsulation in liposomes, microparticles, microcapsules, recombinant cells capable of expressing the antibody, receptor-mediated endocytosis (see, e.g., Wu and Wu, J. Biol. Chem., 1987, 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, Science, 1990, 249:1527-33; Sefton, Crit. Ref Biomed. Eng., 1987, 14:201-40; Buchwald, et al., Surgery, 1980, 88:507-16; and Saudek et al., N. Engl. J. Med., 1989, 321:569-74). In another embodiment, polymeric materials can be used to achieve controlled or sustained release of a prophylactic or therapeutic agent (e.g., an antibody as described herein) or a composition provided herein (see, e.g., Medical Applications of Controlled Release (Langer and Wise, eds., 1974); Controlled Drug Bioavailability, Drug Product Design and Performance (Smolen and Ball, eds., 1984); Ranger and Peppas, J. Macromol. Sci. Rev. Macromol. Chem., 1983, 23:61-126; Levy, et al., Science, 1985, 228:190-92; During, et al., Ann. Neurol., 1989, 25:351-6; Howard, et al., J. Neurosurg., 1989, 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxyethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, supra. Any technique known to one of skill in the art can be used to produce sustained release formulations comprising one or more antibody as described herein (see, e.g., U.S. Pat. No. 4,526,938, PCT publication Nos. WO 91/05548 and WO 96/20698, Ning, et al., Radiotherapy & Oncology, 1996, 39:179-89; Song, et al., PDA J. of Pharma. Sci. & Tech., 1995, 50:372-97; Cleek, et al., Pro. Int'l. Symp. Control. Rel. Bioact. Mater., 1997, 24:853-4; and Lam, et al., Proc. Int'l. Symp. Control Rel. Bioact. Mater., 1997, 24:759-60).

### 5.16 Methods of Using, Administrating and Dosing

In one aspect, provided herein is a method of enhancing the ADCC, CDC, and/or ADCP activity of an antibody.

As another example, C42B and LNCaP cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay can be performed with freshly thawed PBMC (Hemcare, PB009C-3) or NK cells isolated from the frozen PBMC by RoboSep™ Cell Separation Instruments. Isolated NK cells can be either used immediately or primed overnight with low dose IL-2 (1 ng/ml, Miltenyi Biotec). The ratio of effector to target cell per well can be 34:1 for PBMC and 5:1 for isolated NK cells. In some embodiments, anti-PSMA antibodies are tested with final concentrations ranging from 100 nM to 0.01 nM. After effector cells and antibodies are added to target cells, real time imaging can be performed under Incucyte© S3 instrument (Essen BioScience). Total GFP intergraded signal per well can be quantified with Incucyte© software. Data analysis can be performed by Incucyte© software and Prism (GraphPad Software) based on values of quadruplicates. The percentage of cell killing can be calculated as: (1−PSMA mAb/no mAb control)×100%.

As another example, C42B and LNCaP cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay can be performed with freshly thawed PBMC (Hemcare, PB009C-3) or NK cells isolated from the frozen PBMC by RoboSep™ Cell Separation Instruments. Isolated NK cells can be either used immediately or primed overnight with low dose IL-2 (1 ng/ml, Miltenyi Biotec). The ratio of effector to target cell per well can be 34:1 for PBMC and 5:1 for isolated NK cells. In some embodiments, anti-BCMA antibodies are tested with final concentrations ranging from 100 nM to 0.01 nM. After effector cells and antibodies are added to target cells, real time imaging can be performed under Incucyte® S3 instrument (Essen BioScience). Total GFP intergraded signal per well can be quantified with Incucyte® software. Data analysis can be performed by Incucyte® software and Prism (GraphPad Software) based on values of quadruplicates. The percentage of cell killing can be calculated as: (1−BCMA mAb/no mAb control)×100%.

In some embodiments, the antibody provided herein causes at least 10% target cell death via ADCC. In some embodiments, the antibody provided herein causes at least 20% target cell death via ADCC. In some embodiments, the antibody provided herein causes at least 30% target cell death via ADCC. In some embodiments, the antibody provided herein causes at least 40% target cell death via ADCC. In some embodiments, the antibody provided herein causes at least 50% target cell death via ADCC. In some embodiments, the antibody provided herein causes at least 60% target cell death via ADCC. In some embodiments, the antibody provided herein causes at least 70% target cell death via ADCC. In some embodiments, the antibody provided herein causes at least 80% target cell death via ADCC. In some embodiments, the antibody provided herein causes at least 90% target cell death via ADCC. In some embodiments, the antibody provided herein causes at least 95% target cell death via ADCC.

In some embodiments, the CDC activity of anti-CD22 antibodies, anti-GPRC5D antibodies, anti-PSMA antibodies, or anti-BCMA antibodies provided herein are determined by measuring the percentage of target cell death. For example, target cells that endogenously expresses CD22, GPRC5D, PSMA, or BCMA can be cultured in DMEM medium with 10% Fetal Bovine Serum (FBS). In some embodiments, antibodies are added to target cells and incubated for 30 minutes at 37° C., with baby rabbit serum then added to target cells to a final concentration of 10% to provide a source of complement components for CDC. In some embodiments, such mixture is incubated for 4 hours at 37° C. 100 μl of CellTiter-Glo reagent (Promega) can be added to the mixture followed by incubation for 10 minutes at room temperature. Target cell viability can be determined by measuring luminescence with a Tecan SPARK Reader. Target cell death can thus be determined as the difference between 100% and target cell viability.

In some embodiments, the antibody provided herein causes at least 10% target cell death via CDC. In some embodiments, the antibody provided herein causes at least 20% target cell death via CDC. In some embodiments, the antibody provided herein causes at least 30% target cell death via CDC. In some embodiments, the antibody provided herein causes at least 40% target cell death via CDC. In some embodiments, the antibody provided herein causes at least 50% target cell death via CDC. In some embodiments, the antibody provided herein causes at least 60% target cell death via CDC. In some embodiments, the antibody provided herein causes at least 70% target cell death via CDC. In some embodiments, the antibody provided herein causes at least 80% target cell death via CDC. In some embodiments, the antibody provided herein causes at least 90% target cell death via CDC. In some embodiments, the antibody provided herein causes at least 95% target cell death via CDC.

In some embodiments, the antibody provided herein enhances ADCC activity by at least about 2-fold. In some embodiments, the antibody provided herein enhances ADCC activity by at least about 5-fold. In some embodiments, the antibody provided herein enhances ADCC activity by at least about 10-fold. In some embodiments, the antibody provided herein enhances ADCC activity by at least about 20-fold. In some embodiments, the antibody provided herein enhances ADCC activity by at least about 30-fold. In some embodiments, the antibody provided herein enhances ADCC activity by at least about 40-fold. In some embodiments, the antibody provided herein enhances ADCC activity by at least about 50-fold. In some embodiments, the antibody provided herein enhances ADCC activity by at least about 60-fold. In some embodiments, the antibody provided herein enhances ADCC activity by at least about 70-fold. In some embodiments, the antibody provided herein enhances ADCC activity by at least about 80-fold. In some embodiments, the antibody provided herein enhances ADCC activity by at least about 90-fold. In some embodiments, the antibody provided herein enhances ADCC activity by at least about 100-fold. In some embodiments, the antibody provided herein enhances ADCC activity by at least about 200-fold. In some embodiments, the antibody provided herein enhances ADCC activity by at least about 500-fold.

In some embodiments, the antibody provided herein enhances CDC activity by at least about 2-fold. In some embodiments, the antibody provided herein enhances CDC activity by at least about 5-fold. In some embodiments, the antibody provided herein enhances CDC activity by at least about 10-fold. In some embodiments, the antibody provided herein enhances CDC activity by at least about 20-fold. In some embodiments, the antibody provided herein enhances CDC activity by at least about 30-fold. In some embodiments, the antibody provided herein enhances CDC activity by at least about 40-fold. In some embodiments, the antibody provided herein enhances CDC activity by at least about 50-fold. In some embodiments, the antibody provided herein enhances CDC activity by at least about 60-fold. In some embodiments, the antibody provided herein enhances CDC activity by at least about 70-fold. In some embodiments, the antibody provided herein enhances CDC activity by at least about 80-fold. In some embodiments, the antibody provided herein enhances CDC activity by at least about 90-fold. In some embodiments, the antibody provided herein enhances CDC activity by at least about 100-fold. In some embodiments, the antibody provided herein enhances CDC activity by at least about 200-fold. In some embodiments, the antibody provided herein enhances CDC activity by at least about 500-fold.

In some embodiments, the antibody provided herein enhances both ADCC and CDC activity by at least about 2-fold. In some embodiments, the antibody provided herein enhances both ADCC and CDC activity by at least about 5-fold. In some embodiments, the antibody provided herein enhances both ADCC and CDC activity by at least about 10-fold. In some embodiments, the antibody provided herein enhances both ADCC and CDC activity by at least about 20-fold. In some embodiments, the antibody provided herein enhances both ADCC and CDC activity by at least about 30-fold. In some embodiments, the antibody provided herein enhances both ADCC and CDC activity by at least about 40-fold. In some embodiments, the antibody provided herein enhances both ADCC and CDC activity by at least about 50-fold. In some embodiments, the antibody provided herein enhances both ADCC and CDC activity by at least about 60-fold. In some embodiments, the antibody provided herein enhances both ADCC and CDC activity by at least about 70-fold. In some embodiments, the antibody provided herein enhances both ADCC and CDC activity by at least about 80-fold. In some embodiments, the antibody provided herein enhances both ADCC and CDC activity by at least about 90-fold. In some embodiments, the antibody provided herein enhances both ADCC and CDC activity by at least about 100-fold. In some embodiments, the antibody provided herein enhances both ADCC and CDC activity by at least about 200-fold. In some embodiments, the antibody provided herein enhances both ADCC and CDC activity by at least about 500-fold.

In some embodiments, the antibody provided herein has increased capability of hexamerization on a cell surface by at least about 2-fold. In some embodiments, the antibody provided herein has increased capability of hexamerization on a cell surface by at least about 5-fold. In some embodiments, the antibody provided herein has increased capability of hexamerization on a cell surface by at least about 10-fold. In some embodiments, the antibody provided herein has increased capability of hexamerization on a cell surface by at least about 20-fold. In some embodiments, the antibody provided herein has increased capability of hexamerization on a cell surface by at least about 30-fold. In some embodiments, the antibody provided herein has increased capability of hexamerization on a cell surface by at least about 40-fold. In some embodiments, the antibody provided herein has increased capability of hexamerization on a cell surface by at least about 50-fold. In some embodiments, the antibody provided herein has increased capability of hexamerization on a cell surface by at least about 60-fold. In some embodiments, the antibody provided herein has increased capability of hexamerization on a cell surface by at least about 70-fold. In some embodiments, the antibody provided herein has increased capability of hexamerization on a cell surface by at least about 80-fold. In some embodiments, the antibody provided herein has increased capability of hexamerization on a cell surface by at least about 90-fold. In some embodiments, the antibody provided herein has increased capability of hexamerization on a cell surface by at least about 100-fold. In some embodiments, the antibody provided herein has increased capability of hexamerization on a cell surface by at least about 200-fold. In some embodiments, the antibody provided herein has increased capability of hexamerization on a cell surface by at least about 500-fold.

In some embodiments, the antibody provided herein has increased capability of engaging C1q by at least about 2-fold. In some embodiments, the antibody provided herein has increased capability of engaging C1q by at least about 5-fold. In some embodiments, the antibody provided herein has increased capability of engaging C1q by at least about 10-fold. In some embodiments, the antibody provided herein has increased capability of engaging C1q by at least about 20-fold. In some embodiments, the antibody provided herein has increased capability of engaging C1q by at least about 30-fold. In some embodiments, the antibody provided herein has increased capability of engaging C1q by at least about 40-fold. In some embodiments, the antibody provided herein has increased capability of engaging C1q by at least about 50-fold. In some embodiments, the antibody provided herein has increased capability of engaging C1q by at least about 60-fold. In some embodiments, the antibody provided herein has increased capability of engaging C1q by at least about 70-fold. In some embodiments, the antibody provided herein has increased capability of engaging C1q by at least about 80-fold. In some embodiments, the antibody provided herein has increased capability of engaging C1q by at least about 90-fold. In some embodiments, the antibody provided herein has increased capability of engaging C1q by at least about 100-fold. In some embodiments, the antibody provided herein has increased capability of engaging C1q by at least about 200-fold. In some embodiments, the antibody provided herein has increased capability of engaging C1q by at least about 500-fold.

In another aspect, provided herein is a method of treating a disease or disorder in a subject comprising administering to the subject an effective amount of an antibody provided herein. In one embodiment, the disease or disorder is a CD22-mediated disease or disorder. In one embodiment, the disease or disorder is a GPRC5D-mediated disease or disorder. In one embodiment, the disease or disorder is a PSMA-mediated disease or disorder. In one embodiment, the disease or disorder is a BCMA-mediated disease or disorder. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling (see, e.g., Heider, K. H., et al., Blood, 2011, 118(15):4159-68; Carosella, E. D., et al., Adv. Immunol., 2015, 127:33-144; Stathis, S., et al., Invest New Drugs, 2018, 36(5):869-76; Stilgenbauer, S., et al., Leukemia, 2019, 33:2531-5; Gomes, R. G., et al., Hum. Immunol., 2018, 79(6):477-84; Moroso, V., et al., Transplantation, 2015, 99(12):2514-22; Lazarte, J., et al., Hum. Immunol., 2018, 79(8):587-93; Rached, M. R., et al., Eur J Obstet Gynecol ReprodBiol., 2019, 235:36-41; Koc, A., et al., Adv Clin Exp Med., 2018, 27(9):1233-7; Ribeyre, C., et al., Front Immunol., 2018, 9:278; Smith, E. L., et al., Sci TranslMed., 2019, 11(485):eaau7746; Kodama, T., et al., Mol Cancer Ther., 2019, 18(9):1555-64; Cohen, Y., et al., Hematology, 2013, 18(6):348-51).

Also provided herein is a method of treatment of a disease or disorder, wherein the subject is administered one or more therapeutic agents in combination with the antibody provided herein.

In another aspect, provided herein is the use of the antibody provided herein in the manufacture of a medicament for treating a disease or disorder in a subject.

In another aspect, provided herein is the use of a pharmaceutical composition provided herein in the manufacture of a medicament for treating a disease or disorder in a subject.

In a specific embodiment, provided herein is a composition for use in the prevention and/or treatment of a disease or condition comprising an antibody provided herein. In one embodiment, provided herein is a composition for use in the prevention of a disease or condition, wherein the composition comprises an antibody provided herein. In one embodiment, provided herein is a composition for use in the treatment of a disease or condition, wherein the composition comprises an antibody provided herein. In some embodiments, the disease or condition is a CD22-mediated disease. In some embodiments, the disease or condition is a GPRC5D-mediated disease. In one embodiment, the disease or condition is a PSMA-mediated disease. In one embodiment, the disease or condition is a BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephrophathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises an antibody provided herein. In one embodiment, provided herein is a composition for use in the prevention of a symptom of a disease or condition, wherein the composition comprises an antibody provided herein. In one embodiment, provided herein is a composition for use in the treatment of a symptom of a disease or condition, wherein the composition comprises an antibody provided herein. In some embodiments, the disease or condition is a CD22-mediated, GPRCSD-mediated, PSMA-mediated disease, and/or BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephrophathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody provided herein. In one embodiment, provided herein is a method of preventing a disease or condition in a subject, comprising administering an effective amount of an antibody provided herein. In one embodiment, provided herein is a method of treating a disease or condition in a subject, comprising administering an effective amount of an antibody provided herein. In some embodiments, the disease or condition is a CD22-mediated, GPRC5D-mediated, PSMA-mediated disease, and/or BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephrophathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody provided herein. In one embodiment, provided herein is a method of preventing a symptom of a disease or condition in a subject, comprising administering an effective amount of an antibody provided herein. In one embodiment, provided herein is a method of treating a symptom of a disease or condition in a subject, comprising administering an effective amount of an antibody provided herein. In some embodiments, the disease or condition is a CD22-mediated, GPRC5D-mediated, PSMA-mediated disease, and/or BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of an antibody provided herein, or pharmaceutical composition comprising an antibody provided herein. In one aspect, the antibody is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In a one embodiment, the subject is a human. In another embodiment, the subject is a human with a disease or condition.

In some embodiments of each or any of the above- or below-mentioned embodiments, the present binding molecules are used for treating solid tumor cancer. In other embodiments, the present binding molecules are used for treating blood cancer. In other embodiments, the disease or disorder is an autoimmune and inflammatory disease. In other embodiments, the disease or disorder is an infectious disease.

In some embodiments of each or any of the above- or below-mentioned embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments of each or any of the above- or below-mentioned embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments of each or any of the above- or below-mentioned embodiments, the disease or disorder is a hematological cancer, such as leukemia, lymphoma, or myeloma. In some embodiments of each or any of the above- or below-mentioned embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (TLL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments of each or any of the above- or below-mentioned embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments of each or any of the above- or below-mentioned embodiments, the cancer is an adrenal cancer. In some embodiments of each or any of the above- or below-mentioned embodiments, the cancer is an anal cancer. In some embodiments of each or any of the above- or below-mentioned embodiments, the cancer is an appendix cancer. In some embodiments of each or any of the above- or below-mentioned embodiments, the cancer is a bile duct cancer. In some embodiments of each or any of the above- or below-mentioned embodiments, the cancer is a bladder cancer. In some embodiments of each or any of the above- or below-mentioned embodiments, the cancer is a bone cancer. In some embodiments of each or any of the above- or below-mentioned embodiments, the cancer is a brain cancer. In some embodiments of each or any of the above- or below-mentioned embodiments, the cancer is a breast cancer. In some embodiments of each or any of the above- or below-mentioned embodiments, the cancer is a cervical cancer. In some embodiments of each or any of the above- or below-mentioned embodiments, the cancer is a colorectal cancer. In some embodiments of each or any of the above- or below-mentioned embodiments, the cancer is an esophageal cancer. In some embodiments of each or any of the above- or below-mentioned embodiments, the cancer is a gallbladder cancer. In some embodiments of each or any of the above- or below-mentioned embodiments, the cancer is a gestational trophoblastic. In some embodiments of each or any of the above- or below-mentioned embodiments, the cancer is a head and neck cancer. In some embodiments of each or any of the above- or below-mentioned embodiments, the cancer is a Hodgkin lymphoma. In some embodiments of each or any of the above- or below-mentioned embodiments, the cancer is an intestinal cancer. In some embodiments of each or any of the above- or below-mentioned embodiments, the cancer is a kidney cancer. In some embodiments of each or any of the above- or below-mentioned embodiments, the cancer is a leukemia. In some embodiments of each or any of the above- or below-mentioned embodiments, the cancer is a liver cancer. In some embodiments of each or any of the above- or below-mentioned embodiments, the cancer is a lung cancer. In some embodiments of each or any of the above- or below-mentioned embodiments, the cancer is a melanoma. In some embodiments of each or any of the above- or below-mentioned embodiments, the cancer is a mesothelioma. In some embodiments of each or any of the above- or below-mentioned embodiments, the cancer is a multiple myeloma (MM). In some embodiments of each or any of the above- or below-mentioned embodiments, the cancer is a neuroendocrine tumor. In some embodiments of each or any of the above- or below-mentioned embodiments, the cancer is a non-Hodgkin lymphoma. In some embodiments of each or any of the above- or below-mentioned embodiments, the cancer is an oral cancer. In some embodiments of each or any of the above- or below-mentioned embodiments, the cancer is an ovarian cancer. In some embodiments of each or any of the above- or below-mentioned embodiments, the cancer is a pancreatic cancer. In some embodiments of each or any of the above- or below-mentioned embodiments, the cancer is a prostate cancer. In some embodiments of each or any of the above- or below-mentioned embodiments, the cancer is a sinus cancer. In some embodiments of each or any of the above- or below-mentioned embodiments, the cancer is a skin cancer. In some embodiments of each or any of the above- or below-mentioned embodiments, the cancer is a soft tissue sarcoma spinal cancer. In some embodiments of each or any of the above- or below-mentioned embodiments, the cancer is a stomach cancer. In some embodiments of each or any of the above- or below-mentioned embodiments, the cancer is a testicular cancer. In some embodiments of each or any of the above- or below-mentioned embodiments, the cancer is a throat cancer. In some embodiments of each or any of the above- or below-mentioned embodiments, the cancer is a thyroid cancer. In some embodiments of each or any of the above- or below-mentioned embodiments, the cancer is a uterine cancer endometrial cancer. In some embodiments of each or any of the above- or below-mentioned embodiments, the cancer is a vaginal cancer. In some embodiments of each or any of the above- or below-mentioned embodiments, the cancer is a vulvar cancer.

In some embodiments of each or any of the above- or below-mentioned embodiments, the adrenal cancer is an adrenocortical carcinoma (ACC), adrenal cortex cancer, pheochromocytoma, or neuroblastoma. In some embodiments of each or any of the above- or below-mentioned embodiments, the anal cancer is a squamous cell carcinoma, cloacogenic carcinoma, adenocarcinoma, basal cell carcinoma, or melanoma. In some embodiments of each or any of the above- or below-mentioned embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments of each or any of the above- or below-mentioned embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments of each or any of the above- or below-mentioned embodiments, the bladder cancer is transitional cell carcinoma (TCC), papillary carcinoma, flat carcinoma, squamous cell carcinoma, adenocarcinoma, small-cell carcinoma, or sarcoma. In some embodiments of each or any of the above- or below-mentioned embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments of each or any of the above- or below-mentioned embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments of each or any of the above- or below-mentioned embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments of each or any of the above- or below-mentioned embodiments, the cervical cancer is a squamous cell carcinoma, or adenocarcinoma. In some embodiments of each or any of the above- or below-mentioned embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments of each or any of the above- or below-mentioned embodiments, the esophageal cancer is an adenocarcinoma or squamous cell carcinoma. In some embodiments of each or any of the above- or below-mentioned embodiments, the gall bladder cancer is an adenocarcinoma, papillary adenocarcinoma, adenosquamous carcinoma, squamous cell carcinoma, small cell carcinoma, or sarcoma. In some embodiments of each or any of the above- or below-mentioned embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments of each or any of the above- or below-mentioned embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments of each or any of the above- or below-mentioned embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments of each or any of the above- or below-mentioned embodiments, the intestinal cancer is a small intestine cancer, small bowel cancer, adenocarcinoma, sarcoma, gastrointestinal stromal tumors, carcinoid tumors, or lymphoma. In some embodiments of each or any of the above- or below-mentioned embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments of each or any of the above- or below-mentioned embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments of each or any of the above- or below-mentioned embodiments, the liver cancer is a hepatocellular carcinoma (HCC), fibrolamellar HCC, cholangiocarcinoma, angiosarcoma, or liver metastasis. In some embodiments of each or any of the above- or below-mentioned embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments of each or any of the above- or below-mentioned embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments of each or any of the above- or below-mentioned embodiments, the mesothelioma is a pleural mesothelioma, peritoneal mesothelioma, pericardial mesothelioma, or testicular mesothelioma. In some embodiments of each or any of the above- or below-mentioned embodiments, the multiple myeloma is an active myeloma or smoldering myeloma. In some embodiments of each or any of the above- or below-mentioned embodiments, the neuroendocrine tumor is a gastrointestinal neuroendocrine tumor, pancreatic neuroendocrine tumor, or lung neuroendocrine tumor. In some embodiments of each or any of the above- or below-mentioned embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments of each or any of the above- or below-mentioned embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments of each or any of the above- or below-mentioned embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments of each or any of the above- or below-mentioned embodiments, the pancreatic cancer is a pancreatic exocrine gland cancer, pancreatic endocrine gland cancer, or pancreatic adenocarcinoma, islet cell tumor, or neuroendocrine tumor. In some embodiments of each or any of the above- or below-mentioned embodiments, the prostate cancer is a prostate adenocarcinoma, prostate sarcoma, transitional cell carcinoma, small cell carcinoma, or neuroendocrine tumor. In some embodiments of each or any of the above- or below-mentioned embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments of each or any of the above- or below-mentioned embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments of each or any of the above- or below-mentioned embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments of each or any of the above- or below-mentioned embodiments, the spinal cancer is a spinal metastatic tumor. In some embodiments of each or any of the above- or below-mentioned embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments of each or any of the above- or below-mentioned embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments of each or any of the above- or below-mentioned embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments of each or any of the above- or below-mentioned embodiments, the thyroid cancer is a papillary carcinoma, follicular carcinoma, Hurthle cell carcinoma, medullary thyroid carcinoma, or anaplastic carcinoma. In some embodiments of each or any of the above- or below-mentioned embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments of each or any of the above- or below-mentioned embodiments, the vaginal cancer is a squamous cell carcinoma, adenocarcinoma, melanoma, or sarcoma. In some embodiments of each or any of the above- or below-mentioned embodiments, the vulvar cancer is a squamous cell carcinoma or adenocarcinoma.

In some embodiments of each or any of the above- or below-mentioned embodiments, the disease or disorder is caused by a pathogen. In some embodiments of each or any of the above- or below-mentioned embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments of each or any of the above- or below-mentioned embodiments, the infectious disease is Acute Flaccid Myelitis (AFM). In some embodiments of each or any of the above- or below-mentioned embodiments, the infectious disease is Anaplasmosis. In some embodiments of each or any of the above- or below-mentioned embodiments, the infectious disease is Anthrax. In some embodiments of each or any of the above- or below-mentioned embodiments, the infectious disease is Babesiosis. In some embodiments of each or any of the above- or below-mentioned embodiments, the infectious disease is Botulism. In some embodiments of each or any of the above- or below-mentioned embodiments, the infectious disease is Brucellosis. In some embodiments of each or any of the above- or below-mentioned embodiments, the infectious disease is Campylobacteriosis. In some embodiments of each or any of the above- or below-mentioned embodiments, the infectious disease is Carbapenem-resistant Infection. In some embodiments of each or any of the above- or below-mentioned embodiments, the infectious disease is Chancroid. In some embodiments of each or any of the above- or below-mentioned embodiments, the infectious disease is Chikungunya Virus Infection. In some embodiments of each or any of the above- or below-mentioned embodiments, the infectious disease is Chlamydia. In some embodiments of each or any of the above- or below-mentioned embodiments, the infectious disease is Ciguatera. In some embodiments of each or any of the above- or below-mentioned embodiments, the infectious disease is Difficile Infection. In some embodiments of each or any of the above- or below-mentioned embodiments, the infectious disease is Perfringens. In some embodiments of each or any of the above- or below-mentioned embodiments, the infectious disease is Coccidioidomycosis fungal infection. In some embodiments of each or any of the above- or below-mentioned embodiments, the infectious disease is coronavirus. In some embodiments of each or any of the above- or below-mentioned embodiments, the infectious disease is Covid-19 (SARS-CoV-2). In some embodiments of each or any of the above- or below-mentioned embodiments, the infectious disease is Creutzfeldt-Jacob Disease/transmissible spongiform encephalopathy. In some embodiments of each or any of the above- or below-mentioned embodiments, the infectious disease is Cryptosporidiosis (Crypto). In some embodiments of each or any of the above- or below-mentioned embodiments, the infectious disease is Cyclosporiasis. In some embodiments of each or any of the above- or below-mentioned embodiments, the infectious disease is Dengue 1, 2, 3 or 4. In some embodiments of each or any of the above- or below-mentioned embodiments, the infectious disease is Diphtheria. In some embodiments of each or any of the above- or below-mentioned embodiments, the infectious disease is E. coli infection/Shiga toxin-producing (STEC). In some embodiments of each or any of the above- or below-mentioned embodiments, the infectious disease is Eastern Equine Encephalitis. In some embodiments of each or any of the above- or below-mentioned embodiments, the infectious disease is Hemorrhagic Fever (Ebola). In some embodiments of each or any of the above- or below-mentioned embodiments, the infectious disease is Ehrlichiosis. In some embodiments of each or any of the above- or below-mentioned embodiments, the infectious disease is Encephalitis. In some embodiments of each or any of the above- or below-mentioned embodiments, the infectious disease is Arboviral or parainfectious. In some embodiments of each or any of the above- or below-mentioned embodiments, the infectious disease is Non-Polio Enterovirus. In some embodiments of each or any of the above- or below-mentioned embodiments, the infectious disease is D68 Enteroviru (EV-D68). In some embodiments of each or any of the above- or below-mentioned embodiments, the infectious disease is Giardiasis. In some embodiments of each or any of the above- or below-mentioned embodiments, the infectious disease is Glanders. In some embodiments of each or any of the above- or below-mentioned embodiments, the infectious disease is Gonococcal Infection. In some embodiments of each or any of the above- or below-mentioned embodiments, the infectious disease is Granuloma inguinale. In some embodiments of each or any of the above- or below-mentioned embodiments, the infectious disease is Haemophilus Influenza disease Type B (Hib or H-flu). In some embodiments of each or any of the above- or below-mentioned embodiments, the infectious disease is Hantavirus Pulmonary Syndrome (HPS). In some embodiments of each or any of the above- or below-mentioned embodiments, the infectious disease is Hemolytic Uremic Syndrome (HUS). In some embodiments of each or any of the above- or below-mentioned embodiments, the infectious disease is Hepatitis A (Hep A). In some embodiments of each or any of the above- or below-mentioned embodiments, the infectious disease is Hepatitis B (Hep B). In some embodiments of each or any of the above- or below-mentioned embodiments, the infectious disease is Hepatitis C (Hep C). In some embodiments of each or any of the above- or below-mentioned embodiments, the infectious disease is Hepatitis D (Hep D). In some embodiments of each or any of the above- or below-mentioned embodiments, the infectious disease is Hepatitis E (Hep E). In some embodiments of each or any of the above- or below-mentioned embodiments, the infectious disease is Herpes. In some embodiments of each or any of the above- or below-mentioned embodiments, the infectious disease is Herpes Zoster (Shingles). In some embodiments of each or any of the above- or below-mentioned embodiments, the infectious disease is Histoplasmosis infection. In some embodiments of each or any of the above- or below-mentioned embodiments, the infectious disease is Human Immunodeficiency Virus/AIDS (HIV/AIDS). In some embodiments of each or any of the above- or below-mentioned embodiments, the infectious disease is Human Papillomavirus (HPV). In some embodiments of each or any of the above- or below-mentioned embodiments, the infectious disease is Influenza (Flu). In some embodiments of each or any of the above- or below-mentioned embodiments, the infectious disease is Legionellosis (Legionnaires Disease). In some embodiments of each or any of the above- or below-mentioned embodiments, the infectious disease is Leprosy (Hansens Disease). In some embodiments of each or any of the above- or below-mentioned embodiments, the infectious disease is Leptospirosis. In some embodiments of each or any of the above- or below-mentioned embodiments, the infectious disease is Listeriosis (Listeria). In some embodiments of each or any of the above- or below-mentioned embodiments, the infectious disease is Lyme Disease. In some embodiments of each or any of the above- or below-mentioned embodiments, the infectious disease is Lymphogranuloma venereum infection (LGV). In some embodiments of each or any of the above- or below-mentioned embodiments, the infectious disease is Malaria. In some embodiments of each or any of the above- or below-mentioned embodiments, the infectious disease is Measles. In some embodiments of each or any of the above- or below-mentioned embodiments, the infectious disease is Melioidosis. In some embodiments of each or any of the above- or below-mentioned embodiments, the infectious disease is Meningitis (Viral). In some embodiments of each or any of the above- or below-mentioned embodiments, the infectious disease is Meningococcal Disease (Meningitis (Bacterial)). In some embodiments of each or any of the above- or below-mentioned embodiments, the infectious disease is Middle East Respiratory Syndrome Coronavirus (MERS-CoV). In some embodiments of each or any of the above- or below-mentioned embodiments, the infectious disease is Mumps. In some embodiments of each or any of the above- or below-mentioned embodiments, the infectious disease is Norovirus. In some embodiments of each or any of the above- or below-mentioned embodiments, the infectious disease is Pediculosis. In some embodiments of each or any of the above- or below-mentioned embodiments, the infectious disease is Pelvic Inflammatory Disease (PID). In some embodiments of each or any of the above- or below-mentioned embodiments, the infectious disease is Pertussis (Whooping Cough). In some embodiments of each or any of the above- or below-mentioned embodiments, the infectious disease is Plague (Bubonic. In some embodiments of each or any of the above- or below-mentioned embodiments, the infectious disease is Septicemic. In some embodiments of each or any of the above- or below-mentioned embodiments, the infectious disease is Pneumonic). In some embodiments of each or any of the above- or below-mentioned embodiments, the infectious disease is Pneumococcal Disease (Pneumonia). In some embodiments of each or any of the above- or below-mentioned embodiments, the infectious disease is Poliomyelitis (Polio). In some embodiments of each or any of the above- or below-mentioned embodiments, the infectious disease is Powassan. In some embodiments of each or any of the above- or below-mentioned embodiments, the infectious disease is Psittacosis. In some embodiments of each or any of the above- or below-mentioned embodiments, the infectious disease is Pthiriasis. In some embodiments of each or any of the above- or below-mentioned embodiments, the infectious disease is Pustular Rash diseases (Small pox. In some embodiments of each or any of the above- or below-mentioned embodiments, the infectious disease is monkeypox. In some embodiments of each or any of the above- or below-mentioned embodiments, the infectious disease is cowpox). In some embodiments of each or any of the above- or below-mentioned embodiments, the infectious disease is Q-Fever. In some embodiments of each or any of the above- or below-mentioned embodiments, the infectious disease is Rabies. In some embodiments of each or any of the above- or below-mentioned embodiments, the infectious disease is Rickettsiosis (Rocky Mountain Spotted Fever). In some embodiments of each or any of the above- or below-mentioned embodiments, the infectious disease is Rubella (German Measles). In some embodiments of each or any of the above- or below-mentioned embodiments, the infectious disease is Salmonellosis gastroenteritis (Salmonella). In some embodiments of each or any of the above- or below-mentioned embodiments, the infectious disease is Scabies. In some embodiments of each or any of the above- or below-mentioned embodiments, the infectious disease is Scombroid. In some embodiments of each or any of the above- or below-mentioned embodiments, the infectious disease is Sepsis. In some embodiments of each or any of the above- or below-mentioned embodiments, the infectious disease is Severe Acute Respiratory Syndrome (SARS). In some embodiments of each or any of the above- or below-mentioned embodiments, the infectious disease is Shigellosis gastroenteritis (Shigella). In some embodiments of each or any of the above- or below-mentioned embodiments, the infectious disease is Smallpox. In some embodiments of each or any of the above- or below-mentioned embodiments, the infectious disease is Staphyloccal Infection Methicillin-resistant (MRSA). In some embodiments of each or any of the above- or below-mentioned embodiments, the infectious disease is Staphylococcal Food Poisoning Enterotoxin B Poisoning (Staph Food Poisoning). In some embodiments of each or any of the above- or below-mentioned embodiments, the infectious disease is Saphylococcal Infection Vancomycin Intermediate (VISA). In some embodiments of each or any of the above- or below-mentioned embodiments, the infectious disease is Staphylococcal Infection Vancomycin Resistant (VRSA). In some embodiments of each or any of the above- or below-mentioned embodiments, the infectious disease is Streptococcal Disease Group A (invasive) (Strep A (invasive). In some embodiments of each or any of the above- or below-mentioned embodiments, the infectious disease is Streptococcal Disease. In some embodiments of each or any of the above- or below-mentioned embodiments, the infectious disease is Group B (Strep-B). In some embodiments of each or any of the above- or below-mentioned embodiments, the infectious disease is Streptococcal Toxic-Shock Syndrome STSS Toxic Shock. In some embodiments of each or any of the above- or below-mentioned embodiments, the infectious disease is primary. In some embodiments of each or any of the above- or below-mentioned embodiments, the infectious disease is secondary. In some embodiments of each or any of the above- or below-mentioned embodiments, the infectious disease is early latent. In some embodiments of each or any of the above- or below-mentioned embodiments, the infectious disease is late latent. In some embodiments, the infectious disease is congenital. In some embodiments of each or any of the above- or below-mentioned embodiments, the infectious disease is Tetanus Infection. In some embodiments of each or any of the above- or below-mentioned embodiments, the infectious disease is Trichomoniasis. In some embodiments of each or any of the above- or below-mentioned embodiments, the infectious disease is Trichonosis Infection. In some embodiments of each or any of the above- or below-mentioned embodiments, the infectious disease is Tuberculosis (TB). In some embodiments of each or any of the above- or below-mentioned embodiments, the infectious disease is Tuberculosis Latent (LTBI). In some embodiments of each or any of the above- or below-mentioned embodiments, the infectious disease is Tularemia. In some embodiments of each or any of the above- or below-mentioned embodiments, the infectious disease is Typhoid Fever Group D. In some embodiments of each or any of the above- or below-mentioned embodiments, the infectious disease is Vaginosis. In some embodiments of each or any of the above- or below-mentioned embodiments, the infectious disease is Varicella (Chickenpox), Vibrio cholerae (Cholera). In some embodiments of each or any of the above- or below-mentioned embodiments, the infectious disease is Vibriosis (Vibrio). In some embodiments of each or any of the above- or below-mentioned embodiments, the infectious disease is Ebola Virus Hemorrhagic Fever. In some embodiments of each or any of the above- or below-mentioned embodiments, the infectious disease is Lasa Virus Hemorrhagic Fever. In some embodiments of each or any of the above- or below-mentioned embodiments, the infectious disease is Marburg Virus Hemorrhagic Fever. In some embodiments of each or any of the above- or below-mentioned embodiments, the infectious disease is West Nile Virus. In some embodiments of each or any of the above- or below-mentioned embodiments, the infectious disease is Yellow Fever. In some embodiments of each or any of the above- or below-mentioned embodiments, the infectious disease is Yersenia. In some embodiments of each or any of the above- or below-mentioned embodiments, the infectious disease is and Zika Virus Infection.

In some embodiments of each or any of the above- or below-mentioned embodiments, the pathogen is a bacteria. In some embodiments of each or any of the above- or below-mentioned embodiments, the bacteria is a bacteria of a Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio or Yersinia genus. In some embodiments of each or any of the above- or below-mentioned embodiments, the bacteria is a bacteria of the bacillus genus. In some embodiments of each or any of the above- or below-mentioned embodiments, the bacteria is a bacteria of the Bartonella genus. In some embodiments of each or any of the above- or below-mentioned embodiments, the bacteria is a bacteria of the Bordetella genus. In some embodiments of each or any of the above- or below-mentioned embodiments, the bacteria is a bacteria of the Borrelia genus. In some embodiments of each or any of the above- or below-mentioned embodiments, the bacteria is a bacteria of the Brucella genus. In some embodiments of each or any of the above- or below-mentioned embodiments, the bacteria is a bacteria of the Campylobacter genus. In some embodiments of each or any of the above- or below-mentioned embodiments, the bacteria is a bacteria of the Chlamydia genus. In some embodiments of each or any of the above- or below-mentioned embodiments, the bacteria is a bacteria of the Chlamydophila genus. In some embodiments of each or any of the above- or below-mentioned embodiments, the bacteria is a bacteria of the Clostridium genus. In some embodiments of each or any of the above- or below-mentioned embodiments, the bacteria is a bacteria of the Corynebacterium genus. In some embodiments of each or any of the above- or below-mentioned embodiments, the bacteria is a bacteria of the Enterococcus genus. In some embodiments of each or any of the above- or below-mentioned embodiments, the bacteria is a bacteria of the Escherichia genus. In some embodiments of each or any of the above- or below-mentioned embodiments, the bacteria is a bacteria of the Francisella genus. In some embodiments of each or any of the above- or below-mentioned embodiments, the bacteria is a bacteria of the Haemophilus genus. In some embodiments of each or any of the above- or below-mentioned embodiments, the bacteria is a bacteria of the Helicobacter genus. In some embodiments of each or any of the above- or below-mentioned embodiments, the bacteria is a bacteria of the Legionella genus. In some embodiments of each or any of the above- or below-mentioned embodiments, the bacteria is a bacteria of the Leptospira genus. In some embodiments of each or any of the above- or below-mentioned embodiments, the bacteria is a bacteria of the Listeria genus. In some embodiments of each or any of the above- or below-mentioned embodiments, the bacteria is a bacteria of the Mycobacterium genus. In some embodiments of each or any of the above- or below-mentioned embodiments, the bacteria is a bacteria of the Mycoplasma genus. In some embodiments of each or any of the above- or below-mentioned embodiments, the bacteria is a bacteria of the Neisseria genus. In some embodiments of each or any of the above- or below-mentioned embodiments, the bacteria is a bacteria of the Pseudomonas genus. In some embodiments of each or any of the above- or below-mentioned embodiments, the bacteria is a bacteria of the Rickettsia genus. In some embodiments of each or any of the above- or below-mentioned embodiments, the bacteria is a bacteria of the Salmonella genus. In some embodiments of each or any of the above- or below-mentioned embodiments, the bacteria is a bacteria of the Shigella genus. In some embodiments of each or any of the above- or below-mentioned embodiments, the bacteria is a bacteria of the Staphylococcus genus. In some embodiments of each or any of the above- or below-mentioned embodiments, the bacteria is a bacteria of the Streptococcus genus. In some embodiments of each or any of the above- or below-mentioned embodiments, the bacteria is a bacteria of the Treponema genus. In some embodiments of each or any of the above- or below-mentioned embodiments, the bacteria is a bacteria of the Ureaplasma genus. In some embodiments of each or any of the above- or below-mentioned embodiments, the bacteria is a bacteria of the vibrio genus. In some embodiments of each or any of the above- or below-mentioned embodiments, the bacteria is a bacteria of the Yersinia genus.

In some embodiments of each or any of the above- or below-mentioned embodiments, the pathogen is a parasite. In some embodiments of each or any of the above- or below-mentioned embodiments, the parasite is a protozoa, helminth, or ectoparasite. In some embodiments of each or any of the above- or below-mentioned embodiments, the protozoa is an entamoeba, giardia, leishmania, balantidium, plasmodium, or cryptosporidium. In some embodiments of each or any of the above- or below-mentioned embodiments, the helminth is a trematode, cestode, acanthocephalan, or round worm. In some embodiments of each or any of the above- or below-mentioned embodiments, the ectoparasite is an arthropod.

In some embodiments of each or any of the above- or below-mentioned embodiments, the pathogen is a virus. In some embodiments of each or any of the above- or below-mentioned embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picornaviridae, polyomaviridae, poxviridae, reoviridae, retroviridae, rhabdoviridae, or togaviridae family. In some embodiments of each or any of the above- or below-mentioned embodiments, the virus is a virus of the adenoviridae family. In some embodiments of each or any of the above- or below-mentioned embodiments, the virus is a virus of the arenaviridae family. In some embodiments of each or any of the above- or below-mentioned embodiments, the virus is a virus of the astroviridae family. In some embodiments of each or any of the above- or below-mentioned embodiments, the virus is a virus of the bunyaviridae family. In some embodiments of each or any of the above- or below-mentioned embodiments, the virus is a virus of the caliciviridae family. In some embodiments of each or any of the above- or below-mentioned embodiments, the virus is a virus of the coronaviridae family. In some embodiments of each or any of the above- or below-mentioned embodiments, the virus is a virus of the filoviridae family. In some embodiments of each or any of the above- or below-mentioned embodiments, the virus is a virus of the flaviviridae family. In some embodiments of each or any of the above- or below-mentioned embodiments, the virus is a virus of the hepadnaviridae family. In some embodiments of each or any of the above- or below-mentioned embodiments, the virus is a virus of the hepeviridae family. In some embodiments of each or any of the above- or below-mentioned embodiments, the virus is a virus of the orthomyxoviridae family. In some embodiments of each or any of the above- or below-mentioned embodiments, the virus is a virus of the papillomaviridae family. In some embodiments of each or any of the above- or below-mentioned embodiments, the virus is a virus of the paramyxoviridae family. In some embodiments of each or any of the above- or below-mentioned embodiments, the virus is a virus of the parvoviridae family. In some embodiments of each or any of the above- or below-mentioned embodiments, the virus is a virus of the picornaviridae family. In some embodiments of each or any of the above- or below-mentioned embodiments, the virus is a virus of the polyomaviridae family. In some embodiments of each or any of the above- or below-mentioned embodiments, the virus is a virus of the poxviridae family. In some embodiments of each or any of the above- or below-mentioned embodiments, the virus is a virus of the reoviridae family. In some embodiments of each or any of the above- or below-mentioned embodiments, the virus is a virus of the retroviridae family. In some embodiments of each or any of the above- or below-mentioned embodiments, the virus is a virus of the rhabdoviridae family. In some embodiments of each or any of the above- or below-mentioned embodiments, the virus is a virus of the togaviridae family.

In some embodiments of each or any of the above- or below-mentioned embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments of each or any of the above- or below-mentioned embodiments, the virus is an adenovirus. In some embodiments of each or any of the above- or below-mentioned embodiments, the virus is a coronavirus. In some embodiments of each or any of the above- or below-mentioned embodiments, the coronavirus virus is Covid-19 (SARS-CoV-2). In some embodiments of each or any of the above- or below-mentioned embodiments, the virus is a coxsackievirus. In some embodiments of each or any of the above- or below-mentioned embodiments, the virus is a Epstein-Barr virus. In some embodiments of each or any of the above- or below-mentioned embodiments, the virus is a hepatitis A virus. In some embodiments of each or any of the above- or below-mentioned embodiments, the virus is a hepatitis B virus. In some embodiments of each or any of the above- or below-mentioned embodiments, the virus is a hepatitis C virus. In some embodiments of each or any of the above- or below-mentioned embodiments, the virus is a herpes simplex virus type 2. In some embodiments of each or any of the above- or below-mentioned embodiments, the virus is a cytomegalovirus. In some embodiments of each or any of the above- or below-mentioned embodiments, the virus is a human herpes virus type 8. In some embodiments of each or any of the above- or below-mentioned embodiments, the virus is a human immunodeficiency virus. In some embodiments of each or any of the above- or below-mentioned embodiments, the virus is an influenza virus. In some embodiments of each or any of the above- or below-mentioned embodiments, the virus is a measles virus. In some embodiments of each or any of the above- or below-mentioned embodiments, the virus is a mumps virus. In some embodiments of each or any of the above- or below-mentioned embodiments, the virus is a human papillomavirus. In some embodiments of each or any of the above- or below-mentioned embodiments, the virus is a parainfluenza virus. In some embodiments of each or any of the above- or below-mentioned embodiments, the virus is a poliovirus. In some embodiments of each or any of the above- or below-mentioned embodiments, the virus is a rabies virus. In some embodiments of each or any of the above- or below-mentioned embodiments, the virus is a respiratory syncytial virus. In some embodiments of each or any of the above- or below-mentioned embodiments, the virus is a rubella virus. In some embodiments of each or any of the above- or below-mentioned embodiments, the virus is a varicella-zoster virus.

In some embodiments of each or any of the above- or below-mentioned embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-l-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale, Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpura, hepatitis A, hepatitis B, hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, myasthenia gravis, microscopic vasculitis of the kidneys, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis, Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Th1-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, Yersinia and Salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

In some embodiments of each or any of the above- or below-mentioned embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Cytokines and prostaglandins control the inflammatory response, and are released in an ordered and self-limiting cascade into the blood or affected tissues. This release of cytokines and prostaglandins increases the blood flow to the area of injury or infection, and may result in redness and warmth. Some of these chemicals cause a leak of fluid into the tissues, resulting in swelling. This protective process may stimulate nerves and cause pain. These changes, when occurring for a limited period in the relevant area, work to the benefit of the body.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as psoriasis, rheumatoid arthritis, osteoarthritis, psoriatic arthritis, Crohn's disease, asthma, allergies or inflammatory bowel disease, are characterized by chronic inflammation. Inflammatory diseases such as arthritis, related arthritic conditions (e.g., osteoarthritis, rheumatoid arthritis, and psoriatic arthritis), inflammatory bowel disease (e.g., Crohn's disease and ulcerative colitis), sepsis, psoriasis, atopic dermatitis, contact dermatitis, and chronic obstructive pulmonary disease, chronic inflammatory pulmonary diseases are also prevalent and problematic ailments.

Various delivery systems are known and can be used to administer a prophylactic or therapeutic agent (e.g., an antibody provided herein), including, but not limited to, encapsulation in liposomes, microparticles, microcapsules, recombinant cells capable of expressing the antibody, receptor-mediated endocytosis (see, e.g., Wu and Wu, J. Biol. Chem., 1987 262:4429-4432), construction of a nucleic acid as part of a retroviral or other vector, etc. Methods of administering a prophylactic or therapeutic agent (e.g., an antibody provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering an antibody provided herein, care must be taken to use materials to which the antibody does not absorb.

In another embodiment, a prophylactic or therapeutic agent, or a composition provided herein can be delivered in a vesicle, in particular a liposome (see Langer, Science, 1990, 249:1527-1533; Treat, et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, CRC Crit. Ref Biomed. Eng., 1987, 14:20; Buchwald et al., Surgery, 1980, 88:507; Saudek et al., N. Engl. J Med., 1989, 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J., Macromol. Sci. Rev. Macromol. Chem., 1983, 23:61; see also Levy, et al., Science, 1985, 228:190; During, et al., Ann. Neurol., 1989, 25:351; Howard, et al., J. Neurosurg., 1989, 7(1):105; U.S. Pat. Nos. 5,679,377; 5,916,597; 5,912,015; 5,989,463; 5,128,326; PCT Publication No. WO 99/15154; and PCT Publication No. WO 99/20253). Examples of polymers used in sustained release formulations include, but are not limited to, poly(2-hydroxyethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer, Science, 1990, 249:1527-33). Any technique known to one of skill in the art can be used to produce sustained release formulations comprising one or more antibody provided herein. See, e.g., U.S. Pat. No. 4,526,938, PCT publication WO 91/05548, PCT publication WO 96/20698, Ning, et al., Radiotherapy & Oncology, 1996, 39:179-89; Song, et al., PDA J of Pharmaceutical Sci & Technol., 1995, 50:372-97; Cleek, et al., Pro. Int'l. Symp. Control. Rel. Bioact. Mater., 1997, 24:853-54; and Lam, et al., Proc. Int'l. Symp. Control Rel. Bioact. Mater., 1997, 24:759-60, each of which is incorporated herein by reference in their entirety.

In a specific embodiment, where the composition provided herein is a nucleic acid encoding a prophylactic or therapeutic agent (e.g., an antibody provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot, et al., Proc. Natl. Acad. Sci. USA, 1991, 88:1864-8), etc. Alternatively, a nucleic acid can be introduced intracellularly and incorporated within host cell DNA for expression by homologous recombination.

In a specific embodiment, a composition provided herein comprises one, two or more antibodies provided herein. In another embodiment, a composition provided herein comprises one, two or more antibodies provided herein and a prophylactic or therapeutic agent other than an antibody provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the antibody provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. In some embodiments, compositions for intravenous administration are solutions in sterile isotonic aqueous buffer. Where necessary, the composition may also include a solubilizing agent and a local anesthetic such as lignocaine to ease pain at the site of the injection. Such compositions, however, may be administered by a route other than intravenous.

The ingredients of compositions provided herein may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody provided herein can be packaged in a hermetically sealed container such as an ampoule or sachette indicating the quantity of antibody. In one embodiment, the antibody is supplied as a dry sterilized lyophilized powder or water free concentrate in a hermetically sealed container and can be reconstituted, e.g., with water or saline to the appropriate concentration for administration to a subject. The lyophilized antibody can be stored at between 2 and 8° C. in its original container and the antibody can be administered within 12 hours, such as within 6 hours, within 5 hours, within 3 hours, or within 1 hour after being reconstituted. In an alternative embodiment, an antibody provided herein is supplied in liquid form in a hermetically sealed container indicating the quantity and concentration of the antibody.

The compositions provided herein can be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody provided herein), or a composition provided herein that will be effective in the prevention and/or treatment of a disease or condition can be determined by standard clinical techniques. In addition, in vitro assays may optionally be employed to help identify optimal dosage ranges. In some embodiments, the precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of an antibody provided herein to a patient is intranasal, intramuscular, intravenous, subcutaneous, or a combination thereof, but other routes described herein are also acceptable. Each dose may or may not be administered by an identical route of administration. In some embodiments, an antibody provided herein may be administered via multiple routes of administration simultaneously or subsequently to other doses of the same or a different antibody provided herein.

In certain embodiments, the antibody provided herein are administered prophylactically or therapeutically to a subject. The antibody provided herein can be prophylactically or therapeutically administered to a subject so as to prevent, lessen or ameliorate a disease or symptom thereof.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EMBODIMENTS

This invention provides the following non-limiting embodiments.

In one set of embodiments (embodiment set A), provided are:

A1. A binding molecule comprising an antigen binding domain and an Fc region, wherein
(i) the antigen binding domain is biparatopic or bi-specific; and
(ii) the Fc region comprises K248E and T437R mutations (RE mutations), wherein amino acid residue numbering is according to the EU numbering system;
wherein the binding molecule has increased capability of hexamerization on a cell surface, and/or increased capability of engaging C1q.

A2. The binding molecule of embodiment A1, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

A3. The binding molecule of embodiment A2, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

A4. The binding molecule of any one of embodiments A1 to A3, wherein the oligosaccharide covalently attached to the Fc region via the N297 residue thereof does not comprise a core fucose residue.

A5. The binding molecule of any one of embodiments A1 to A4, wherein the binding molecule is a biparatopic or bispecific antibody.

A6. A population of binding molecules comprising the binding molecule of any one of embodiments A1 to A5.

A7. A population of binding molecules, wherein each binding molecule comprising an antigen binding domain and an Fc region; wherein:
(i) the antigen binding domain is biparatopic or bispecific;
(ii) the Fc region comprises K248E and T437R mutations (RE mutations), wherein amino acid residue numbering is according to the EU numbering system; and
(iii) less than 80% of the oligosaccharides covalently attached to the population of the binding molecules via the N297 residue of the Fc region comprise a core fucose residue.

A8. A population of binding molecules,
wherein each binding molecule comprises an antigen binding domain and an Fc region;
wherein the antigen binding domain of at least 10% of the binding molecules in the population is biparatopic or bispecific;
wherein the Fc region of each binding molecule comprises K248E and T437R mutations (RE mutations), wherein amino acid residue numbering is according to the EU numbering system; and
wherein less than 80% of the oligosaccharides covalently attached to the population of the binding molecules via the N297 residue of the Fc region comprise a core fucose residue.

A9. The population of the binding molecules of embodiment A8, wherein the antigen binding domain of at least 20% of the binding molecules in the population is biparatopic or bispecific.

A10. The population of the binding molecules of embodiment A8, wherein the antigen binding domain of at least 30% of the binding molecules in the population is biparatopic or bispecific.

A11. The population of the binding molecules of embodiment A8, wherein the antigen binding domain of at least 40% of the binding molecules in the population is biparatopic or bispecific.

A12. The population of the binding molecules of embodiment A8, wherein the antigen binding domain of at least 50% of the binding molecules in the population is biparatopic or bispecific.

A13. The population of the binding molecules of embodiment A8, wherein the antigen binding domain of at least 60% of the binding molecules in the population is biparatopic or bispecific.

A14. The population of the binding molecules of embodiment A8, wherein the antigen binding domain of at least 70% of the binding molecules in the population is biparatopic or bispecific.

A15. The population of the binding molecules of embodiment A8, wherein the antigen binding domain of at least 80% of the binding molecules in the population is biparatopic or bispecific.

A16. The population of the binding molecules of embodiment A8, wherein the antigen binding domain of at least 90% of the binding molecules in the population is biparatopic or bispecific.

A17. The population of the binding molecules of any one of embodiments A7 to A16, wherein less than 70% of the oligosaccharides covalently attached to the population of the binding molecules via the N297 residue of the Fc region comprise a core fucose residue.

A18. The population of the binding molecules of any one of embodiments A7 to A16, wherein less than 60% of the oligosaccharides covalently attached to the population of the binding molecules via the N297 residue of the Fc region comprise a core fucose residue.

A19. The population of the binding molecules of any one of embodiments A7 to A16, wherein less than 50% of the oligosaccharides covalently attached to the population of the binding molecules via the N297 residue of the Fc region comprise a core fucose residue.

A20. The population of the binding molecules of any one of embodiments A7 to A16, wherein less than 40% of the oligosaccharides covalently attached to the population of the binding molecules via the N297 residue of the Fc region comprise a core fucose residue.

A21. The population of the binding molecules of any one of embodiments A7 to A16, wherein less than 30% of the oligosaccharides covalently attached to the population of the binding molecules via the N297 residue of the Fc region comprise a core fucose residue.

A22. The population of the binding molecules of any one of embodiments A7 to A16, wherein less than 20% of the oligosaccharides covalently attached to the population of the binding molecules via the N297 residue of the Fc region comprise a core fucose residue.

A23. The population of the binding molecules of any one of embodiments A7 to A16, wherein less than 10% of the oligosaccharides covalently attached to the population of the binding molecules via the N297 residue of the Fc region comprise a core fucose residue.

A24. The population of the binding molecules of any one of embodiments A7 to A23, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

A25. The population of the binding molecules of any one of embodiments A7 to A24, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

A26. The population of the binding molecules of any one of embodiments A7 to A25, wherein the binding molecules are biparatopic or bispecific antibodies.

A27. The population of the binding molecules of any one of embodiments A7 to A26, wherein the binding molecules are produced by expressing a polynucleotide encoding the binding molecules or a fragment thereof in a host cell that is deficient in adding a fucose to the oligosaccharide attached to the Fc region of an antibody.

A28. The population of the binding molecules of embodiment A27, wherein the host cell has reduced GDP-mannose 4,6-dehydratase (GMD) activity or reduced α-1,6 fucosyltransferase activity.

A29. The binding molecule of any one of embodiments A1 to A5, wherein the binding molecule has enhanced ADCC, enhanced CDC, and/or enhanced ADCP.

A30. The population of the binding molecules of any one of embodiments A6 to A28, wherein the population of the binding molecules has enhanced ADCC, enhanced CDC, and/or enhanced ADCP.

A31. A nucleic acid encoding the binding molecule of any one of embodiments A1 to A5 or A29.

A32. A vector comprising the nucleic acid of embodiment A31.

In another set of embodiments (embodiment set B), provided are:

B1. A pharmaceutical composition comprising a binding molecule and a pharmaceutically acceptable excipient, wherein the binding molecule comprises an antigen binding domain and an Fc region; wherein:
(i) the antigen binding domain is biparatopic or bispecific; and
(ii) the Fc region comprises K248E and T437R mutations (RE mutations), wherein amino acid residue numbering is according to the EU numbering system;
wherein the binding molecule has increased capability of hexamerization on a cell surface, and/or increased capability of engaging C1q.

B2. The pharmaceutical composition of embodiment B1, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

B3. The pharmaceutical composition of embodiment B2, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

B4. The pharmaceutical composition of any one of embodiments B1 to B3, wherein the oligosaccharide covalently attached to the Fc region via the N297 residue thereof does not comprise a core fucose residue.

B5. The pharmaceutical composition of any one of embodiments B1 to B4, wherein binding molecule is a biparatopic or bispecific antibody.

B6. The pharmaceutical composition of any one of embodiments B1 to B5, wherein the binding molecule has enhanced ADCC, enhanced CDC, and/or enhanced ADCP.

B7. A pharmaceutical composition comprising a population of binding molecules and a pharmaceutically acceptable excipient, wherein each binding molecule comprises an antigen binding domain and an Fc region; wherein:
(i) the antigen binding domain is biparatopic or bispecific;
(ii) the Fc region comprises K248E and T437R mutations (RE mutations), wherein amino acid residue numbering is according to the EU numbering system; and
(iii) less than 80% of the oligosaccharides covalently attached to the population of the binding molecules via the N297 residue of the Fc region comprise a core fucose residue.

B8. A pharmaceutical composition comprising a population of binding molecules and a pharmaceutically acceptable excipient,
wherein each binding molecule comprises an antigen binding domain and an Fc region,
wherein the antigen binding domain of at least 10% of the binding molecules in the population is biparatopic or bispecific;
wherein the Fc region of each binding molecule comprises K248E and T437R mutations (RE mutations), wherein amino acid residue numbering is according to the EU numbering system; and
wherein less than 80% of the oligosaccharides covalently attached to the population of the binding molecules via the N297 residue of the Fc region comprise a core fucose residue.

B9. The pharmaceutical composition of embodiment B8, wherein the antigen binding domain of at least 20% of the binding molecules in the population is biparatopic or bispecific.

B10. The pharmaceutical composition of embodiment B8, wherein the antigen binding domain of at least 30% of the binding molecules in the population is biparatopic or bispecific.

B11. The pharmaceutical composition of embodiment B8, wherein the antigen binding domain of at least 40% of the binding molecules in the population is biparatopic or bispecific.

B12. The pharmaceutical composition of embodiment B8, wherein the antigen binding domain of at least 50% of the binding molecules in the population is biparatopic or bispecific.

B13. The pharmaceutical composition of embodiment B8, wherein the antigen binding domain of at least 60% of the binding molecules in the population is biparatopic or bispecific.

B14. The pharmaceutical composition of embodiment B8, wherein the antigen binding domain of at least 70% of the binding molecules in the population is biparatopic or bispecific.

B15. The pharmaceutical composition of embodiment B8, wherein the antigen binding domain of at least 80% of the binding molecules in the population is biparatopic or bispecific.

B16. The pharmaceutical composition of embodiment B8, wherein the antigen binding domain of at least 90% of the binding molecules in the population is biparatopic or bispecific.

B17. The pharmaceutical composition of any one of embodiments B7 to B16, wherein less than 70% of the oligosaccharides covalently attached to the population of the binding molecules via the N297 residue of the Fc region comprise a core fucose residue.

B18. The pharmaceutical composition of any one of embodiments B7 to B16, wherein less than 60% of the oligosaccharides covalently attached to the population of the binding molecules via the N297 residue of the Fc region comprise a core fucose residue.

B19. The pharmaceutical composition of any one of embodiments B7 to B16, wherein less than 50% of the oligosaccharides covalently attached to the population of the binding molecules via the N297 residue of the Fc region comprise a core fucose residue.

B20. The pharmaceutical composition of any one of embodiments B7 to B16, wherein less than 40% of the oligosaccharides covalently attached to the population of the binding molecules via the N297 residue of the Fc region comprise a core fucose residue.

B21. The pharmaceutical composition of any one of embodiments B7 to B16, wherein less than 30% of the oligosaccharides covalently attached to the population of the binding molecules via the N297 residue of the Fc region comprise a core fucose residue.

B22. The pharmaceutical composition of any one of embodiments B7 to B16, wherein less than 20% of the oligosaccharides covalently attached to the population of the binding molecules via the N297 residue of the Fc region comprise a core fucose residue.

B23. The pharmaceutical composition of any one of embodiments B7 to B16, wherein less than 10% of the oligosaccharides covalently attached to the population of the binding molecules via the N297 residue of the Fc region comprise a core fucose residue.

B24. The pharmaceutical composition of any one of embodiments B7 to B23, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

B25. The pharmaceutical composition of any one of embodiments B7 to B24, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

B26. The pharmaceutical composition of any one of embodiments B7 to B25, wherein the binding molecules are biparatopic or bispecific antibodies.

B27. The pharmaceutical composition of any one of embodiments B7 to B26, wherein the population of the binding molecules has enhanced ADCC, enhanced CDC, and/or enhanced ADCP.

In another set of embodiments (embodiment set C), provided are:

C1. A method of making a population of binding molecules, comprising expressing a polynucleotide encoding the binding molecules or a fragment thereof in a host cell that is deficient in adding a fucose residue to an oligosaccharide attached to an antibody via the N297 residue, wherein the population of the binding molecules comprises a binding molecule comprising an antigen binding domain and an Fc region, wherein
(i) the antigen binding domain is biparatopic or bispecific; and
(ii) the Fc region comprises K248E and T437R mutations (RE mutations), wherein amino acid residue numbering is according to the EU numbering system;
wherein the binding molecule has increased capability of hexamerization on a cell surface, and/or increased capability of engaging C1q.

C2. The method of embodiment C1, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

C3. The method of embodiment C2, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

C4. The method of any one of embodiments C1 to C3, wherein the first domain and the second domain bind to two different epitopes of the same antigen or different antigen(s).

C5. The method of any one of embodiments C1 to C4, wherein the binding molecule is a biparatopic or bispecific antibody.

C6. The method of any one of embodiments C1 to C5, wherein the binding molecule has enhanced CDC.

C7. A method of making a population of binding molecules, comprising expressing a polynucleotide encoding the binding molecules or a fragment thereof in a host cell that is deficient in adding a fucose residue to an oligosaccharide attached to an antibody via N297 residue, wherein each binding molecule comprises an antigen binding domain and an Fc region; wherein:
(i) the antigen binding domain is biparatopic or bispecific;
(ii) the Fc region comprises K248E and T437R mutations (RE mutations), wherein amino acid residue numbering is according to the EU numbering system; and
(iii) less than 80% of the oligosaccharides covalently attached to the population of the binding molecules via the N297 residue of the Fc region comprise a core fucose residue.

C8. A method of making a population of binding molecules, comprising expressing a polynucleotide encoding the binding molecules or a fragment thereof in a host cell that is deficient in adding a fucose residue to an oligosaccharide attached to an antibody via N297 residue,
wherein each binding molecule comprises an antigen binding domain and an Fc region;
wherein the antigen binding domain of at least 10% of the binding molecules in the population is biparatopic or bispecific;
wherein the Fc region of each binding molecule comprises K248E and T437R mutations (RE mutations), wherein amino acid residue numbering is according to the EU numbering system; and
wherein less than 80% of the oligosaccharides covalently attached to the population of the binding molecules via the N297 residue of the Fc region comprise a core fucose residue.

C9. The method of embodiment C7 or C8, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

C10. The method of embodiment C7 or C8, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment

C11. The method of embodiment C7 or C8, wherein the binding molecules are biparatopic or bispecific antibodies.

C12. The method of any one of embodiments C1 to C11, wherein the host cell has reduced α-1,6 fucosyltransferase activity.

C13. The method of any one of embodiments C1 to C11, wherein the host cell has reduced GDP-mannose 4,6-dehydratase activity.

C14. The method of any one of embodiments C1 to C11, wherein the gene encoding α-1,6 fucosyltransferase is mutated, expressed at a lower than normal level, or knocked out in the host cell.

C15. The method of any one of embodiments C1 to C11, wherein the gene encoding GDP-mannose 4,6-dehydratase is mutated, expressed at a lower than normal level, or knocked out in the host cell.

In another set of embodiments (embodiment set D), provided are:

D1. A method for enhancing CDC, ADCP, and/or ADCC in a system, comprising contacting the system with a binding molecule comprising an antigen binding domain and an Fc region, wherein
(i) the antigen binding domain is biparatopic or bispecific; and
(ii) the Fc region comprises K248E and T437R mutations (RE mutations), wherein amino acid residue numbering is according to the EU numbering system,
wherein the binding molecule has increased capability of hexamerization on a cell surface, and/or increased capability of engaging C1q.

D2. A method of modulating an immunity in a host, comprising administering a binding molecule comprising an antigen binding domain and an Fc region, wherein
(i) the antigen binding domain is biparatopic or bispecific; and
(ii) the Fc region comprises K248E and T437R mutations (RE mutations), wherein amino acid residue numbering is according to the EU numbering system,
wherein the binding molecule has increased capability of hexamerization on a cell surface, and/or increased capability of engaging C1q.

D3. A method of treating a disease or disorder in a subject comprising administering a binding molecule comprising an antigen binding domain and an Fc region, wherein
(i) the antigen binding domain is biparatopic or bispecific; and
(ii) the Fc region comprises K248E and T437R mutations (RE mutations), wherein amino acid residue numbering is according to the EU numbering system,
wherein the binding molecule has increased capability of hexamerization on a cell surface, and/or increased capability of engaging C1q.

D4. The method of any one of embodiments D1 to D3, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

D5. The method of embodiment D4, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

D6. The method of any one of embodiments D1 to D5, wherein the binding molecule is a biparatopic or bispecific antibody.

D7. The method of any one of embodiments D1 to D6, wherein the oligosaccharide covalently attached to the Fc region via the N297 residue thereof does not comprise a core fucose residue.

D8. A method for enhancing CDC, ADCP, and/or ADCC in a system, comprising contacting the system with a population of binding molecules, wherein each binding molecule comprising an antigen binding domain and an Fc region; wherein:
(i) the antigen binding domain is biparatopic or bispecific;
(ii) the Fc region comprises K248E and T437R mutations (RE mutations), wherein amino acid residue numbering is according to the EU numbering system; and
(iii) less than 80% of the oligosaccharides covalently attached to the population of the binding molecules via the N297 residue of the Fc region comprise a core fucose residue.

D9. A method for enhancing CDC, ADCP, and/or ADCC in a system, comprising contacting the system with a population of binding molecules,
wherein each binding molecule comprises an antigen binding domain and an Fc region;
wherein the antigen binding domain of at least 10% of the binding molecules in the population is biparatopic or bispecific;
wherein the Fc region of each binding molecule comprises K248E and T437R mutations (RE mutations), wherein amino acid residue numbering is according to the EU numbering system; and
wherein less than 80% of the oligosaccharides covalently attached to the population of the binding molecules via the N297 residue of the Fc region comprise a core fucose residue.

D10. A method of modulating an immunity in a host, comprising administering a population of binding molecules, wherein each binding molecule comprising an antigen binding domain and an Fc region; wherein:
(i) the antigen binding domain is biparatopic or bispecific;
(ii) the Fc region comprises K248E and T437R mutations (RE mutations), wherein amino acid residue numbering is according to the EU numbering system; and
(iii) less than 80% of the oligosaccharides covalently attached to the population of the binding molecules via the N297 residue of the Fc region comprise a core fucose residue.

D11. A method of modulating an immunity in a host, comprising administering a population of binding molecules,
wherein each binding molecule comprises an antigen binding domain and an Fc region;
wherein the antigen binding domain of at least 10% of the binding molecules in the population is biparatopic or bispecific;
wherein the Fc region of each binding molecule comprises K248E and T437R mutations (RE mutations), wherein amino acid residue numbering is according to the EU numbering system; and
wherein less than 80% of the oligosaccharides covalently attached to the population of the binding molecules via the N297 residue of the Fc region comprise a core fucose residue.

D12. A method of treating a disease or disorder in a subject comprising administering a population of binding molecules, wherein each binding molecule comprising an antigen binding domain and an Fc region; wherein:
(i) the antigen binding domain is biparatopic or bispecific;
(ii) the Fc region comprises K248E and T437R mutations (RE mutations), wherein amino acid residue numbering is according to the EU numbering system; and
(iii) less than 80% of the oligosaccharides covalently attached to the population of the binding molecules via the N297 residue of the Fc region comprise a core fucose residue.

D13. A method of treating a disease or disorder in a subject comprising administering a population of binding molecules,
wherein each binding molecule comprises an antigen binding domain and an Fc region;
wherein the antigen binding domain of at least 10% of the binding molecules in the population is biparatopic or bispecific;
wherein the Fc region of each binding molecule comprises K248E and T437R mutations (RE mutations), wherein amino acid residue numbering is according to the EU numbering system; and
wherein less than 80% of the oligosaccharides covalently attached to the population of the binding molecules via the N297 residue of the Fc region comprise a core fucose residue.

D14. The method of any one of embodiments D9, D11, and D13, wherein the antigen binding domain of at least 20% of the binding molecules in the population is biparatopic or bispecific.

D15. The method of any one of embodiments D9, D11, and D13, wherein the antigen binding domain of at least 30% of the binding molecules in the population is biparatopic or bispecific.

D16. The method of any one of embodiments D9, D11, and D13, wherein the antigen binding domain of at least 40% of the binding molecules in the population is biparatopic or bispecific.

D17. The method of any one of embodiments D9, D11, and D13, wherein the antigen binding domain of at least 50% of the binding molecules in the population is biparatopic or bispecific.

D18. The method of any one of embodiments D9, D11, and D13, wherein the antigen binding domain of at least 60% of the binding molecules in the population is biparatopic or bispecific.

D19. The method of any one of embodiments D9, D11, and D13, wherein the antigen binding domain of at least 70% of the binding molecules in the population is biparatopic or bispecific.

D20. The method of any one of embodiments D9, D11, and D13, wherein the antigen binding domain of at least 80% of the binding molecules in the population is biparatopic or bispecific.

D21. The method of any one of embodiments D9, D11, and D13, wherein the antigen binding domain of at least 90% of the binding molecules in the population is biparatopic or bispecific.

D22. The method of any one of embodiments D8 to D21, wherein less than 70% of the oligosaccharides covalently attached to the population of the binding molecules via the N297 residue of the Fc region comprise a core fucose residue.

D23. The method of any one of embodiments D8 to D21, wherein less than 60% of the oligosaccharides covalently attached to the population of the binding molecules via the N297 residue of the Fc region comprise a core fucose residue.

D24. The method of any one of embodiments D8 to D21, wherein less than 50% of the oligosaccharides covalently attached to the population of the binding molecules via the N297 residue of the Fc region comprise a core fucose residue.

D25. The method of any one of embodiments D8 to D21, wherein less than 40% of the oligosaccharides covalently attached to the population of the binding molecules via the N297 residue of the Fc region comprise a core fucose residue.

D26. The method of any one of embodiments D8 to D21, wherein less than 30% of the oligosaccharides covalently attached to the population of the binding molecules via the N297 residue of the Fc region comprise a core fucose residue.

D27. The method of any one of embodiments D8 to D21, wherein less than 20% of the oligosaccharides covalently attached to the population of the binding molecules via the N297 residue of the Fc region comprise a core fucose residue.

D28. The method of any one of embodiments D8 to D21, wherein less than 10% of the oligosaccharides covalently attached to the population of the binding molecules via the N297 residue of the Fc region comprise a core fucose residue.

D29. The method of any one of embodiments D8 to D28, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

D30. The method of embodiment D29, wherein:
(i) each of the first domain and the second domain is a Fab fragment, a scFv, a single domain antigen binding fragment, or an engineered binding fragment;
(ii) the first domain is a Fab fragment and the second domain is a scFv;
(iii) the first domain is a Fab fragment and the second domain is a single domain antigen binding fragment;
(iv) the first domain is a Fab fragment and the second domain is an engineered binding fragment;
(v) the first domain is a scFv and the second domain is a single domain antigen binding fragment;
(vi) the first domain is a scFv and the second domain is an engineered binding fragment; or
(vii) the first domain is a single domain antigen binding fragment and the second domain is an engineered binding fragment.

D31. The method of any one of embodiments D8 to D30, wherein the binding molecules are biparatopic or bispecific antibodies.

In another set of embodiments (embodiment set E), provided are:

E1. A binding molecule comprising a first means for enhancing CDC and a second means for enhancing CDC.

E2. The binding molecule of embodiment E1, further comprising a third means for enhancing ADCC.

E3. The binding molecule of embodiment E2, further comprising a fourth means for enhancing ADCP.

E4. The binding molecule of any one of embodiments E1 to E3, wherein the first means increases hexamerization of the binding molecule on a cell surface, and/or increases C1q engagement by the binding molecule.

E5. The binding molecule of any one of embodiments E1 to E4, wherein the first means enhances CDC, ADCP, and/or ADCC.

E6. The binding molecule of any one of embodiments E1 to E5, wherein the binding molecule is a biparatopic or bispecific antibody.

E7. A population of binding molecules comprising the binding molecule of any one of embodiments E1 to E6.

E8. A method of making a population of binding molecules, comprising
(i) a step for introducing K248E and T437R mutations (RE mutations) in the Fc region of the binding molecules; and
(ii) a step for producing the population of binding molecules with reduced amount of core fucoses in the oligosaccharides attached to the binding molecules via the N297 residue,
wherein the binding molecules comprise a biparatopic or bispecific antigen binding domain.

E9. A population of binding molecules produced according to the method of embodiment E8.

E10. A method for enhancing CDC, ADCP, and/or ADCC in a system, comprising contacting the system with the binding molecule of any one of embodiments E1 to E6 or the population of the binding molecules of embodiment E7 or E9.

E11. A method of modulating an immunity in a host, comprising administering the binding molecule of any one of embodiments E1 to E6 or the population of the binding molecules of embodiment E7 or E9.

E12. A method of treating a disease or disorder in a subject comprising administering the binding molecule of any one of embodiments 1 to 6 or the population of the binding molecules of embodiment E7 or E9.

### 7. EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### Example 1—ADCC and CDC Functional Analysis of Antibodies with Modified Constant Regions

### Example 1.1 Anti-CD37 Antibodies

Eight different types of anti-CD37 antibodies were tested for their ability to mediate tumor cell killing via CDC in the cell lines CARNAVAL, which expresses high levels of CD37, and JEKO-1, which expresses lower levels of CD37. Briefly, for each type of anti-CD37 antibody, an antibody with low fucosylation (i.e., T26B375.CLF, T26B382.CLF, T26B386.CLF, T26B379.CLF, T26B373.CLF, T26B388.CLF, T26B385.CLF and T26B374.CLF, respectively), an antibody comprising S239D/I332E (Xencor) mutations (i.e., T26B612, T26B613, T26B614, T26B615, T26B608, T26B609, T26B610 and T26B611, respectively), an antibody comprising RE mutations (i.e., T26B461, T26B463, T26B465, T26B462, T26B459, T26B466, T26B464 and T26B460, respectively), and an antibody comprising RE mutations with low fucosylation (i.e., T26B461.CLF, T26B463.CLF, T26B465.CLF, T26B462.CLF, T26B459.CLF, T26B466.CLF, T26B464.CLF and T26B460.CLF, respectively) were generated. Anti-CD37 antibodies were added to the CARNAVAL target cells and incubated for 30 minutes at 37° C. Baby rabbit serum was then added to target cells to a final concentration of 10% to provide a source of complement components for CDC. The mixture was incubated for 4 hours at 37° C. 100 μl of CellTiter-Glo reagent (Promega) was added to the mixture followed by incubation for 10 minutes at room temperature. Target cell viability was determined by measuring luminescence with a Tecan SPARK Reader and reported in Relative Luminescence Units (RLU).

Anti-CD37 antibodies and their variants with modified constant regions are summarized in Table 2 below.

TABLE 2
Anti-CD37 antibodies and their variants

| | | | | Antibodies |
|---|---|---|---|---|
| | IgG1 | | | with RE |
| | antibodies | Antibodies | Antibodies | mutations and |
| | with low | with Xencor | with RE | low |
| | fucosylation | mutations | mutations | fucosylation |
| 1 | T26B375.CLF | T26B612 | T26B461 | T26B461.CLF |
| 2 | T26B382.CLF | T26B613 | T26B463 | T26B463.CLF |
| 3 | T26B386.CLF | T26B614 | T26B465 | T26B465.CLF |
| 4 | T26B379.CLF | T26B615 | T26B462 | T26B462.CLF |
| 5 | T26B373.CLF | T26B608 | T26B459 | T26B459.CLF |
| 6 | T26B388.CLF | T26B609 | T26B466 | T26B466.CLF |
| 7 | T26B385.CLF | T26B610 | T26B464 | T26B464.CLF |
| 8 | T26B374.CLF | T26B611 | T26B460 | T26B460.CLF |

The amino acid sequences of heavy chain and light chain of exemplary anti-CD37 antibodies are summarized in Table 3 and Table 4 below.

TABLE 3
Heavy chain of Anti-CD37 antibodies

| Anti- | | SEQ |
|---|---|---|
| body | | ID |
| Name | Heavy Chain | NO: |
| T26B373 | QVQLQESGPGLVKPSETLSLTCTVSGGSISSGVYYW | 1 |
| | AWIRQPPGKGLELIGTFYYSGSTYYDSSLRSRVTIS | |
| | VDTSKNQFSLKLSSVTAADTAVYYCARQAGDFDYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPELLGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALPAPIEKTISKAKGQPREPQVYTLPPSREEMTK | |
| | NQVSLTCLVKGFYPSDIAVEWESNGQPENNYKTTPP | |
| | VLDSDGSFFLYSKLTVDKSRWQQGNVFSCSVMHEAL | |
| | HNHYTQKSLSLSPGK | |
| T26B374 | QLQLQESGPGLVKPSETLSLTCTVSGGSISSSPYYW | 2 |
| | GWIRQPPGKGLEWIGSFYYGGSNYYNPSLKSRVTIS | |
| | ADTSKNQFSLKLSSVTAADTAVYYCARQAGDWDYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPELLGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALPAPIEKTISKAKGQPREPQVYTLPPSREEMTK | |
| | NQVSLTCLVKGFYPSDIAVEWESNGQPENNYKTTPP | |
| | VLDSDGSFFLYSKLTVDKSRWQQGNVFSCSVMHEAL | |
| | HNHYTQKSLSLSPGK | |
| T26B375 | QLQLQESGPGLVKPSETLSLTCTVSGGSISSGIYNW | 3 |
| | GWIRQPPGKGLEWIGNFQYSGITYYNPSLKSRVTIS | |
| | VDTSKNQFSLQLSSVTAADTAVYYCARQAGDFDYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPELLGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALPAPIEKTISKAKGQPREPQVYTLPPSREEMTK | |
| | NQVSLTCLVKGFYPSDIAVEWEPPSNGQPENNYKTT | |
| | VLDSDGSFFLYSKLTVDKSRWQQGNVFSCSVMHEAL | |
| | HNHYTQKSLSLSPGK | |
| T26B379 | QVQLQESGPGLVKPSETLSLTCTVSGGSISSSIYYW | 4 |
| | AWIRQPPGKGLEWIGTIYYGGSPYYSPSLKSRVTIS | |
| | IDTSKSQFSLRLTSVTVADTAVYYCARRAGDFDYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPELLGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALPAPIEKTISKAKGQPREPQVYTLPPSREEMTK | |
| | NQVSLTCLVKGFYPSDIAVEWESNGQPENNYKTTPP | |
| | VLDSDGSFFLYSKLTVDKSRWQQGNVFSCSVMHEAL | |
| | HNHYTQKSLSLSPGK | |
| T26B382 | QVQLVESGGGLVKPGGSLRLSCAASGFTFSDYYMTW | 5 |
| | IRQAPGKGLEWVSYISSSGITKYYADFVKGRFTISR | |
| | DNAKNSLYLQMNSLRAEDTAVYYCARDRDRQWLLEF | |
| | DYWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVD | |
| | KKVEPKSCDKTHTCPPCPAPELLGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALPAPIEKTISKAKGQPREPQVYTLPPSRE | |
| | EMTKNQVSLTCLVKGFYPSDIAVEWESNGQPENNYK | |
| | TTPPVLDSDGSFFLYSKLTVDKSRWQQGNVFSCSVM | |
| | HEALHNHYTQKSLSLSPGK | |
| T26B385 | QVQLQESGPGLVKPSETLSLTCIVSGGSVSSRNYYW | 6 |
| | GWIRQPPGKGLEWIGRIYYSGNTNYNPSLKSRVTIS | |
| | VDTSKNQFSLKLSSVTAADTAVYYCARWAGEIDYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPELLGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALPAPIEKTISKAKGQPREPQVYTLPPSREEMTK | |
| | NQVSLTCLVKGFYPSDIAVEWESNGQPENNYKTTPP | |
| | VLDSDGSFFLYSKLTVDKSRWQQGNVFSCSVMHEAL | |
| | HNHYTQKSLSLSPGK | |
| T26B386 | QVQLQESGPGLVKPSETLSLSCTVSGGSLNSYNYYW | 7 |
| | GWVRQPPGKGLEWIGTIYSSGSAYYNPSLKSRFTIS | |
| | VATSKNQFSLRLSSVTAADTAVYYCARGYRNSWYAL | |
| | FEYWGQGTLVTVSSASTKGPSVFPLAPSSKSTSGGT | |
| | AALGCLVKDYFPEPVTVSWNSGALTSGVHTFPAVLQ | |
| | SSGLYSLSSVVTVPSSSLGTQTYICNVNHKPSNTKV | |
| | DKKVEPKSCDKTHTCPPCPAPELLGGPSVFLFPPKP | |
| | KDTLMISRTPEVTCVVVDVSHEDPEVKFNWYVDGVE | |
| | VHNAKTKPREEQYNSTYRVVSVLTVLHQDWLNGKEY | |
| | KCKVSNKALPAPIEKTISKAKGQPREPQVYTLPPSR | |
| | EEMTKNQVSLTCLVKGFYPSDIAVEWESNGQPENNY | |
| | KTTPPVLDSDGSFFLYSKLTVDKSRWQQGNVFSCSV | |
| | MHEALHNHYTQKSLSLSPGK | |
| T26B388 | QVQLQQWGAGLLKPSETLSLTCAVYGGSFSDYYWSW | 8 |
| | IRQPPGKGLEWIGEIDHSGSTDYNPSLKSRVTISVD | |
| | TSKNQFSLKLSSVTAADTAVYYCARSMYYDIWTGYH | |
| | GAFDIWGQGTMVTVSSASTKGPSVFPLAPSSKSTSG | |
| | GTAALGCLVKDYFPEPVTVSWNSGALTSGVHTFPAV | |
| | LQSSGLYSLSSVVTVPSSSLGTQTYICNVNHKPSNT | |
| | KVDKKVEPKSCDKTHTCPPCPAPELLGGPSVFLFPP | |
| | KPKDTLMISRTPEVTCVVVDVSHEDPEVKFNWYVDG | |
| | VEVHNAKTKPREEQYNSTYRVVSVLTVLHQDWLNGK | |
| | EYKCKVSNKALPAPIEKTISKAKGQPREPQVYTLPP | |
| | SREEMTKNQVSLTCLVKGFYPSDIAVEWESNGQPEN | |
| | NYKTTPPVLDSDGSFFLYSKLTVDKSRWQQGNVFSC | |
| | SVMHEALHNHYTQKSLSLSPGK | |

TABLE 4
Light chain of Anti-CD37 antibodies

| Anti- | | SEQ |
|---|---|---|
| body | | ID |
| Name | Light Chain | NO: |
| T26B373 | DIQMTQSPSTLSASVGDRVTITCRASQSTSSWLAWY | 9 |
| | QQKPGKAPKLLIYKTSSLESGVPSRFSGSGSGTEFT | |
| | LTISSLQPDDFATYYCQQYNSYSFGQGTKVEIKRTV | |
| | AAPSVFIFPPSDEQLKSGTASVVCLLNNFYPREAKV | |
| | QWKVDNALQSGNSQESVTEQDSKDSTYSLSSTLTLS | |
| | KADYEKHKVYACEVTHQGLSSPVTKSFNRGEC | |
| T26B374 | DIQMTQSPSTLSASVGDRVTITCRASQSISSWLAWY | 10 |
| | QQKPGKAPKLLIYKASSLESGVPSRFSGSRSGTEFT | |
| | LTISSLQPDDFATYYCQQYNSWTFGQGTKVEIKRTV | |
| | AAPSVFIFPPSDEQLKSGTASVVCLLNNFYPREAKV | |
| | QWKVDNALQSGNSQESVTEQDSKDSTYSLSSTLTLS | |
| | KADYEKHKVYACEVTHQGLSSPVTKSFNRGEC | |
| T26B375 | DIQMTQSPSTLSASVGDRVTITCRASQSISSWLAWY | 11 |
| | QQKPGKAPKLLIYKASSLESGVPSRFSGSGSGTEFT | |
| | LTISSLQPDDFATYYCQQYNSYTFGQGTKLEIKRTV | |
| | AAPSVFIFPPSDEQLKSGTASVVCLLNNFYPREAKV | |
| | QWKVDNALQSGNSQESVTEQDSKDSTYSLSSTLTLS | |
| | KADYEKHKVYACEVTHQGLSSPVTKSFNRGEC | |
| T26B379 | DIQMTQSPSTLSASVGDRVTITCRASQSISSWLAWY | 12 |
| | QQKPGKAPKLLIFKTSSLESGVPSRFSGSGSGTEFT | |
| | LTISSLQPDDFATYYCQQYNSYSFGGGTKVEIKRTV | |
| | AAPSVFIFPPSDEQLKSGTASVVCLLNNFYPREAKV | |
| | QWKVDNALQSGNSQESVTEQDSKDSTYSLSSTLTLS | |
| | KADYEKHKVYACEVTHQGLSSPVTKSFNRGEC | |
| T26B382 | QSALTQPASVSGSPGQSITISCTGTSSDVGGYNYVS | 13 |
| | WYQQHPSKAPKLMIYDVSNRPSGVSNRFSGSKSGNT | |
| | ASLTISGLQAEDEADYYCSSYTSSSTLVVFGGGTKL | |
| | TVLGQPKAAPSVTLFPPSSEELQANKATLVCLISDF | |
| | YPGAVTVAWKADSSPVKAGVETTTPSKQSNNKYAAS | |
| | SYLSLTPEQWKSHRSYSCQVTHEGSTVEKTVAPTEC | |
| | S | |
| T26B385 | DIQMTQSPSTLSASVGDRVTITCRASQSLSSWLAWY | 14 |
| | QQKPGKAPKLLIFKTSSLESGVPSRFSGSGSGTEFT | |
| | LTISSLQPDDFAIYYCQQYNSYIFGQGTRLEIKRTV | |
| | AAPSVFIFPPSDEQLKSGTASVVCLLNNFYPREAKV | |
| | QWKVDNALQSGNSQESVTEQDSKDSTYSLSSTLTLS | |
| | KADYEKHKVYACEVTHQGLSSPVTKSFNRGEC | |
| T26B386 | SSELTQDPAVSVALGQTVRITCQGDSLRNYYASWYQ | 15 |
| | QKPGQAPVLVFYGKDNRPSGIPDRFSGSTSGNTASL | |
| | TITGAQAQDEADYYCNSRDSSGDHLVFGGGTKLTVL | |
| | GQPKAAPSVTLFPPSSEELQANKATLVCLISDFYPG | |
| | AVTVAWKADSSPVKAGVETTTPSKQSNNKYAASSYL | |
| | SLTPEQWKSHRSYSCQVTHEGSTVEKTVAPTECS | |
| T26B388 | DIQMTQSPSSLSASVGDRVTITCRASQGIRNDLGWY | 16 |
| | QQKPGKAPKRLIYAASSLQGGVPSRFSGSGSGTEFT | |
| | LTISSLQPEDFATYYCLQHYTYPLTFGGGTKVEIKR | |
| | TVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |

The amino acid sequences of the VH CDRs and VL CDRs of exemplary anti-CD37 antibodies are summarized in Table 5 to Table 14 below.

TABLE 5

| Anti- | | SEQ | | SEQ | | SEQ |
|---|---|---|---|---|---|---|
| body | HCDR1 | ID | HCDR2 | ID | HCDR3 | ID |
| Name | (Abm) | NO: | (Abm) | NO: | (Abm) | NO: |
| T26B373 | GGSISSGVYY | 17 | TFYYSGST | 18 | QAGDFDY | 19 |
| | WA | | Y | | | |
| T26B374 | GGSISSSPYY | 20 | SFYYGGSN | 21 | QAGDWDY | 22 |
| | WG | | Y | | | |
| T26B375 | GGSISSGIYN | 23 | NFQYSGIT | 24 | QAGDFDY | 25 |
| | WG | | Y | | | |
| T26B379 | GGSISSSIYY | 26 | TIYYGGSP | 27 | RAGDFDY | 28 |
| | WA | | Y | | | |
| T26B382 | GFTFSDYYMT | 29 | YISSSGIT | 30 | DRDRQWL | 31 |
| | | | KY | | LEFDY | |
| T26B385 | GGSVSSRNYY | 32 | RIYYSGNT | 33 | WAGEIDY | 34 |
| | WG | | N | | | |
| T26B386 | GGSLNSYNYY | 35 | TIYSSGSA | 36 | GYRNSWY | 37 |
| | WG | | Y | | ALFEY | |
| T26B388 | GGSFSDYYWS | 38 | EIDHSGST | 39 | SMYYDIW | 40 |
| | | | D | | TGYHGAF | |
| | | | | | DI | |

TABLE 6

| Anti- | | SEQ | | SEQ | | SEQ |
|---|---|---|---|---|---|---|
| body | HCDR1 | ID | HCDR2 | ID | HCDR3 | ID |
| Name | (KABAT) | NO: | (KABAT) | NO: | (KABAT) | NO: |
| T26B373 | SGVYYWA | 41 | TFYYSGSTYYD | 42 | QAGDFDY | 43 |
| | | | SSLRS | | | |
| T26B374 | SSPYYWG | 44 | SFYYGGSNYYN | 45 | QAGDWDY | 46 |
| | | | PSLKS | | | |
| T26B375 | SGIYNWG | 47 | NFQYSGITYYN | 48 | QAGDFDY | 49 |
| | | | PSLKS | | | |
| T26B379 | SSIYYWA | 50 | TIYYGGSPYYS | 51 | RAGDFDY | 52 |
| | | | PSLKS | | | |
| T26B382 | DYYMT | 53 | YISSSGITKYY | 54 | DRDRQWL | 55 |
| | | | ADFVKG | | LEFDY | |
| T26B385 | SRNYYWG | 56 | RIYYSGNTNYN | 57 | WAGEIDY | 58 |
| | | | PSLKS | | | |
| T26B386 | SYNYYWG | 59 | TIYSSGSAYYN | 60 | GYRNSWY | 61 |
| | | | PSLKS | | ALFEY | |
| T26B388 | DYYWS | 62 | EIDHSGSTDYN | 63 | SMYYDIW | 64 |
| | | | PSLKS | | TGYHGAF | |
| | | | | | DI | |

TABLE 7

| Anti- | | SEQ | | SEQ | | SEQ |
|---|---|---|---|---|---|---|
| body | HCDR1 | ID | HCDR2 | ID | HCDR3 | ID |
| Name | (CHOTHIA) | NO: | (CHOTHIA) | NO: | (CHOTHIA) | NO: |
| T26B373 | GGSISSGVY | 65 | YYSGS | 66 | QAGDFDY | 67 |
| T26B374 | GGSISSSPY | 68 | YYGGS | 69 | QAGDWDY | 70 |
| T26B375 | GGSISSGIY | 71 | QYSGI | 72 | QAGDFDY | 73 |
| T26B379 | GGSISSSIY | 74 | YYGGS | 75 | RAGDFDY | 76 |
| T26B382 | GFTFSDY | 77 | SSSGIT | 78 | DRDRQWL | 79 |
| | | | | | LEFDY | |
| T26B385 | GGSVSSRNY | 80 | YYSGN | 81 | WAGEIDY | 82 |
| T26B386 | GGSLNSYNY | 83 | YSSGS | 84 | GYRNSWY | 85 |
| | | | | | ALFEY | |
| T26B388 | GGSFSDY | 86 | DHSGS | 87 | SMYYDIW | 88 |
| | | | | | TGYHGAFDI | |

TABLE 8

| Anti- | | QSE | | SEQ | | SEQ |
|---|---|---|---|---|---|---|
| body | HCDR1 | ID | HCDR2 | ID | HCDR3 | ID |
| Name | (IMGT) | NO: | (IMGT) | NO: | (IMGT) | NO: |
| T26B373 | GGSISSGVY | 89 | FYYSGST | 90 | ARQAGDFDY | 91 |
| | Y | | | | | |
| T26B374 | GGSISSSPYY | 92 | FYYGGS | 93 | ARQAGDWDY | 94 |
| | | | N | | | |
| T26B375 | GGSISSGIYN | 95 | FQYSGIT | 96 | ARQAGDFDY | 97 |
| T26B379 | GGSISSSIYY | 98 | IYYGGSP | 99 | ARRAGDFDY | 100 |
| T26B382 | GFTFSDYY | 101 | ISSSGITK | 102 | ARDRDRQWL | 103 |
| | | | | | LEFDY | |
| T26B385 | GGSVSSRNY | 104 | IYYSGNT | 105 | ARWAGEIDY | 106 |
| | Y | | | | | |
| T26B386 | GGSLNSYNY | 107 | IYSSGSA | 108 | ARGYRNSWY | 109 |
| | Y | | | | ALFEY | |
| T26B388 | GGSFSDYY | 110 | IDHSGST | 111 | ARSMYYDIW | 112 |
| | | | | | TGYHGAFDI | |

TABLE 9

| Anti- | | SEQ | | SEQ | | SEQ |
|---|---|---|---|---|---|---|
| body | HCDR1 | ID | HCDR2 | ID | HCDR3 | ID |
| Name | (CONTACT) | NO: | (CONTACT) | NO: | (CONTACT) | NO: |
| T26B373 | SSGVYY | 113 | LIGTFYYSG | 114 | ARQAGDFD | 115 |
| | WA | | STY | | | |
| T26B374 | SSSPYYW | 116 | WIGSFYYGG | 117 | ARQAGDWD | 118 |
| | G | | SNY | | | |
| T26B375 | SSGIYNW | 119 | WIGNFQYSG | 120 | ARQAGDFD | 121 |
| | G | | ITY | | | |
| T26B379 | SSSIYYW | 122 | WIGTIYYGG | 123 | ARRAGDFD | 124 |
| | A | | SPY | | | |
| T26B382 | SDYYMT | 125 | WVSYISSSG | 126 | ARDRDRQWL | 127 |
| | | | ITKY | | LEFD | |
| T26B385 | SSRNYY | 128 | WIGRIYYSG | 129 | ARWAGEID | 130 |
| | WG | | NTN | | | |
| T26B386 | NSYNYY | 131 | WIGTIYSSG | 132 | ARGYRNSWY | 133 |
| | WG | | SAY | | ALFE | |
| T26B388 | SDYYWS | 134 | WIGEIDHSG | 135 | ARSMYYDIW | 136 |
| | | | STD | | TGYHGAFD | |

TABLE 10

| Anti- | | SEQ | | SEQ | | SEQ |
|---|---|---|---|---|---|---|
| body | LCDR1 | ID | LCDR2 | ID | LCDR3 | ID |
| Name | (Abm) | NO: | (Abm) | NO: | (Abm) | NO: |
| T26B373 | RASQSTSSWLA | 137 | KTSSLES | 138 | QQYNSYS | 139 |
| T26B374 | RASQSISSWLA | 140 | KASSLES | 141 | QQYNSWT | 142 |
| T26B375 | RASQSISSWLA | 143 | KASSLES | 144 | QQYNSYT | 145 |
| T26B379 | RASQSISSWLA | 146 | KTSSLES | 147 | QQYNSYS | 148 |
| T26B382 | TGTSSDVGGYN | 149 | DVSNRPS | 150 | SSYTSSS | 151 |
| | YVS | | | | TLVV | |
| T26B385 | RASQSLSSWLA | 152 | KTSSLES | 153 | QQYNSYI | 154 |
| T26B386 | QGDSLRNYYAS | 155 | GKDNRPS | 156 | NSRDSSG | 157 |
| | | | | | DHLV | |
| T26B388 | RASQGIRNDLG | 158 | AASSLQG | 159 | LQHYTYP | 160 |
| | | | | | LT | |

TABLE 11

| Anti- | | SEQ | | SEQ | | SEQ |
|---|---|---|---|---|---|---|
| body | LCDR1 | ID | LCDR2 | ID | LCDR3 | ID |
| Name | (KABAT) | NO: | (KABAT) | NO: | (KABAT) | NO: |
| T26B373 | RASQSTSSWLA | 161 | KTSSLES | 162 | QQYNSYS | 163 |
| T26B374 | RASQSISSWLA | 164 | KASSLES | 165 | QQYNSWT | 166 |
| T26B375 | RASQSISSWLA | 167 | KASSLES | 168 | QQYNSYT | 169 |
| T26B379 | RASQSISSWLA | 170 | KTSSLES | 171 | QQYNSYS | 172 |
| T26B382 | TGTSSDVGGYN | 173 | DVSNRPS | 174 | SSYTSSS | 175 |
| | YVS | | | | TLVV | |
| T26B385 | RASQSLSSWLA | 176 | KTSSLES | 177 | QQYNSYI | 178 |
| T26B386 | QGDSLRNYYAS | 179 | GKDNRPS | 180 | NSRDSSG | 181 |
| | | | | | DHLV | |
| T26B388 | RASQGIRNDLG | 182 | AASSLQG | 183 | LQHYTYP | 184 |
| | | | | | LT | |

TABLE 12

| Anti- | | SEQ | | SEQ | | SEQ |
|---|---|---|---|---|---|---|
| body | LCDR1 | ID | LCDR2 | ID | LCDR3 | ID |
| Name | (CHOTHIA) | NO: | (CHOTHIA) | NO: | (CHOTHIA) | NO: |
| T26B373 | RASQSTSSWLA | 185 | KTSSLES | 186 | QQYNSYS | 187 |
| T26B374 | RASQSISSWLA | 188 | KASSLES | 189 | QQYNSWT | 190 |
| T26B375 | RASQSISSWLA | 191 | KASSLES | 192 | QQYNSYT | 193 |
| T26B379 | RASQSISSWLA | 194 | KTSSLES | 195 | QQYNSYS | 196 |
| T26B382 | TGTSSDVGGYN | 197 | DVSNRPS | 198 | SSYTSSS | 199 |
| | YVS | | | | TLVV | |
| T26B385 | RASQSLSSWLA | 200 | KTSSLES | 201 | QQYNSYI | 202 |
| T26B386 | QGDSLRNYYAS | 203 | GKDNRPS | 204 | NSRDSSG | 205 |
| | | | | | DHLV | |
| T26B388 | RASQGIRNDLG | 206 | AASSLQG | 207 | LQHYTYP | 208 |
| | | | | | LT | |

TABLE 13

| Anti- | | SEQ | LCDR2 | SEQ | | SEQ |
|---|---|---|---|---|---|---|
| body | LCDR1 | ID | | ID | LCDR3 | ID |
| Name | (IMGT) | NO: | (IMGT) | NO: | (IMGT) | NO: |
| T26B373 | QSTSSW | 209 | KTS | 210 | QQYNSYS | 211 |
| T26B374 | QSISSW | 212 | KAS | 213 | QQYNSWT | 214 |
| T26B375 | QSISSW | 215 | KAS | 216 | QQYNSYT | 217 |
| T26B379 | QSISSW | 218 | KTS | 219 | QQYNSYS | 220 |
| T26B382 | SSDVGGYNY | 221 | DVS | 222 | SSYTSSSTLVV | 223 |
| T26B385 | QSLSSW | 224 | KTS | 225 | QQYNSYI | 226 |
| T26B386 | SLRNYY | 227 | GKD | 228 | NSRDSSGDHLV | 229 |
| T26B388 | QGIRND | 230 | AAS | 231 | LQHYTYPLT | 232 |

TABLE 14

| Anti- | | SEQ | | SEQ | | SEQ |
|---|---|---|---|---|---|---|
| body | LCDR1 | ID | LCDR2 | ID | LCDR3 | ID |
| Name | (CONTACT) | NO: | (CONTACT) | NO: | (CONTACT) | NO: |
| T26B373 | SSWLAWY | 233 | LLIYKTSSLE | 234 | QQYNSY | 235 |
| T26B374 | SSWLAWY | 236 | LLIYKASSLE | 237 | QQYNSW | 238 |
| T26B375 | SSWLAWY | 239 | LLIYKASSLE | 240 | QQYNSY | 241 |
| T26B379 | SSWLAWY | 242 | LLIFKTSSLE | 243 | QQYNSY | 244 |
| T26B382 | VGGYNYV | 245 | LMIYDVSNRP | 246 | SSYTSS | 247 |
| | SWY | | | | STLV | |
| T26B385 | SSWLAWY | 248 | LLIFKTSSLE | 249 | QQYNSY | 250 |
| T26B386 | NYYASWY | 251 | LVFYGKDNRP | 252 | NSRDSS | 253 |
| | | | | | GDHL | |
| T26B388 | RNDLGWY | 254 | RLIYAASSLQ | 255 | LQHYTY | 256 |
| | | | | | PL | |

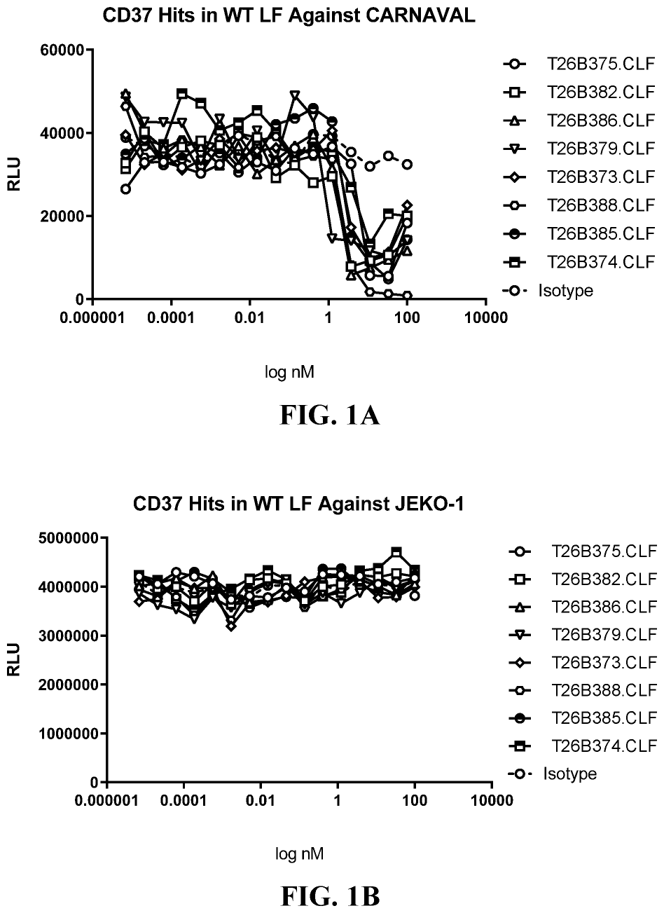

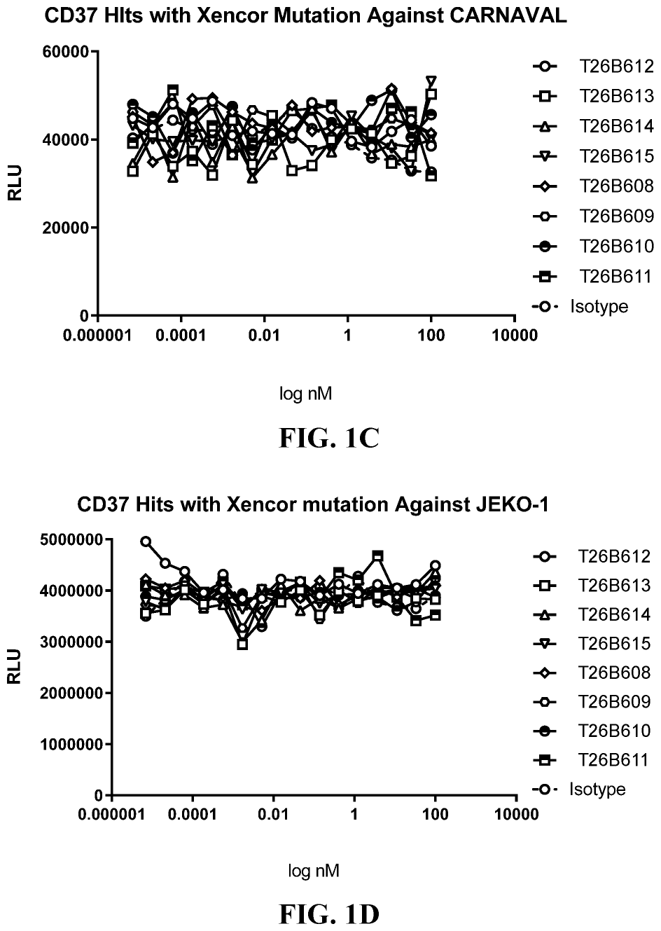

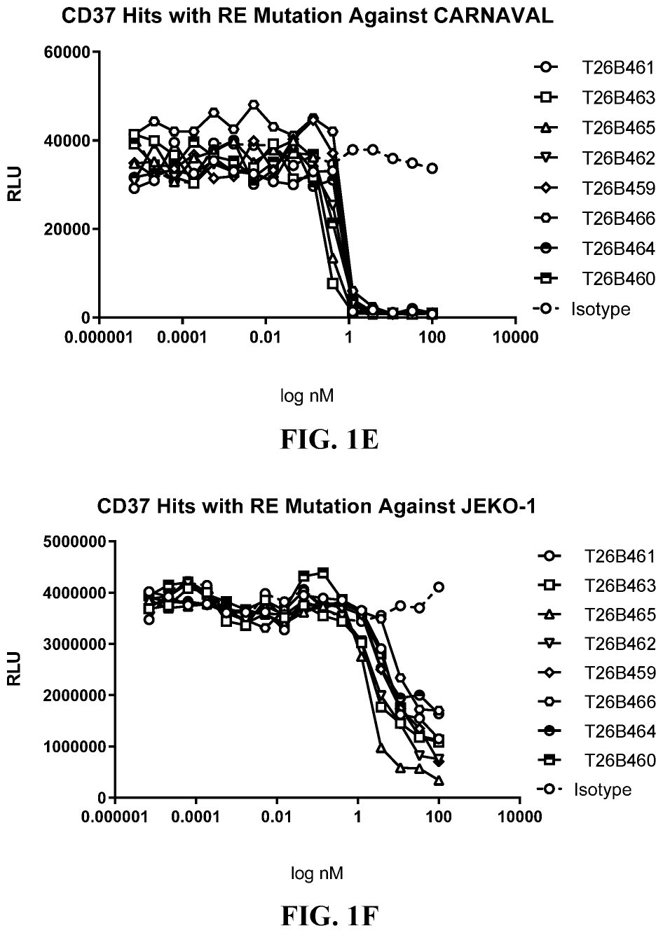

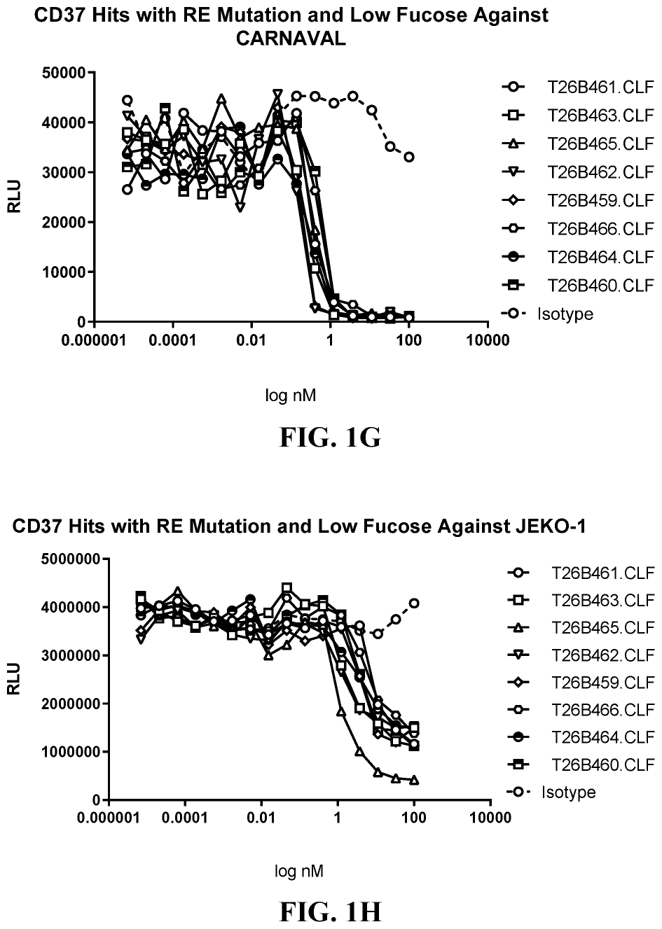

The anti-CD37 antibodies with low fucosylation showed CDC activity against CARNAVAL cells but not JEKO-1 cells (FIGS. 1A and 11B). Xencor mutations in these antibodies ablated their CDC activity against CARNAVAL cells significantly (FIG. 1C). RE mutations further increased the CDC potency of these antibodies against CARNAVAL cells, while conferring these antibodies CDC activity against JEKO-1 cells (FIGS. 1E and 1F). Importantly, the potentiation of CDC activity by RE mutations were unaffected when antibodies with low fucosylation were used (FIGS. 1G and 1H) compared to those with normal fucosylation (FIGS. 1E and 1F).

Taken together, these results indicated that the CDC potentiation characteristics of the RE mutations are not target dependent. They also showed that, while the S239D/I332E mutations were shown to improve ADCC activity of antibodies (Lazar, G. A. et al., supra), they negatively affect CDC. In contrast, the RE mutations can enhance CDC activity of antibodies. This potentiation is not affected by low fucosylation of the antibodies, which can potentiate their ADCC activity.

### Example 1.2 Anti-GPRC5D Antibodies

To further confirm that the CDC potentiation characteristics of the RE mutations in the Fc region are general properties of the mutations, effects of the RE mutations were investigated in a third target cell line, H929, expressing GPRC5D. Briefly, a wildtype IgG1 antibody with low fucosylation, GC5B747.CLF, and its counterpart with RE mutations, GC5B752.CLF, were added to the H929 target cells and incubated for 30 minutes at 37° C. Baby rabbit serum was then added to target cells to a final concentration of 10% to provide a source of complement components for CDC. The mixture was incubated for 4 hours at 37° C. 100 μl of CellTiter-Glo reagent (Promega) was added to the mixture, followed by incubation for 10 minutes at room temperature. Target cell viability was determined by measuring luminescence with a Tecan SPARK Reader and reported in Relative Luminescence Units (RLU).

The amino acid sequences of heavy chain and light chain of exemplary anti-GPRC5D antibodies are summarized in Table 15 and Table 16 below.

TABLE 15
Heavy chain of Anti-GPRC5D antibodies

| Anti- | | SEQ |
|---|---|---|
| body | | ID |
| Name | Heavy Chain | NO: |
| GC5B747 | QLQLQESGPGLVKPSETLSLTCTVSGGSLSS | 257 |
| | SSYWWGWTRQPPGRGLEWIGTMYYSGNIYYN | |
| | PSLQSRATISVDTSKNQFSLKLSSVTAADTA | |
| | VYYCARHVGYSYGRRFWYFDLWGRGTLVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNT | |
| | KVDKKVEPKSCDKTHTCPPCPAPELLGGPSV | |
| | FLFPPKPKDTLMISRTPEVTCVVVDVSHEDP | |
| | EVKFNWYVDGVEVHNAKTKPREEQYNSTYRV | |
| | VSVLTVLHQDWLNGKEYKCKVSNKALPAPIE | |
| | KTISKAKGQPREPQVYTLPPSREEMTKNQVS | |
| | LTCLVKGFYPSDIAVEWESNGQPENNYKTTP | |
| | PVLDSDGSFFLYSKLTVDKSRWQQGNVFSCS | |
| | VMHEALHNHYTQKSLSLSPG | |
| GC5B752 | QLQLQESGPGLVKPSETLSLTCTVSGGSLSS | 258 |
| | SSYWWGWTRQPPGRGLEWIGTMYYSGNIYYN | |
| | PSLQSRATISVDTSKNQFSLKLSSVTAADTA | |
| | VYYCARHVGYSYGRRFWYFDLWGRGTLVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNT | |
| | KVDKKVEPKSCDKTHTCPPCPAPELLGGPSV | |
| | FLFPPKPEDTLMISRTPEVTCVVVDVSHEDP | |
| | EVKFNWYVDGVEVHNAKTKPREEQYNSTYRV | |
| | VSVLTVLHQDWLNGKEYKCKVSNKALPAPIE | |
| | KTISKAKGQPREPQVYTLPPSREEMTKNQVS | |
| | LTCLVKGFYPSDIAVEWESNGQPENNYKTTP | |
| | PVLDSDGSFFLYSKLTVDKSRWQQGNVFSCS | |
| | VMHEALHNHYRQKSLSLSPG | |

TABLE 16
Light chain of Anti-GPRC5D antibodies

| Anti- | | SEQ |
|---|---|---|
| body | | ID |
| Name | Light Chain | NO: |
| GC5B747 | EIVLTQSPATLSLSPGERATLSCRASQSVSS | 259 |
| | YLAWYQQKPGQAPRLLIYDASNRATGIPARF | |
| | SGSGSGTDFTLTISSLEPEDFAVYYCQQRSN | |
| | WPPTFGQGTKVEIKRTVAAPSVFIFPPSDEQ | |
| | LKSGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| GC5B752 | EIVLTQSPATLSLSPGERATLSCRASQSVSS | 260 |
| | YLAWYQQKPGQAPRLLIYDASNRATGIPARF | |
| | SGSGSGTDFTLTISSLEPEDFAVYYCQQRSN | |
| | WPPTFGQGTKVEIKRTVAAPSVFIFPPSDEQ | |
| | LKSGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |

GC5B747 and GC5B752 share the same amino acid sequences of the VH CDRs and VL CDRs, which are summarized in Table 17 below.

TABLE 17

| | SEQ | | SEQ | | SEQ |
|---|---|---|---|---|---|
| HCDR1 | ID | HCDR2 | ID | HCDR3 | ID |
| (Abm) | NO: | (Abm) | NO: | (Abm) | NO: |
| GGSLSSSSYWW | 261 | TMYYSGNIY | 262 | HVGYSYGRR | 263 |
| G | | | | FWYFDL | |
| | SEQ | | SEQ | | SEQ |
| HCDR1 | ID | HCDR2 | ID | HCDR3 | ID |
| (KABAT) | NO: | (KABAT) | NO: | (KABAT) | NO: |
| SSSYWWG | 264 | TMYYSGNIY | 265 | HVGYSYGRR | 266 |
| | | YNPSLQS | | FWYFDL | |
| | SEQ | | SEQ | | SEQ |
| HCDR1 | ID | HCDR2 | ID | HCDR3 | ID |
| (CHOTHIA) | NO: | (CHOTHIA) | NO: | (CHOTHIA) | NO: |
| GGSLSSSSY | 267 | YYSGN | 268 | HVGYSYGRR | 269 |
| | SEQ | | SEQ | FWYFDL | QSE |
| HCDR1 | ID | HCDR2 | ID | HCDR3 | ID |
| (IMGT) | NO: | (IMGT) | NO: | (IMGT) | NO: |
| GGSLSSSSYW | 270 | MYYSGNI | 271 | ARHVGYSYG | 272 |
| | | | | RRFWYFDL | |
| | SEQ | | SEQ | | SEQ |
| HCDR1 | ID | HCDR2 | ID | HCDR3 | ID |
| (CONTACT) | NO: | (CONTACT) | NO: | (CONTACT) | NO: |
| SSSSYWWG | 273 | WIGTMYYSG | 274 | ARHVGYSYG | 275 |
| | | NIY | | RRFWYFD | |
| | SEQ | | SEQ | | QSE |
| LCDR1 | ID | LCDR2 | ID | LCDR3 | ID |
| (Abm) | NO: | (Abm) | NO: | (Abm) | NO: |
| RASQSVSSYLA | 276 | DASNRAT | 277 | QQRSNWPPT | 278 |
| | SEQ | | SEQ | | SEQ |
| LCDR1 | ID | LCDR2 | ID | LCDR3 | ID |
| (KABAT) | NO: | (KABAT) | NO: | (KABAT) | NO: |
| RASQSVSSYLA | 279 | DASNRAT | 280 | QQRSNWPPT | 281 |
| | SEQ | | SEQ | | SEQ |
| LCDR1 | ID | LCDR2 | ID | LCDR3 | ID |
| (CHOTHIA) | NO: | (CHOTHIA) | NO: | (CHOTHIA) | NO: |
| RASQSVSSYLA | 282 | DASNRAT | 283 | QQRSNWPPT | 284 |
| | SEQ | | SEQ | | SEQ |
| LCDR1 | ID | LCDR2 | ID | LCDR3 | ID |
| (IMGT) | NO: | (IMGT) | NO: | (IMGT) | NO: |
| QSVSSY | 285 | DAS | 286 | QQRSNWPPT | 287 |
| | SEQ | | SEQ | | SEQ |
| LCDR1 | ID | LCDR2 | ID | LCDR3 | ID |
| (CONTACT) | NO: | (CONTACT) | NO: | (CONTACT) | NO: |
| SSYLAWY | 288 | LLIYDASNRA | 289 | QQRSNWPP | 290 |

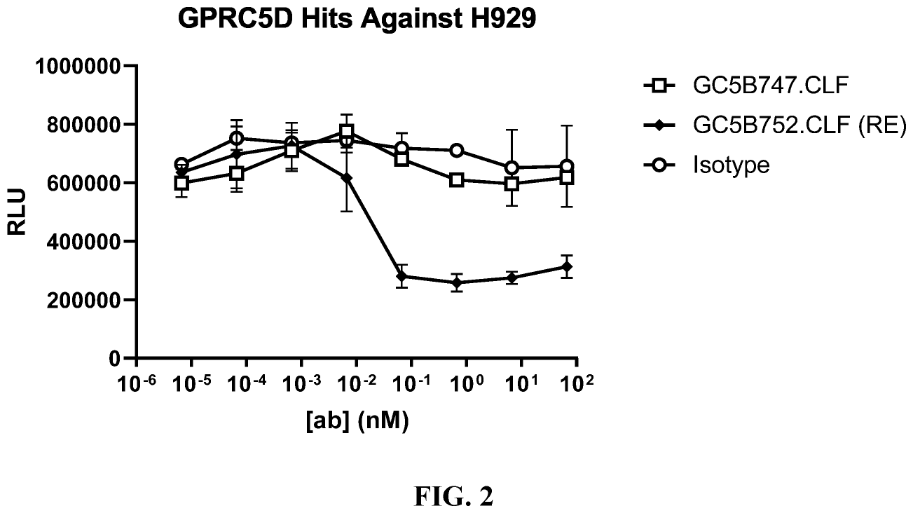

The wildtype GC5B747.CLF antibody did not show any CDC activity against H929 cells, while the GC5B752.CLF antibody comprising RE mutations showed significantly enhanced CDC activity against H929 cells (FIG. 2).

These results showed that the RE mutations in the Fc region consistently potentiate CDC activity of antibodies across multiple target cell lines. Such CDC potentiation is therefore not a target-specific phenomenon but a general characteristic of the RE mutations that can be applied to enhance Fc effector function in general.

### Example 1.3 Anti-KLK2 Antibodies

Anti-KLK2 antibodies and their variants with modified constant regions (i.e., with L234A/L235A/D265S mutations, low fucosylated and/or with K248E and T437R (RE) mutations) were generated and tested for their ability to mediate tumor cell killing via ADCC in the Vertebral-Cancer of the Prostate (VCaP) cell line, a cell line established from prostate cancer tissue, and these antibodies are summarized in Table 18 below.

TABLE 18
Anti-KLK2 antibodies and their variants

| Antibody | Description | EC50 (M) | 95% CI (M) |
|---|---|---|---|
| KL2B870 | IgG1, K248E, | 1.001 × 10⁻⁹ | 5.149 × 10⁻¹⁰ to |
| (KL2B30 in hlgG1-RE) | T437R (RE) | | 2.221 × 10⁻⁹ |
| KL2B871 | L234A, L235A, | | |
| (KL2B30 in hlgG1-AAS) | D265S | | |
| KL2B872 | Wildtype IgG1 | | |
| (KL2B30 in hlgG1) | | | |
| KL2B872.CLF | IgG1, low | 4.118 × 10⁻¹⁰ | 1.747 × 10⁻¹⁰ to |
| | fucosylation | | 8.869 × 10⁻¹⁰ |
| KL2B870.CLF | IgG1, K248E, | 3.542 × 10⁻¹⁰ | 1.789 × 10¹⁰ to |
| | T437R (RE), | | 6.006 × 10⁻¹⁰ |
| | low fucosylation | | |

The parental antibody KL2B30 was modified in its Fc region to introduce the L234A, L235A and D265S mutations (AAS mutations), which resulted in an Fc region that does not bind Fc receptors. The resulting KL2B871 was generated as a negative control. The KL2B30 antibody was modified in its Fc region to introduce the K248E and T437R mutations (RE mutations), and the resulting antibody is KL2B870. KL2B870 and KL2B872 antibodies were expressed in fucosylation-deficient cells to produce antibodies with low fucosylation (e.g., expressing these antibodies in fucosylation-deficient Chinese Hamster Ovary cells produces antibodies with less than 10% fucosylation), which were designated as KL2B870.CLF and KL2B872.CLF, respectively (see Table 18).

The amino acid sequences of heavy chain and light chain of exemplary anti-KLK2 antibodies are summarized in Table 19 and Table 20 below.

TABLE 19
Heavy chain of Anti-KLK2 antibodies

| Anti- | | SEQ |
|---|---|---|
| body | | ID |
| Name | Heavy Chain | NO: |
| KL2B870 | QVQLQESGPGLVKPSETLSLTCTVSGGSISS | 291 |
| | YYWSWIRQPPGKGLEWIGYIYYSGSTNYNPS | |
| | LKSRVTISVDTSKNQFSLKLSSVTAADTAVY | |
| | YCAGTTIFGVVTPNFYYGMDVWGQGTTVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNT | |
| | KVDKKVEPKSCDKTHTCPPCPAPELLGGPSV | |
| | FLFPKPEDTLMISRTPEVTCVVVDVSHEDPE | |
| | PVKFNWYVDGVEVHNAKTKPREEQYNSTYRV | |
| | VSVLTVLHQDWLNGKEYKCKVSNKALPAPIE | |
| | KTISKAKGQPREPQVYTLPPSREEMTKNQVS | |
| | LTCLVKGFYPSDIAVEWESNGQPENNYKTTP | |
| | PVLDSDGSFFLYSKLTVDKSRWQQGNVFSCS | |
| | VMHEALHNHYRQKSLSLSPG | |
| KL2B871 | QVQLQESGPGLVKPSETLSLTCTVSGGSISS | 292 |
| | YYWSWIRQPPGKGLEWIGYIYYSGSTNYNPS | |
| | LKSRVTISVDTSKNQFSLKLSSVTAADTAVY | |
| | YCAGTTIFGVVTPNFYYGMDVWGQGTTVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNT | |
| | KVDKKVEPKSCDKTHTCPPCPAPEAAGGPSV | |
| | FLFPPKPKDTLMISRTPEVTCVVVSVSHEDP | |
| | EVKFNWYVDGVEVHNAKTKPREEQYNSTYRV | |
| | VSVLTVLHQDWLNGKEYKCKVSNKALPAPIE | |
| | KTISKAKGQPREPQVYTLPPSREEMTKNQVS | |
| | LTCLVKGFYPSDIAVEWESNGQPENNYKTTP | |
| | PVLDSDGSFFLYSKLTVDKSRWQQGNVFSCS | |
| | VMHEALHNHYTQKSLSLSPG | |
| KL2B872 | QVQLQESGPGLVKPSETLSLTCTVSGGSISS | 293 |
| | YYWSWIRQPPGKGLEWIGYIYYSGSTNYNPS | |
| | LKSRVTISVDTSKNQFSLKLSSVTAADTAVY | |
| | YCAGTTIFGVVTPNFYYGMDVWGQGTTVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNT | |
| | KVDKKVEPKSCDKTHTCPPCPAPELLGGPSV | |
| | FLFPPKPKDTLMISRTPEVTCVVVDVSHEDP | |
| | EVKFNWYVDGVEVHNAKTKPREEQYNSTYRV | |
| | VSVLTVLHQDWLNGKEYKCKVSNKALPAPIE | |
| | KTISKAKGQPREPQVYTLPPSREEMTKNQVS | |
| | LTCLVKGFYPSDIAVEWESNGQPENNYKTTP | |
| | PVLDSDGSFFLYSKLTVDKSRWQQGNVFSCS | |
| | VMHEALHNHYTQKSLSLSPG | |

TABLE 20
Light chain of Anti-KLK2 antibodies

| Anti- | | SEQ |
|---|---|---|
| body | | ID |
| Name | Light Chain | NO: |
| KL2B870 | DIQMTQSPSFLSASVGDRVTITCRASQGISS | 294 |
| | YLAWYQQKPGKAPKFLIYAASTLQSGVPSRF | |
| | SGSGSGTEFTLTISSLQPEDFATYYCQQLNS | |
| | YPLTFGGGTKVEIKRTVAAPSVFIFPPSDEQ | |
| | LKSGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| KL2B871 | DIQMTQSPSFLSASVGDRVTITCRASQGISS | 295 |
| | YLAWYQQKPGKAPKFLIYAASTLQSGVPSRF | |
| | SGSGSGTEFTLTISSLQPEDFATYYCQQLNS | |
| | YPLTFGGGTKVEIKRTVAAPSVFIFPPSDEQ | |
| | LKSGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| KL2B872 | DIQMTQSPSFLSASVGDRVTITCRASQGISS | 296 |
| | YLAWYQQKPGKAPKFLIYAASTLQSGVPSRF | |
| | SGSGSGTEFTLTISSLQPEDFATYYCQQLNS | |
| | YPLTFGGGTKVEIKRTVAAPSVFIFPPSDEQ | |
| | LKSGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |

KL2B870, KL2B871, and KL2B872 share the same amino acid sequences of the VH CDRs and VL CDRs, which are summarized in Table 21 below.

TABLE 21

| | SEQ | | SEQ | | SEQ |
|---|---|---|---|---|---|
| HCDR1 | ID | HCDR2 | ID | HCDR3 | ID |
| (Abm) | NO: | (Abm) | NO: | (Abm) | NO: |
| GGSISSYYWS | 297 | YIYYSGSTN | 298 | TTIFGVVTP | 299 |
| | SEQ | | SEQ | NFYYGMDV | SEQ |
| HCDR1 | ID | HCDR2 | ID | HCDR3 | ID |
| (KABAT) | NO: | (KABAT) | NO: | (KABAT) | NO: |
| SYYWS | 300 | YIYYSGSTN | 301 | TTIFGVVTP | 302 |
| | | YNPSLKS | | NFYYGMDV | |
| | SEQ | | SEQ | | SEQ |
| HCDR1 | ID | HCDR2 | ID | HCDR3 | ID |
| (CHOTHIA) | NO: | (CHOTHIA) | NO: | (CHOTHIA) | NO: |
| GGSISSY | 303 | YYSGS | 304 | TTIFGVVTP | 305 |
| | SEQ | | SEQ | NFYYGMDV | SEQ |
| HCDR1 | ID | HCDR2 | ID | HCDR3 | ID |
| (IMGT) | NO: | (IMGT) | NO: | (IMGT) | NO: |
| GGSISSYY | 306 | IYYSGST | 307 | AGTTIFGVVT | 308 |
| | | | | PNFYYGMDV | |
| | SEQ | | SEQ | | SEQ |
| HCDR1 | ID | HCDR2 | ID | HCDR3 | ID |
| (CONTACT) | NO: | (CONTACT) | NO: | (CONTACT) | NO: |
| SSYYWS | 309 | WIGYIYYSGS | 310 | AGTTIFGVVT | 311 |
| | | TN | | PNFYYGMD | |
| | SEQ | | SEQ | | SEQ |
| LCDR1 | ID | LCDR2 | ID | LCDR3 | ID |
| (Abm) | NO: | (Abm) | NO: | (Abm) | NO: |
| RASQGISSYL | 312 | AASTLQS | 313 | QQLNSYPLT | 314 |
| A | | | | | |
| | SEQ | | SEQ | | SEQ |
| LCDR1 | ID | LCDR2 | ID | LCDR3 | ID |
| (KABAT) | NO: | (KABAT) | NO: | (KABAT) | NO: |
| RASQGISSYL | 315 | AASTLQS | 316 | QQLNSYPLT | 317 |
| A | | | | | |
| | SEQ | | SEQ | | SEQ |
| LCDR1 | ID | LCDR2 | ID | LCDR3 | ID |
| (CHOTHIA) | NO: | (CHOTHIA) | NO: | (CHOTHIA) | NO: |
| RASQGISSYL | 318 | AASTLQS | 319 | QQLNSYPLT | 320 |
| A | | | | | |
| | SEQ | | SEQ | | SEQ |
| LCDR1 | ID | LCDR2 | ID | LCDR3 | ID |
| (IMGT) | NO: | (IMGT) | NO: | (IMGT) | NO: |
| QGISSY | 321 | AAS | 322 | QQLNSYPLT | 323 |
| | SEQ | | SEQ | | SEQ |
| LCDR1 | ID | LCDR2 | ID | LCDR3 | ID |
| (CONTACT) | NO: | (CONTACT) | NO: | (CONTACT) | NO: |
| SSYLAWY | 324 | FLIYAASTLQ | 325 | QQLNSYPL | 326 |

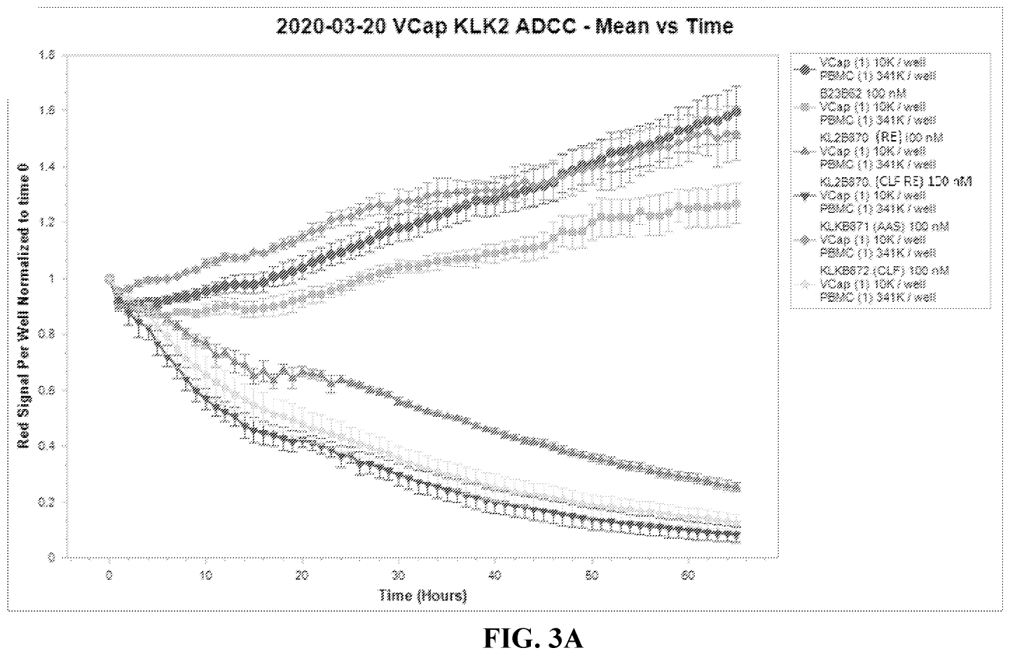

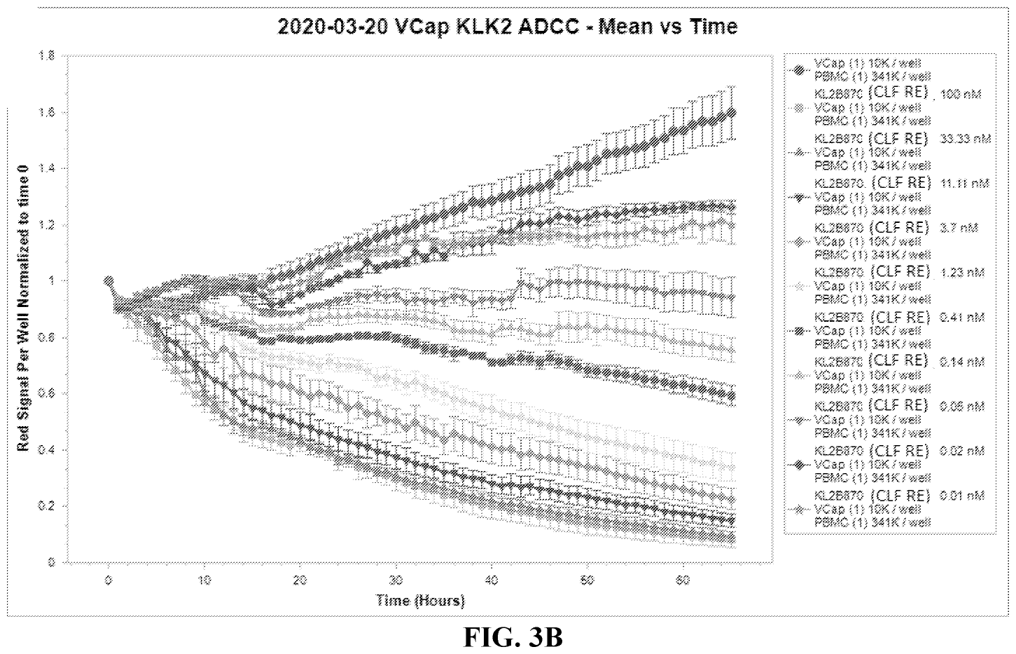

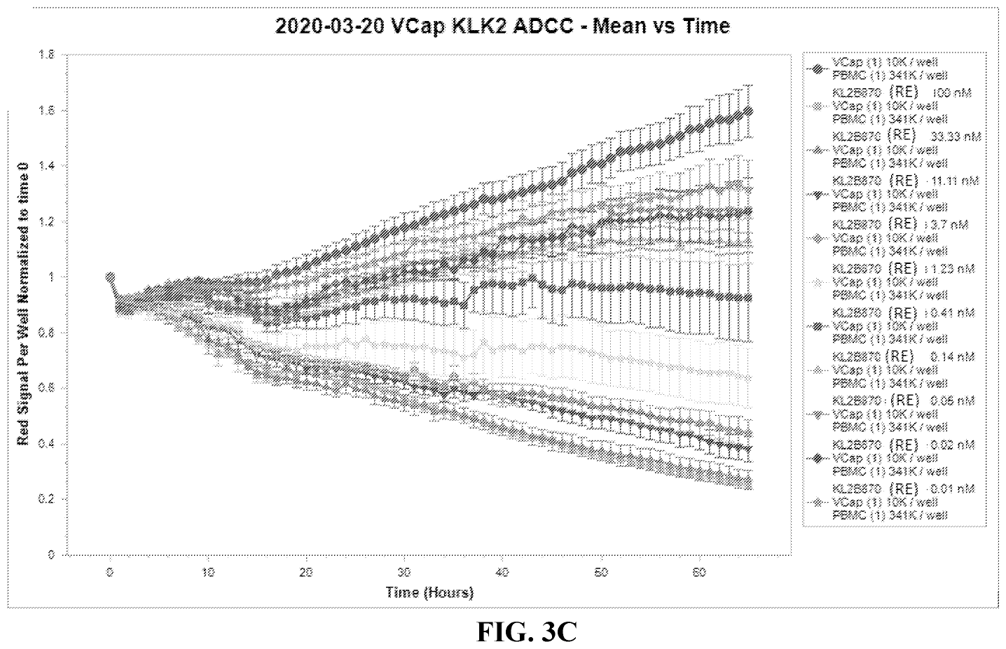

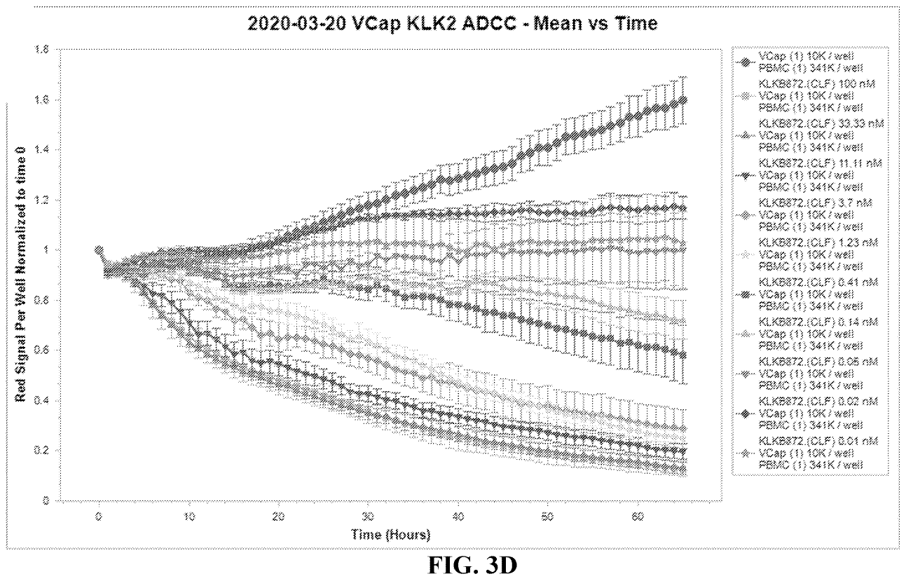

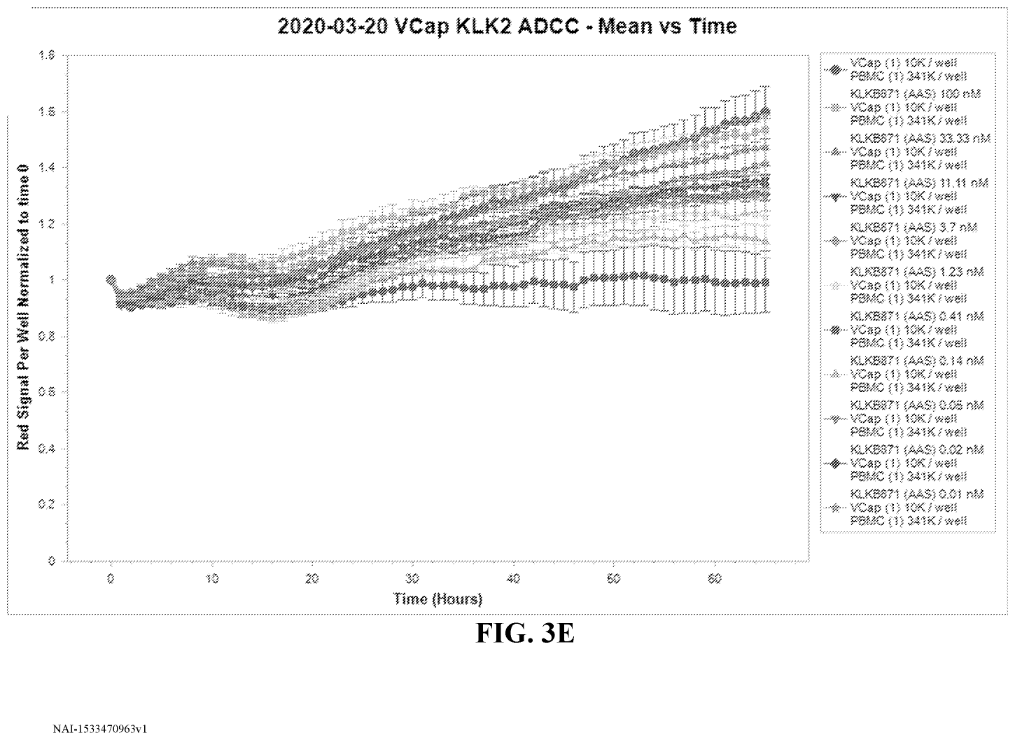

VCaP cells stably transfected with Nuclight Red (Incucyte®, Essen Bioscience) were plated at 10,000 cells per well in a 384-well plate (Perkin Elmer ViewPlate) in clear media (RPMI 1641+10% FBS, Thermo Fisher Scientific) to allow for cell adherence overnight. ADCC assay was performed with freshly thawed PBMC (Hemcare, PB009C-3). The ratio of effector to target cell per well was 34:1 for PBMCs as effector cells. KLK2 antibodies were tested with final concentrations ranging from 100 nM to 0.01 nM. After effector cells and antibodies were added to target cells, real time imaging was performed under Incucyte© S3 instrument (Essen BioScience) (FIGS. 3A-3E). Total red intergraded signal per well was quantified with Incucyte© software. Data analysis were performed by Incucyte© software and Prism (GraphPad Software) based on values of quadruplicates. The percentage of cell killing was calculated as: (1−KLK2 mAb/no mAb control)×100%.

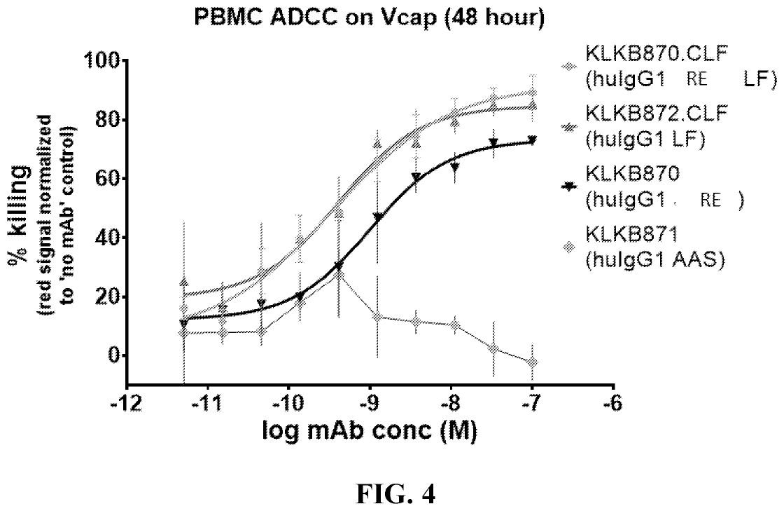

KLK2 antibodies demonstrated obvious dose dependent ADCC activities by PBMC on VCaP cells. The kinetic showed that ADCC activities initiated immediately after addition of effector cells and antibodies and continued with time (FIG. 4). KLKB870.CLF and KLKB872.CLF antibodies showed the highest ADCC activity with approximately 89% and 85% cell killing, followed by approximately 73% by KLKB870. KLKB87, the IgG1 antibody with AAS mutations, did not demonstrate any ADCC activity (FIG. 4). Dose-response curve generated at a certain time point (48 hours after effector cells and antibodies were added to target cells) showed the killing EC50 is approximately 354 pM for KLKB870.CLF, the antibody with low fucosylation. The EC50 for normal fucosylation antibody KLKB870 was approximately lnM (Table 3).

### Example 1.4 Anti-HLA-G Antibodies

To test the ability of anti-HLA-G antibodies to mediate tumor cell killing via ADCC in the choriocarcinoma cell line JEG-3 (ATCC HTB-36), which endogenously expresses HLA-G, antibodies were added to JEG-3 cells labeled with BATDA dye (Perkin Elmer cat. #C136-100), which can unidirectionally penetrate into the cells. Upon cell lysis, the dye is released into the solution containing Europium which reacts with the dye to form a fluorescent chelate, whose fluorescence signal can be measured. PBMCs cultured overnight were added at an E:T ratio of 50:1 to JEG-3 cells at 5,000 cells/well and the mixture was incubated for 4 hours at 37° C. The cell mixture was added at 1:10 into Europium solution, incubated for 15 min at room temperature and fluorescence at 610 nm was monitored to determine signal. The fluorescence signal for 100% killing was determined using a well containing BATDA-labeled target cells mixed with Triton-X 100 detergent.

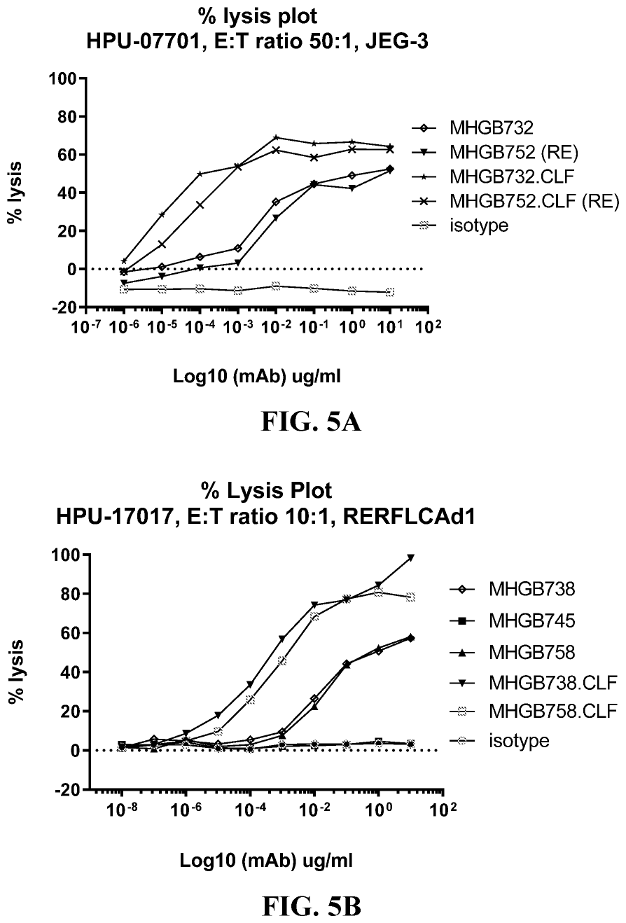

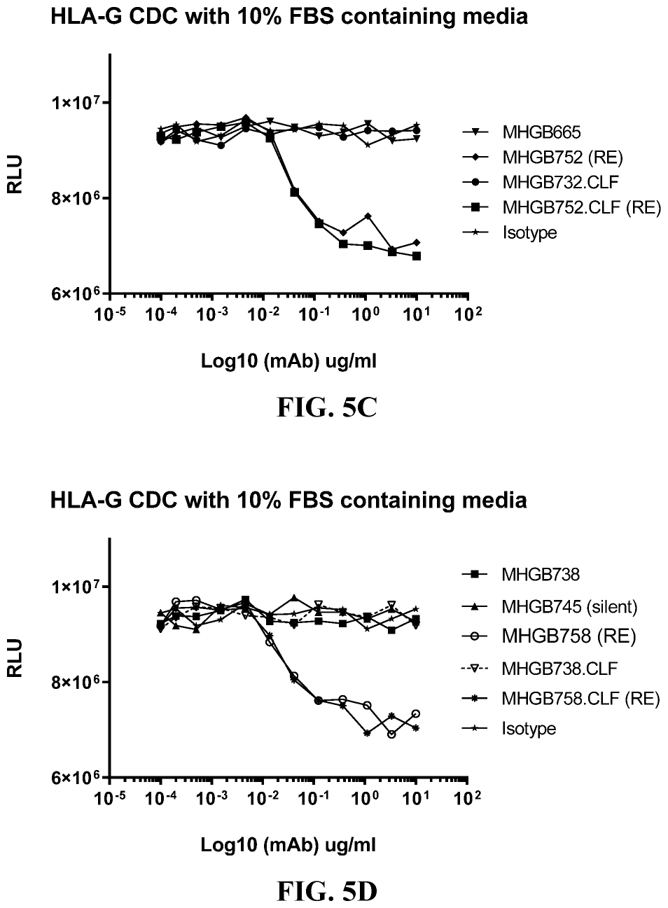

Since the anti-HLA-G Abs could display ADCC in vitro, whether this activity could be enhanced was explored. Several studies showed that antibodies having less than 10% terminal fucosylated Fc regions display enhanced effector function due to higher affinity binding to Fc receptors. Thus, anti-HLA-G antibodies MHGB732 and MHGB738 were generated in a low fucose Chinese Hamster Ovary (CHO) host to produce an antibody with less than 10% fucosylation (MHGB732.CLF and MHGB738.CLF) (Table 22, FIGS. 5A-5D). As a negative control, a version of MHGB738 with an Fc region that could not bind Fc receptors was generated, and this antibody was called MHGB745.

The normal fucose and low fucose antibodies were tested for their abilities to induce NK cell-based ADCC against either JEG-3 cells (FIG. 5A) or against RERF-LC-Ad-1 cells (human lung adenocarcinoma cell line, JCRB1020) (FIG. 5B). Antibodies with low fucosylation were generated by expression of the constructs encoding the heavy chain and light chain in CHO cells with low expression levels of the fucosyltransferase enzyme, leading to production of antibodies having less than 10% fucosylation.

The ADCC activity assay was performed as described above. The ratio of effector cells to target cells is shown in FIGS. 5A and 5B. Both the MHGB745 and the isotype control did not induce ADCC in the assay. The two IgG1 antibodies, MHGB732 and MHGB738 could induce ADCC while the same antibodies having Fc regions with low fucosylation displayed approximately 10-fold enhanced ADCC activity. This showed that ADCC enhancement could be obtained by use of an antibody with low fucosylation.

Next the abilities of these antibodies to mediate CDC were tested (FIGS. 5C and 5D). Briefly, assays were run in 10% FBS containing DMEM (JEG-3) or RPMI (RERF-LC-Ad-1). Antibodies were added to target cells and incubated for 30 minutes at 37° C. After incubation, 15-20% (stock concentration) of rabbit complement (Cedarlane cat. #CL3441-S) and heat inactivated complement was added to the wells respectively to a volume of 25 μl/well. The mixture was incubated for 4-12 hours at 37° C. Target cell lysis was detected by addition of 100 μl of CellTitre-Glo (Promega cat. #G9242) reagent followed by incubation for 10 minutes at room temperature. Luminescence was monitored using a Tecan Microplate reader SPARK©. The two IgG1 antibodies, MHGB732 and MHGB738 did not mediate CDC. Since the IgG1 antibodies could not mediate CDC, the variable regions thereof were cloned into an IgG1 Fc harboring the K248E and T437R mutations (RE mutations), which were shown to specifically enhance CDC activity. These antibodies, having the identical variable regions as their IgG1 counterparts, could mediate CDC activity. As a control, anti-HLA-G antibody MHGB665 was also tested in the CDC activity assay. Whether the RE Fc variants would impact the ADCC activity enhancement in antibodies with low fucosylation and whether Fc regions with low fucosylation would impact the CDC activity of the RE Fc variants were examined. Antibodies bearing the RE mutations produced in a low fucose host (with less than 10% fucosylation), MHGB752 and MHGB758 had identical ADCC activities to the IgG1 antibodies MHGB732 and MHGB738 with low fucosylation (FIGS. 5A and 5B). Analogously, antibodies bearing the RE mutations produced in a low fucose host had identical CDC activities to the same antibodies produced in a normal fucose host (FIGS. 5C and 5D).

Antibodies MHGB732 and MHGB738 and their variants with modified constant regions are summarized in Table 22 below.

TABLE 22
Antibodies MHGB732 and MHGB738 and their variants

| Antibody Name | Description |
|---|---|
| MHGB732 | IgG1 |
| MHGB738 | IgG1 |
| MHGB745 | L234A, L235A, D265S |
| MHGB752 | IgG1, K248E, T437R (RE) |
| MHGB758 | IgG1, K248E, T437R (RE) |
| MHGB732.CLF | IgG1, low fucosylation |
| MHGB738.CLF | IgG1, low fucosylation |
| MHGB752.CLF | IgG1, K248E, T437R (RE), |
| | low fucosylation |
| MHGB758.CLF | IgG1, K248E, T437R (RE), |
| | low fucosylation |

The amino acid sequences of heavy chain and light chain of exemplary anti-HLA-G antibodies are summarized in Table 23 and Table 24 below.

TABLE 23
Heavy chain of Anti-HLA-G antibodies

| Anti | | SEQ |
|---|---|---|
| body | | ID |
| Name | Heavy Chain | NO: |
| MHGB732 | QVQLQQSGPGLVKPSQTLSLTCAISGDSVSSN | 327 |
| | SAAWNWIRQSPSRGLEWLGRTYYRSKWYNDYA | |
| | VSVKSRITINPDTSKNQISLQLNSVTPEDTAV | |
| | YYCAGDRRYGIVGLPFAYWGQGTLVTVSSAST | |
| | KGPSVFPLAPSSKSTSGGTAALGCLVKDYFPE | |
| | PVTVSWNSGALTSGVHTFPAVLQSSGLYSLSS | |
| | VVTVPSSSLGTQTYICNVNHKPSNTKVDKKVE | |
| | PKSCDKTHTCPPCPAPELLGGPSVFLFPPKPK | |
| | DTLMISRTPEVTCVVVDVSHEDPEVKFNWYVD | |
| | GVEVHNAKTKPREEQYNSTYRVVSVLTVLHQD | |
| | WLNGKEYKCKVSNKALPAPIEKTISKAKGQPR | |
| | EPQVYTLPPSREEMTKNQVSLTCLVKGFYPSD | |
| | IAVEWESNGQPENNYKTTPPVLDSDGSFFLYS | |
| | KLTVDKSRWQQGNVFSCSVMHEALHNHYTQKS | |
| | LSLSPG | |
| MHGB738 | QVQLQQSGPGLVKPSQTLSLTCAISGDSVSSN | 328 |
| | RAAWNWIRQSPSRGLEWLGRTYYRSKWYNDYA | |
| | VSVKSRITINPDTSKNQISLQLNSVTPEDTAV | |
| | YYCARVRPGIPFDYWGQGTPVTVSSASTKGPS | |
| | VFPLAPSSKSTSGGTAALGCLVKDYFPEPVTV | |
| | SWNSGALTSGVHTFPAVLQSSGLYSLSSVVTV | |
| | PSSSLGTQTYICNVNHKPSNTKVDKKVEPKSC | |
| | DKTHTCPPCPAPELLGGPSVFLFPPKPKDTLM | |
| | ISRTPEVTCVVVDVSHEDPEVKFNWYVDGVEV | |
| | HNAKTKPREEQYNSTYRVVSVLTVLHQDWLNG | |
| | KEYKCKVSNKALPAPIEKTISKAKGQPREPQV | |
| | YTLPPSREEMTKNQVSLTCLVKGFYPSDIAVE | |
| | WESNGQPENNYKTTPPVLDSDGSFFLYSKLTV | |
| | DKSRWQQGNVFSCSVMHEALHNHYTQKSLSLS | |
| | PG | |
| MHGB745 | NDYAVSVKSRITINPDTSKNQISLQLNSVTPE | 329 |
| | DTAVYYCARVRPGIPFDYWGQGTPVTVSSAST | |
| | KGPSVFPLAPSSKSTSGGTAALGCLVKDYFPE | |
| | PVTVSWNSGALTSGVHTFPAVLQSSGLYSLSS | |
| | VVTVPSSSLGTQTYICNVNHKPSNTKVDKKVE | |
| | PKSCDKTHTCPPCPAPEAAGGPSVFLFPPKPK | |
| | DTLMISRTPEVTCVVVSVSHEDPEVKFNWYVD | |
| | GVEVHNAKTKPREEQYNSTYRVVSVLTVLHQD | |
| | WLNGKEYKCKVSNKALPAPIEKTISKAKGQPR | |
| | EPQVYTLPPSREEMTKNQVSLTCLVKGFYPSD | |
| | IAVEWESNGQPENNYKTTPPVLDSDGSFFLYS | |
| | KLTVDKSRWQQGNVFSCSVMHEALHNHYTQKS | |
| | LSLSPG | |
| MHGB752 | QVQLQQSGPGLVKPSQTLSLTCAISGDSVSSN | 330 |
| | SAAWNWIRQSPSRGLEWLGRTYYRSKWYNDYA | |
| | VSVKSRITINPDTSKNQISLQLNSVTPEDTAV | |
| | YYCAGDRRYGIVGLPFAYWGQGTLVTVSSAST | |
| | KGPSVFPLAPSSKSTSGGTAALGCLVKDYFPE | |
| | PVTVSWNSGALTSGVHTFPAVLQSSGLYSLSS | |
| | VVTVPSSSLGTQTYICNVNHKPSNTKVDKKVE | |
| | PKSCDKTHTCPPCPAPELLGGPSVFLFPPKPE | |
| | DTLMISRTPEVTCVVVDVSHEDPEVKFNWYVD | |
| | GVEVHNAKTKPREEQYNSTYRVVSVLTVLHQD | |
| | WLNGKEYKCKVSNKALPAPIEKTISKAKGQPR | |
| | EPQVYTLPPSREEMTKNQVSLTCLVKGFYPSD | |
| | IAVEWESNGQPENNYKTTPPVLDSDGSFFLYS | |
| | KLTVDKSRWQQGNVFSCSVMHEALHNHYRQKS | |
| | LSLSPG | |
| MHGB758 | QVQLQQSGPGLVKPSQTLSLTCAISGDSVSSN | 331 |
| | RAAWNWIRQSPSRGLEWLGRTYYRSKWYNDYA | |
| | VSVKSRITINPDTSKNQISLQLNSVTPEDTAV | |
| | YYCARVRPGIPFDYWGQGTPVTVSSASTKGPS | |
| | VFPLAPSSKSTSGGTAALGCLVKDYFPEPVTV | |
| | SWNSGALTSGVHTFPAVLQSSGLYSLSSVVTV | |
| | PSSSLGTQTYICNVNHKPSNTKVDKKVEPKSC | |
| | DKTHTCPPCPAPELLGGPSVFLFPPKPEDTLM | |
| | ISRTPEVTCVVVDVSHEDPEVKFNWYVDGVEV | |
| | HNAKTKPREEQYNSTYRVVSVLTVLHQDWLNG | |
| | KEYKCKVSNKALPAPIEKTISKAKGQPREPQV | |
| | YTLPPSREEMTKNQVSLTCLVKGFYPSDIAVE | |
| | WESNGQPENNYKTTPPVLDSDGSFFLYSKLTV | |
| | DKSRWQQGNVFSCSVMHEALHNHYRQKSLSLS | |
| | PG | |

TABLE 24
Light chain of Anti-HLA-G antibodies

| Anti- | | SEQ |
|---|---|---|
| body | | ID |
| Name | Light Chain | NO: |
| MHGB732 | DIVMTQSPDSLAVSLGERATINCKSSQSVLHS | 332 |
| | SNNKNYLTWFQQKPGQPPKLLIYWASTRESGV | |
| | PDRFSGSGSGTDFTLTISSLQAEDVAVYYCHQ | |
| | YYSTPPTFGQGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADY | |
| | EKHKVYACEVTHQGLSSPVTKSFNRGEC | |
| MHGB738 | DIVMTQSPDSLAVSLGERATINCKSSQSVLFS | 333 |
| | SNNKNYLAWYQQKPGQPPKLLIYWASTRESGV | |
| | PDRFSGSVSGTDFTLTISSLQAEDVAVYYCQQ | |
| | YHSTPWTFGQGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADY | |
| | EKHKVYACEVTHQGLSSPVTKSFNRGEC | |
| MHGB745 | DIVMTQSPDSLAVSLGERATINCKSSQSVLFS | 334 |
| | SNNKNYLAWYQQKPGQPPKLLIYWASTRESGV | |
| | PDRFSGSVSGTDFTLTISSLQAEDVAVYYCQQ | |
| | YHSTPWTFGQGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADY | |
| | EKHKVYACEVTHQGLSSPVTKSFNRGEC | |
| MHGB752 | DIVMTQSPDSLAVSLGERATINCKSSQSVLHS | 335 |
| | SNNKNYLTWFQQKPGQPPKLLIYWASTRESGV | |
| | PDRFSGSGSGTDFTLTISSLQAEDVAVYYCHQ | |
| | YYSTPPTFGQGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADY | |
| | EKHKVYACEVTHQGLSSPVTKSFNRGEC | |
| MHGB758 | DIVMTQSPDSLAVSLGERATINCKSSQSVLFS | 336 |
| | SNNKNYLAWYQQKPGQPPKLLIYWASTRESGV | |
| | PDRFSGSVSGTDFTLTISSLQAEDVAVYYCQQ | |
| | YHSTPWTFGQGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADY | |
| | EKHKVYACEVTHQGLSSPVTKSFNRGEC | |

MHGB732 and MHGB752 share the same amino acid sequences of the VH CDRs and VL CDRs, while MHGB738, MHGB745, and MHGB758 share the same amino acid sequences of the VH CDRs and VL CDRs, which are all summarized in Table 25 below.

TABLE 25

| Anti- | | SEQ | | SEQ | | SEQ |
|---|---|---|---|---|---|---|
| body | HCDR1 | ID | HCDR2 | ID | HCDR3 | ID |
| Name | (Abm) | NO: | (Abm) | NO: | (Abm) | NO: |
| MHGB73 | GDSVSSNSA | 337 | RTYYRSKWY | 338 | DRRYGIVGL | 339 |
| 2 and | AWN | | ND | | PFAY | |
| MHGB75 | | | | | | |
| 2 | | | | | | |
| MHGB73 | GDSVSSNRA | 340 | RTYYRSKWY | 341 | VRPGIPFDY | 342 |
| 8, | AWN | | ND | | | |
| MHGB74 | | | | | | |
| 5, and | | | | | | |
| MHGB75 | | | | | | |
| 8 | | | | | | |
| | | SEQ | | SEQ | | SEQ |
| | HCDR1 | ID | HCDR2 | ID | HCDR3 | ID |
| | (KABAT) | NO: | (KABAT) | NO: | (KABAT) | NO: |
| MHGB73 | SNSAAWN | 343 | RTYYRSKWY | 344 | DRRYGIVGL | 345 |
| 2 and | | | NDYAVSVKS | | PFAY | |
| MHGB75 | | | | | | |
| 2 | | | | | | |
| MHGB73 | SNRAAWN | 346 | RTYYRSKWY | 347 | VRPGIPFDY | 348 |
| 8, | | | NDYAVSVKS | | | |
| MHGB74 | | | | | | |
| 5, and | | | | | | |
| MHGB75 | | | | | | |
| 8 | | | | | | |
| | | SEQ | | SEQ | | SEQ |
| | HCDR1 | ID | HCDR2 | ID | HCDR3 | ID |
| | (CHOTHIA) | NO: | (CHOTHIA) | NO: | (CHOTHIA) | NO: |
| MHGB73 | GDSVSSNSA | 349 | YYRSKWY | 350 | DRRYGIVGL | 351 |
| 2 and | | | | | PFAY | |
| MHGB75 | | | | | | |
| 2 | | | | | | |
| MHGB73 | GDSVSSNRA | 352 | YYRSKWY | 353 | VRPGIPFDY | 354 |
| 8, | | | | | | |
| MHGB74 | | | | | | |
| 5, and | | | | | | |
| MHGB75 | | | | | | |
| 8 | | | | | | |
| | | SEQ | | SEQ | | SEQ |
| | HCDR1 | ID | HCDR2 | ID | HCDR3 | ID |
| | (IMGT) | NO: | (IMGT) | NO: | (IMGT) | NO: |
| MHGB73 | GDSVSSNSA | 355 | TYYRSKWYN | 356 | AGDRRYGIV | 357 |
| 2 and | A | | | | GLPFAY | |
| MHGB75 | | | | | | |
| 2 | | | | | | |
| MHGB73 | GDSVSSNRA | 358 | TYYRSKWYN | 359 | ARVRPGIPF | 360 |
| 8, | A | | | | DY | |
| MHGB74 | | | | | | |
| 5, and | | | | | | |
| MHGB75 | | | | | | |
| 8 | | | | | | |
| | | SEQ | | SEQ | | SEQ |
| | HCDR1 | ID | HCDR2 | ID | HCDR3 | ID |
| | (CONTACT) | NO: | (CONTACT) | NO: | (CONTACT) | NO: |
| MHGB73 | SSNSAAWN | 361 | WLGRTYYRS | 362 | AGDRRYGIV | 363 |
| 2 and | | | KWYND | | GLPFA | |
| MHGB75 | | | | | | |
| 2 | | | | | | |
| MHGB73 | SSNRAAWN | 364 | WLGRTYYRS | 365 | ARVRPGIPF | 366 |
| 8, | | | KWYND | | D | |
| MHGB74 | | | | | | |
| 5, and | | | | | | |
| MHGB75 | | | | | | |
| 8 | | | | | | |
| | | SEQ | | SEQ | | SEQ |
| | LCDR1 | ID | LCDR2 | ID | LCDR3 | ID |
| | (Abm) | NO: | (Abm) | NO: | (Abm) | NO: |
| MHGB73 | KSSQSVLHS | 367 | WASTRES | 368 | HQYYSTPPT | 369 |
| 2 and | SNNKNYLT | | | | | |
| MHGB75 | | | | | | |
| 2 | | | | | | |
| MHGB73 | KSSQSVLFS | 370 | WASTRES | 371 | QQYHSTPWT | 372 |
| 8, | SNNKNYLA | | | | | |
| MHGB74 | | | | | | |
| 5, and | | | | | | |
| MHGB75 | | | | | | |
| 8 | | | | | | |
| | | SEQ | | SEQ | | SEQ |
| | LCDR1 | ID | LCDR2 | ID | LCDR3 | ID |
| | (KABAT) | NO: | (KABAT) | NO: | (KABAT) | NO: |
| MHGB73 | KSSQSVLHS | 373 | WASTRES | 374 | HQYYSTPPT | 375 |
| 2 and | SNNKNYLT | | | | | |
| MHGB75 | | | | | | |
| 2 | | | | | | |
| MHGB73 | KSSQSVLFS | 376 | WASTRES | 377 | QQYHSTPWT | 378 |
| 8, | SNNKNYLA | | | | | |
| MHGB74 | | | | | | |
| 5, and | | | | | | |
| MHGB75 | | | | | | |
| 8 | | | | | | |
| | | SEQ | | SEQ | | SEQ |
| | LCDR1 | ID | LCDR2 | ID | LCDR3 | ID |
| | (CHOTHIA) | NO: | (CHOTHIA) | NO: | (CHOTHIA) | NO: |
| MHGB73 | KSSQSVLHS | 379 | WASTRES | 380 | HQYYSTPPT | 381 |
| 2 and | SNNKNYLT | | | | | |
| MHGB75 | | | | | | |
| 2 | | | | | | |
| MHGB73 | KSSQSVLFS | 382 | WASTRES | 383 | QQYHSTPWT | 384 |
| 8, | SNNKNYLA | | | | | |
| MHGB74 | | | | | | |
| 5, and | | | | | | |
| MHGB75 | | | | | | |
| 8 | | | | | | |
| | | SEQ | | SEQ | | SEQ |
| | LCDR1 | ID | LCDR2 | ID | LCDR3 | ID |
| | (IMGT) | NO: | (IMGT) | NO: | (IMGT) | NO: |
| MHGB73 | QSVLHSSNN | 385 | WAS | 386 | HQYYSTPPT | 387 |
| 2 and | KNY | | | | | |
| MHGB75 | | | | | | |
| 2 | | | | | | |
| MHGB73 | QSVLFSSNN | 388 | WAS | 389 | QQYHSTPWT | 390 |
| 8, | KNY | | | | | |
| MHGB74 | | | | | | |
| 5, and | | | | | | |
| MHGB75 | | | | | | |
| 8 | | | | | | |
| | | SEQ | | SEQ | | SEQ |
| | LCDR1 | ID | LCDR2 | ID | LCDR3 | ID |
| | (CONTACT) | NO: | (CONTACT) | NO: | (CONTACT) | NO: |
| MHGB73 | LHSSNNKNY | 391 | LLIYWASTR | 392 | HQYYSTPP | 393 |
| 2 and | LTWF | | E | | | |
| MHGB75 | | | | | | |
| 2 | | | | | | |
| MHGB73 | LFSSNNKNY | 394 | LLIYWASTR | 395 | QQYHSTPW | 396 |
| 8, | LAWY | | E | | | |
| MHGB74 | | | | | | |
| 5, and | | | | | | |
| MHGB75 | | | | | | |
| 8 | | | | | | |

### Example 1.5 Anti-PSMA Antibodies

Anti-PSMA antibodies PSMB896 and PSMB898 were modified in its Fc region to introduce the K248E and T437R mutations (RE mutations). The anti-PSMA antibodies with RE mutations were expressed in fucosylation-deficient cells to produce antibodies with low fucosylation. The resulting antibody for PSMB896 is PSMB952; the resulting antibody for PSMB898 is PSMB956. The binding and thermal stability of PSMB952 and PSMB956 were assayed. The results indicate that low fucosylation and RE mutations do not impact these biophysical characteristics of the PSMB896 and PSMB898 antibodies.

C42B and LNCaP cells stably transfected with GFP were plated at 9,000 cells per well in a 384-well plate (Perkin Elmer ViewPlate) in clear media (RPMI 1641+10% FBS, Thermo Fisher Scientific) to allow for cell adherence overnight. ADCC assay was performed with freshly thawed PBMC (Hemcare, PB009C-3) or NK cells isolated from the frozen PBMC by RoboSep™ Cell Separation Instruments. Isolated NK cells were either used immediately or primed overnight with low dose IL-2 (1 ng/ml, Miltenyi Biotec). The ratio of effector to target cell per well was 34:1 for PBMC and 5:1 for isolated NK cells. The PSMB952 and PSMB956 antibodies were tested with final concentrations ranging from 100 nM to 0.01 nM. After effector cells and antibodies were added to target cells, real time imaging was performed under Incucyte© S3 instrument (Essen BioScience). Total GFP intergraded signal per well was quantified with Incucyte© software. Data analysis was performed by Incucyte© software and Prism (GraphPad Software) based on values of quadruplicates. The percentage of cell killing was calculated as: (1−PSMA mAb/no mAb control)×100%.

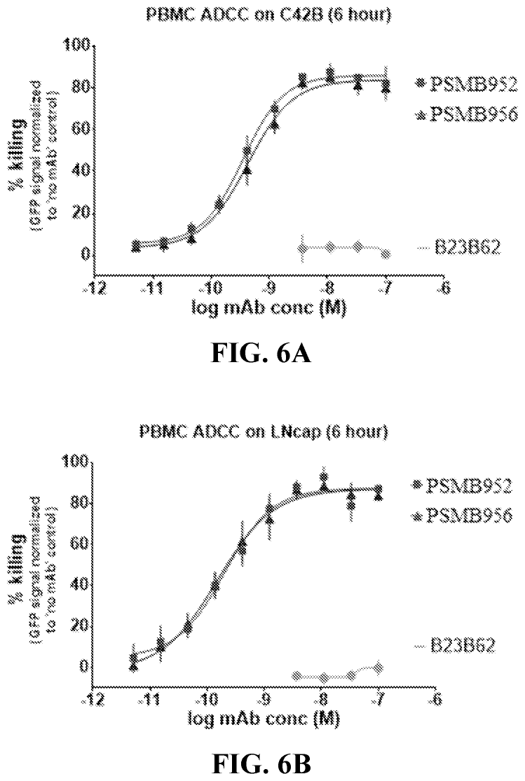

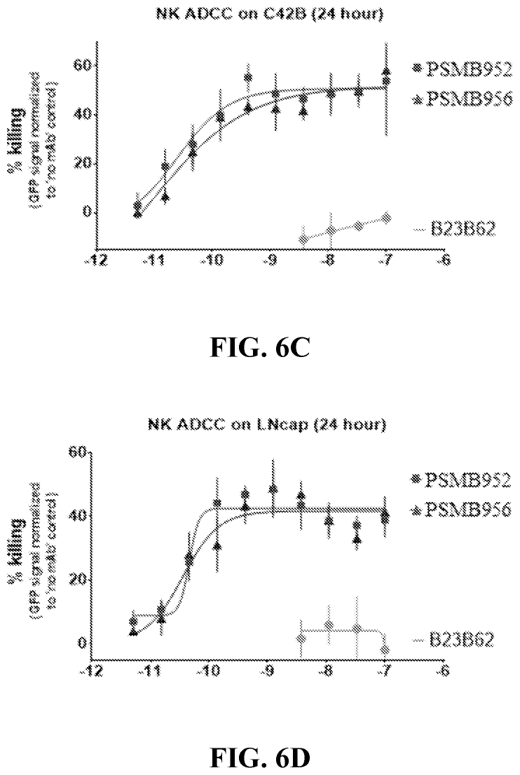

The PSMB952 and PSMB956 antibodies demonstrated obvious dose dependent ADCC activities by effector cells on C42B and LNCaP cells (FIGS. 6A-6D). The kinetic showed that ADCC activities initiated immediately after addition of effector cells and antibodies and continued with time. The maximal ADCC activity are ˜97% for PBMC and 40-50% for purified NK cells.

Dose-response curves generated at certain time points (6 hour for PBMC, 24 hour for NK cells) showed the killing EC50 is in the range of 10⁻¹⁰˜10⁻¹¹ M (Table 26).

TABLE 26
In vitro ADCC killing EC50 (M) by PBMC at 6 hour
and NK cells at 24 hour on C42B and LNCaP cells

| Target cell | Effector cell | PSMB952 | PSMB956 |
|---|---|---|---|
| C42B | PBMC | 3.51e−010 | 4.231e−010 |
| LNCaP | PBMC | 2.011e−010 | 1.519e−010 |
| C42B | NK | 2.453e−011 | 9.956e−012 |
| LNCaP | NK | 4.585e−011 | 3.579e−011 |

### Example 1.6 Anti-BCMA Antibodies

Exemplary anti-BCMA antibody BCMB519 is modified in its Fc region to introduce the RE mutations (K248E and T437R mutations). The anti-BCMA antibody with the RE mutations are expressed in fucosylation-deficient cells to produce antibodies with low fucosylation. The binding and thermal stability of the resulting antibodies are assayed.

C42B and LNCaP cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay is performed with freshly thawed PBMC (Hemcare, PB009C-3) or NK cells isolated from the frozen PBMC by RoboSep™ Cell Separation Instruments. Isolated NK cells are either used immediately or primed overnight with low dose IL-2 (1 ng/ml, Miltenyi Biotec). The ratio of effector to target cell per well is 34:1 for PBMC and 5:1 for isolated NK cells. The anti-BCMA antibodies with low fucosylation and RE mutations are tested with final concentrations ranging from 100 nM to 0.01 nM. After effector cells and antibodies are added to target cells, real time imaging is performed under Incucyte® S3 instrument (Essen BioScience). Total GFP intergraded signal per well is quantified with Incucyte® software. Data analysis is performed by Incucyte® software and Prism (GraphPad Software) based on values of quadruplicates. The percentage of cell killing is calculated as: (1−BCMA mAb/no mAb control)×100%.

Next, the abilities of these antibodies to mediate CDC are tested. Briefly, assays are run in 10% FBS containing DMEM (JEG-3) or RPMI (RERF-LC-Ad-1). Antibodies are added to target cells and incubated for 30 minutes at 37° C. After incubation, 15-20% (stock concentration) of rabbit complement (Cedarlane cat. #CL3441-S) and heat inactivated complement are added to the wells respectively to a volume of 25 μl/well. The mixture is incubated for 4-12 hours at 37° C. Target cell lysis is detected by addition of 100 μl of CellTitre-Glo (Promega cat. #G9242) reagent followed by incubation for 10 minutes at room temperature. Luminescence is monitored using a Tecan Microplate reader SPARK®.

### Example 2—ADCC and CDC Functional Analysis of Biparatopic or Bispecific Antibodies

### Example 2.1 Anti-GPRC5D Antibodies

A pair of non-competing GPRC5D binders are formatted as biparatopic antibodies using IgG1 constant domains with or without the RE mutations.

First of all, the biparatopic anti-GPRC5D antibodies are tested for their binding profiles on wildtype MM.1R cells, as well as GPRC5D-negative MM.1R cells (MM.1R GPRC5D knockout (KO)). The biparatopic anti-GPRC5D antibodies are also tested for their binding profiles on wildtype H929 cells, as well as GPRC5D-negative H929 cells (H929 GPRC5D KO). The binding profiles of an isotype control antibody are also tested. Specifically, cells are incubated with varying concentrations of the tested antibodies followed by detection of binding with Alexa Fluor 647-conjugated anti-human Fc reagent. The samples are processed on an Intellicyt iQue 3 flow cytometer to measure fluorescent intensity and then plotted in Graph Pad Prism.

The biparatopic anti-GPRC5D antibodies are expected to bind to wildtype MM.1R cells and H929 cells, which are GPRC5D-positive. The biparatopic anti-GPRC5D antibodies are expected to not bind to GPRC5D-negative MM.1R cells and H929 cells. The isotype control antibody is expected to not bind to any of the four types of cells.

Second, the biparatopic anti-GPRC5D antibodies are tested for CDC activities against four different cell lines. The four cell lines are: GPRC5D-positive MM.1R target cells, GPRC5D-positive H929 target cells, GPRC5D-positive JIM3 target cells, as well as GPRC5D-negative MM.1R cells (MM.1R GPRC5D KO). The CDC activities of an isotype control antibody against the four different cell lines are also tested. Specifically, the target cells are incubated with the tested antibodies for 24 hours in the presence of 40% normal human serum. Equal volumetric amounts of Cell TiterGlo (Promega) is then added to the treated target cells. Percent viability is measured from the luminescent signal and calculated by dividing the highest antibody concentration (100 nM) by vehicle control and multiplying by 100. The values are plotted with a log transformed x-axis with a 4-parameter non-linear regression curve fit.

The biparatopic anti-GPRC5D antibodies are expected to have potent CDC activities in all GPRC5D-positive cell lines but not in GPRC5D-negative H929 cells. The isotype control antibody is expected to show no CDC activity.

Third, the biparatopic anti-GPRC5D antibodies are tested for ADCC activities against five different cell lines. The five cell lines are: GPRC5D-positive MM.1R target cells, GPRC5D-positive MM.1S target cells, GPRC5D-positive H929 target cells, BCMA-negative H929 cells (H929 BCMA KO), as well as GPRC5D-negative H929 cells (H929 GPRC5D KO). The ADCC activities of an isotype control antibody against the five different cell lines are also tested. Specifically, the target cells are incubated with the tested antibodies for 48 hours in the presence of primary human NK cells. Primary human NK cells from two healthy donors are tested, and the ratio of effector to target cell per well is 5:1. The treated target cells are stained with a live/dead-dye and evaluated for percentage of dead and CFSE+ cells (% cytotoxicity) and plotted with a log transformed x-axis with a 4-parameter non-linear regression curve fit.

The biparatopic anti-GPRC5D antibodies are expected to elicit ADCC cytotoxicity against all GPRC5D-positive cell lines but not against GPRC5D-negative H929 cells. The isotype control antibody is expected to show no ADCC activity.

Fourth, the biparatopic anti-GPRC5D antibodies are tested for ADCP activities against four different cell lines. The four cell lines are: GPRC5D-positive H929 target cells, BCMA-negative H929 cells (H929 BCMA KO), GPRC5D-positive JIM3 target cells, as well as GPRC5D-negative H929 cells (H929 GPRC5D KO). The ADCP activities of an isotype control antibody against the four different cell lines are also tested. Specifically, monocytes from two healthy donors are differentiated into macrophages by adding macrophage colony-stimulating factor, followed by either interferon gamma (M1) or interleukin-4 (M2) for 24 hours. The target cells are then incubated with the tested antibodies for 2 hours in the presence of primary human macrophages. The ratio of effector to target cell per well is 3:1. The treated target cells are stained with a live/dead-dye and an anti-CD11b antibody. ADCP activities are calculated as a percentage from total live events that are dual positive for CD11b and CFSE by flow cytometry.

The biparatopic anti-GPRC5D antibodies are expected to elicit ADCP cytotoxicity against all GPRC5D-positive cell lines but not against GPRC5D-negative H929 cells. The isotype control antibody is expected to show no ADCP activity.

### Example 2.2 Anti-CD22 Antibodies

A pair of non-competing CD22 binders are formatted as biparatopic antibodies using IgG1 constant domains with or without the RE mutations. This pair of antibodies (with and without RE mutations) are then tested for CDC activities against the CD22-expressing Daudi cell line in the presence of 40% normal human serum. After 12 hours of incubation, cell viability is measured by Cell TiterGlo (Promega) and percent lysis is then calculated.

The same antibodies are then tested for ADCC activities against the CD22-expressing Daudi target cells in the presence of purified NK cells from a normal donor. The Daudi target cells are loaded with BATDA, a cell-labeling reagent that is released upon cell death and can be detected in the cell culture supernatant after forming a fluorescent chelate. After 12 hours of incubation, cell viability is measured by the amount of BATDA released and percent lysis is then calculated.

### Example 2.3 Anti-HLA-G Antibodies

A pair of non-competing HLA-G binders were formatted as biparatopic antibodies using IgG1 constant domains with or without the RE mutations.

First of all, the biparatopic anti-HLA-G antibodies are tested for their binding profiles on HLA-G-positive and HLA-G-negative target cells. The binding profiles of an isotype control antibody are also tested. Specifically, cells are incubated with varying concentrations of the tested antibodies followed by detection of binding with Alexa Fluor 647-conjugated anti-human Fc reagent. The samples are processed on an Intellicyt iQue 3 flow cytometer to measure fluorescent intensity and then plotted in Graph Pad Prism.

The biparatopic anti-HLA-G antibodies are expected to bind to HLA-G-positive target cells. The biparatopic anti-HLA-G antibodies are expected to not bind to HLA-G-negative target cells. The isotype control antibody is expected to not bind to any of the target cells.

Second, the biparatopic anti-HLA-G antibodies are tested for CDC activities against HLA-G-positive and HLA-G-negative target cells. The CDC activities of an isotype control antibody against the same target cells are also tested. Specifically, the target cells are incubated with the tested antibodies for 24 hours in the presence of 40% normal human serum. Equal volumetric amounts of Cell TiterGlo (Promega) is then added to the treated target cells. Percent viability is measured from the luminescent signal and calculated by dividing the highest antibody concentration (100 nM) by vehicle control and multiplying by 100. The values are plotted with a log transformed x-axis with a 4-parameter non-linear regression curve fit.

The biparatopic anti-HLA-G antibodies are expected to have potent CDC activities in all HLA-G-positive target cells but not in HLA-G-negative target cells. The isotype control antibody is expected to show no CDC activity.

Third, the biparatopic anti-HLA-G antibodies are tested for ADCC activities against HLA-G-positive and HLA-G-negative target cells. The ADCC activities of an isotype control antibody against the same target cells are also tested. Specifically, the target cells are incubated with the tested antibodies for 48 hours in the presence of primary human NK cells. Primary human NK cells from two healthy donors are tested, and the ratio of effector to target cell per well is 5:1. The treated target cells are stained with a live/dead-dye and evaluated for percentage of dead and CFSE+ cells (% cytotoxicity) and plotted with a log transformed x-axis with a 4-parameter non-linear regression curve fit.

The biparatopic anti-HLA-G antibodies are expected to elicit ADCC cytotoxicity against all HLA-G-positive target cells but not against HLA-G-negative target cells. The isotype control antibody is expected to show no ADCC activity.

Fourth, the biparatopic anti-HLA-G antibodies are tested for ADCP activities against HLA-G-positive and HLA-G-negative target cells. The ADCP activities of an isotype control antibody against the same target cells are also tested. Specifically, monocytes from two healthy donors are differentiated into macrophages by adding macrophage colony-stimulating factor, followed by either interferon gamma (M1) or interleukin-4 (M2) for 24 hours. The target cells are then incubated with the tested antibodies for 2 hours in the presence of primary human macrophages. The ratio of effector to target cell per well is 3:1. The treated target cells are stained with a live/dead-dye and an anti-CD11b antibody. ADCP activities are calculated as a percentage from total live events that are dual positive for CD11b and CFSE by flow cytometry.

The biparatopic anti-HLA-G antibodies are expected to elicit ADCP cytotoxicity against all HLA-G-positive target cells but not against HLA-G-negative target cells. The isotype control antibody is expected to show no ADCP activity.

### Example 2.4 Anti-PSMA Antibodies

A pair of non-competing PSMA binders were formatted as biparatopic antibodies using IgG1 constant domains with or without the RE mutations. This pair of antibodies (with and without RE mutations) were then tested for CDC activities against the PSMA-expressing LNCaP cell line in the presence of 40% normal human serum.

The anti-PSMA antibodies were added to the LNCaP cell at 20,000 cells/well and incubated for 30 minutes at 37° C. Normal human serum was then added to target cells to a final concentration of 40% to provide a source of complement components for CDC. The mixture was incubated for 17 hours at 37° C. 100 μl of CellTiter-Glo reagent (Promega) was added to the mixture followed by 2 minutes shaking on orbital place shaker and 10 minutes incubation at room temperature. Target cell viability was determined by measuring luminescence with a Tecan SPARK Reader and reported in Relative Luminescence Units (RLU).

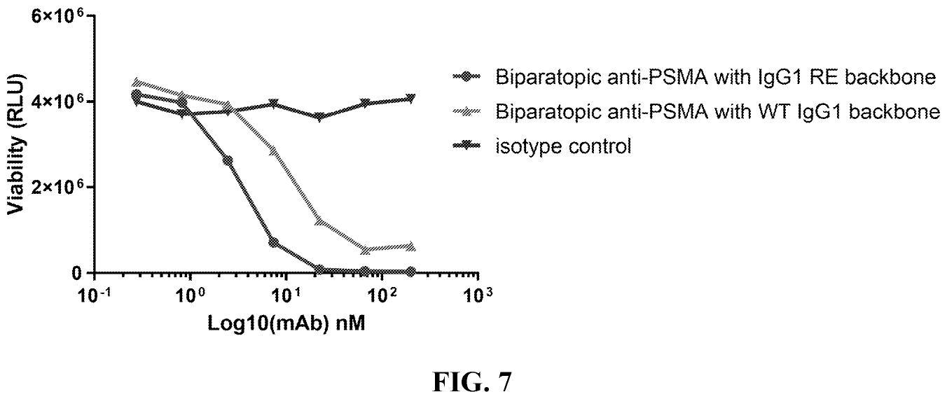

The biparatopic antibody with the RE mutations incorporated into the IgG1 constant domain show more potent CDC activities than the same antibody with the wild-type IgG1 constant domain (FIG. 7).

The same pair of anti-PSMA antibodies were then tested for ADCC activities against the LNCaP cell line using purified NK from a normal donor as effectors.

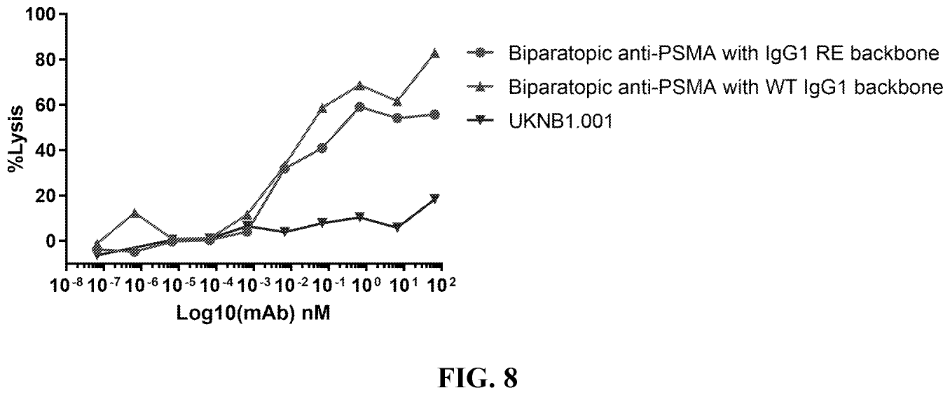

The biparatopic antibody with the RE mutations incorporated into the IgG1 constant domain show similar levels of ADCC activities as the same antibody with the wild-type IgG1 constant domain (FIG. 8).

These results indicate that the biparatopic antibody design can increase the overall efficacy (increased maximum kill) of a given binder and that this augmentation is compatible with the RE mutations.

### Example 2.5 Anti-BCMA Antibodies

A pair of non-competing BCMA binders are formatted as biparatopic antibodies using IgG1 constant domains with or without the RE mutations. This pair of antibodies (with and without RE mutations) are then tested for CDC activities against the BCMA-expressing LNCaP cell line in the presence of 40% normal human serum. After 12 hours of incubation, cell viability is measured by Cell TiterGlo (Promega) and percent lysis is then calculated.

The same antibodies are then tested for ADCC activities against the BCMA-expressing LNCaP target cells in the presence of purified NK cells from a normal donor. The LNCaP target cells are loaded with BATDA, a cell-labeling reagent that is released upon cell death and can be detected in the cell culture supernatant after forming a fluorescent chelate. After 12 hours of incubation, cell viability is measured by the amount of BATDA released and percent lysis is then calculated.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A binding molecule comprising an antigen binding domain and an Fc region, wherein
(i) the antigen binding domain is biparatopic or bi-specific; and
(ii) the Fc region comprises K248E and T437R mutations (RE mutations), wherein amino acid residue numbering is according to the EU numbering system; wherein the binding molecule has increased capability of hexamerization on a cell surface, and/or increased capability of engaging C1q, and
wherein the antigen binding domain comprises a VH region and a VL region contained in a heavy chain and a light chain respectively selected from:
the heavy chain of SEQ ID NO: 257 and the light chain of SEQ ID NO: 259;
the heavy chain of SEQ ID NO: 258 and the light chain of SEQ ID NO: 260;
the heavy chain of SEQ ID NO: 291 and the light chain of SEQ ID NO: 294; or
the heavy chain of SEQ ID NO: 327 and the light chain of SEQ ID NO: 332.

2. The binding molecule of claim 1, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

3. The binding molecule of claim 2, wherein
(a) each of the first domain and the second domain is a Fab fragment, a scFv, a single domain antigen binding fragment, or an engineered binding fragment;
(b) the first domain is a Fab fragment and the second domain is a scFv;
(c) the first domain is a Fab fragment and the second domain is a single domain antigen binding fragment;
(d) the first domain is a Fab fragment and the second domain is an engineered binding fragment;
(e) the first domain is a scFv and the second domain is a single domain antigen binding fragment;
(f) the first domain is a scFv and the second domain is an engineered binding fragment; or
(g) the first domain is a single domain antigen binding fragment and the second domain is an engineered binding fragment.

4. The binding molecule of claim 1, wherein the oligosaccharide covalently attached to the Fc region via the N297 residue thereof does not comprise a core fucose residue.

5. The binding molecule of claim 1, wherein the binding molecule is a biparatopic or bispecific antibody.

6. The binding molecule of claim 1, wherein the binding molecule has enhanced antibody-dependent cellular cytotoxicity (ADCC), enhanced complement-dependent cytotoxicity (CDC), and/or enhanced antibody-dependent cellular phagocytosis (ADCP).

7.-12. (canceled)

13. A population of binding molecules comprising the binding molecule of claim 1.

14. A population of binding molecules, wherein each binding molecule comprises an antigen binding domain and an Fc region; wherein
the antigen binding domain is biparatopic or bispecific;
the Fc region comprises K248E and T437R mutations (RE mutations), wherein amino acid residue numbering is according to the EU numbering system; and
less than 80% of the oligosaccharides covalently attached to the population of binding molecules via the N297 residue of the Fc region comprise a core fucose residue,
wherein the antigen binding domain comprises a VH region and a VL region contained in a heavy chain and a light chain respectively selected from:
the heavy chain of SEQ ID NO: 257 and the light chain of SEQ ID NO: 259;
the heavy chain of SEQ ID NO: 258 and the light chain of SEQ ID NO: 260;
the heavy chain of SEQ ID NO: 291 and the light chain of SEQ ID NO: 294; or
the heavy chain of SEQ ID NO: 327 and the light chain of SEQ ID NO: 332.

15. A population of binding molecules, wherein
(i) each binding molecule comprises an antigen binding domain and an Fc region;
(ii) the antigen binding domain of at least 10% of the binding molecules in the population is biparatopic or bispecific;
(iii) the Fc region of each binding molecule comprises K248E and T437R mutations (RE mutations), wherein amino acid residue numbering is according to the EU numbering system; and
(iv) less than 80% of the oligosaccharides covalently attached to the population of binding molecules via the N297 residue of the Fc region comprise a core fucose residue,
wherein the antigen binding domain comprises a VH region and a VL region contained in a heavy chain and a light chain respectively selected from:
the heavy chain of SEQ ID NO: 257 and the light chain of SEQ ID NO: 259;
the heavy chain of SEQ ID NO: 258 and the light chain of SEQ ID NO: 260;
the heavy chain of SEQ ID NO: 291 and the light chain of SEQ ID NO: 294; or
the heavy chain of SEQ ID NO: 327 and the light chain of SEQ ID NO: 332.

16. The population of binding molecules of claim 15, wherein
the antigen binding domain of at least 20% of the binding molecules in the population is biparatopic or bispecific;
the antigen binding domain of at least 30% of the binding molecules in the population is biparatopic or bispecific;
the antigen binding domain of at least 40% of the binding molecules in the population is biparatopic or bispecific;
the antigen binding domain of at least 50% of the binding molecules in the population is biparatopic or bispecific;
the antigen binding domain of at least 60% of the binding molecules in the population is biparatopic or bispecific;
the antigen binding domain of at least 70% of the binding molecules in the population is biparatopic or bispecific;
the antigen binding domain of at least 80% of the binding molecules in the population is biparatopic or bispecific; or
the antigen binding domain of at least 90% of the binding molecules in the population is biparatopic or bispecific.

17. The population of binding molecules of claim 14, wherein
less than 70% of the oligosaccharides covalently attached to the population of binding molecules via the N297 residue of the Fc region comprise a core fucose residue;
less than 60% of the oligosaccharides covalently attached to the population of binding molecules via the N297 residue of the Fc region comprise a core fucose residue;
less than 50% of the oligosaccharides covalently attached to the population of binding molecules via the N297 residue of the Fc region comprise a core fucose residue;
less than 40% of the oligosaccharides covalently attached to the population of binding molecules via the N297 residue of the Fc region comprise a core fucose residue;
less than 30% of the oligosaccharides covalently attached to the population of binding molecules via the N297 residue of the Fc region comprise a core fucose residue;
less than 20% of the oligosaccharides covalently attached to the population of binding molecules via the N297 residue of the Fc region comprise a core fucose residue; or
less than 10% of the oligosaccharides covalently attached to the population of binding molecules via the N297 residue of the Fc region comprise a core fucose residue.

18. The population of binding molecules of claim 14, wherein the antigen binding domain comprises a first domain comprising a first VH region and a first VL region, a first single domain antigen binding fragment, and/or a first engineered binding fragment; and a second domain comprising a second VH region and a second VL region, a second single domain antigen binding fragment, and/or a second engineered binding fragment; and wherein the first domain and the second domain bind to two different epitopes of the same antigen or different antigen(s).

19. The population of binding molecules of claim 14, wherein
(a) each of the first domain and the second domain is a Fab fragment, a scFv, a single domain antigen binding fragment, or an engineered binding fragment;
(b) the first domain is a Fab fragment and the second domain is a scFv;
(c) the first domain is a Fab fragment and the second domain is a single domain antigen binding fragment;
(d) the first domain is a Fab fragment and the second domain is an engineered binding fragment;
(e) the first domain is a scFv and the second domain is a single domain antigen binding fragment;
(f) the first domain is a scFv and the second domain is an engineered binding fragment; or
(g) the first domain is a single domain antigen binding fragment and the second domain is an engineered binding fragment.

20. The population of binding molecules of claim 14, wherein the binding molecules are biparatopic or bispecific antibodies.

21. The population of binding molecules of claim 14, wherein the binding molecules are produced by expressing a polynucleotide encoding the binding molecules or a fragment thereof in a host cell that is deficient in adding a fucose to the oligosaccharide attached to the Fc region of an antibody.

22. The population of binding molecules of claim 21, wherein the host cell has reduced GDP-mannose 4,6-dehydratase (GMD) activity or reduced α-1,6 fucosyltransferase activity.

23. The population of binding molecules of claim 13, wherein the population of binding molecules has enhanced ADCC, enhanced CDC, and/or enhanced ADCP.

24. A nucleic acid encoding the binding molecule of claim 1.

25. A vector comprising the nucleic acid of claim 24.

26. A method of making the binding molecule of claim 1, comprising:
expressing a polynucleotide encoding one or more of the binding molecule or a fragment thereof in a host cell that is deficient in adding a fucose residue to an oligosaccharide attached to an antibody via the N297 residue.

27-33. (canceled)
